(19) United States
(12) Patent Application Publication
(10) Pub. No.: US 2026/0258429 A1
(43) Pub. Date: Sep. 3, 2026

(54) PLUS SENSE SINGLE STRANDED RNA VECTORS WITH HAIRPIN-LIKE INSERTS

(71) Applicants: SILVEC BIOLOGICS INC., Gaithersburg, MD (US); UNIVERSITY OF MARYLAND, COLLEGE PARK, College Park, MD (US)

(72) Inventors: Anne Elizabeth SIMON, Upper Marlboro, MD (US); Jason Michael NEEDHAM, Greenbelt, MD (US); Chanyong JANG, Rockville, MD (US); Osama Osman Abdel Aziz ATALLAH, Greenbelt, MD (US); Feng GAO, Gaithersburg, MD (US)

(21) Appl. No.: 19/552,563

(22) Filed: Feb. 27, 2026

Related U.S. Application Data
(60) Provisional application No. 63/764,707, filed on Feb. 28, 2025.
(60) Provisional application No. 63/890,555, filed on Sep. 30, 2025.

(51) Int. Cl. C12N 15/82; C12N 15/113

(52) U.S. Cl. CPC C12N 15/8203; C12N 15/113; C12N 2310/14; C12N 2310/531

(57) ABSTRACT

The present disclosure relates to a viral vector having an exogenous RNA segment with a hairpin-like structure, for example having two or more base-paired regions separated by one or more non-base-paired regions. The exogenous RNA segment may have a secondary structure, minimum free energy, average positional entropy or other attributes within specified ranges, or with values similar to one or more hairpin-like structures of a reference wild type virus.

### CROSS-REFERENCE TO RELATED APPLICATIONS

This application claims priority from and the benefit to U.S. Provisional Patent Application Ser. No. 63/764,707, filed Feb. 28, 2025, and U.S. Provisional Patent Application Ser. No. 63/890,555, filed Sep. 30, 2025, both of which are incorporated herein by reference.

### STATEMENT REGARDING FEDERALLY SPONSORED RESEARCH OR DEVELOPMENT

This invention was made with government support under National Science Foundation (NSF) number 312990-00001; USDA ECDRE 308291-00001; and, USDA ECDRE 2020-08455. The United States government has certain rights in this invention.

### REFERENCE TO SEQUENCE LISTING

The contents of the electronic sequence listing named “P93245505US09_96683581_SequenceListing.xml” was created on Feb. 20, 2026, is 145,898 bytes in size, and is herein incorporated by reference in its entirety. All sequences in the sequence listing and in the specification are RNA sequences. Thus, the nucleotide symbol “t” should be construed as “uracil” pursuant to WIPO ST.26 paragraph 14.

### FIELD OF THE INVENTION

The present disclosure relates to the field of RNA virus based vectors having an exogenous RNA segment. The vector may be suitable for introducing a therapeutic agent, such as a peptide, or a small RNA, into a host.

### BACKGROUND OF THE INVENTION

The following description is not an admission that anything described below or in the referenced publications is common general knowledge or citable as prior art in any country.

Both general and highly targeted anti-microbial agents have been developed for animals (e.g., humans) whose circulatory systems provide a delivery system for widespread application throughout the animal. In contrast, much less research has been conducted to develop general or targeted therapeutic agents for plants. Among other factors, the lack of an animal-like circulatory system complicates delivery throughout the host plant. This is especially problematic in large, long-lived trees (e.g., citrus), where injection of anti-microbial agents may be rapidly diluted. As a result, few solutions exist for treating plant infections or conditions beyond external application of pesticides to control the pathogen's vector, applications to strengthen a plant's health in general, or application of short-duration agents targeting the pathogen, or a vector of the pathogen.

Plant industries are at substantial risk from various pathogens. In the citrus industry, Huanglongbing (HLB), also known as Citrus Greening, is the most serious disease globally. HLB is associated with three species of the bacterium Candidatus Liberibacter spp. (asiaticus, africanus, and americanus) and is transmitted by two psyllid species, Asian citrus psyllid (ACP) (Diaphorina citri, Kuwayama) and African citrus psyllid (Trioza erytreae, Del Guercio). HLB is graft-transmissible and spreads naturally when a bacteria-containing psyllid feeds on a citrus tree and deposits the pathogenic bacteria into the phloem where the bacteria reproduce. The infected tree reacts by producing excessive callose and phloem protein 2 (PP2) in its phloem in order to isolate the bacteria, which restricts the flow of photoassimilates and can ultimately kill the tree. While the diseased fruit pose no health threat to humans, HLB has devastated millions of acres of citrus groves throughout the world. In the United States alone, ACP and CL asiaticus (CLas) have decimated the Florida citrus industry, causing billions of dollars of crop losses within a very short time span. Moreover, HLB has spread into every citrus producing region in the United States. Most infected trees die within a few years after infection, and fruit develops misshapen and off flavored years before tree death and is unsuitable for consumption. According to the United States Department of Agriculture (USDA), the entire citrus industry is at substantial risk.

Consideration of plant physiology aids in the development and implementation of strategies for managing plant diseases and conditions. The vascular system of plants is the key conduit for sugars and amino acids, as well as signaling molecules such as small ribonucleic acids (RNAs), messenger RNAs (mRNAs), proteins, peptides and hormones, which are required for a large number of developmental processes and responses to biotic and abiotic stress (Lee, J. Y. and Frank, M. (2018), Plasmodesmata in phloem: different gateways for different cargoes, Curr Opin Plant Biol 43:119-124; Tugeon, R. and Wolf, S. (2009), Phloem Transport: Cellular Pathways and Molecular Trafficking, Ann Rev Plant Biol 60:207-221). Many mRNAs comprise a portion of these transiting molecules, and thousands of companion cell mRNAs can be isolated from neighboring enucleated sieve elements, where they are transported bidirectionally by osmotically generated hydrostatic pressure from source (sugar generating) tissue to sink (sugar utilizing) tissue such as roots and shoot tips (Folimonova, S. Y. and Tilsner, J. (2018), Hitchhikers, highway tools and roadworks: the interactions of plant viruses with the phloem, Curr Opin Plant Biol 43:82-88; Ham, B. K. and Lucas, W. J. (2017), Phloem-Mobile RNAs as Systemic Signaling Agents, Annual Rev Plant Biol 68:173-195).

Plant viruses, many of which move through the plant as a ribonucleoprotein complex (vRNP), have evolved to use the same pathway as used by mobile endogenous RNAs. Plant viruses can accumulate in substantial amounts, and most initiate infection in epidermal or mesophyll cells and then move cell-to-cell through highly selective intercellular connectors called plasmodesmata, which allow for continuity between the cytoplasm of neighboring cells (FIG. 1; see also Lee, J. Y. and Frank, M. (2018), Plasmodesmata in phloem: different gateways for different cargoes, Curr Opin Plant Biol 43:119-124; Schoelz, J. E. et al. (2011), Intracellular transport of plant viruses: finding the door out of the cell, Mol Plant 4:813-831). Long-distance systemic movement (leaf-to-leaf) requires that the virus, in the form of RNA-protein complexes or RNA-encapsidated inside virions, enters companion cells, where replication takes place, followed by progeny exit into sieve elements by transiting through the specialized, branched plasmodesmata that connect companion cells and sieve elements. Once tubular sieve elements are reached, viruses move passively with the phloem photoassimilate stream and establish systemic infections upon exiting (Folimonova, S. Y. and Tilsner, J. (2018), Hitchhikers, highway tolls and roadworks: the interactions of plant viruses with the phloem, Curr Opin Plant Biol 43:82-88).

Delivering engineered therapeutic agents into plants is an established means of introducing traits such as resistance to pathogens or other desired properties into plants for research purposes. Various methods of providing vectors to plants are known in the art. This is often achieved by delivery of an RNA virus vector (virus engineered to contain exogenous RNA) into a plant cell's nucleus by Agrobacteria tumefactions-mediated “agroinfiltration,” which may result in a modification of that cell's genome, or by delivering the virus vector directly into a cell's cytoplasm, which results in infection without a requirement for plant genomic modification. In the case of agroinfiltration of RNA viruses, the cDNA of the viral genome is incorporated into the T-DNA, which Agrobacteria delivers into the plants. Such T-DNA includes further regulatory DNA components (e.g., promoter for cellular DNA-dependent RNA polymerase), which allow for transcription of the DNA to generate the full-length viral RNA genome within plant cells. The incorporated DNA version of the RNA virus that contains therapeutic DNA inserts is transcribed into the viral RNA within the plant cells, after which the virus behaves like a normal RNA virus (amplification and movement).

To act as an effective vector, a virus should be engineered to accept inserts without disabling its functionality and to ensure that the engineered virus is able to accumulate systemically in the host to a level sufficient to deliver and in some cases express peptides or proteins from the insert(s). These inserts, whether composed of open reading frames (ORFs) that will be translated into proteins, or non-coding RNA that will be used for a beneficial function, should be delivered into the targeted tissue in a manner that is effective and sufficiently non-toxic to the host or to any downstream consumption of the host, or to the environment. In addition, the virus vector must be non-toxic to the host plant and is preferably not capable of being transmitted from plant to plant. However, only a limited number of viral vectors exist that meet at least some of the above criteria and some are available for only certain plants (e.g., citrus tristeza virus for citrus) since most plant viruses have a very limited range of hosts. Moreover, maintaining stability of short sequences inserted into a vector, particularly for long periods (e.g., months or years), has raised numerous challenges. Conventional virus vectors quickly (i.e., within a few days or a few weeks) evolve or mutate, discarding all or part of the inserted sequences given that the native virus without inserts is generally more fit than the virus vector with the inserts.

CTV is a member of the genus Closterovirus. It has a flexuous rod-shaped virion composed of two capsid proteins with dimensions of 2000 nm long and 12 nm in diameter. With a genome of over 19 kb, CTV (and other Closteroviruses) are among the largest known RNA viruses that infect plants. Prior studies have demonstrated that CTV-based vectors can express engineered inserts in plant cells (U.S. Pat. No. 8,389,804; US 20100017911 A1).

International Publication Number WO 2020/102210, Plant Vectors, Compositions and Uses Relating Thereto, published on May 22, 2020, describes an RNA vector derived from citrus yellow vein associated virus (CYVaV) or one of its relatives.

International Publication Number WO 2021/097086, Plant Vectors, Compositions and Uses Relating Thereto, published on May 20, 2021, further describes an RNA vector derived from citrus yellow vein associated virus (CYVaV) or one of its relatives.

International Publication Number WO 2022/246005, Plant Vectors, Compositions and Uses Relating Thereto, published on Nov. 24, 2022, describes an RNA vector having an siRNA insert effective against a plant bacterial pathogen. The vector may be derived from CYVaV, a relative of CYVaV, or another plant virus such as tobacco rattle virus (TRV).

Relatives of CYVaV include other umbravirus-like associated RNAs (ulaRNA) as described in Structural Analysis and Whole Genome Mapping of a New Type of Plant Virus Subviral RNA: Umbravirus-Like Associated RNAs, Liu J. et al., Viruses 2021,13, 646, which is incorporated herein by reference. The ulaRNAs identified in this paper are referred to as Group 2 umbravirus-like viruses (ULVs) in this specification.

Some relatives of CYVaV are also described in −1 Programmed ribosomal frameshifting in Class 2 umbravirus-like RNAs use multiple long-distance interactions to shift between active and inactive structures and destabilize the frameshift stimulating element, Mikkelsen A. et al., Nucleic Acids Res. 2023 Oct. 27; 51(19):10700-10718.

### SUMMARY OF THE INVENTION

This specification describes a synthetic plus-sense single stranded RNA segment and a vector derived from a plus-sense single stranded RNA virus comprising the synthetic segment. This specification also describes methods of modifying an RNA molecule, for example a viral vector, by insertion of one or more heterologous segments. This specification also describes a method of designing a sequence or hairpin-like structure for insertion into a plus-sense single stranded RNA vector.

In some embodiments, one or more heterologous segments comprises a) two or more base-paired regions, b) one or more non-base-paired regions separating the base-paired regions, and c) an apical loop at the end of one of the based paired regions. In some embodiments, the one or more heterologous segment(s) are designed according to one or more parameters described herein, for example a minimum free energy and/or average positional entropy within defined limits. In some embodiments, the heterologous segment has a minimum free energy in kcal/mol in a range of about −5 to +15, or in a range of about −10 to +10, from −0.44 multiplied by the number of bases of the heterologous RNA segment minus 1.89 or, if the heterologous RNA segment has more than 150 nucleotides, a minimum free energy in kcal/mol in a range of about −20 to +20 from −0.44 multiplied by the number of bases of the heterologous RNA segment minus 1.89.

In some embodiments, the heterologous segment has an average positional entropy (APE) less than 0.75, 0.65, 0.5, 0.4, 0.39, 0.36 or 0.32. In some embodiments, the APE is greater than 0.01, 0.02 0.03, 0.04, 0.05, 0.06 or 0.07. In some embodiments, the APE is from 0.01 to 0.75, 0.01 to 0.65, 0.01 to 0.5, 0.01 to 0.4, 0.01 to 0.39, 0.01 to 0.36 or 0.01 to 0.32. In some embodiments, the APE is from 0.02 to 0.75, 0.02 to 0.65, 0.02 to 0.5, 0.02 to 0.4, 0.02 to 0.39, 0.02 to 0.36 or 0.02 to 0.32. In some embodiments, the APE is from 0.03 to 0.75, 0.03 to 0.65, 0.03 to 0.5, 0.03 to 0.4, 0.03 to 0.39, 0.03 to 0.36 or 0.03 to 0.32. In some embodiments, the APE is from 0.04 to 0.75, 0.04 to 0.65, 0.04 to 0.5, 0.04 to 0.4, 0.04 to 0.39, 0.04 to 0.36 or 0.04 to 0.32. In some embodiments, the APE is from 0.05 to 0.75, 0.05 to 0.65, 0.05 to 0.5, 0.05 to 0.4, 0.05 to 0.39, 0.05 to 0.36 or 0.05 to 0.32. In some embodiments, the APE is from 0.06 to 0.75, 0.06 to 0.65, 0.06 to 0.5, 0.06 to 0.4, 0.06 to 0.39, 0.06 to 0.36 or 0.06 to 0.32. In some embodiments, the APE is from 0.07 to 0.75, 0.07 to 0.65, 0.07 to 0.5, 0.07 to 0.4, 0.07 to 0.39, 0.07 to 0.36 or 0.07 to 0.32, or 0.05 to 0.39 or 0.05 to 0.32.

In some embodiments, the base-paired regions of the heterologous segment have 19 or less base pairs or 17 or less base pairs or 13 or less base pairs or 10 or less base pairs.

In some embodiments, the heterologous segment has no more than 4 consecutive A:U base pairs.

In some embodiments, the heterologous segment has no more than 4, or no more than 3 consecutive G:C base pairs.

In some embodiments, the heterologous segment has no more than 21 or no more than 20 bases collectively on both sides in a non-base paired region of the heterologous segment.

In some embodiments, a) each base-paired region of the heterologous segment has an APE of less than 0.8 or less than 0.6 and/or b) the DG of a region including two non-base paired regions and one base-paired region between them is not positive.

In some embodiments, the heterologous segment has between 40 and 300, 40 and 200, 60 and 300 or 60 and 200 bases.

In some embodiments, the heterologous segment has a standard deviation of PE less than 0.5 or less than 0.4.

In some embodiments, less than 20% or 15% or less than 10% of the bases of the heterologous segment have a PE of greater than 1.0

In some embodiments, the largest PE of any base of the heterologous segment is not greater than 2.0 or not greater than 1.5.

In some embodiments, the heterologous segment replaces a hairpin-like region of the RNA virus.

In some embodiments, the heterologous segment has a minimum free energy within 10 kcal/mol, or within 5 kcal/mol of a naturally occurring hairpin-like structure of similar size, i.e. of a size within 10% of the size of the heterologous segment, or of a line of best fit between multiple wild type hairpin-like structures. Optionally, for long heterologous RNA segments, for example segments with 150 nucleotides or more, the heterologous segment has a minimum free energy within 20 kcal/mol of a naturally occurring hairpin-like structure of similar size, i.e. of a size within 10% of the size of the heterologous segment, or of a line of best fit between multiple hairpin-like structures.

In some embodiments, a heterologous segment has an MFE in kcal/mol in a range of (−0.27x plus 10) to (−0.66x−10), where x is the number of bases in the heterologous segment other than in an apical loop. In some embodiments, the heterologous segment has an MFE in a range of −0.3x to −0.66x, −0.3x to −0.55x or −0.3x to −0.49x). In some embodiments, a target MFE is similar to an MFE/length of at least four naturally occurring hairpin-like regions in the RNA virus.

In some embodiments, the segment derived from the RNA virus has a sequence identity of at least 50%, at least 60%, at least 70%, at least 80%, at least 90% or at least 95% with the RNA virus. In some embodiments, the segment derived from the RNA virus is a viral vector or a live attenuated vaccine known in the art.

In some embodiments, the RNA virus is a Group IV positive-sense single stranded RNA virus according to the Baltimore classification. In some embodiments, the RNA virus in DENV2, Foxtail Mosaic Virus, SARS-CoV-2, EVA71, VEEV, Zika Virus, WNV, PEMV2, TCV, Powassan Virus, YFV, GULV1, TBSV, or HCV.

In some embodiments, the portion derived from an RNA virus is derived from a plant virus such as an umbra-like virus (ULV, alternatively called an umbralike associated RNA or ulaRNA), citrus tristeza virus (CTV), potato virus X (PVX) or tobacco rattle virus (TRV). In some embodiments, the portion derived from an RNA virus is derived from a Class 2 ULV. In some embodiments, the portion derived from an RNA virus is derived from CY1.

In some embodiments, the RNA virus is an animal virus. In some embodiments, the RNA virus is a live attenuated virus of a vaccine. In some embodiments, the RNA virus is an enterovirus, for example poliovirus or CVB3.

In some embodiments, the heterologous segment comprises an active portion (alternatively called a targeting portion). In some embodiments, the active portion comprises an RNA bioactive molecule, such as a sequence that generates one or more siRNAs. In some embodiments, the active portion is effective against a plant pathogen or suppresses expression of a gene in a plant. In some embodiments, the active portion targets a susceptibility gene of a host plant. In some embodiments, the active portion is complementary to a host microRNA. In some embodiments, the active portion encodes a peptide.

In some embodiments, the heterologous segment has an active portion provided on a first side of the heterologous segment. For example the first side may have a coding or non-coding sequence that targets a pathogen or gene of a host. In some embodiments, a sequence on a second side of the heterologous segment is less than 90%, or less than 85%, or less than 70%, but more than 50%, more than 60%, more than 65%, or more than 75%, complementary with the sequence of the first side.

In some embodiments, the RNA molecule, the heterologous segment or the method are not disclosed in U.S. provisional application 63/338,290 or 63/343,168. In some embodiments, the heterologous segment is not a lock and dock and/or does not have a tertiary interaction with itself or with the RNA virus. In some embodiments, the heterologous segment is not attached to a truncated structure of the RNA virus. In some embodiments, the RNA virus is not a Class 2 ulaRNA.

In some embodiments, a design process includes selecting a sequence for a hairpin-like region to provide a selected percentage complementarity, adjusting the sequence if required to produce a selected MFE or MFE/length, and adjusting the sequence if required to produce a selected APE. In some embodiments, these steps are done in the order that they are listed in the preceding sentence.

This specification also describes a ULV, ulaRNA, Class 2 ULV, CY1, CTV, PVX or TRV or other plus sense single stranded RNA vector comprising a heterologous RNA segment targeted against a host plant susceptibility gene, wherein the heterologous RNA segment has one or more characteristics, for example an MFE and/or APE, of the heterologous segments described herein.

This specification also describes a live attenuated virus comprising a heterologous segment wherein the heterologous segment is complementary to a microRNA of the animal and has one or more characteristics, for example an MFE and/or APE, of the heterologous segments described herein.

### BRIEF DESCRIPTION OF THE DRAWINGS

The priority application of the present application contains at least one drawing/photograph executed in color. Copies of such color drawing(s) will be provided by the United States Patent & Trademark Office upon request and payment of the required fee.

FIGS. 1A-1C show that CY1 and CY2 are closely related Group 2/Class 2 ULVs. FIG. 1A. Genome organization of ULVs. Group 2/Class 1 members only encode two replication-required proteins and have extensive 3′ UTRs. Group 2/Class 2 members that infect dicots (except CY1) have an additional ORF (ORF5) that overlaps with the end of the RdRp ORF in the −1 frame. Monocot-infecting Class 2 members have an addition embedded ORF of different lengths. Group 2/Class 3 members have at least one additional ORF that are unrelated to each other and ORF5. FIG. 1B. Maximum likelihood phylogenetic tree based on RdRp nucleotide sequences. Branch numbers indicate bootstrap support in percentage out of 1000 replicates. The scale bar denotes nucleotide substitutions per site. The tree is mid-point rooted. Dicot-infecting and monocot-infecting Class 2 ULVs separate into different clades with the exception of parsley umbra-like virus (PULV), which is closer to an ancestral viral molecule that possibly gave rise to all Class 2 ULVs. FIG. 1C. Left, schematic secondary structure of CY1 and CY2. CY2 shares a similar overall secondary structure and contains two large insertions (shaded). Approximate location of start codons and stop codons are indicated by arrows.

FIG. 2 illustrates schematically a comparison of the CY1 RNA structure with structures for other Class 2 ULVs. Designations of CY1 structures (Pr, H5, H4a and H4b) are highly conserved and denoted for each genome structure. Inserted segments not found in CY1 are shown in dark grey. Open circle, closed circle and star denote ORF1 initiation site, ORF1 termination site and ORF2 termination site, respectively. Open triangle and closed triangle denote start site and termination site for ORF5, respectively.

FIG. 3 illustrates schematically a comparison of the CYVaV (CY1) and CYVaV-Delta (CY2) structures.

FIG. 4 shows modifications to an unstable insert that produced a stable insert

FIGS. 5A-5E illustrate stabilization of siRNA targeting GFP in CY1 genome. FIGS. 5A-5C show the complete hairpin structures targeting GFP mRNA in Nicotiana benthamiana isolate 16C, which expresses GFP. FIG. 5A: 21 nt GFPsh (sh: small hairpin) with the stabilizing loop sequence (ucaagag) from the pSuper vector (Brummelkamp T R, et al, 2002 DOI: 10.1126/science.1068999). The minimum free energy (ΔG) of the structure is −39.30 kcal/mol. The polynucleotide sequence shown in FIG. 5A is tgaagcggcacgact tcttcaatcaagagatgaagaagtcgtgccgcttca (SEQ ID NO:68). FIG. 5B: 26 nt GFPsh with the same loop sequence as in A (ΔG is −51.20 kcal/mole. The polynucleotide sequence shown in FIG. 5B is tgaagcggcacgacttcttcaagagcatcaagagagctctt gaagaagtcgtgccgcttca (SEQ ID NO:69). FIG. 5C: 30 nt GFPsh. The loop sequence is gaauuc and ΔG is −63.80 kcal/mol. Finding PCR bands smaller than expected indicates that these hairpin structures are not stable after systemic infection. Furthermore, the relative band intensity of the small band (vector with insert deleted) and the bigger band (vector with intact insert) in the PCR results suggests that structural stability of the hairpin is reversely correlated with its maintenance in the vector after systemic infection. The polynucleotide sequences shown in FIG. 5C are tgaagcggcacgacttcttca agagcgccaga (SEQ ID NO:70) and tctggcgctcttgaagaagtcgtgccgcttca (SEQ ID NO:71). FIG. 5D: The secondary structure of CY1 established by SHAPE data (left) and the reference structure to be mimicked, CY2220-2280 (in dashed gray box, right). The ΔG of this structure is −31.50 kcal/mol. The polynucleotide sequence shown in FIG. 5D is ggttagggtaactcacataccttcttccataactggaaaaggtcgtgtgagcaacctaacc (SEQ ID NO:72). FIG. 5E: GFPmmck59. Total 59 nt mimicked structure includes 28 nt siRNA of sense orientation (circled in gray). GFPmmck59 (tgaagcggcacgacttctt caagagcgataactcgccttgacagaagtccaacgcttca (SEQ ID NO:73)) was inserted at position 2304 in CY1 (arrow), was stable after systemic infection, and was functional in silencing GFP (gray in picture indicates tissue where GFP is silenced). The RT-PCR result and the sequencing chromatogram proved the exceptional stability of the insert after systemic infection. *The minimum free energy was calculated using RNAfold Webserver: http://rna.tbi.univie.ac.at//cgi-bin/RNAWebSuite/RNAfold.cgi*. The polynucleotide sequence shown in FIG. 5E is:

(SEQ ID NO: 8)
aaaaggtcgtgtgagcaacctaaccagttaatgtaggtgtctttccgta
tgaagcggcacgacttcttcaagagcgataactcgccttgacagaagtc
caacgcttcatctagtcaggatggtaagcaacccgtttatctgtacggc
gctcacccgtgggtaggaag.

FIGS. 6A-6D demonstrate the stability of two exemplary mimic inserts in the CY1 genome. FIG. 6A: Locations of the inserts in one of the two-insert constructs. The natural hairpin at 2219 is deleted (i.e. bases 2220-2280 are deleted) and one of the two mimic inserts (targeting Callose Synthase 7) is inserted at that location (i.e. between wild type 2219 and 2281). The second mimic insert (targeting CTV) is at 2304 (wild type numbering). The sequence of Mmck6.1 is SEQ ID NO:9. The sequence of CS7-V2.2 is SEQ ID NO:10. FIG. 6B: In vitro translation of this construct (gray asterisks) in wheat germ extracts compared with WT CY1. FIG. 6C: Sequencing the population of the two-hairpin construct at 4 weeks post infiltration.

The polynucleotide sequence of F3-C3#3 is:

(SEQ ID NO: 11)
actagaggaagtgttgacgaaatgtaatgttccagttatggaatcatta
atttcgtccatcacttcctacggaaagacacctacattaactggtatca
tttgcagacggaagattggcagttatgccaaatcttcgtctgcccattg
ataccaac.

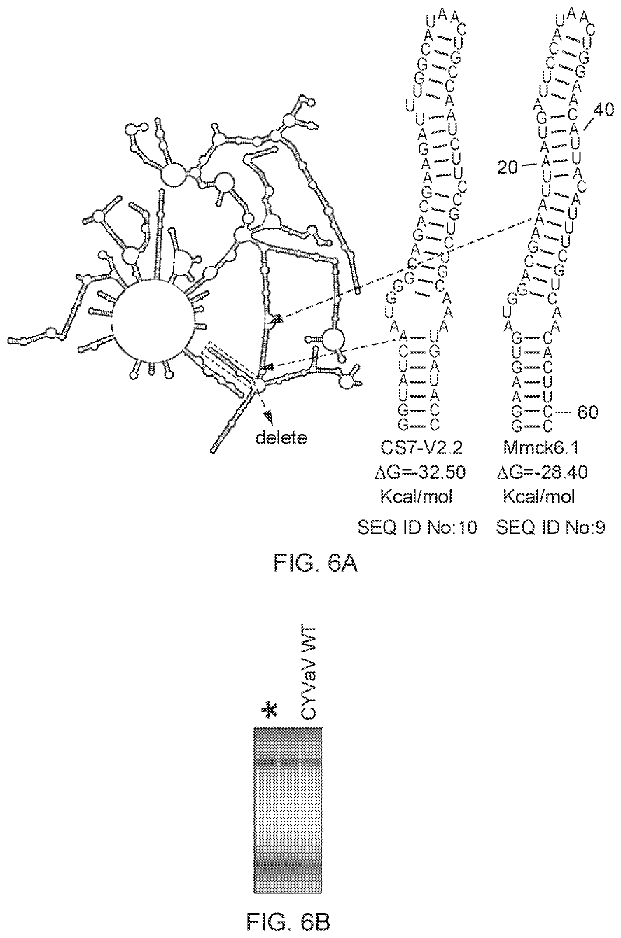

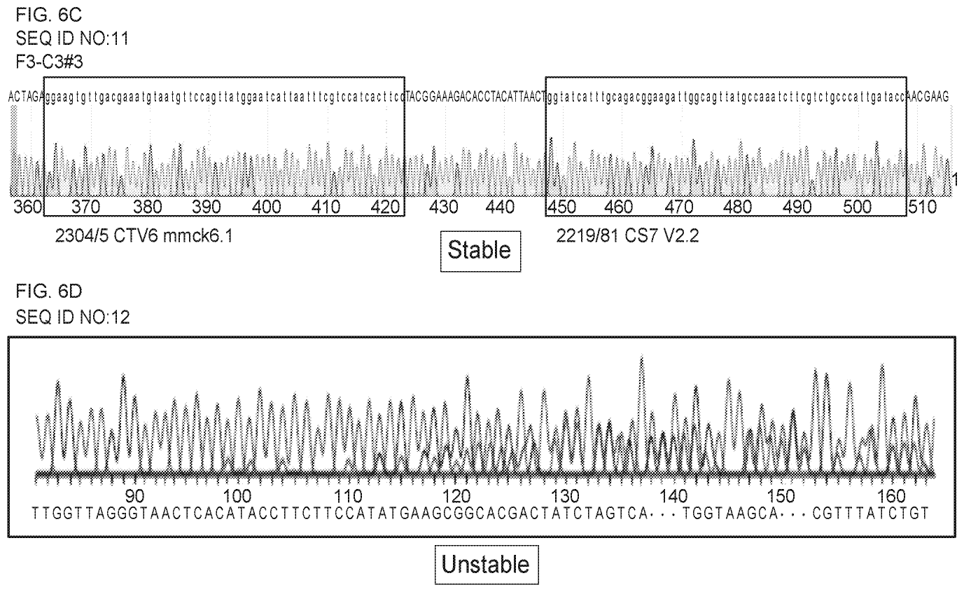

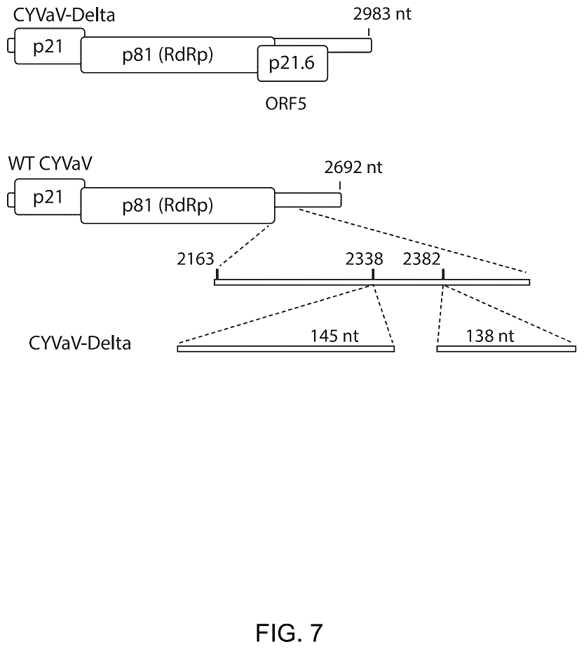

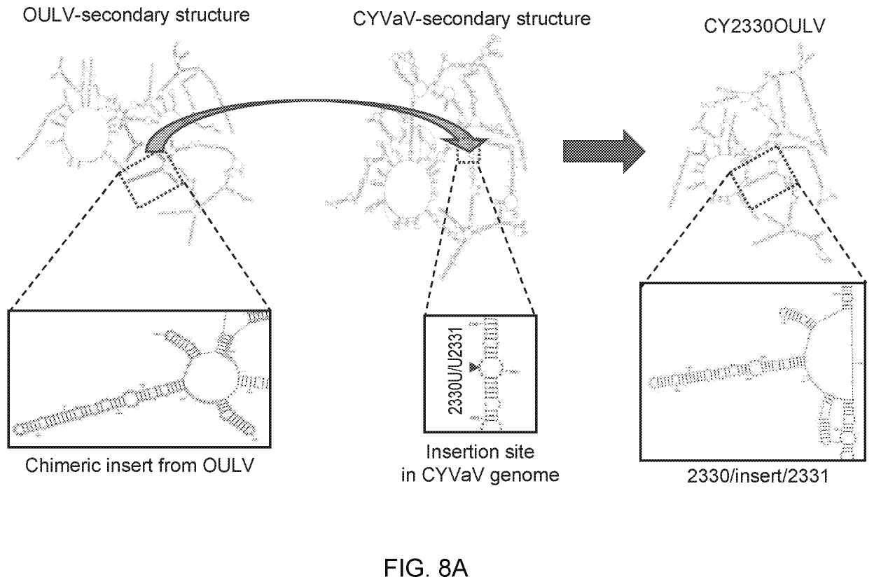

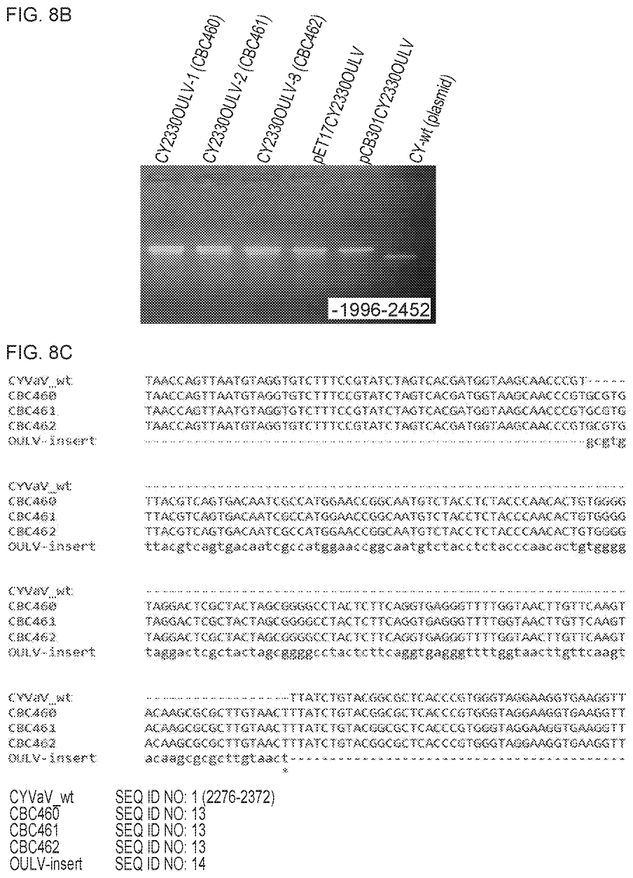

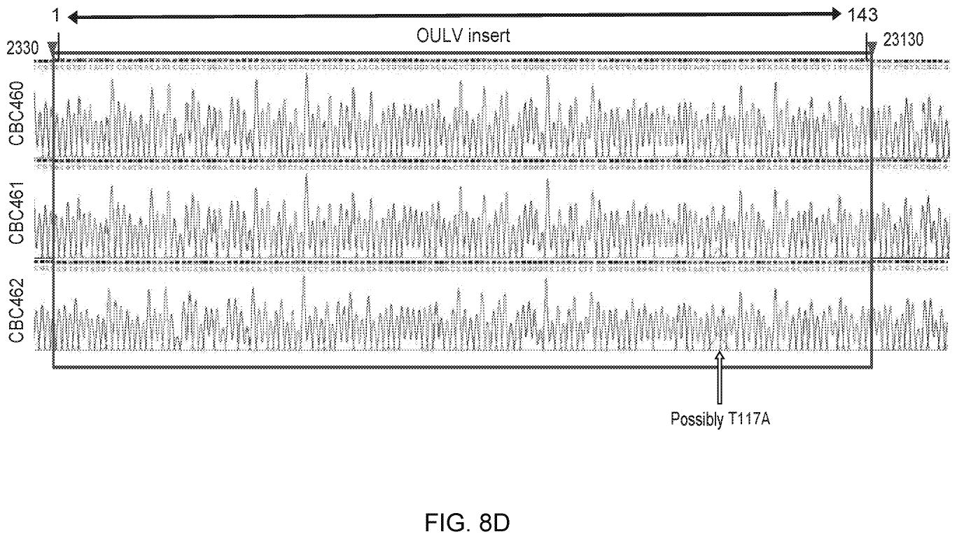

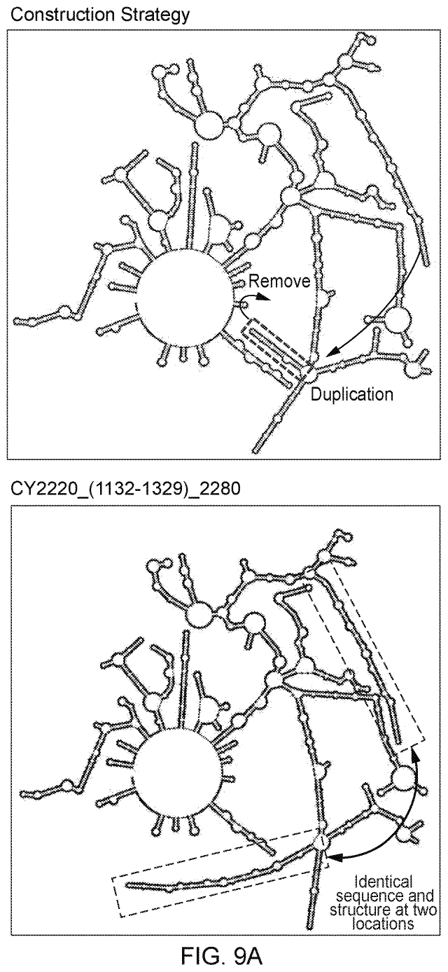

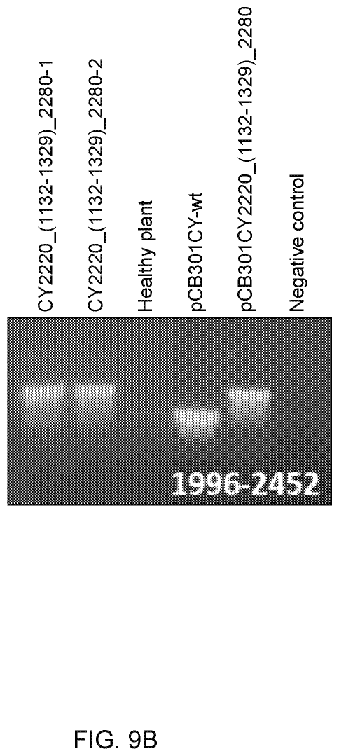

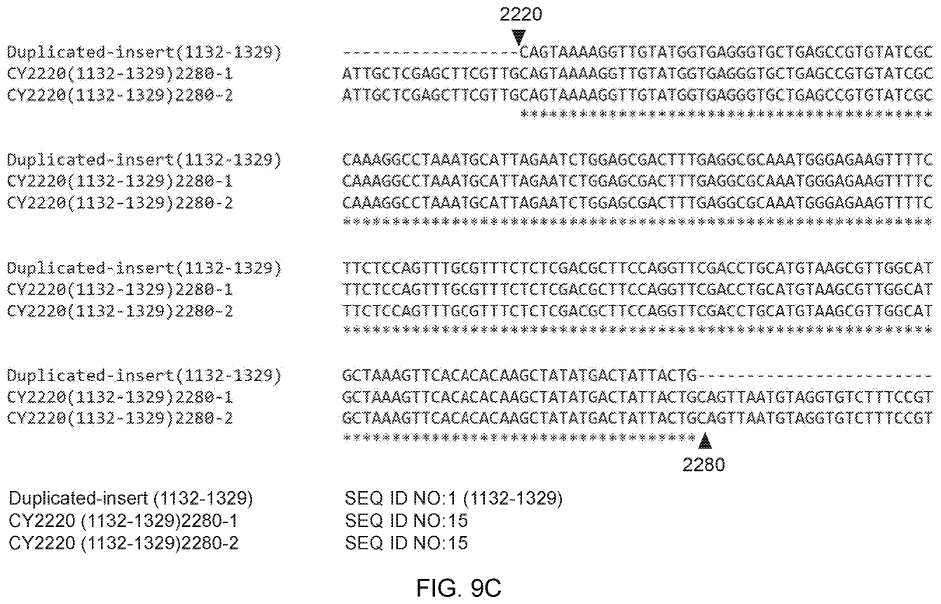

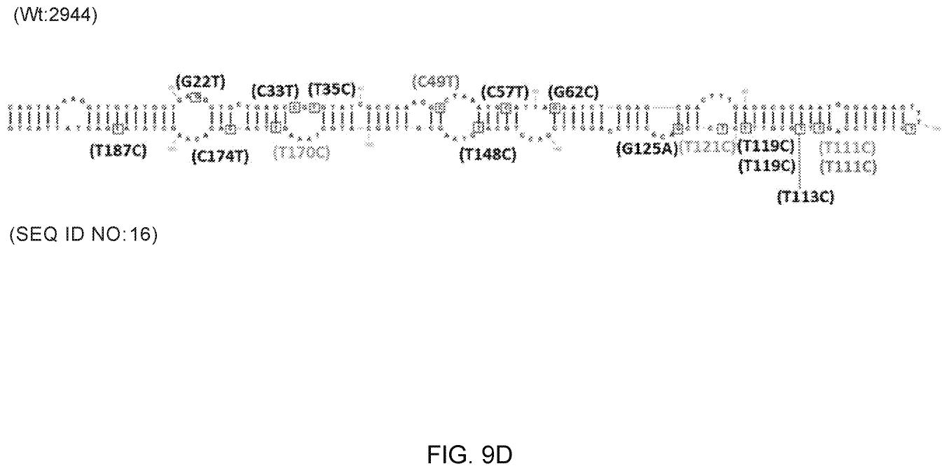

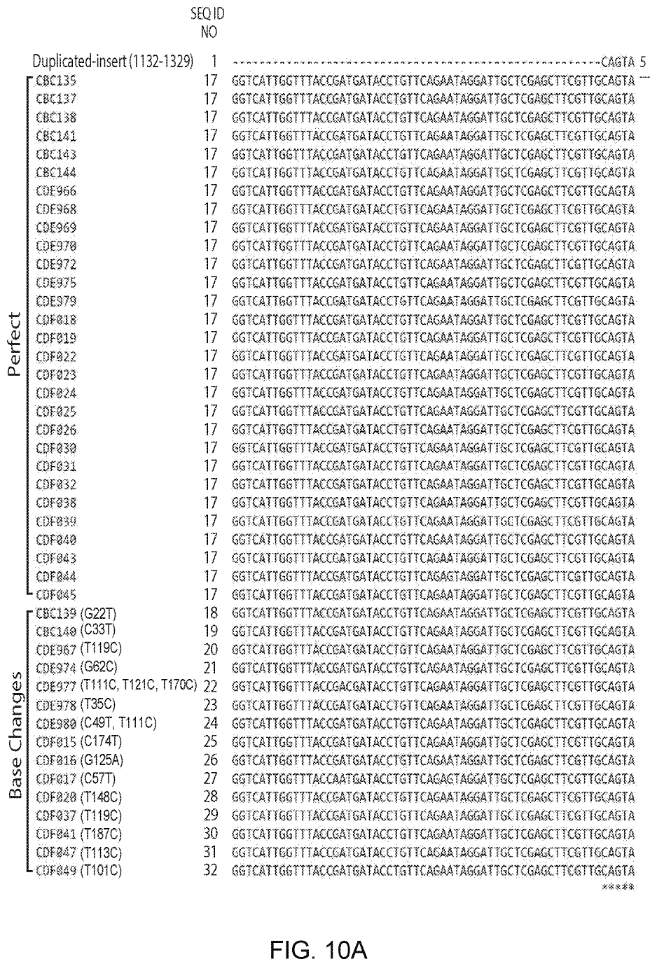

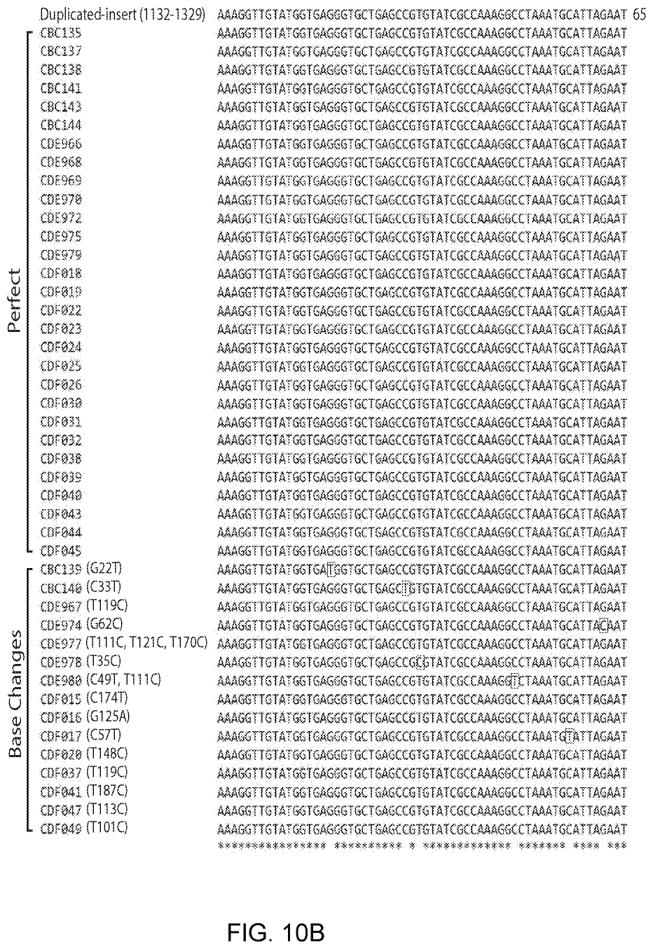

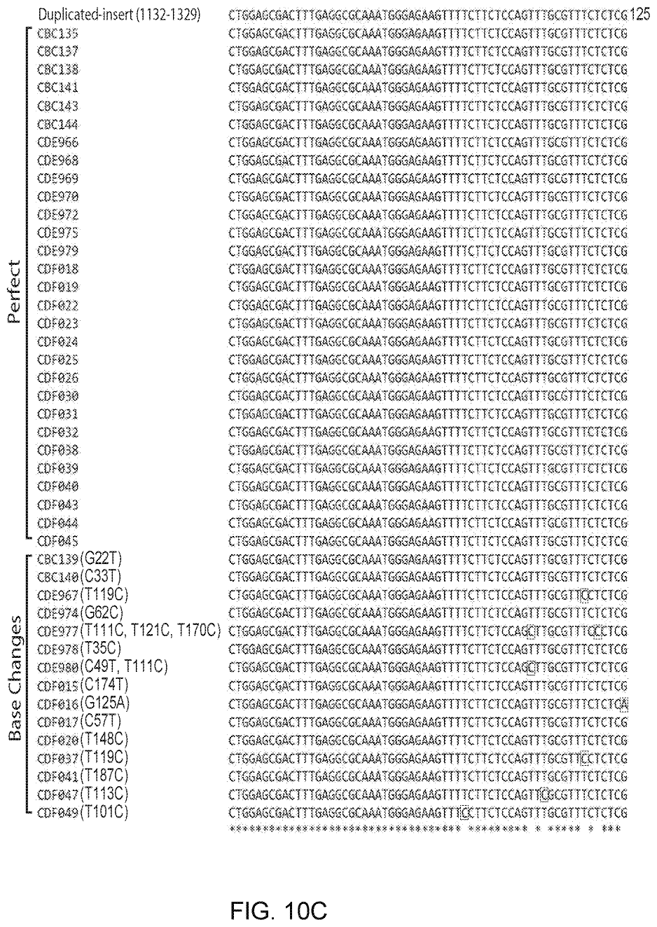

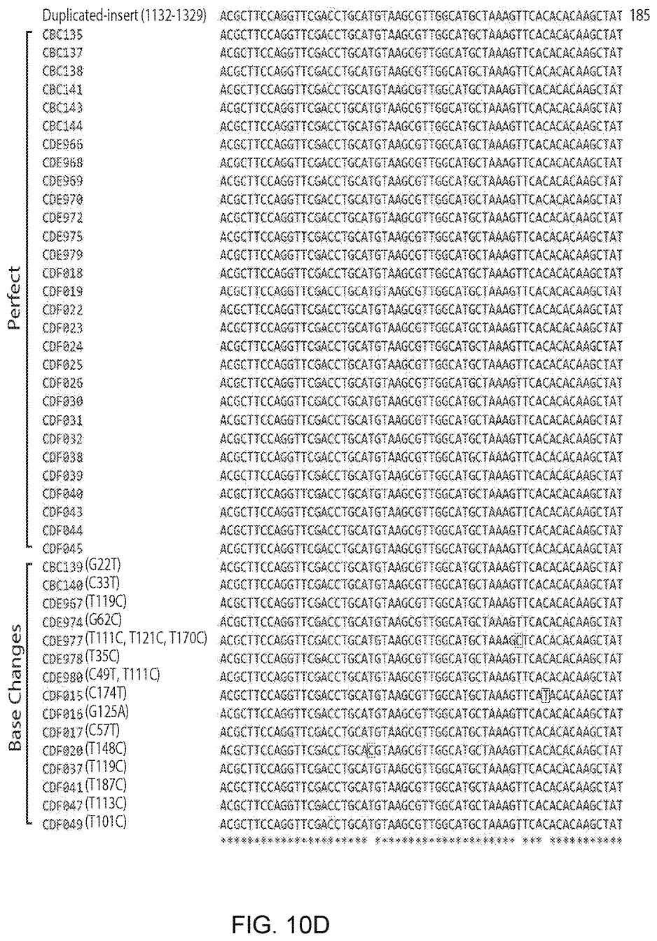

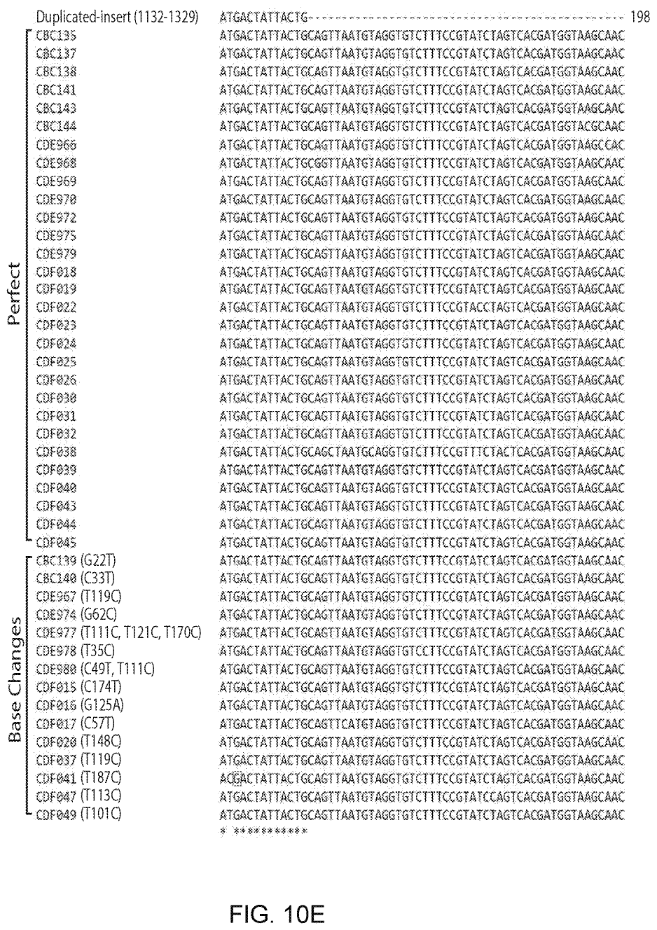

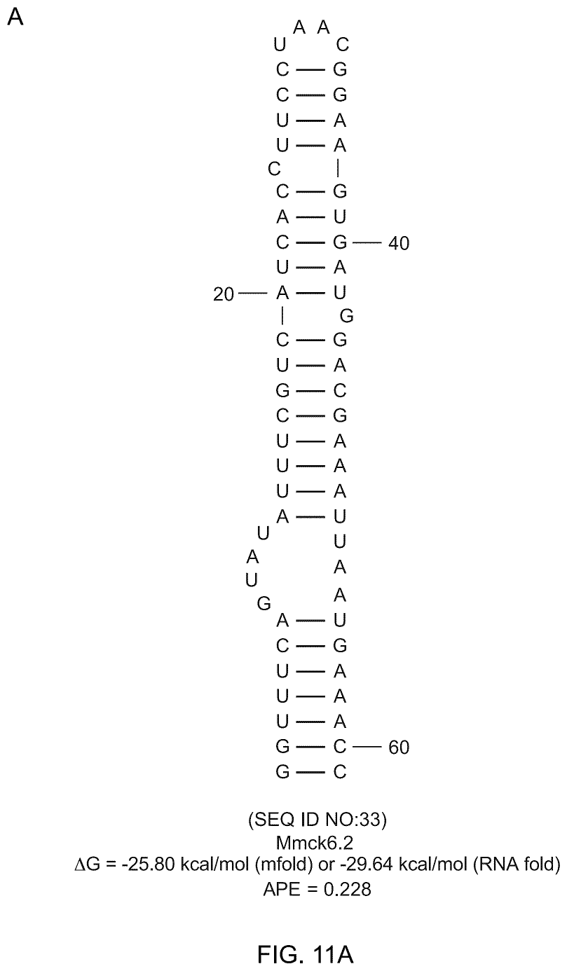

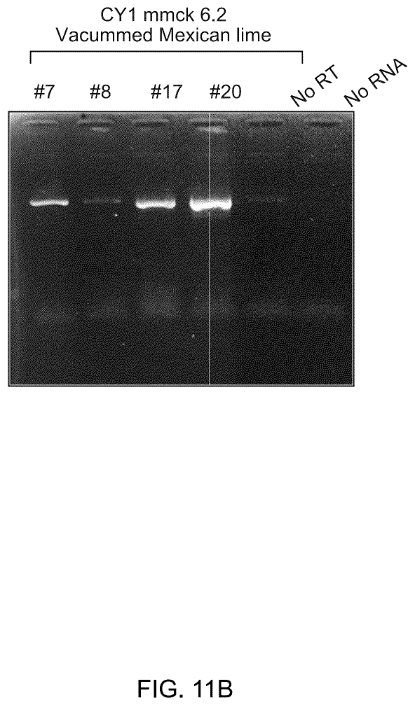

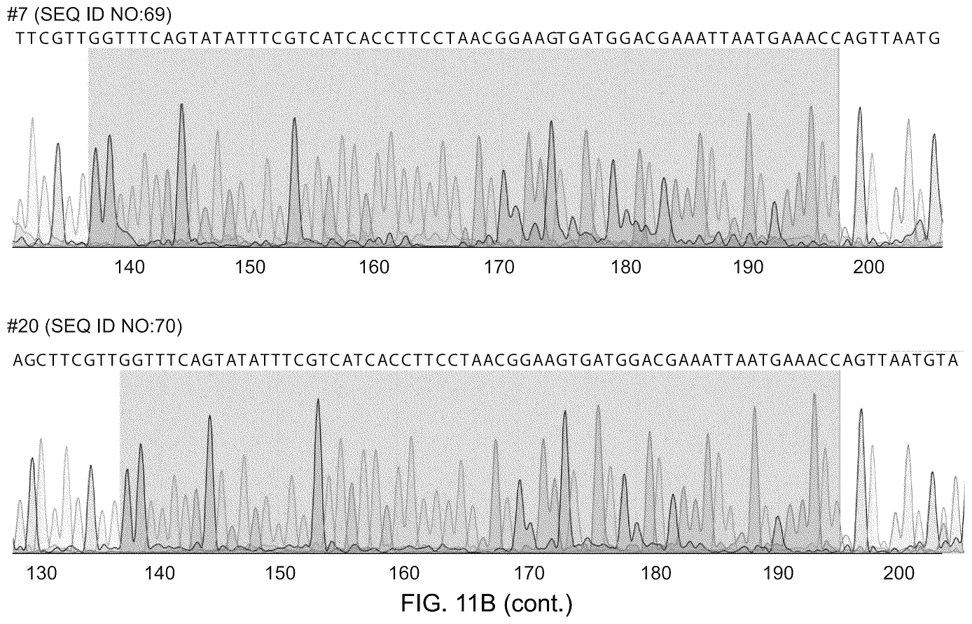

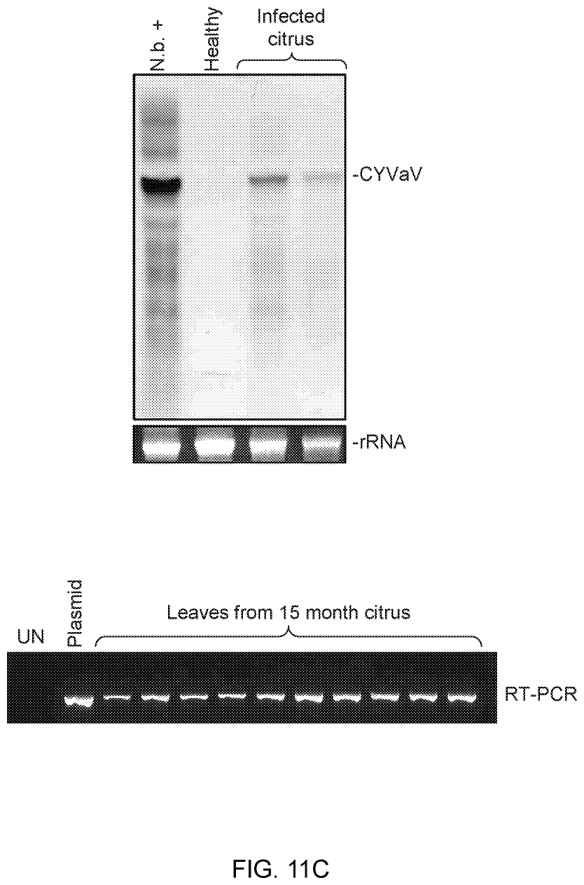

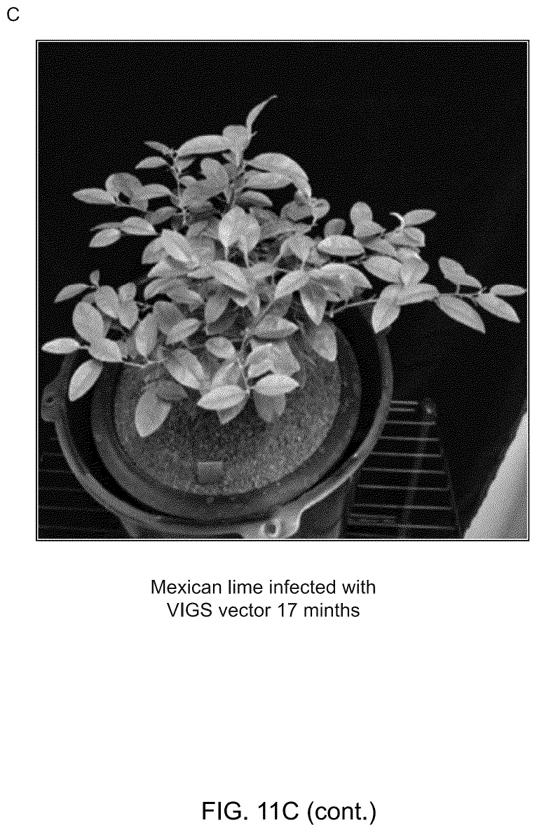

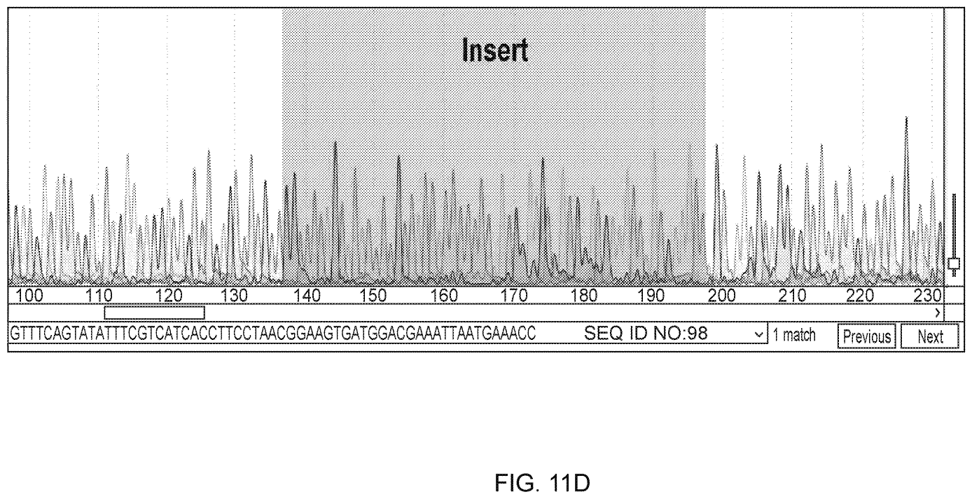

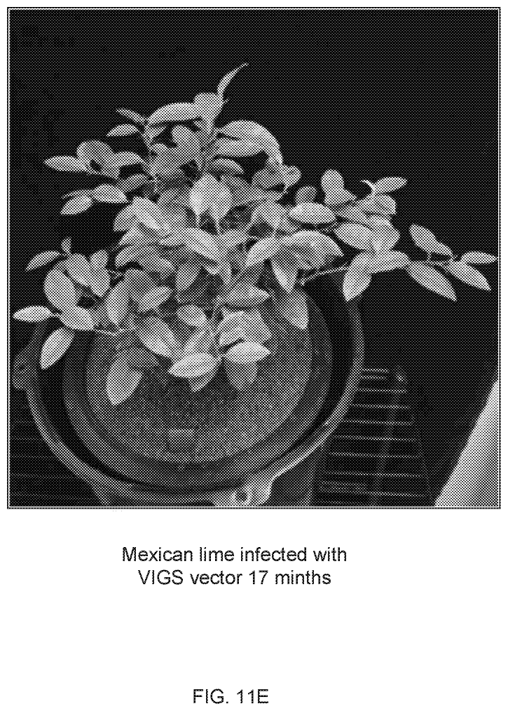

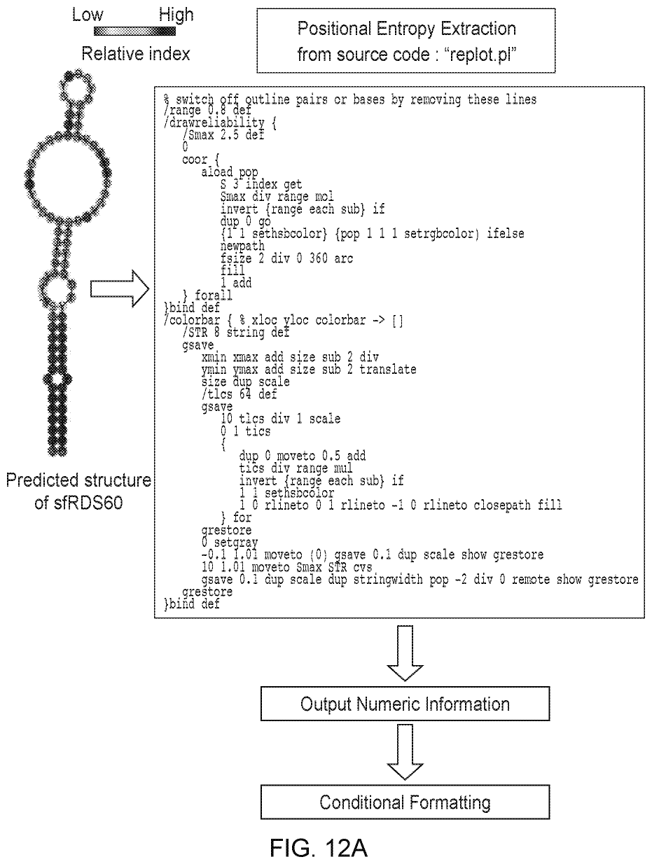

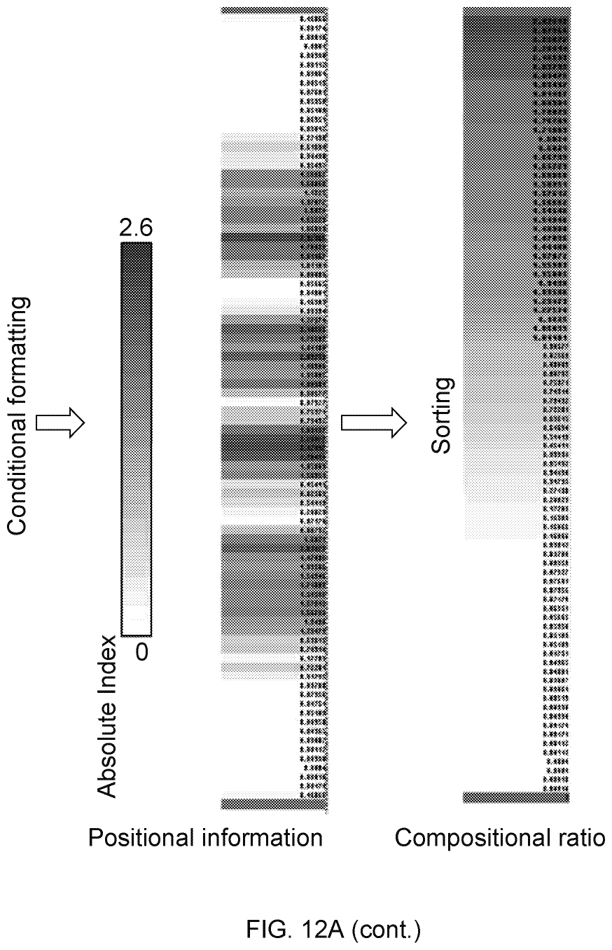

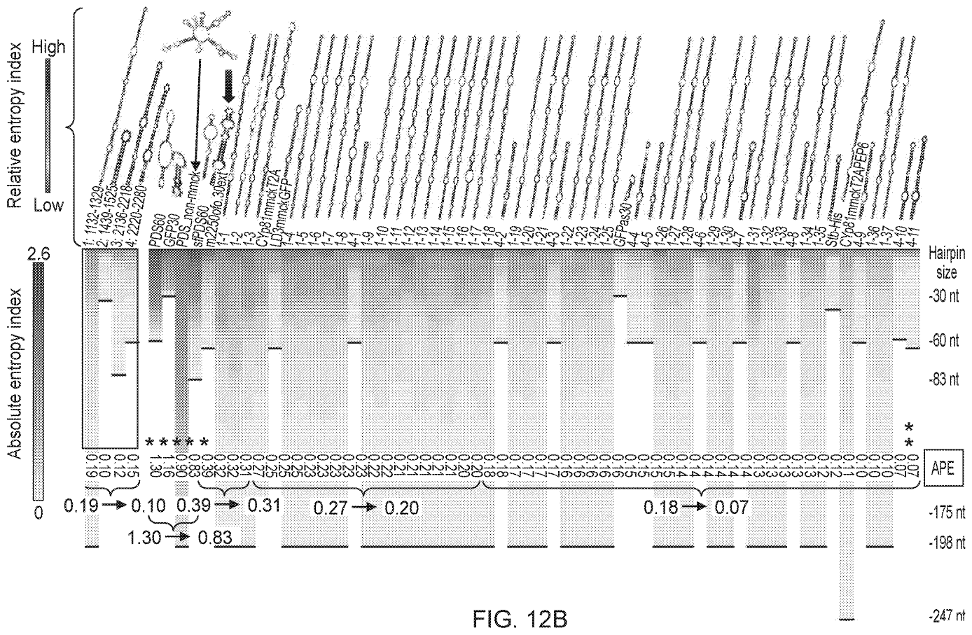

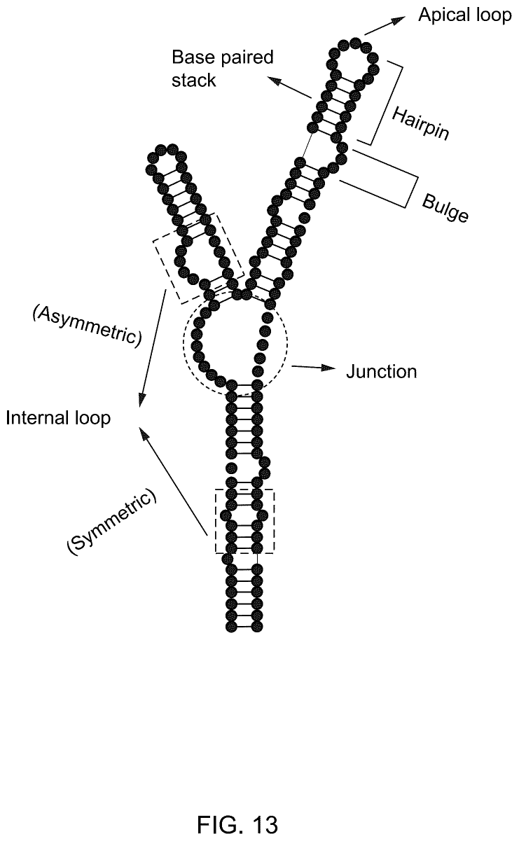

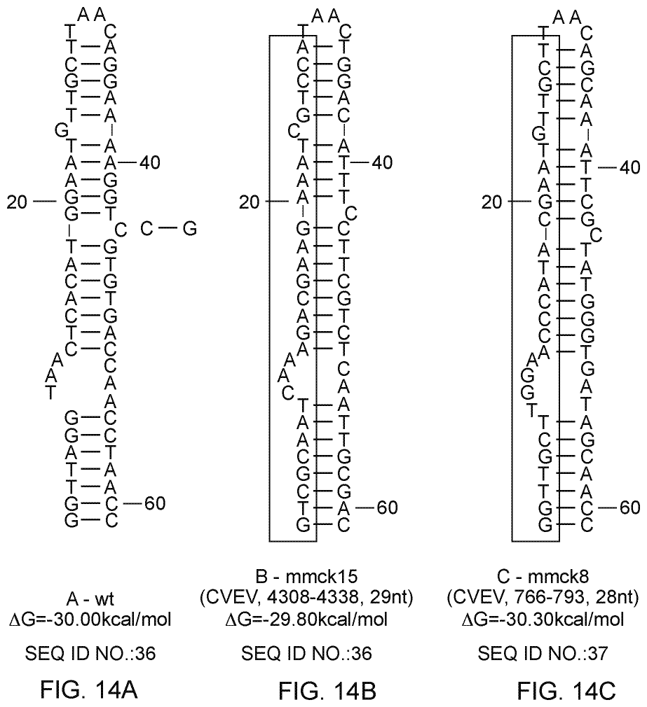

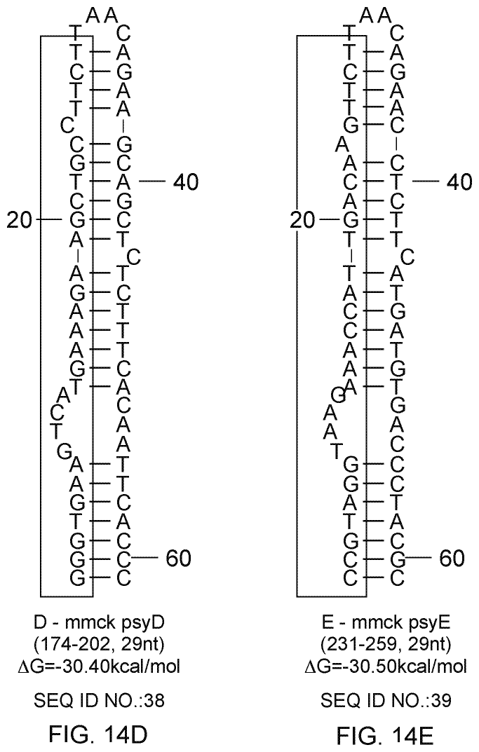

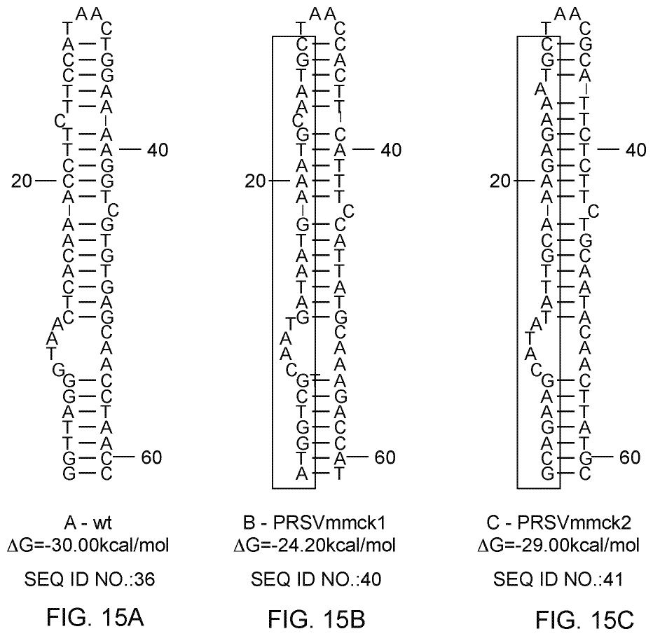

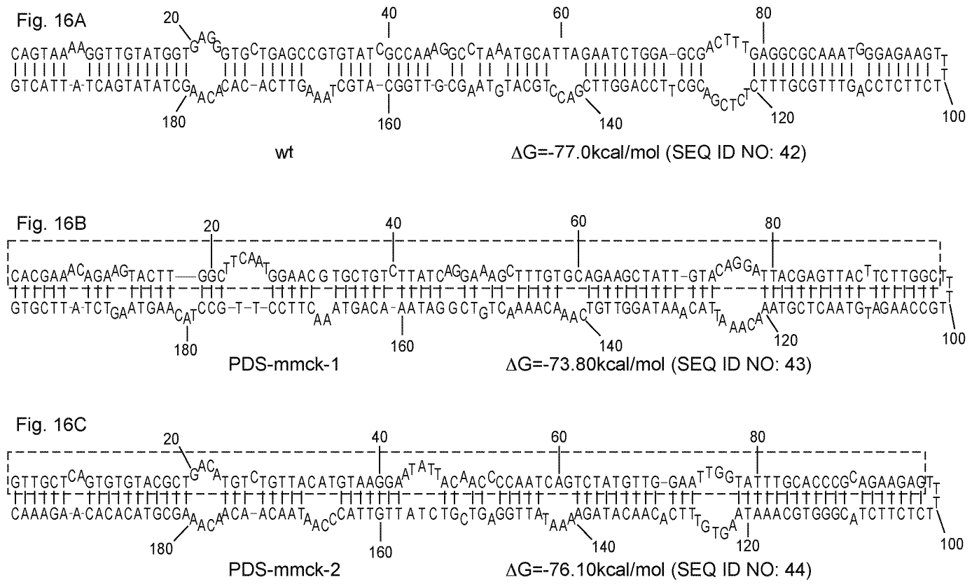

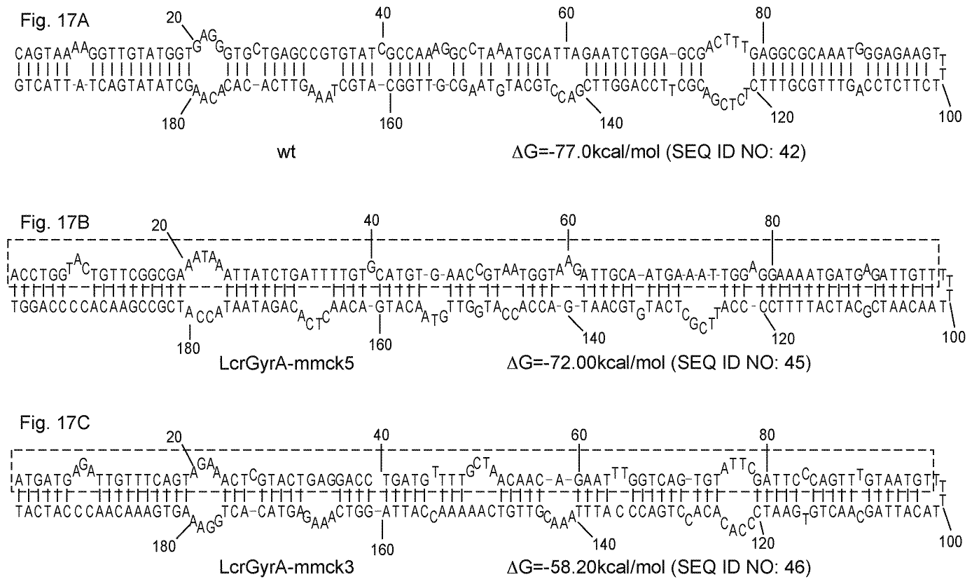

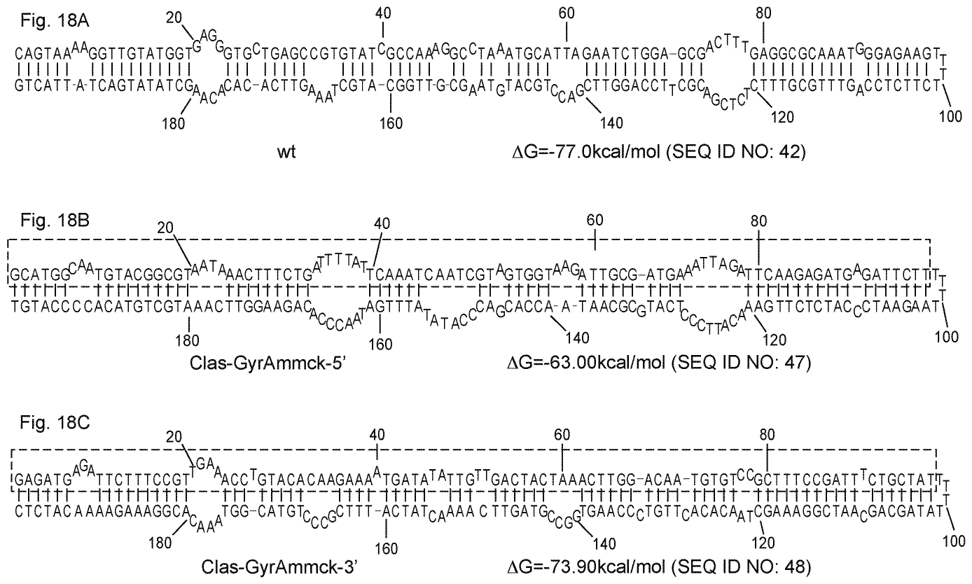

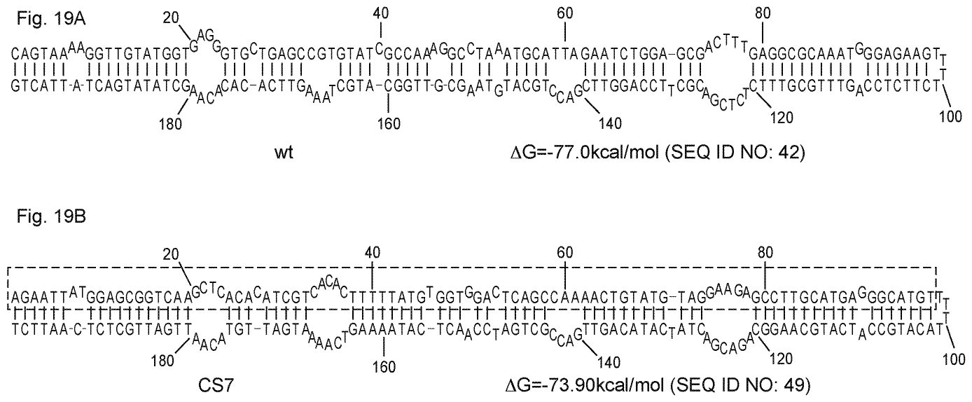

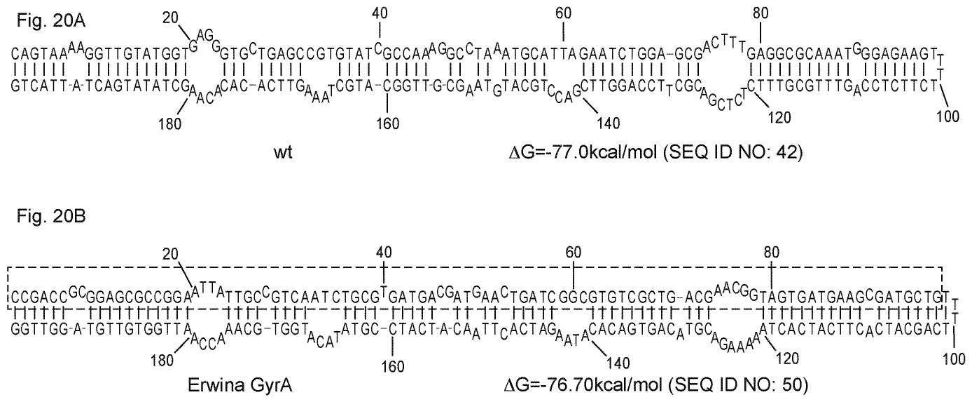

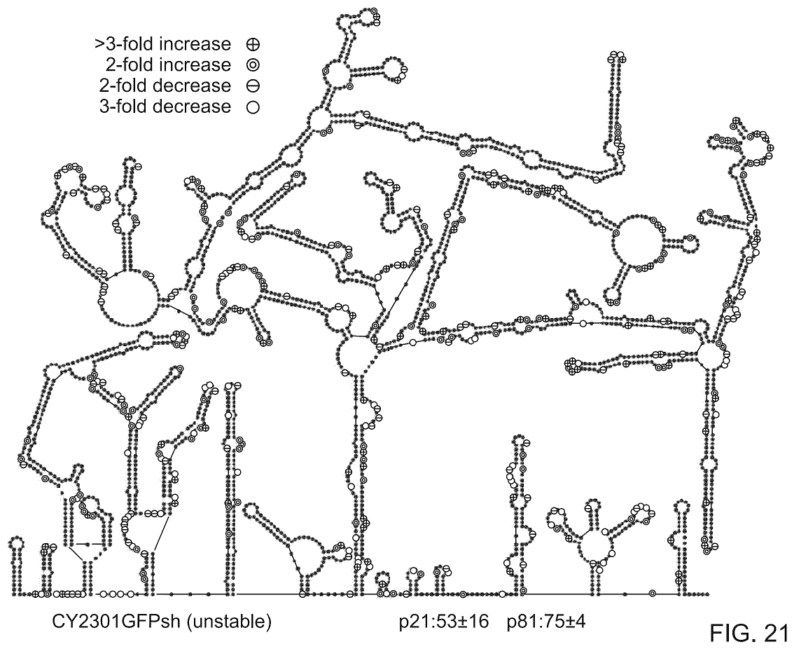

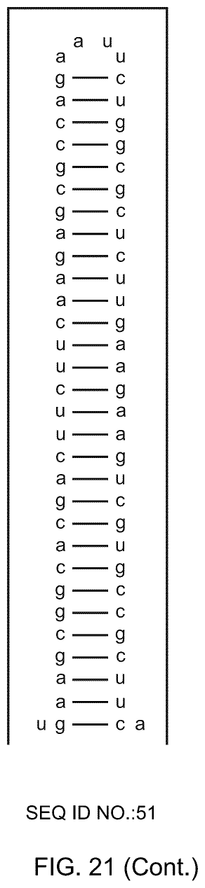

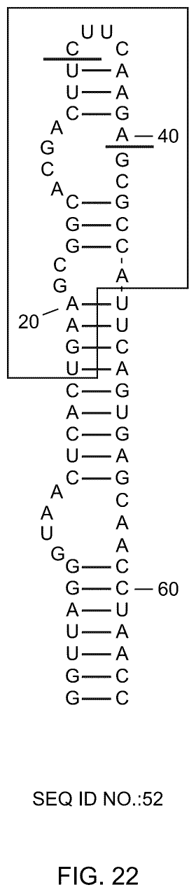

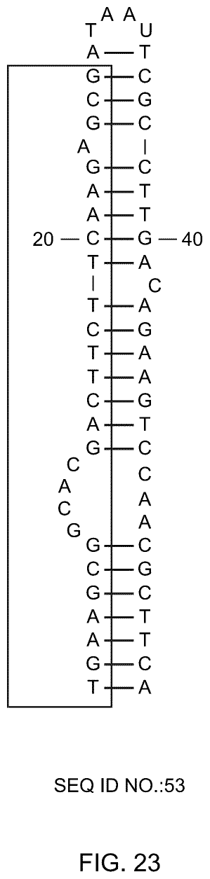

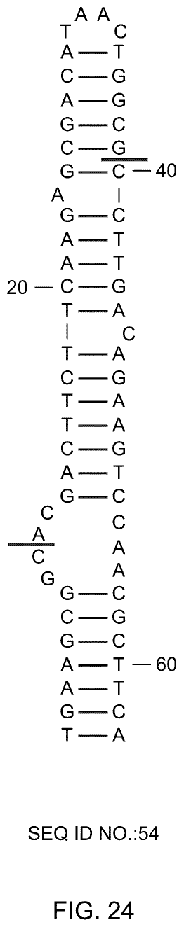

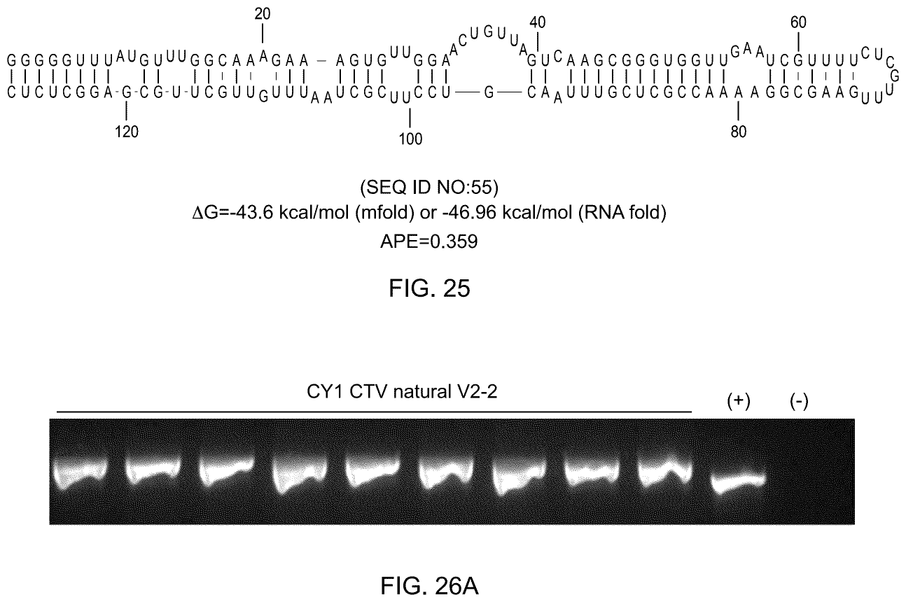

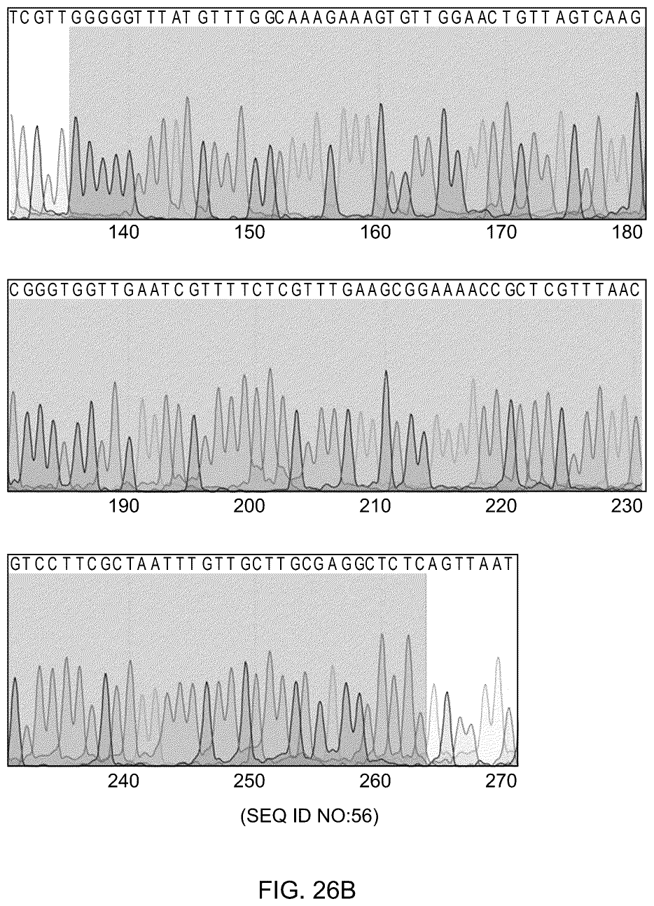

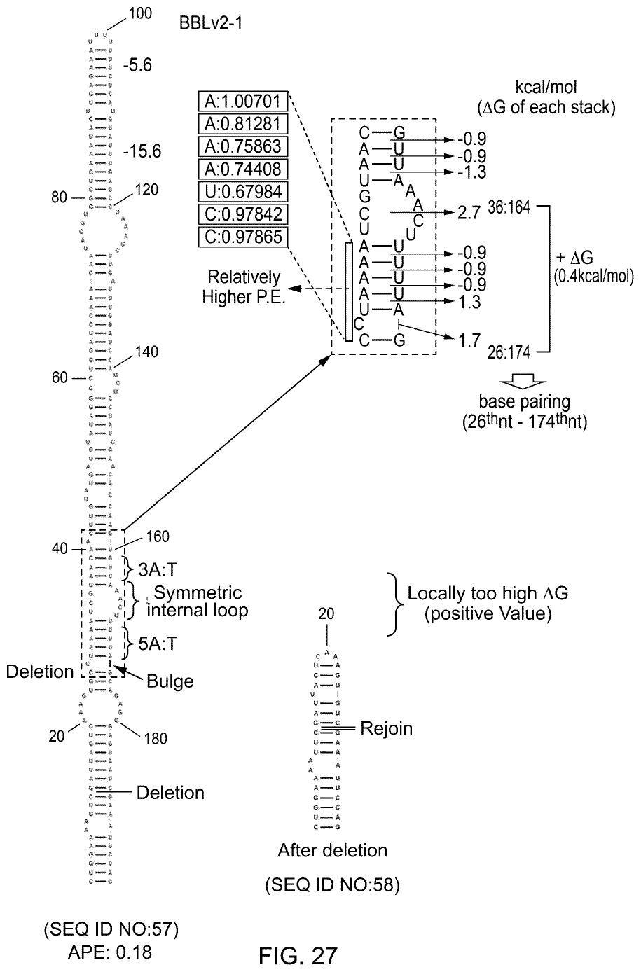

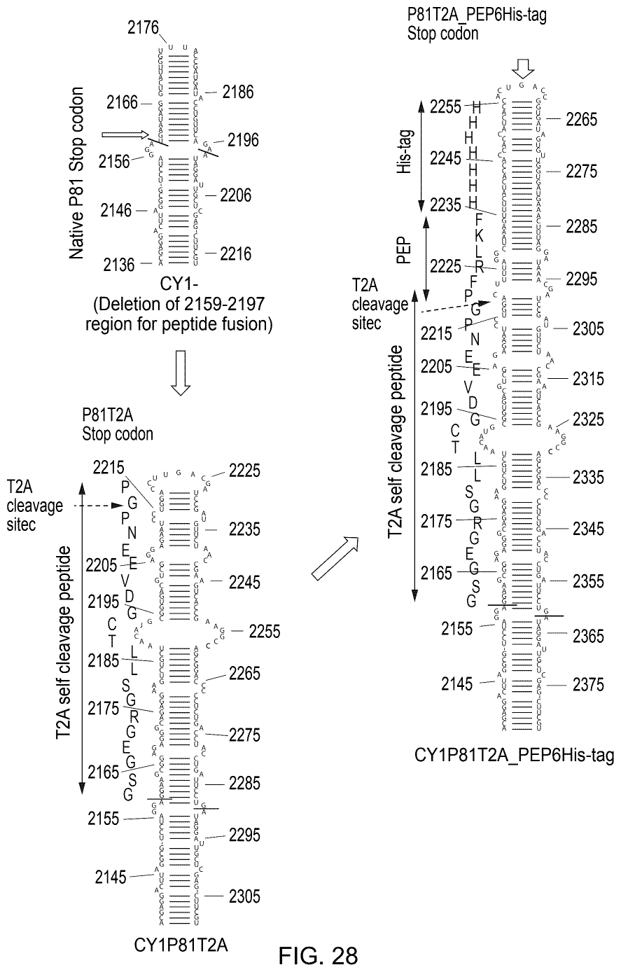

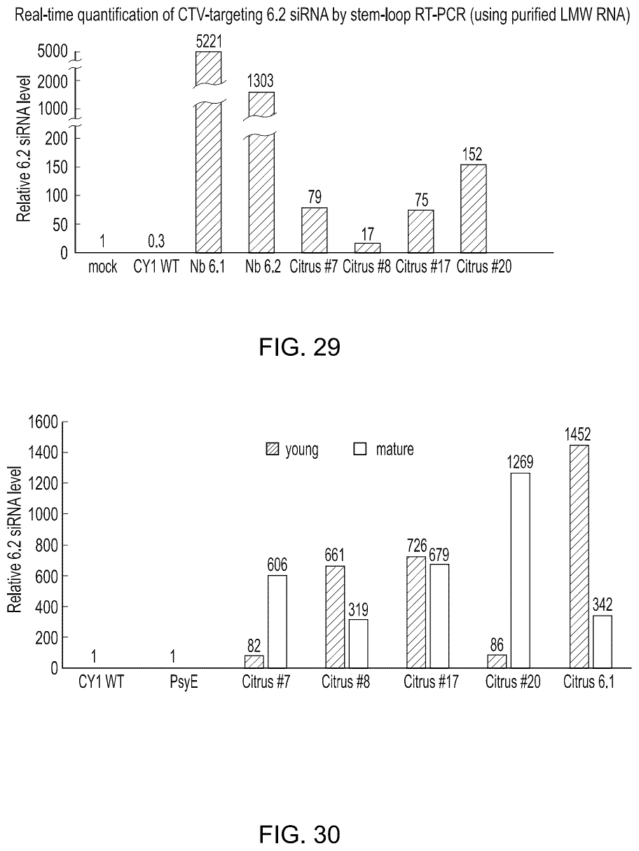

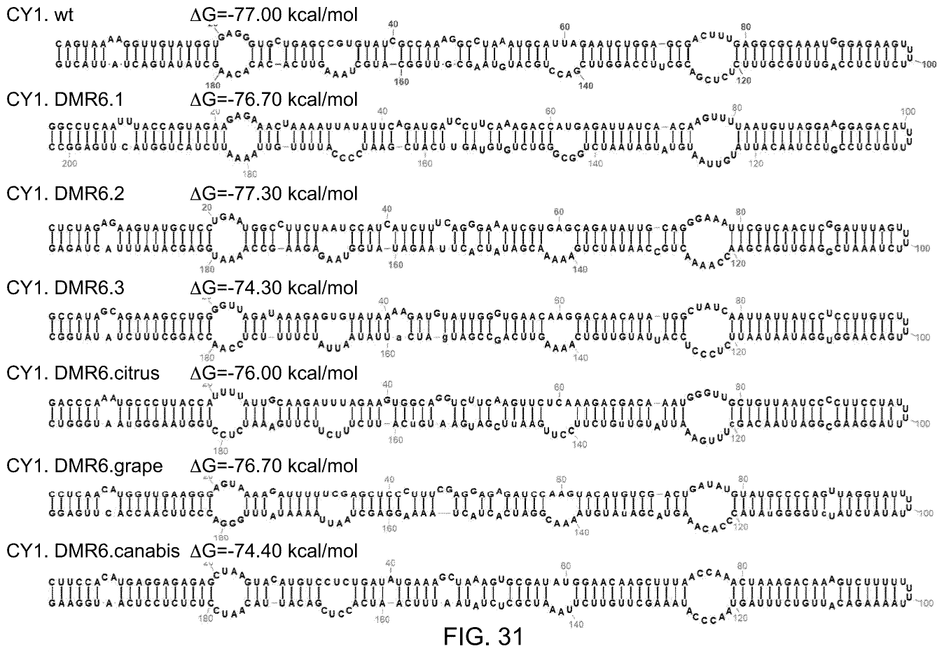

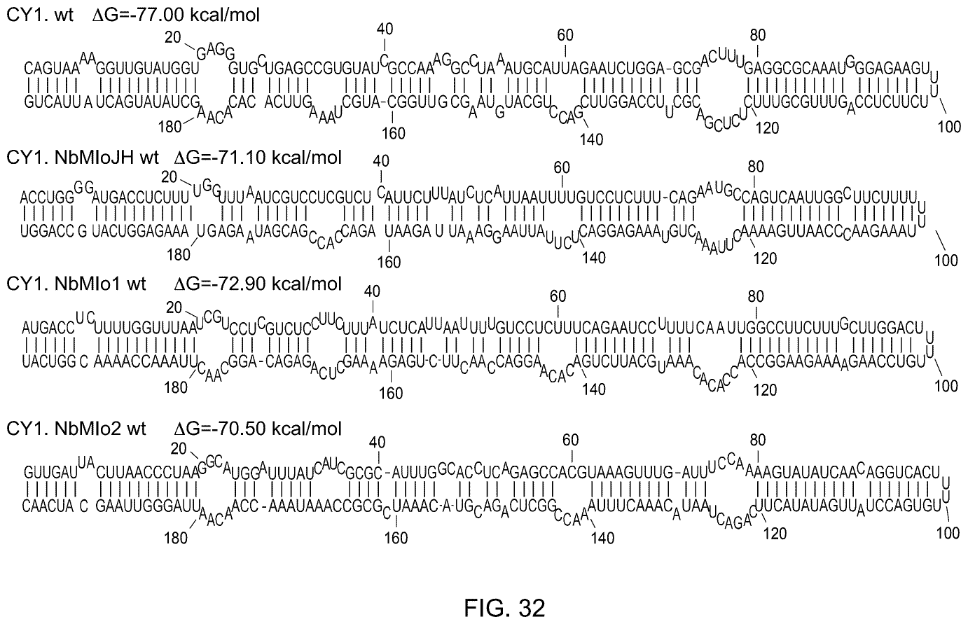

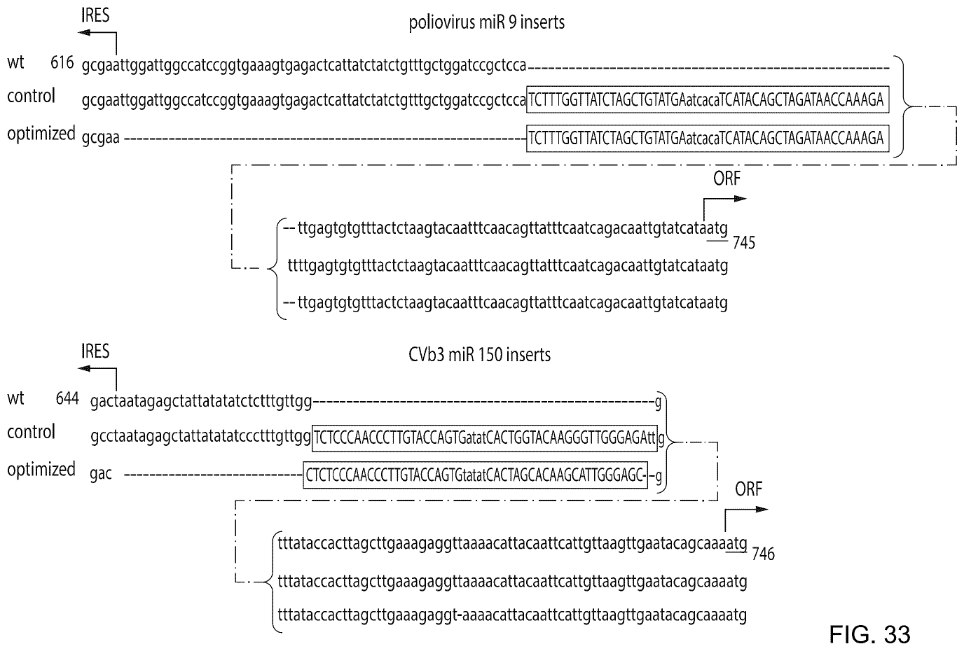

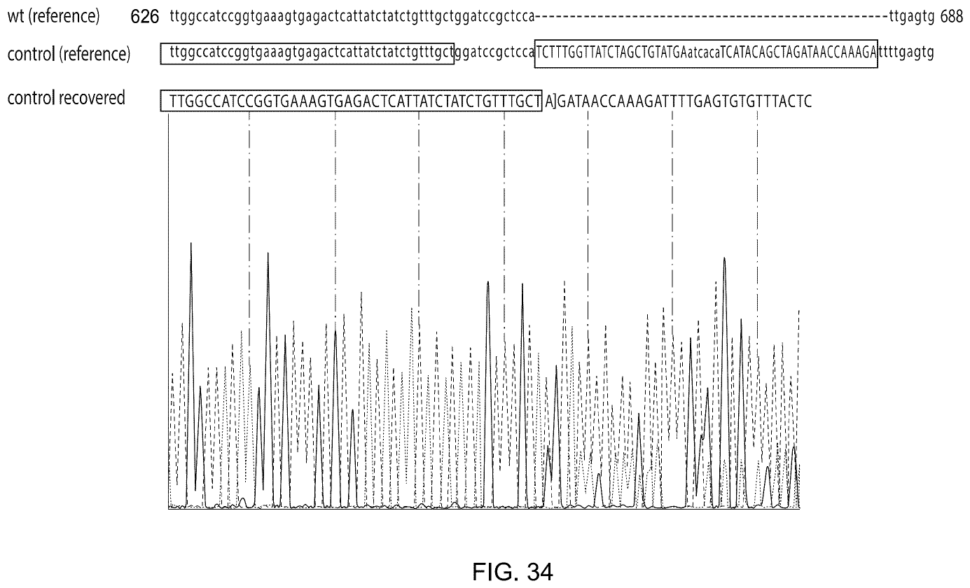

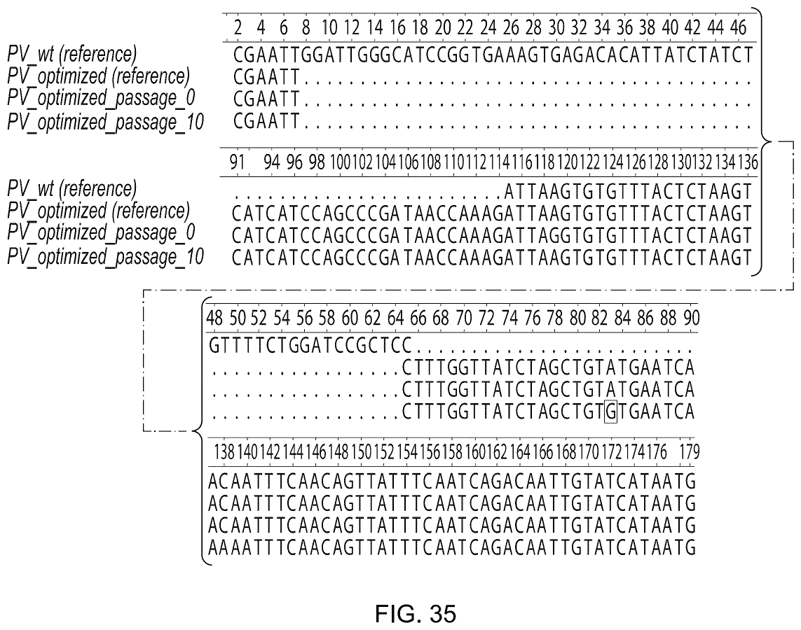

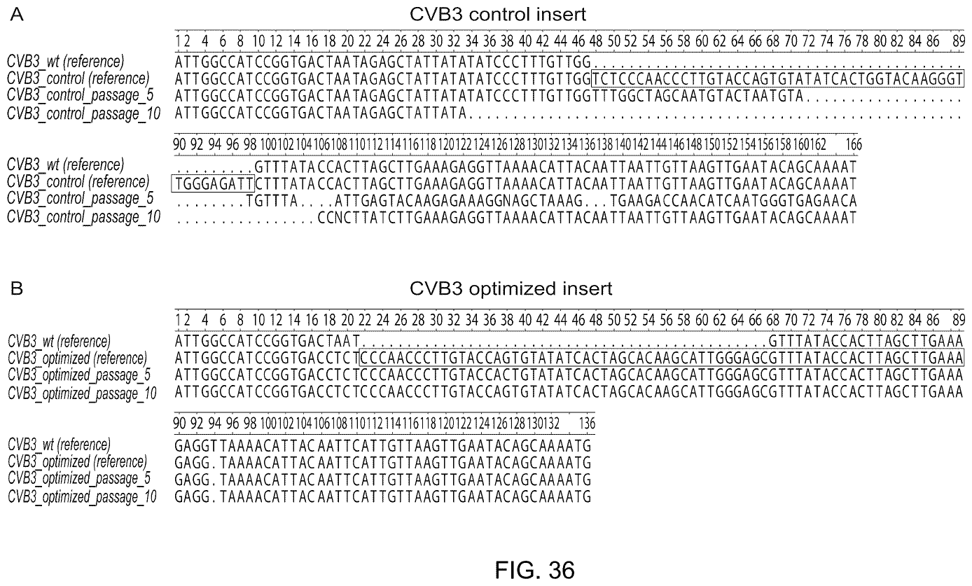

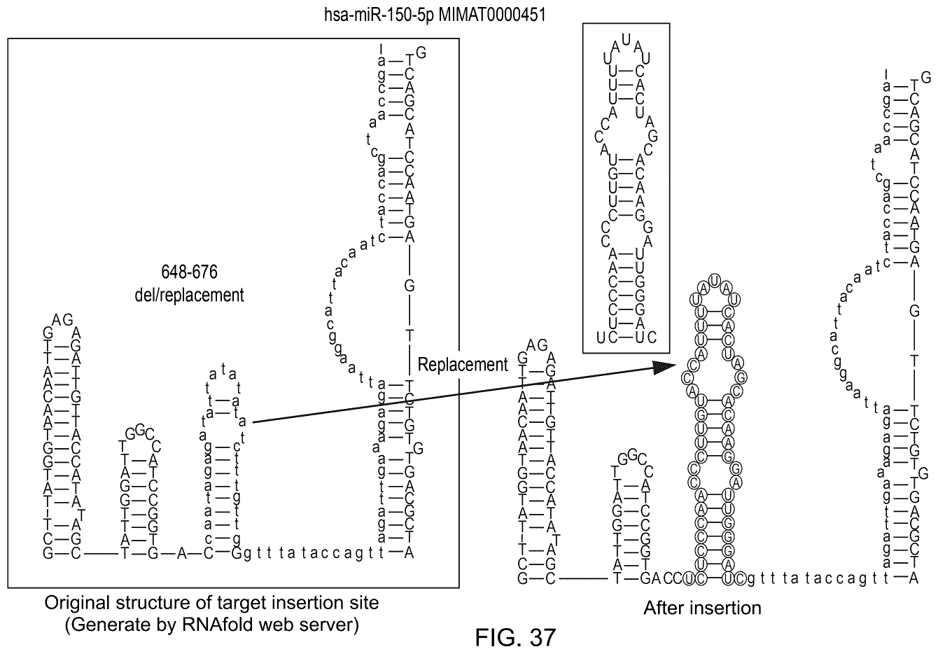

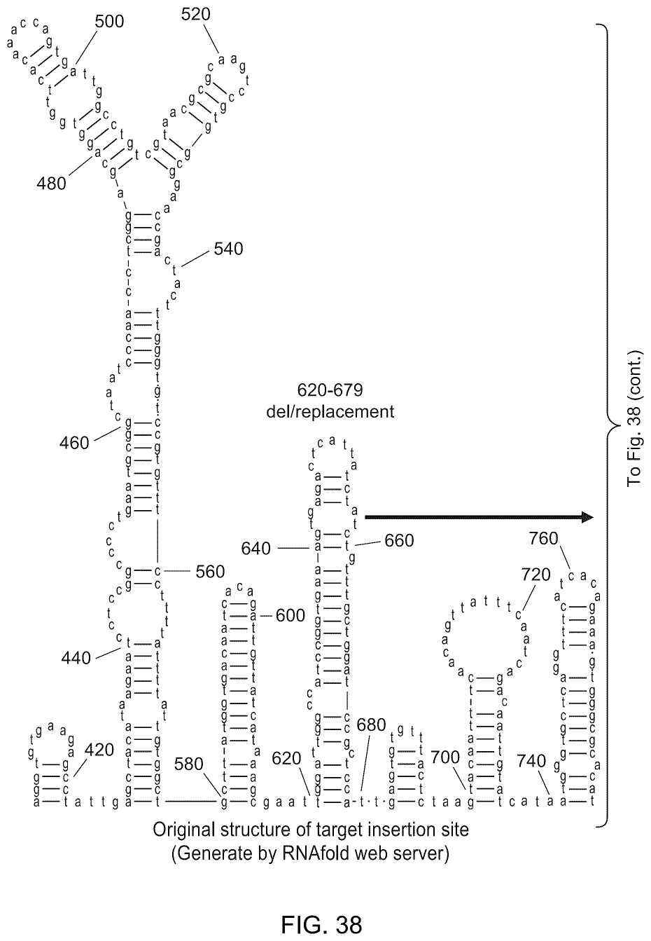

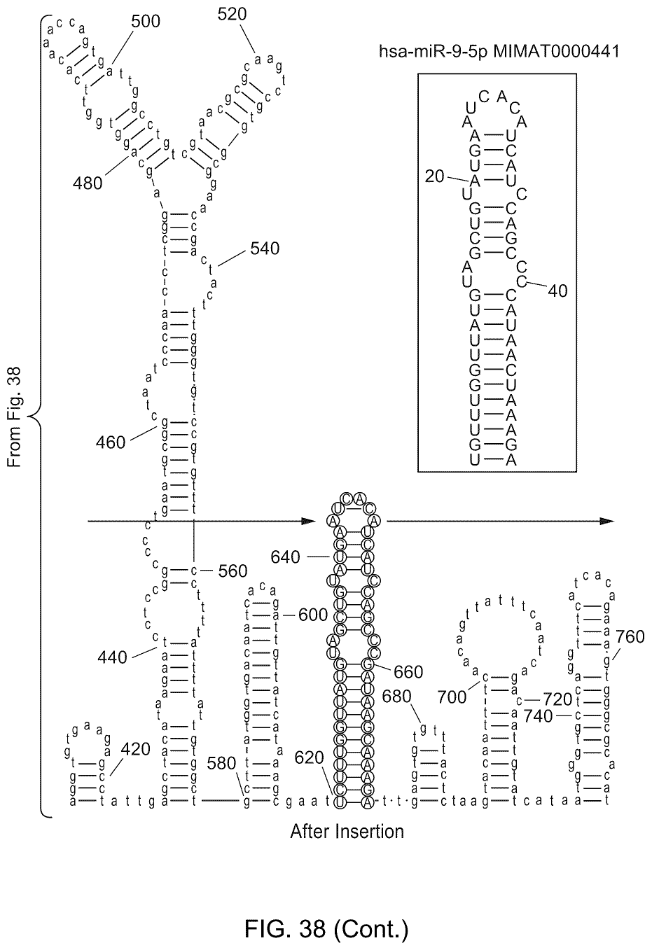

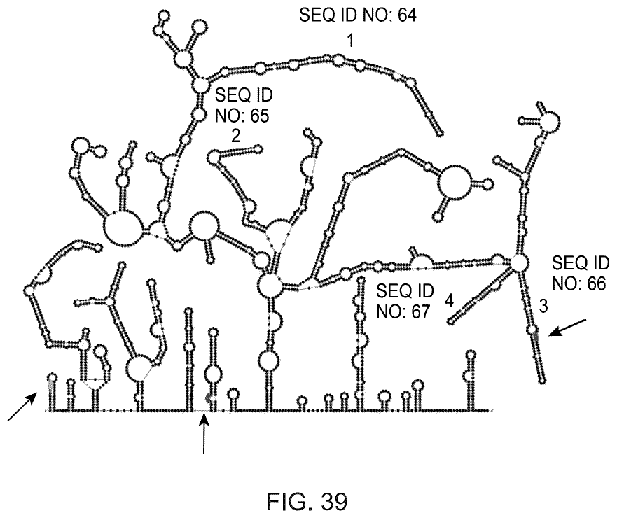

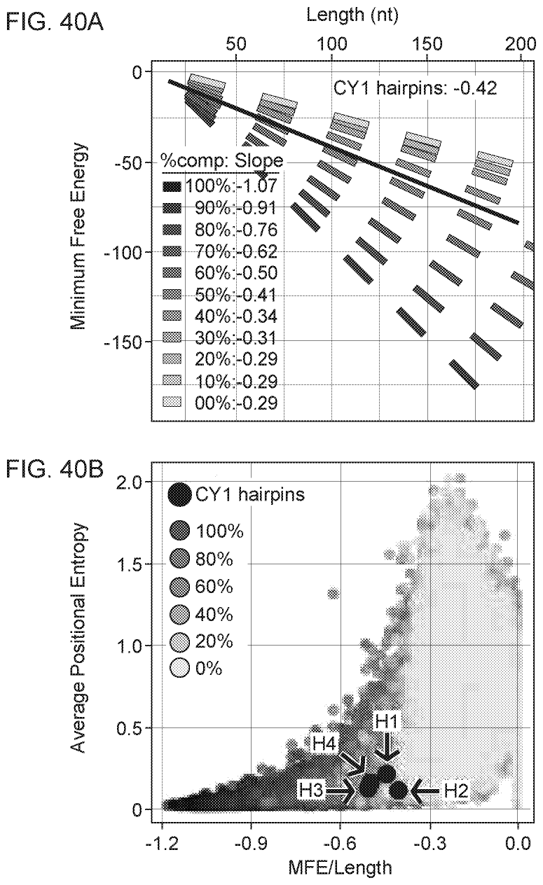

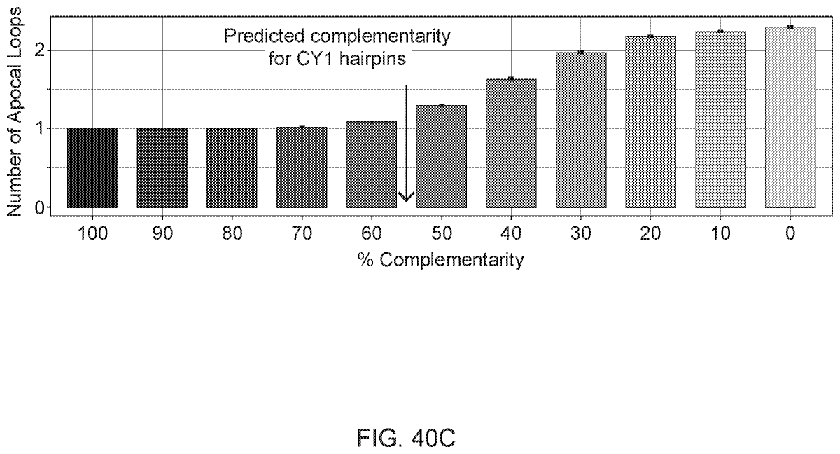

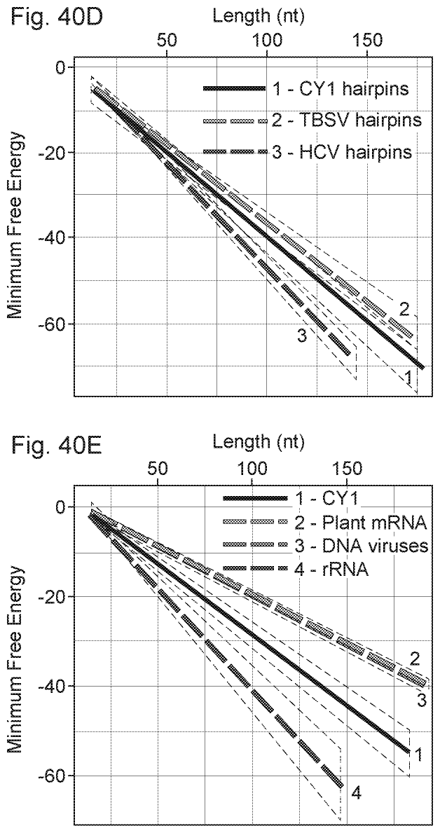

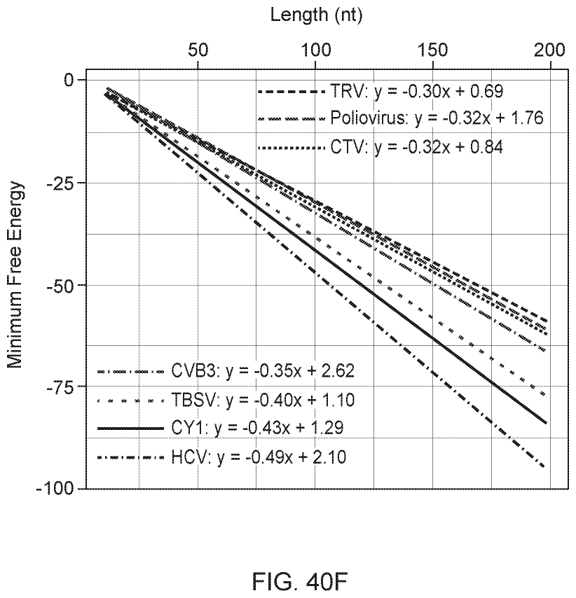

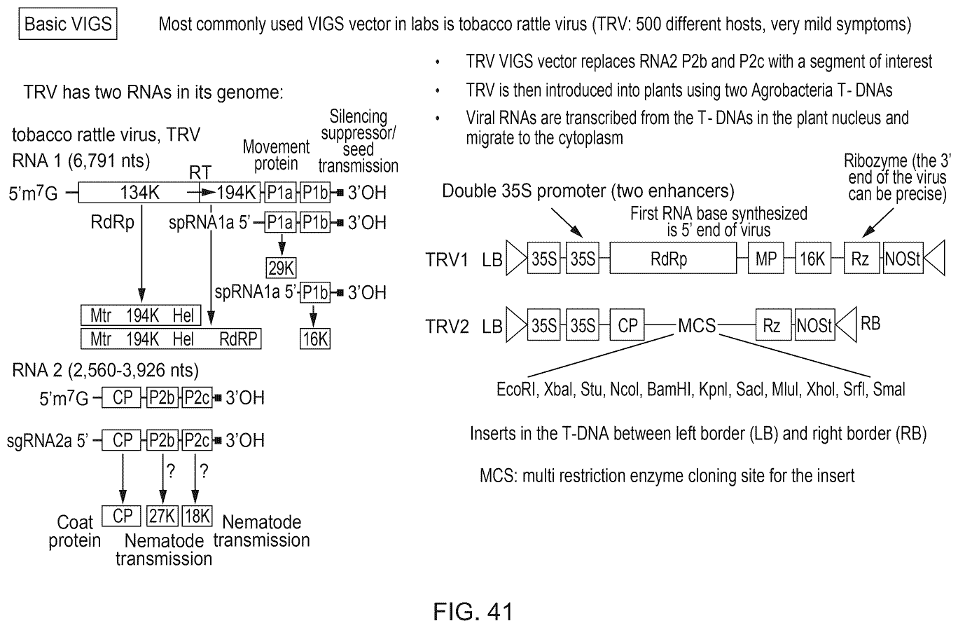

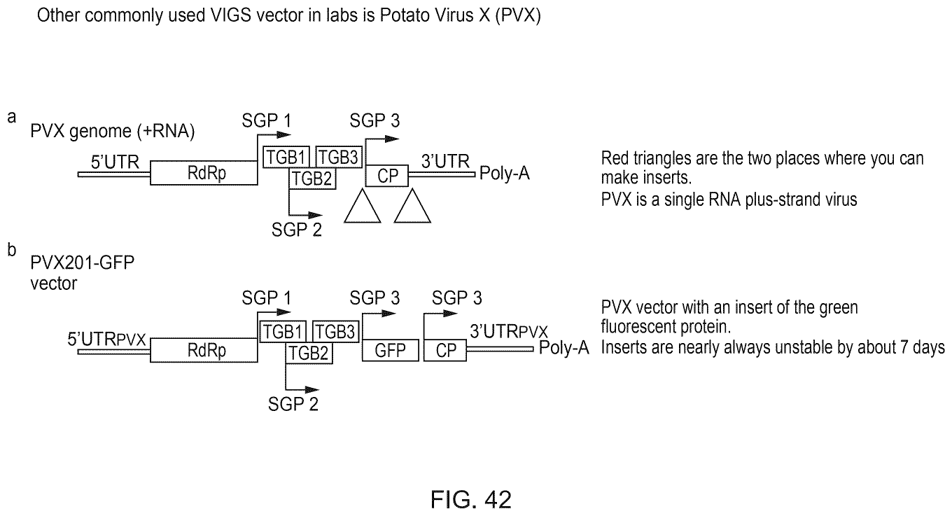

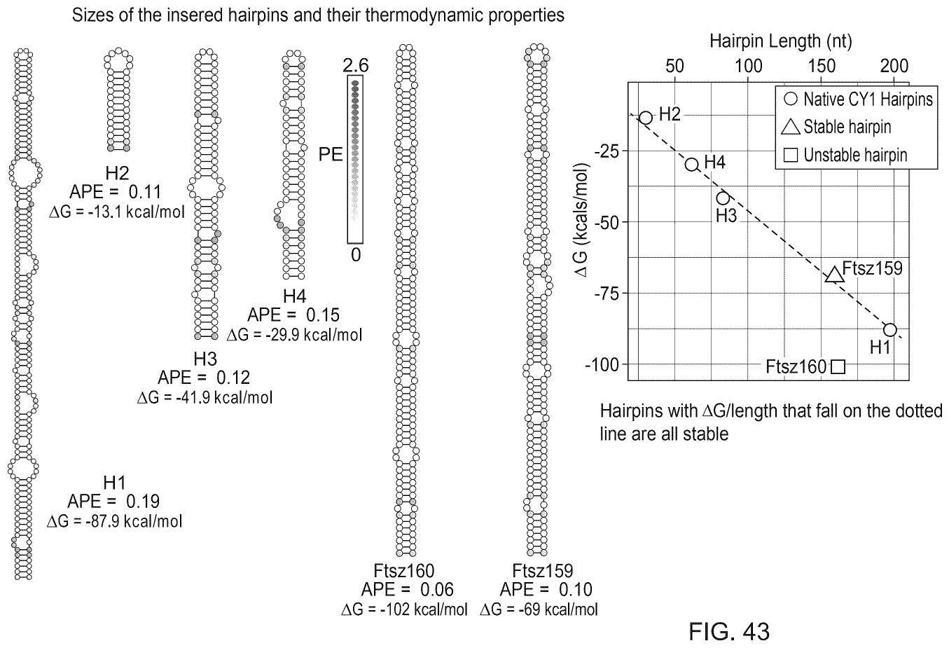

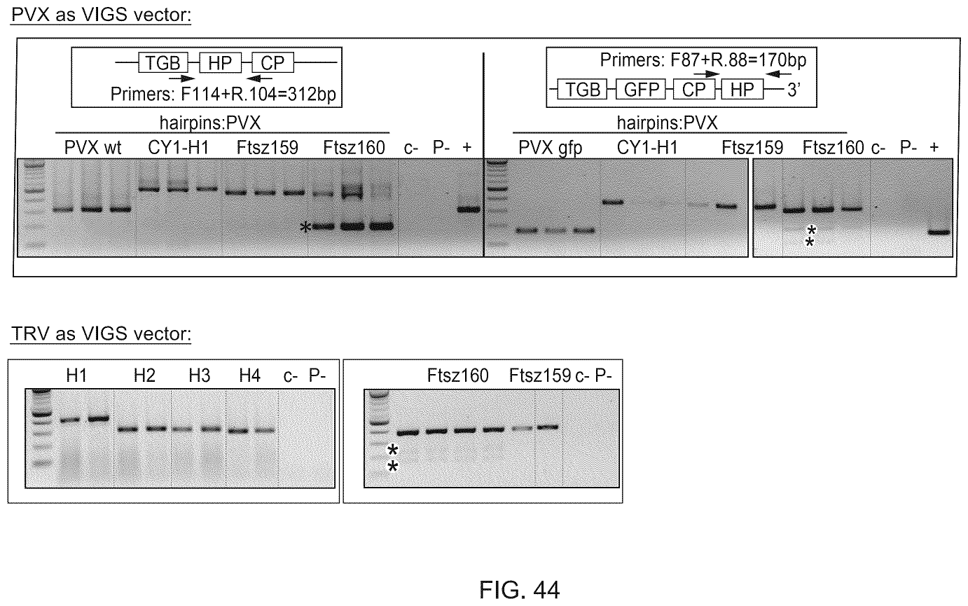

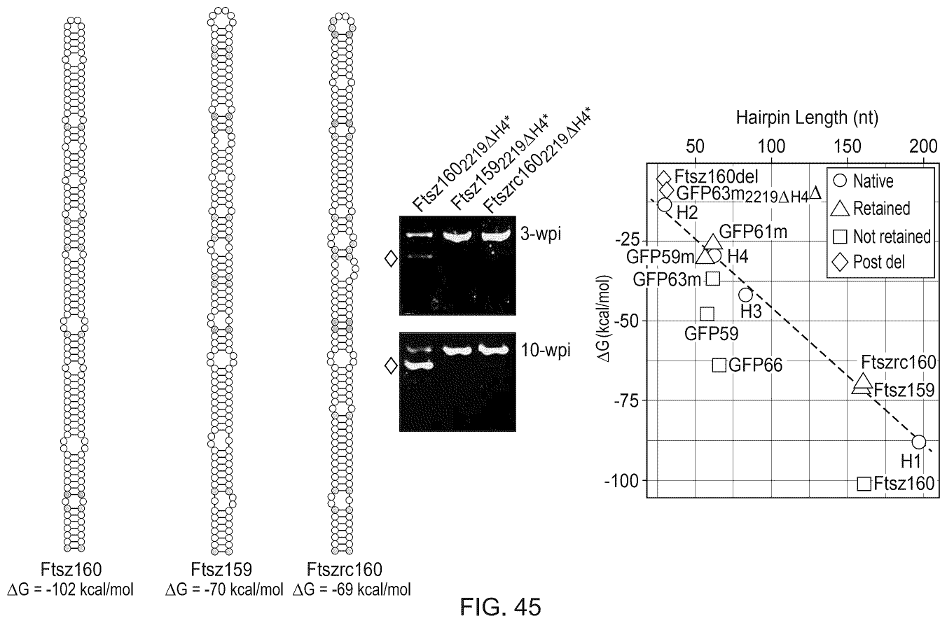

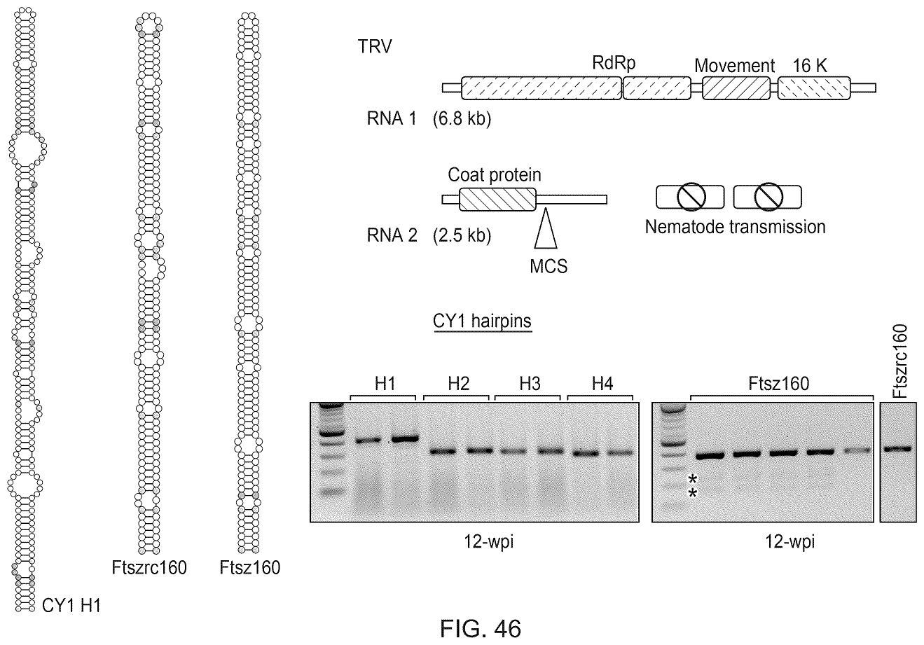

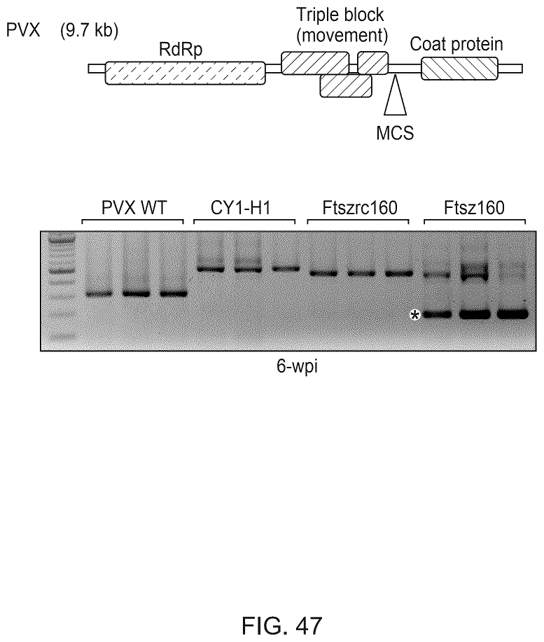

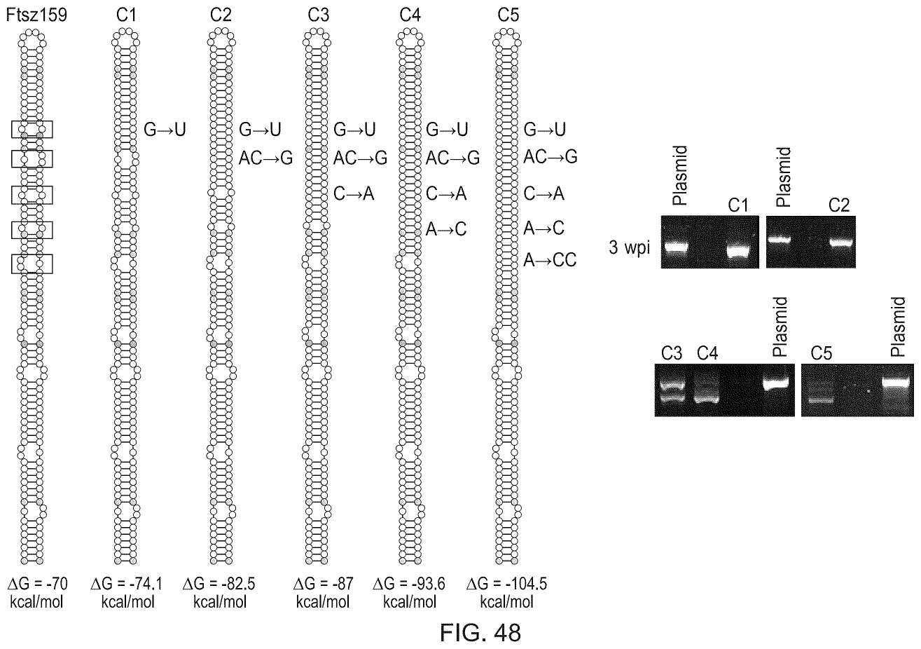

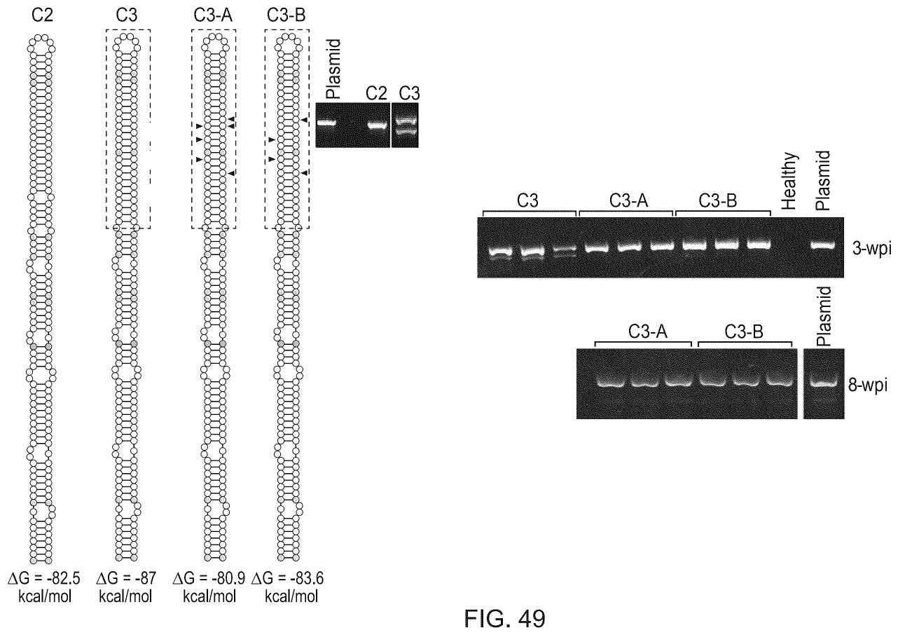

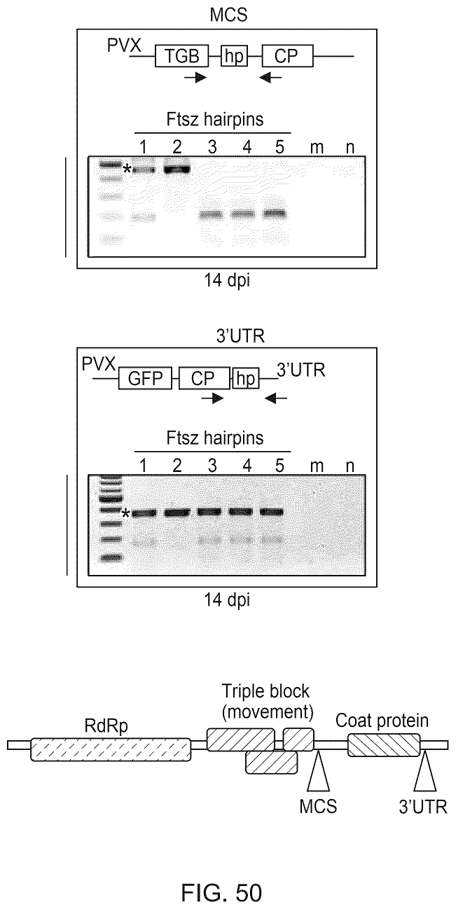

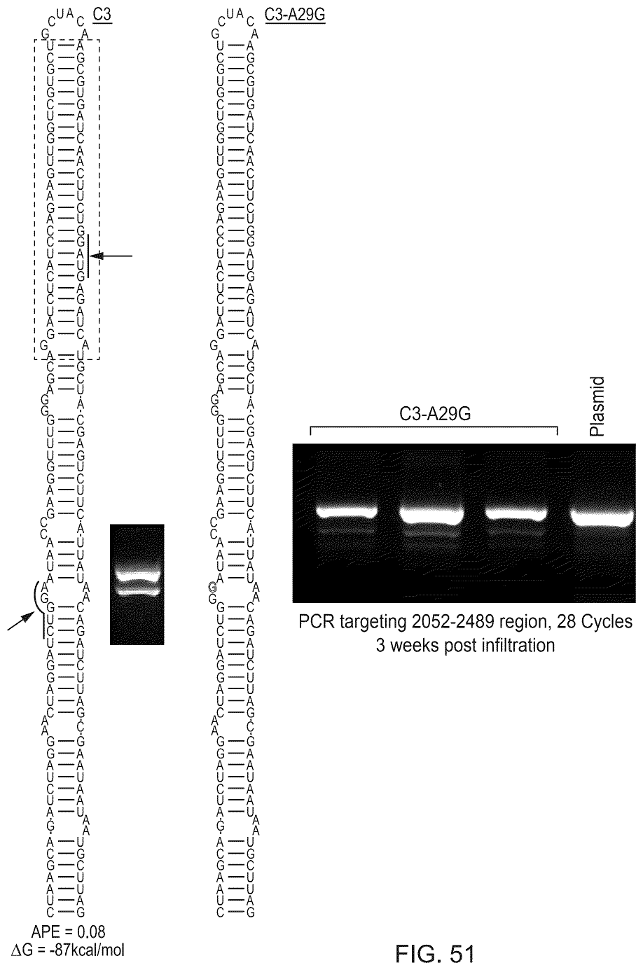

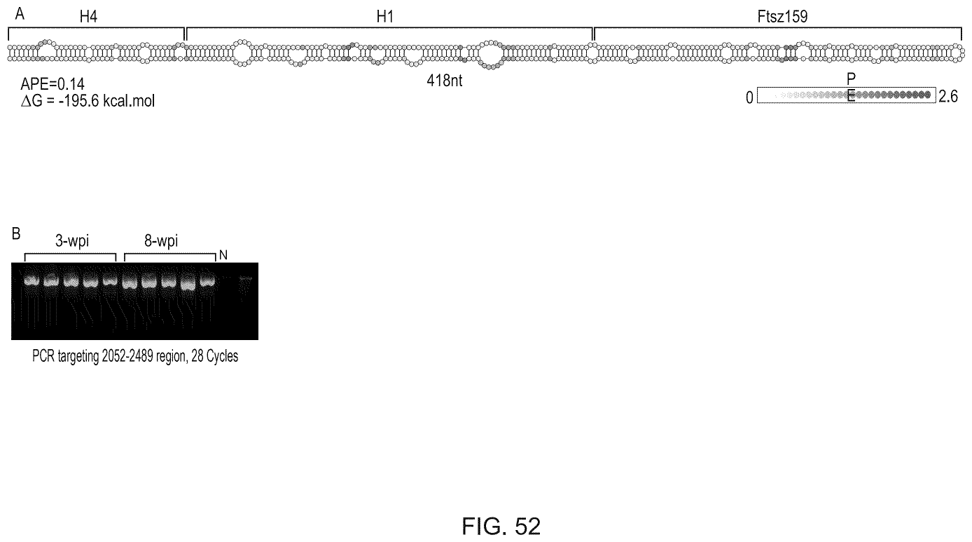

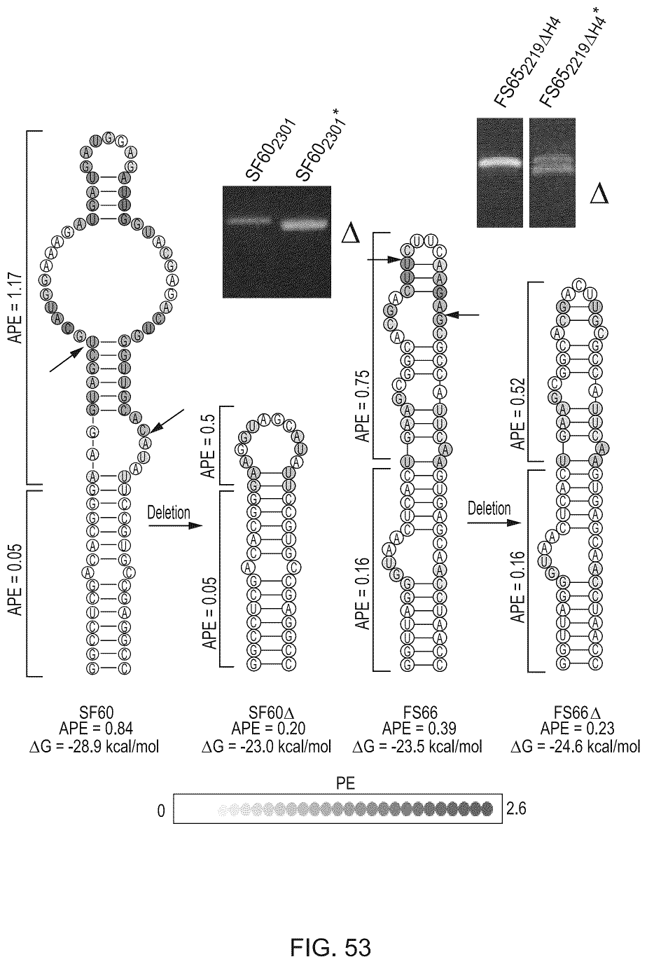

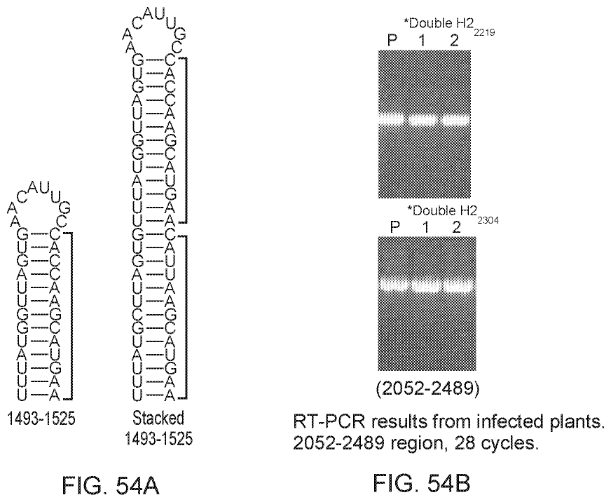

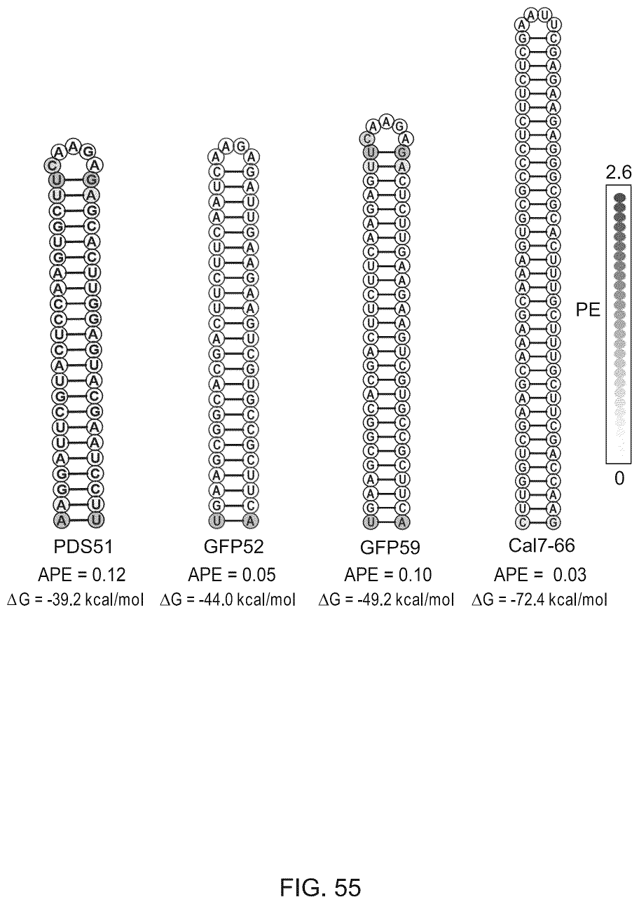

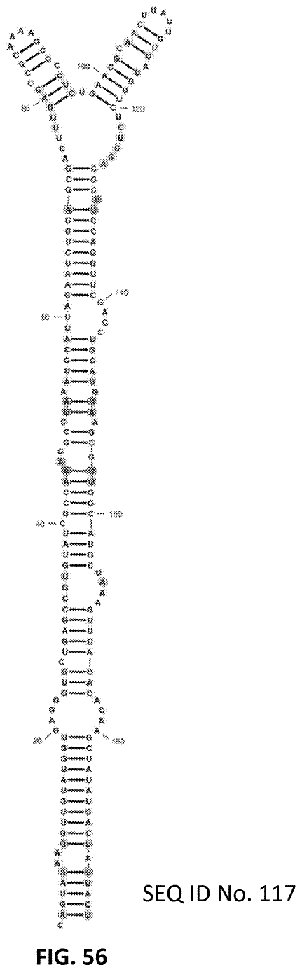

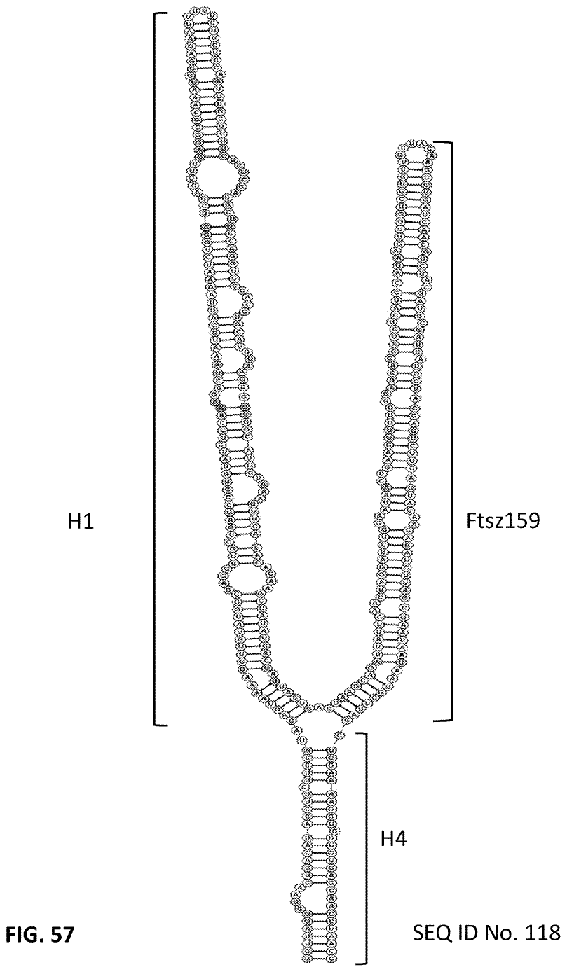

The polynucleotide sequence shown in FIG. 6D is:

(SEQ ID NO: 12)
ttggttagggtaactcacataccttcttccatatgaagcggcacgacta
tctagtcannntggtaagcannncgtttatctgt.

No variants were detectable. Many more combinations of two-insert hairpins were made and all were stable. As shown in FIG. 6C, RT-PCR should indicate a single amplified product, with batch sequencing showing little to no heterogeneity. For comparison, sequencing of an unstable construct is shown in FIG. 6D.

FIG. 7 illustrates wild-type (WT) CY1 (alternatively called CYVaV) and its nearest relative, CY2 (alternatively called CYVaV-Delta), from cannabis. CY2 has two inserts and other changes that allows it to encode an additional ORF.

FIGS. 8A-8D illustrate the stability of a sequence copied from a region of opuntia umbra-like virus (OULV) (OULV 2349-2491, 143 nt, ΔG is −55.60 kcal/mol) and inserted into the CY1 genome. Referring to FIG. 8A, left image, the secondary structure of OULV RNA genome is shown; the enlarged chimeric sequence (marked with light-brown circle) and its secondary structure is in the dashed box below. Referring to FIG. 8A, middle image, the secondary structure of CY1 is shown; the enlarged structure of the insertion site in CY1 genome is in the dashed box below and the insertion point is marked with a gray arrow. Referring to FIG. 8A, right image, the secondary structure of CY23300ULV (CY1 with OULV 2349-291 inserted between 2330/2331 of CY1) is shown; the enlarged secondary structure of CY1 2330 location with chimeric OULV insert is in the dashed box is shown. RT-PCR results using the cDNA of the three infected plants by CY23300ULV are shown in FIG. 8B. Samples are labeled as CY23300ULV-1, CY23300ULV-2, and CY23300ULV-3, respectively, with sequencing identification presented in parentheses. PCR was performed using a primer set targeting the region from 1996 to 2452 of CY1 genome. pET17CY23300ULV and pCB301CY23300ULV were used for positive control. Sequence alignment results of the three samples are shown in FIG. 8C. The presented sequence of CY1_wt is SEQ ID NO:1 (2276-2372). The presented sequences of CBC460, CBC461 and CBC462 are identical (SEQ ID NO:13):

taaccagttaatgtaggtgtctttccgtatctagtcacgatggtaagca
acccgtgcgtgttacgtcagtgacaatcgccatggaaccggcaatgtct
acctctacccaacactgtggggtaggactcgctactagcggggcctact
cttcaggtgagggttttggtaacttgttcaagtacaagcgcgcttgtaa
ctttatctgtacggcgctcacccgtgggtaggaaggtgaaggtt

The presented sequence of the OULV-insert is (SEQ ID NO:14):

tgcgtgttacgtcagtgacaatcgccatggaaccggcaatgtctacctc
tacccaacactgtggggtaggactcgctactagcggggcctactcttca
ggtgagggttttggtaacttgttcaagtacaagcgcgcttgtaact

Chromatogram images of the three samples above are shown in FIG. 8D. The overall sequencing quality was very stable, except the thymine (T) at position 117 of the insert. The chromatogram signal showed weak noise of adenine (A), which may indicate that a small population of CY23300ULV may have A instead of T at position 117.

FIGS. 9A-9D illustrate the stability of duplicated internal structure (1132-1329) of CY1 at 2220/2280 location in CY1 genome at 8 weeks after systemic infection. Referring to FIG. 9A, left image, the schematic image for the construction strategy is shown; the original hairpin-like structure between the location 2220 and 2280 was removed and the long-hairpin-like structure spanning from nucleotide positions 1132 to 1329 was cloned at the deleted region. Referring to FIG. 9A, right image, the overall structure of CY2220_(1132-1329)_2280 is shown, which is the outcome of the left image. Referring to FIG. 9B, RT-PCR using systemically infected tissues by CY2220_(1132-1329)_2280 is shown. In the gel image, the size of PCR bands from infected tissues were identical to the positive control (plasmid construct). PCR was performed using a primer set targeting the region from positions 1996 to 2452. Referring to FIG. 9C, sequencing results are shown of PCR products and their alignment to the original sequence, CYVaV 1132-1329. The presented sequence of the duplicated-insert (1132-1329) corresponds to SEQ ID NO:1 (1132-1329). The presented sequence of CY2220 (1132-1329)2280-1 and CY2220 (1132-1329)2280-2 are identical (SEQ ID NO:15):

attgctcgagcttcgttgcaguaaaagguuguauggugagggugcugag
ccguguaucgccaaaggccuaaaugcauuagaaucuggagcacuuugag
gcgcaaaugggagaaguuuucuucuccaguuugcguuucucucgacgcu
uccagguucgaccugcauguaagcguuggcaugcuaaaguucacacaca
agcuauaugacuauuacugcagttaatgtaggtgtctttccgt

Referring to FIG. 9D, for more detailed information about the stability of the internally duplicated CYVaV construct, the PCR products from infected tissue were cloned into pMiniT2.0 cloning vector and a total of 44 clones were sequenced to get higher resolution for virus population after long-term infection. The sequencing information revealed that most of the population of the virus was very stable. The internally copied-insert of 29 clones out of 44 samples was identical to the original sequence. 13 out of 44 clones only had single nucleotide changes. The single base change written in black parentheses indicates that the base change happened in a single clone. Among the 13 clones, 2 clones had the same base change at nucleotide location 119 [(T119C)X2]. Multiple base changes in individual clones were marked as the same color. 1 out of 44 clones had two base changes and was marked by orange color, C49T, T111C. 1 out of 44 clones had three base changes and was marked by green color, T111C, T121C and T120C. Except for T113C and T187C, most of the base changes happened near the boundary between loop and stem. There was no major deletion or substitution from all of the samples. The polynucleotide sequence presented in FIG. 9D (SEQ ID NO:16):

cagtaaaaggttgtatggtgagggtgctgagccgtgtatcgccaaaggc
ctaaatgcattagaatctggagcgactttgaggcgcaaatgggagaagt
tttcttctccagtttgcgtttctctcgacgcttccaggttcgacctgca
tgtaagcgttggcatgctaaagttcacacacaagctatatgactattac
tg

FIGS. 10A-10E show sequence alignment results of samples of FIGS. 9A-9E thereof. The presented sequence of the Duplicated-insert (1132-1329) corresponds to SEQ ID NO:1 (1132-1329). The presented sequences of CBC135, CBC135, CBC137, CBC138, CBC142, CBC143, CBC144, CDE966, CDE968, CDE969, CDE970, CDE972, CDE975, CDE979, CDF018, CDF019, CDF022, CDF023, CDF024, CDF025, CDF026, CDF030, CDF031, CDF032, CDF038, CDF039, CDF040, CDF043, CDF044, and CDF045 are identical (SEQ ID NO:17):

ggtcattggtttaccgatgatacctgttcagaataggattgctcgagct
tcgttgcagtaaaaggttgtatggtgagggtgctgagccgtgtatcgcc
aaaggcctaaatgcattagaatctggagcgactttgaggcgcaaatggg
agaagttttcttctccagtttgcgtttctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CBC139 (G22T) is (SEQ ID NO:18):

ggtcattggtttaccgatgatacctgttcagaataggattgctcgagct
tcgttgcagtaaaaggttgtatggtgatggtgctgagccgtgtatcgcc
aaaggcctaaatgcattagaatctggagcgactttgaggcgcaaatggg
agaagttttcttctccagtttgcgtttctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CBC140 (C33T) is (SEQ ID NO:19):

ggtcattggtttaccgatgatacctgttcagaataggattgctcgagct
tcgttgcagtaaaaggttgtatggtgagggtgctgagctgtgtatcgcc
aaaggcctaaatgcattagaatctggagcgactttgaggcgcaaatggg
agaagttttcttctccagtttgcgtttctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDE967 (T119C) is (SEQ ID NO:20):

ggtcattggtttaccgatgatacctgttcagaataggattgctcgagct
tcgttgcagtaaaaggttgtatggtgagggtgctgagccgtgtatcgcc
aaaggcctaaatgcattagaatctggagcgactttgaggcgcaaatggg
agaagttttcttctccagtttgcgttcctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDE974 (G62C) is (SEQ ID NO:21):

ggtcattggtttaccgatgatacctgttcagaataggattgctcgagct
tcgttgcagtaaaaggttgtatggtgagggtgctgagccgtgtatcgcc
aaaggcctaaatgcattacaatctggagcgactttgaggcgcaaatggg
agaagttttcttctccagtttgcgtttctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDE977 (T111C, T121C, T170C) is (SEQ ID NO:22):

ggtcattggtttaccgatgatacctgttcagaataggattgctcgagct
tcgttgcagtaaaaggttgtatggtgagggtgctgagccgtgtatcgcc
aaaggcctaaatgcattagaatctggagcgactttgaggcgcaaatggg
agaagttttcttctccagcttgcgtttccctcgacgcttccaggttcga
cctgcatgtaagcgttggcatgctaaagctcacacacaagctatatgac
tattactgcagttaatgtaggtgtctttccgtatctagtcacgatggta
agcaac

The presented sequence of CDE978 (T35C) is (SEQ ID NO:23):

ggtcattggtttaccgatgatacctgttcagaataggattgctcgagct
tcgttgcagtaaaaggttgtatggtgagggtgctgagccgcgtatcgcc
aaaggcctaaatgcattagaatctggagcgactttgaggcgcaaatggg
agaagttttcttctccagtttgcgtttctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDE980 (C49T, T111C) is (SEQ ID NO:24):

ggtcattggtttaccgatgatacctgttcagaataggattgctcgagcttcgttgcagtaaaaggttgtatggtgagggtg
ctgagccgtgtatcgccaaaggtctaaatgcattagaatctggagcgactttgaggcgcaaatgggagaagttttcttc
tccagcttgcgtttctctcgacgcttccaggttcgacctgcatgtaagcgttggcatgctaaagttcacacacaagctata
tgactattactgcagttaatgtaggtgtctttccgtatctagtcacgatggtaagcaac

The presented sequence of CDF015 (C174T) is (SEQ ID NO:25):

ggtcattggtttaccgatgatacctgttcagaataggattgctcgagcttcgttgcagtaaaaggttgtatggtgagggtg
ctgagccgtgtatcgccaaaggcctaaatgcattagaatctggagcgactttgaggcgcaaatgggagaagttttcttc
tccagtttgcgtttctctcgacgcttccaggttcgacctgcatgtaagcgttggcatgctaaagttcatacacaagctatat
gactattactgcagttaatgtaggtgtctttccgtatctagtcacgatggtaagcaac

The presented sequence of CDF016 (G125A) is (SEQ ID NO:26):

ggtcattggtttaccgatgatacctgttcagaataggattgctcgagcttcgttgcagtaaaaggttgtatggtgagggtg
ctgagccgtgtatcgccaaaggcctaaatgcattagaatctggagcgactttgaggcgcaaatgggagaagttttcttc
tccagtttgcgtttctctcaacgcttccaggttcgacctgcatgtaagcgttggcatgctaaagttcacacacaagctata
tgactattactgcagttaatgtaggtgtctttccgtatctagtcacgatggtaagcaac

The presented sequence of CDF017 (C57T) is (SEQ ID NO:27):

ggtcattggtttaccgatgatacctgttcagaataggattgctcgagcttcgttgcagtaaaaggttgtatggtgagggtg
ctgagccgtgtatcgccaaaggcctaaatgtattagaatctggagcgactttgaggcgcaaatgggagaagttttcttc
tccagtttgcgtttctctcgacgcttccaggttcgacctgcatgtaagcgttggcatgctaaagttcacacacaagctata
tgactattactgcagttaatgtaggtgtctttccgtatctagtcacgatggtaagcaac

The presented sequence of CDF020 (T148C) is (SEQ ID NO:28):

ggtcattggtttaccgatgatacctgttcagaataggattgctcgagcttcgttgcagtaaaaggttgtatggtgagggtg
ctgagccgtgtatcgccaaaggcctaaatgcattagaatctggagcgactttgaggcgcaaatgggagaagttttcttc
tccagtttgcgtttctctcgacgcttccaggttcgacctgcacgtaagcgttggcatgctaaagttcacacacaagctata
tgactattactgcagttaatgtaggtgtctttccgtatctagtcacgatggtaagcaac

The presented sequence of CDF037 (T119C) is (SEQ ID NO:29):

ggtcattggtttaccgatgatacctgttcagaataggattgctcgagcttcgttgcagtaaaaggttgtatggtgagggtg
ctgagccgtgtatcgccaaaggcctaaatgcattagaatctggagcgactttgaggcgcaaatgggagaagttttcttc
tccagtttgcgttcctctcgacgcttccaggttcgacctgcatgtaagcgttggcatgctaaagttcacacacaagctata
tgactattactgcagttaatgtaggtgtctttccgtatctagtcacgatggtaagcaac

The presented sequence of CDF041 (T187C) is (SEQ ID NO:30):

ggtcattggtttaccgatgatacctgttcagaataggattgctcgagcttcgttgcagtaaaaggttgtatggtgagggtg
ctgagccgtgtatcgccaaaggcctaaatgcattagaatctggagcgactttgaggcgcaaatgggagaagttttcttc
tccagtttgcgtttctctcgacgcttccaggttcgacctgcatgtaagcgttggcatgctaaagttcacacacaagctata
cgactattactgcagttaatgtaggtgtctttccgtatctagtcacgatggtaagcaac

The presented sequence of CDF047 (T113C) is (SEQ ID NO:31):

ggtcattggtttaccgatgatacctgttcagaataggattgctcgagcttcgttgcagtaaaaggttgtatggtgagggtg
ctgagccgtgtatcgccaaaggcctaaatgcattagaatctggagcgactttgaggcgcaaatgggagaagttttcttc
tccagttcgcgtttctctcgacgcttccaggttcgacctgcatgtaagcgttggcatgctaaagttcacacacaagctata
tgactattactgcagttaatgtaggtgtctttccgtatctagtcacgatggtaagcaac

The presented sequence of CDF049 (T101C) is (SEQ ID NO:32):

Ggtcattggtttaccgatgatacctgttcagaataggattgctcgagcttcgttgcagtaaaaggttgtatggtgagggt
gctgagccgtgtatcgccaaaggcctaaatgcattagaatctgg4agcgactttgaggcgcaaatgggagaagtttc
cttctccagtttgcgtttctctcgacgcttccaggttcgacctgcatgtaagcgttggcatgctaaagttcacacacaagc
tatatgactattactgcagttaatgtaggtgtctttccgtatctagtcacgatggtaagcaac

FIG. 11A shows wild-type hairpin-like structures of CY1 ranging in size from 33 to 198 nt (labeled 1 to 4) of FIG. 49 and shows the sequence and secondary structure of a hairpin-like insert mmck6.2 (SEQ ID NO:33).

FIG. 11B shows RT-PCR analysis and Sanger sequencing chromatographs from Mexican lime leaves infected with recombinant CYVaV1 having mmck6.2 after 12 months.

FIG. 11C shows: Top Northern blot of RNA extracted from two infected citrus plants after about one year of infection with recombinant CY1 including mmck6.2. Bottom. Reverse-transcriptase (RT)-PCR is used to amplify a segment of the infecting VIGS vector. For this plant, 10 leaves were assayed. Only a single product of the correct size was found after 15 months of infection. FIG. 11D shows Total RNA from young leaves of the infected plant was subjected to batch sequencing of the insert region. Top box is from 15-month citrus showing no indication of single “double peaks” that would denote base changes or multiple double peaks that would indicate a portion of the population contain deletions. In addition, at least 10 clones were generated for individual sequencing and no changes from the initial VIGS vector were found. FIG. 11E. Photo of infected Mexican lime plant. Nucleotide sequence of insert: gtttcagtatttcgtcatcaccttcctaacggaagtgatggacgaaattaatgaaacc (SEQ ID NO:34).

FIG. 12A. Flow chart for how the data in B was generated. Note that color coding shown on the inserts (from RNAFold) are relative entropy values for the specific insert and go from low entropy to high entropy. Positional entropy values for each nucleotide, obtained from the folding program, go from 0 (white) to 2.6 (dark gray) and are absolute values that allow for comparisons between hairpins. Positional entropy values for individual residues are then rearranged for better visualization of the entire insert (far right). FIG. 12B. APE values for various hairpin-like structures (and some additional non-hairpin-like structures) inserted into CY1. Sorted positional entropy is shown for each insert (dark gray to lightest gray). The first four hairpin-like structures (boxed) were natural duplicates of hairpin-like structures 1 through 4. Other hairpin-like structures and non-hairpin-like inserts are arranged in order of descending APE (from 1.30 to 0.07). Mimic hairpin-like structures begin with a number (1 or 4) that identifies the hairpin-like structure being mimicked. Thick lines beneath each lane denote the length of each hairpin (number of nucleotides shown at right). Asterisks denote inserts that were not stable in the VIGS vector. Double asterisk denotes a hairpin-like structure that had three consecutive G:C pairs at the base and five consecutive G:C pairs in the apical region contributing to the very low APE. Deletion of two nucleotides on the 5′ side and two nucleotide changes (G to C) on the 3′ side of the hairpin-like structure in the apical region that eliminated the 5 consecutive G-C pairs generated a stable hairpin-like structure with no consecutive apical G:C pairs and the APE value changed to 0.15. Thick black arrow denotes an unstable hairpin-like insert with a relatively low APE value of 0.39 that contained a large number of higher entropy nucleotides.

FIG. 13 shows some elements of RNA secondary structure.

FIGS. 14A-14E shows sequences and secondary structure of wild type CY1 hairpin-like structure 4 of FIG. 49 (SEQ ID NO:35) (FIG. 14A) and exogenous hairpin-like structures mmck15 (SEQ ID NO:36) (FIG. 14B); mmck8 (SEQ ID NO:37) (FIG. 14C); mmckpsvD (SEQ ID NO:38) (FIG. 14D); and mmckpsvE (SEQ ID NO:39) (FIG. 14E).

FIGS. 15A-15C show sequences and secondary structure of wild type CY1 hairpin-like structure 4 of FIG. 49 (SEQ ID NO:35) (FIG. 15A) and exogenous hairpin-like structures PRSVmmck1 (SEQ ID NO:40) (FIG. 15B) and PSRVmmck2 (SEQ ID NO:41) (FIG. 15C).

FIGS. 16A-16C show sequences and secondary structure of wild type CY1 hairpin-like structure 1 of FIG. 49 (SEQ ID NO:42) (FIG. 16A) and exogenous hairpin-like structures PDS-mmck-1 (SEQ ID NO:43) (FIG. 16B) and PDS-mmck-2 (SEQ ID NO:44) (FIG. 16C).

FIGS. 17A-17C show sequences and secondary structure of wild type CY1 hairpin-like structure 1 of FIG. 49 (SEQ ID NO:42) (FIG. 17A) and exogenous hairpin-like structures LcrGyr-mmck5 (SEQ ID NO:45) (FIG. 17B) and LcrGyr-mmck3 (SEQ ID NO:46) (FIG. 17C).

FIGS. 18A-18C show sequences and secondary structure of A—wild type CY1 hairpin-like structure 1 of FIG. 49 (SEQ ID NO:42) (FIG. 18A) and exogenous hairpin-like structures B—Clas-GyrAmmck-5′ (SEQ ID NO:47) (FIG. 18B) and C—Clas-GyrAmmck-3′ (SEQ ID NO:48) (FIG. 18C).

FIGS. 19A-19B show sequences and secondary structure of wild type CY1 hairpin-like structure 1 of FIG. 49 (SEQ ID NO:42) (FIG. 19A) and exogenous hairpin-like structures of CS 7 (SEQ ID NO:49) (FIG. 19B).

FIGS. 20A-20B show sequences and secondary structure of wild type CY1 hairpin-like structure 1 of FIG. 49 (SEQ ID NO:42) (FIG. 20A) and exogenous hairpin-like structures B—Erwinia GyrA (SEQ ID NO:50) (FIG. 20B).

FIG. 21 shows SHAPE changes resulting from insertion of CY2301GFP30sh into CY1. The presented polynucleotide sequence is SEQ ID NO:51.

FIG. 22 shows the sequence and secondary structure of M2250GFP30ext, an unstable insert targeting GFP. The presented polynucleotide sequence is (SEQ ID NO:52):

gguuaggguaacucacugaagcggcacgacuucuucaagagcgccauuc
agugagcaaccuaacc.

FIG. 23 shows the sequence and secondary structure of GFPmmck59, a stable insert targeting GFP. The presented polynucleotide sequence is (SEQ ID NO:53):

tgaagcggcacgacttcttcaagagcgataactcgccttgacagaagtc
caacgcttca.

FIG. 24 shows the sequence and secondary structure of GFPmmck63, an unstable insert targeting GFP. The presented polynucleotide sequence is (SEQ ID NO:54):

tgaagcggcacgacttcttcaagagcgccataatcggcgccttgacaga
agtccaacgcttca.

FIG. 25 shows the sequence and secondary structure of CTV-insert-natural-V2.2, a naturally occurring hairpin-like structure in CTV. The presented polynucleotide sequence is (SEQ ID NO:55):

ggggguuuauguuuggcaaagaaaguguuggaacuguuagucaagcggg
ugguugaaucguuuucucguuugaagcggaaaaccgcucguuuaacguc
cuucgcuaauuuguugcuugcgaggcucuc

FIGS. 26A and 26B show RT-PCR and Sanger data for recombinant CYVaV1 with insert-natural-V2.2 infecting N. benthamiana after 3 weeks. The presented polynucleotide sequence is (SEQ ID NO:56):

tcgttgggggtttatgtttgcaaagaaagtgttggaactgttagtcaag
cgggtggttgaatcgtttctcgtttgaagcggaaaaccgctcgtttaac
gtccttcgctaatttgttgcttgcgaggctctcagttaat

FIG. 27 shows an unstable insert BBLv2-1 (SEQ ID NO. 57 and SEQ ID NO:58).

FIG. 28 shows the addition of a heterologous insert including a cleavage site and a peptide into the RdRp of CY1.

FIG. 29 shows siRNA generated from CTV targeting inserts quantified by stem-loop RT-PCR from leaves collected from locations in multiple plants.

FIG. 30 shows siRNA generated from CTV-targeting inserts quantified by stem-loop RT-PCR from young and mature leaves collected from citrus plants.

FIG. 31 shows a wild type hairpin-like structure of CY1 and hairpin-like structures targeted against DMR6 genes.

FIG. 32 shows a wild type hairpin-like structure of CY1 and hairpin-like structures targeted against MLO genes

FIG. 33, for poliovirus and CVB3, a wild type region and two inserts were designed, one stabilized according to the methods described herein which includes adjustment of the hairpin stability and in these examples surrounding sequence modification, and one control construct with straightforward fully base-paired hairpin insertion in the viral genome.

FIG. 34 shows, for the control poliovirus construct, sequence heterogeneity observed in passage 0.

FIG. 35 shows, for the stabilized insert in the poliovirus genome, sequence heterogenicity observed in passages 0 and 10.

FIG. 36 shows, for the control insert in the CVB3 genome, sequence heterogenicity observed in passages 0, 5 and 10 and for the stabilized insert in the CVB3 genome, sequence heterogenicity observed in passages 0 and 10.

FIG. 37 shows the secondary structure of the CVB3 insert and the location of the deletions and insertions.

FIG. 38 shows the secondary structure of the poliovirus insert and the location of the deletions and insertions.

FIG. 39 shows the secondary structure of full-length CY1 wherein numbers 1 to 4 denote natural CY1 hairpin-like structures.

FIGS. 40A-40F show an analysis of thermodynamic characteristics of various substructures. FIG. 40A shows the relationship between MFE and length of 38,577 random substructures of various lengths and complementarity. Slopes for each complementarity set are shown and graphed along with the line generated by CY1 hairpin-like structures (solids line). FIG. 40B shows a graph comparing between MFE/length and the average PE of the random substructures. The four CY1 hairpin-like substructures used as natural insertions are shown as dots labelled H1, H2, H3, H4. FIG. 40C shows the number of apical loops present in the predicted folding of the randomly generated substructures. Based on the slope found for CY1 hairpin-like structures in FIG. 40A, the estimated position for CY1 hairpin-like structures is indicated by an arrow. Error bars represent standard error of 3,500 hairpins. FIG. 40D shows the relationship between MFE and length of the CY1 hairpin-like structures (solid line), TBSV hairpin-like substructures (upper dashed line) and HCV hairpin-like substructures (lowed dashed line) from solved secondary structures. FIG. 40E shows the relationship between MFE and length of CY1 hairpin-like structures (solid line) and human ribosomal RNA (lower dashed line) from solved structures, along with predicted hairpin-like structures found in RNA transcribed from DNA viruses and plant mRNA (overlapping upper dashed line). Shaded areas in FIG. 40D and FIG. 40E represent standard error of the linear regression. FIG. 40F shows linear regressions of MFE/length of hairpin-like structures in the predicted structures of CY1 and other +RNA viruses.

FIG. 41 shows the genomic arraignment of a Tobacco Rattle Virus (TRV) vector, insert locations, and a method of infecting plants with the vector.

FIG. 42 shows the genomic arraignment of a Potato Virus X (PVX) vector, insert locations, and a method of infecting plants with the vector.

FIG. 43 shows three hairpin-like regions, H1-H4, from CY1 and two synthetic hairpin-like inserts Ftsz102 and Ftsz69.

FIG. 44 shows stability or instability of the hairpin-like regions of FIG. 43 when inserted in PVX (upper panels) and TRV (lower panels).

FIG. 45 shows three more synthetic hairpin-like inserts Ftsz160, Ftsz159 and Ftszrc160.

FIG. 46 shows the stability or instability H1-H4, Ftszrc160 and Ftsz160 as inserts in a tobacco rattle virus (TRV) vector.

FIG. 47 shows results of testing H1, Ftszrc160 and Ftsz160 in a potato virus X (PVX) vector.

FIG. 48 shows results of an experiment that examined small incremental differences in hairpin thermodynamic stability and their effect on maintenance in virus CY1.

FIG. 49 shows stabilizing changes made to one of the unstable inserts (C3) of FIG. 48.

FIG. 50 shows the effects of making hairpins gradually less thermodynamically stable (while maintaining a low value for the average positional entropy).

FIG. 51 shows an increase in stability upon deleting a repeated base sequence from an insert.

FIG. 52 shows a large, stable, insert made by stacking three smaller stable inserts.

FIG. 53 shows deletions of parts of hairpins with high APE.

FIG. 54 shows the stability of the stacked form of a fully base paired structure (but for the apical loop) that defined as stable previously.

FIG. 55 shows another fully base-paired (but for the apical loop) hairpin PDS51 that was found to be stable for 3 weeks when inserted at position 2301 of CY1.

FIG. 56 shows the secondary structure of branched insert m2.

FIG. 57 shows the secondary structure of branched insert H4+(H1/Ftsz159).

### DETAILED DESCRIPTION OF EMBODIMENTS

### Definitions

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. For example, the term “a virus” includes a single virus as well as a plurality or population of the virus. Generally, nomenclatures utilized in connection with, and techniques of molecular biology, protein and oligonucleotide or polynucleotide chemistry and hybridization described herein are those well-known and commonly used in the art.

The term “or” is generally employed in its sense including “and/or” unless the content clearly dictates otherwise.

The recitation of numerical ranges by endpoints herein includes all numbers and fractions subsumed within that range (e.g., 1 to 5 includes 1, 1.5, 2, 2.75, 3, 3.90, 4, and 5).

As used herein, the term “targeting” and variations thereof, refers to a sequence that inhibits a pathogen or modifies a biological function in a host. In some embodiments, a targeting sequence specifically binds, or produces in the host a molecule that specifically binds, a target sequence, molecule or cell. “Specifically binding” refers to having greater affinity to a particular molecule or cell of interest relative to alternative sequences, molecules or cell types. In some embodiments, a targeting sequence encodes a peptide, for example an antimicrobial peptide. In some embodiments, a targeting sequence produces a bioactive RNA molecule, for examples an siRNA.

The terms “mutation” or “modification” as used herein refers to insertions, substitutions or deletions of one or more nucleotides within a sequence.

The term “sequence identity” as used herein, unless stated otherwise, refers to the percentage of sequence identity between two amino acid sequences or two nucleic acid sequences. To determine the percent identity of two amino acid sequences or of two nucleic acid sequences, the sequences are aligned for optimal comparison purposes (e.g. gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=[number of identical overlapping positions]/[total number of positions]×100%). In one embodiment, the two sequences are the same length. The determination of percent identity between two sequences can also be accomplished using a mathematical algorithm. One non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990. BLAST nucleotide searches can be performed with the NBLAST nucleotide program parameters set, e.g. for score=100, wordlength=12 to obtain nucleotide sequences homologous to a nucleic acid molecules of the present disclosure. BLAST protein searches can be performed with the XBLAST program parameters set, e.g. to score-50, wordlength=3 to obtain amino acid sequences homologous to a protein molecule of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules. When utilizing BLAST, Gapped BLAST, and PSI-Blast programs, the default parameters of the respective programs (e.g. of XBLAST and NBLAST) can be used (see, e.g. the NCBI website). Another non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

The term “effective amount” as used herein refers to any amount of a compound or a composition that is sufficient to generate a desired response, including but not limited to preventing or reducing the chance of disease onset, slowing disease progression, and alleviating symptoms.

The term “wildtype” or “native” as used herein refers to a reference genotype, including a genotype that predominates in a natural population or a laboratory population as compared to a natural or a laboratory mutant form, and also includes an isolate or consensus sequence.

The term “functional variant” as used herein includes modifications of the polypeptide sequences disclosed herein that perform substantially the same function as the polypeptide molecules disclosed herein in substantially the same way. For example, a nucleotide sequence encoding the functional variant may comprise sequences having at least 50%, at least 60%, at least 70%, at least 80%, or at least 90%, or at least 95% sequence identity to the sequences disclosed herein. The functional variant may also comprise conservatively substituted amino acid sequences of the sequences disclosed herein.

The terms “heterologous segment” or “heterologous RNA segment” as used herein refer to a nucleotide sequence that is not naturally present in a particular reference molecule, for example the genome of a wild type virus, from which a vector or other RNA molecule containing the heterologous segment may be derived. The heterologous segment may contain an active portion which may be derived from, or may be complementary to, or a sequence present in another organism, for example a host of the virus or a pathogen of the host. The heterologous segment may also contain a portion that completes a hairpin-like structure with the active portion. The heterologous segment may provide any coding or non-coding function appropriate for a particular application. The terms “heterologous” and “exogenous” have been used herein interchangeably.

The term “homolog” as used herein refers to the same sequence in a related species such as the same sequence in a different virus. Typically homologs share a high degree of sequence identity, such as at least 50%, 60%, 70% or more. The homology between two sequences that are derived from species which are more closely related is typically higher than from more distantly related species.

As used herein, the terms “vector” or “RNA vector” refer to an RNA molecule containing a heterologous segment with an active portion that performs a desired function in a host. The desired function may involve, for example, regulation of the host (e.g. host gene downregulation), inhibiting a pathogen of the host (optionally including in the case of plants and insect feeding on the plant), or modifying replication of the vector in the host (i.e. attenuating replication of the vector itself as in a live attenuated vaccine). The RNA vector may be, for example, a viral vector or a sub-viral vector. In some examples, the RNA vector is a plus-sense single stranded RNA, wherein the term single stranded RNA may include folded RNA with double stranded or base-paired regions. The RNA vector may be derived from a virus or from sub-viral RNA. For example, an RNA vector may be derived from a plant virus such as CY1, CY2, another ULV, CTV, PVX or, TRV.

As used herein, a “host” refers to a cell, tissue or organism capable of being infected by and capable of replicating a nucleic acid. A host may include a whole plant, a plant organ, plant tissue, a plant protoplast, and a plant cell. A plant organ refers to a distinct and visibly differentiated part of a plant, such as root, stem, leaf, seed, graft or scion. Plant tissue refers to any tissue of a plant in whole or in part. Protoplast refers to an isolated cell without cell walls, having the potency for regeneration into cell culture, tissue or whole plant. Plant cell refers to the structural and physiological unit of plants, consisting of a protoplast and the cell wall. Alternatively, a “host” may include an animal, such as a human, or an organ or cell thereof.

As used herein, “nucleic acid sequence,” “polynucleotide,” “nucleotide” and “oligonucleotide” are used interchangeably and refer to a polymeric form of nucleotides of any length. Polynucleotides may have any three-dimensional structure, and may perform any function. A “gene” refers to a polynucleotide containing at least one open reading frame that is capable of encoding a particular polypeptide sequence. “Expression” refers to the process by which a polynucleotide is transcribed into mRNA and/or the process by which the transcribed mRNA is translated into peptides, polypeptides, or proteins.

RNA sequences presented in this specification or the figures may use either “t” or “u” to denote uracil. Sequences may be presented in upper case or lower case letters. There is no significance attached to the case (upper case or lower case) of the sequence unless noted in the description of the sequence.

A vector “derived from” a particular molecule, for example the genomic RNA of a virus, means that the vector contains genetic elements or sequence portions from such molecule. In some embodiments, the vector comprises a replicase open reading frame (ORF) from such molecule. The resulting vector may be capable of replicating in plant or animal cells. A vector may be constructed from the molecule from which it is derived. One or more heterologous segment(s) may be added as an additional sequence to the vector. The backbone of a vector (i.e. excluding any heterologous segments) may have 60% or more, 70% or more, 75% or more, 80% or more, 85% or more, 90% or more, 95% or more or 98% or more sequence identity with the RNA molecule (e.g. the genomic RNA of a wild type virus) from which it is derived.

As used herein, the terms “infection” or “capable of infecting,” with respect to a vector of the present invention, includes the ability of such vector to transfer or introduce its nucleic acid into a host, such that the nucleic acid or portion(s) thereof is replicated and/or proteins or other agents are synthesized or delivered in the host. Infection also includes the ability of a selected nucleic acid sequence to integrate into a genome of a target host.

As used herein, the term “endogenous” refers to a polypeptide, nucleic acid or gene that is expressed by a host.

As used herein, the term “hairpin” refers to a primarily base paired segment of RNA that comprises a fully base-paired region (alternatively called a “stack”) and a non-base paired region (alternatively called “an apical loop”) between the bases on opposite sides of the stack.

As used herein, the term “hairpin-like structure” refers to a primarily base paired segment of RNA having multiple stacks separated by non-base-paired regions, alternatively called “bulges” or “internal loops” (sometimes called “loops” for brevity when the distinction from an apical loop is apparent from the context), and an apical loop between the bases on opposite sides of the stacks. A loop may be symmetric, with an equal number of bases on opposite sides of the loop, or asymmetric, with a different number of bases on opposite sides of the loop. The term “bulge” may be used as an alternative name for a loop, or may be used in some contexts to refer specifically to an asymmetric loop having one or more non-paired bases on only one side. Most hairpin-like structures are linear (i.e. unbranched or without junctions) but in some embodiments a hairpin-like structure is Y-shaped, T-shaped or otherwise branched. The linear branches of a branched structure may also be hairpin-like structures themselves.

As used herein, the term “junction” refers to a non-base-paired region that separates three or more stacks. A simple junction separating three stacks may create, for example, a Y-shaped or T-shaped secondary structure. A stack connected to a junction may be part of a hairpin, a hairpin-like structure, or a stem.

As used herein, the term “stem” refers to a structure that extends from a junction that does not terminate in an apical loop. A stem may connect a junction to another junction, or may connect a junction to the single stranded regions between stems (i.e., single stranded regions that are not apical loops, internal loops or junctions). Referring to FIG. 53, the junction separates a stem below the junction from two hairpin-like structures above the junction.

As used herein, the term “movement protein” refers to a protein(s) required for cell-to-cell and/or long distance movement in a plant. “Coat protein” refers to protein(s) comprising or building the virus coat.

As used herein, “virus” includes traditional or conventional viruses and related infectious agents that might or might not satisfy all elements of some definitions of a virus, for example because they do not encode a coat protein and/or a movement protein. For example, plant viruses include ULV, for example CY1. Virus also includes derivatives, sub viruses, variants or parts of a virus, for example defective RNA, subgenomic RNA and RNA multimers (e.g. dimers or trimers), that may be infectious in a host alone or in the presence of the full virus or a helper virus.

The terms “about”, “substantially”, “essentially”, “approximately” and similar terms as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies. Further, unless stated otherwise or indicated by the context, elements not explicitly modified by the terms “about”, “substantially” and “approximately” include a similar reasonable amount of deviation. In the context of sequence identity, sequence described using the terms “about”, “substantially”, “essentially”, “approximately” and similar terms, or not explicitly modified by these terms, include sequences having 70% or more, 80% or more, 90% or more, or 95% or more sequence identity with a reference sequences, unless stated otherwise or implied otherwise, for example because a larger degree of identity is required to provide a stated function or structure.

As used herein, the term “secondary structure” describes the folding of single stranded RNA into base-paired and non-base-paired regions. Hairpins, hairpin-like structures and junctions are elements of the secondary structure of RNA molecules. An RNA molecule may also have a tertiary structure. The tertiary structure includes interactions, i.e. base pairing, of segments of the RNA separated in a manner other than by way of junctions or apical loops. In some cases the tertiary structure includes long-distance interactions between RNA segments separated by many bases and/or intermediate hairpins or hairpin-like structures.

The term “RNA bioactive molecule” or “bioactive RNA” refers to any biologically active RNA molecule. In an embodiment, the RNA bioactive molecule is an RNAi effector molecule for inducing an RNA interference response. In an embodiment, the RNAi effector molecule is short interfering RNA (siRNA), microRNA (miRNA), long hairpin (lhRNA), short hairpin (shRNA), double stranded (dsRNA), or anti-sense RNA. In an embodiment, the RNAi effector molecule is dsRNA. In another embodiment, the RNAi effector molecule is lhRNA. The terms “RNA interference,” “interfering RNA” or “RNAi” refer to single-stranded RNA or double-stranded RNA (dsRNA) that is capable of reducing or inhibiting expression of a target nucleic acid.

The term “antisense RNA” refers to a single stranded RNA that is complementary to messenger RNA and that hybridizes with the messenger RNA blocking translation into protein.

The term “long hairpin RNA” or “lhRNA” as used herein refers to a long inhibitor RNA that can be used to reduce or inhibit expression of a target nucleic acid by RNA interference. LhRNA are typically single stranded with secondary structure (hairpin) and longer than 60 nucleotides. Total length may be 1000 base pairs or more.

The term “siRNA” or “siRNA oligonucleotide” refers to a short inhibitory RNA that can be used to reduce or inhibit nucleic acid expression of a specific nucleic acid by RNA interference. The siRNA can be, for example, a duplex, a short RNA hairpin (shRNA) or a microRNA (miRNA).

### Stabilizing Inserts in RNA

Inserts may be added to RNA for various purposes. In one application, inserts added to an RNA virus may be used to deliver siRNAs into plants. Sequences inserted into RNA virus genomes are converted into siRNAs when viruses are targeted by plant RNA silencing defenses. This technique has been used in labs to down-regulate the expression of specific genes for short periods of time, for days to a few weeks, after which the virus replicates into a form without the inserted sequence. In animal viruses, previous inserts may be maintained for only one passage. For use in agricultural or animal treatments it is desirable for the inserted sequence to be maintained by the virus for a longer period of time.

RNA folds to contain partially base-paired substructures within a complete secondary structure of the RNA. In at least some cases, an RNA structure can assume multiple secondary structures, of which the most frequently appearing secondary structure is considered to be the characteristic secondary structure or shape of the RNA. In some cases, the RNA may also need to be unfolded at times, for example during replication. The overall time that a sequence spends in a shape, for example its characteristic shape, reflects the thermodynamic stability of that shape. The thermodynamic stability of a substructure is affected by Gibbs free energy (also called minimum free energy or MFE or DeltaG), which may be related to hydrogen bonds and base stacking. The thermodynamic stability is also affected by the positional entropy (PE) of its RNA nucleotides. PE=0 if a nucleotide is always paired with the same partner or always unpaired. PE=2.6 if a nucleotide has multiple possible partners. The average positional entropy (APE) of an RNA substructure may also be calculated.

Without intended to be limited by theory, it may be that viral genomes have evolved such that RNA substructures, such as unbranched hairpin-like structures, have thermodynamic properties, such as ranges of APE and MFE/length, that make the genomic RNA a fit template for replication and/or other functions. Accordingly, natural substructures in both coding and non-coding regions should already possess the appropriate thermodynamic properties, as non-persistence of at least coding-region substructures would be lethal. When engineered inserts without the appropriate thermodynamic properties are added to a viral genome, the genome rapidly evolves (possibly due to an evolved degree of an error prone quality of the RdRp) to eliminate or modify the insert to restore fitness, and the evolved virus may out-compete the engineered virus and become dominant in a population of the virus in a host. Engineered inserts without the appropriate thermodynamic properties may also cause undesirable conformation changes in the secondary structure of the vector near the insertion site. Conversely, if the engineered insert has appropriate thermodynamic properties, it will be retained in the viral genome for a longer period of time in the host. We also predict that other types of RNA, possibly due to other evolutionary factors or requirements of fitness, have evolved such that their RNA substructures also have thermodynamic properties, such as ranges of APE and MFE/length, that make them fit for their required functions. Engineered inserts having appropriate thermodynamic properties are stable when inserted in non-functional regions of a viral genome, vector backbone or other functional nucleotide.

Analysis of wild type plus sense RNA substructures also suggests that the overall architecture of these substructures includes a continuous alternation between short base-paired regions and short unpaired regions (i.e. bulges, internal loops and apical loops). For example, in CY1 substructures the longest consecutive base-paired stretch has 13 base-pairs. There are only 6 regions in the genome with more than 10 non-Watson-Crick paired residues, the longest of which spans 21 bases. While it is not necessary for an engineered insert to exactly mimic the arrangement of base-paired regions and unpaired regions in a wild-type genome, the engineered insert may optionally have a continuous alternation between short base-paired regions (i.e. 13 base-pairs or less) and short (i.e. 21 residues or less or 10 residues or less) unpaired regions.

### Vectors and Heterologous Segments

This specification describes heterologous segments (alternatively called inserts) provided in RNA vectors that are stable in replication, for example more stable than fully base paired hairpins carrying the same active portion. The vectors are typically stable in replication (i.e. progeny of the vector carry the heterologous insert with minimal mutation) for 3 weeks or more but many embodiments are stable for longer periods of time, for example 3 months or more. The inserts are described as “hairpin-like” to indicate that they include base-paired segments but are not fully base-paired beyond an apical loop.

Accordingly, provided herein is a ribonucleic acid (RNA) vector comprising a segment derived from a virus and a heterologous segment(s) inserted therein, wherein the heterologous segment comprises a) two or more base-paired regions, b) one or more non-base-paired regions separating the base-paired regions, and c) an apical loop at the end of one of the based paired regions. The heterologous segment is designed according to one or more parameters or rules described herein.

In some embodiments, a hairpin-like region of an RNA vector includes a hairpin-like heterologous segment appended to a truncated native hairpin-like segment. In these cases, the parameters or rules described herein may be applied to the appended heterologous segment alone, or to the entire resulting hairpin-like segment combining the heterologous segment and the truncated native segment.

In some embodiments, the parameters or rules are defined with reference to one or more hairpin-like regions in a wild type RNA virus from which the RNA vector is derived. However, in other embodiments, the parameters or rules are determined without reference to the wild type RNA virus from which the RNA vector is derived.

The inserts are sometimes referred to as “mimics”. One or more aspects of the inserts were developed initially through studies of some naturally occurring hairpin-like segments in some umbravirus-like viruses (ULVs). However, these studies were later expanded to include investigations or other viruses and to synthetic hairpin-like segments. Accordingly, the term “mimic” is now used loosely and no longer implies that there is any similar naturally occurring hairpin-like structure.

Because of the path of development indicated above, and because many studies of ULVs are recent, this specification includes an extensive discussion of ULV. However, the invention is not limited to ULV unless expressly stated.

Plant RNA viruses typically encode multiple proteins including one or more subunits of the RdRp, one or more movement proteins, a coat protein and a silencing suppressor. Plant RNA virus proteins can also have more than a single function.

One notable exception is the umbraviruses. Umbraviruses do not encode a coat protein or a silencing suppressor. However, umbraviruses are always found in nature in association with a specific helper virus from the luteovirus, polerovirus or enamovirus genera. The umbravirus uses the coat protein of the helper virus to produce a capsid/virion. The umbravirus also benefits from the silencing suppressor of the helper virus. However, the umbravirus produces its own movement proteins, one of which the helper virus uses to extend its own ability to colonize a plant.

Another notable exception are umbravirus-like viruses (ULV). ULV are plus-sense viral or sub-viral RNAs that infect plants. ULV have some similarities with umbraviruses, for example an RdRp that is generated by −1 ribosomal frameshifting. ULV may also have an umbravirus-related 3′ terminal structure. However, ULV do not encode umbravirus-related movement proteins. At least some ULV do not encode any movement proteins. ULV are not umbraviruses or otherwise in the family Tombusviridae. However, since the taxonomic analysis of at least some ULV is recent, some ULV (in particular some Group 1 ULV or Group 2/Class 1 ULVs as defined further below) may have been previously identified as umbraviruses.

Although some ULV have been described previously as subviral RNA, or may be subviral RNA according to some definitions for example because they do not form virions or encode coat proteins or other proteins characteristic of most plant viruses, the word “virus” as used herein will include sub viral RNA unless stated otherwise. The term “subviral RNA” may be used, for example, to refer to truncated progeny of ULVs such as defective RNA (D-RNA) and subgenomic (sgRNA).

Based on phylogenic analysis, ULVs can be divided into two groups. Group 1 is a catch-all grouping currently containing: AgULV or Ageratum virus 1 (e.g. GenBank OP660856); ArULV (alternatively called AULV) or Arborcitae umbravirus-like virus (e.g. GenBank OQ102001); GULV-2 and GULV-3 or Grapevine umbravirus-like virus (e.g. Gen Bank OR947507); and GuiULV (alternatively called GPpTV1) or Guiyang Paspalum umbravirus-like virus (e.g. GenBank OM514399). Group 2 ULV include, among others, the viruses (or sub-viral RNA) that were previously referred to as umbravirus-like associated RNA (ulaRNA) in Structural Analysis and Whole Genome Mapping of a New Type of Plant Virus Subviral RNA: Umbravirus-Like Associated RNAs, Liu J. et al., Viruses 2021,13, 646. Group 1 ULV are more closely related to umbraviruses than Group 2 ULV. Group 1 ULVs have lengths from 3.3-4.3 kb. AgULV has been found in plants with a helper virus. AULV has been found in plants without a helper virus.

Group 2 ULVs, are viral or subviral RNAs with monopartite, plus-strand genomes of about 2.7 to 4.5 kb. Group 2 ULVs code for at least two proteins which are both required for replication. One protein is a 5′ proximal product (expressed from open reading frame 1, ORF1). The other protein is the RdRp, which is generated by −1PRF extension of the ORF1 protein. Many Group 2 ULVs, including WULV, PUV, BabVQ, CY1, CY2, FULV, OULV, PULV, MULV and JgULV have been found in natural hosts in the absence of a helper virus. All dicot-infecting Class 2/Group2 ULVs known to date have been found in plants in the absence of a helper virus.

Group 2 ULVs can be separated into 3 classes. Class 1 do not encode any additional proteins beyond their replication required proteins and have an extensive 3′ UTR. Class 3 ULVs have one or two additional ORFs at least one of which overlaps the RdRp ORF. Class 2 members that infect dicot hosts contain one additional ORF (optionally called ORF5) that also partially overlaps the RdRp ORF. Class 2 members that infect monocot hosts have a third ORF (optionally called ORF5 and is the same protein as found in the dicot-infecting Class 2 members) overlapping the RdRp and also most have an additional ORF (optionally called ORF6) embedded within ORF5. One exception to the description above is citrus yellow vein associated virus 1 (CY1). CY1 is a dicot Class 2 ULV that does not have ORF5 due to two large deletions and other mutations. CoULV, found in Taro, is an exception among monocot-infecting Class 2 ULV that does not contain ORF6.

Group 2 ULV (and some Group 1 ULV) are further distinct from umbraviruses because umbraviruses have an intergenic region (typically 100-300 nt) between their replication required ORFs and any additional ORFs. Group 2 ULV either have no ORFs beyond their replication required ORFs, or have a third ORF adjacent the RdRP ORF. Some Group 2 ULV also have a fourth ORF. There are no material intergenic region between the ORFs of most ULV, AgULV being an exception.

Class 1 ULV, examples of isolates in GenBank, and their host plants include: a) PMeV2 (KT921785)-papaya; b) PUV (MT113180)-papaya; and c) BabVQ (MT113182)-babaco. It is noted that some Class 1 ULVs have been previously described as umbraviruses, which is currently known to be incorrect.

Class 2 dicot infecting ULVs, examples of isolates in GenBank, and their host plants include: a) citrus yellow vein associated virus CY1 (JX101610, MZ330089, MZ330113)-citrus; b) citrus yellow vein associated virus 2 CY2 (MT893741, MT893740)-hemp; c) fig umbra-like virus FULV (MW480892, MW480893)-fig; d) opuntia umbra-like virus OULV (MH579715, MT909563, MT909562)-cactus; e) parsley umbra-like virus PULV (OM419177)-parsley.

Class 2 monocot infecting ULVs, and examples of isolates in GenBank, include: a) Teosinte umbra-like virus TULV (OK018180)-teosinte; b) Johnson grass umbra-like virus JgULV (OM937760)-Johnsongrass; c) Ethiopian maize-associated virus EMaV2 (MF415880) and EMAV1 (MN715238)-corn; e) sugarcane umbra-like virus SULV (MN868593)-sugarcane; f) maize umbra-like virus MULV (OM937759)-corn.

Class 3 ULVs, and examples of isolates in GenBank, include: a) SbaVA (MK211274)-strawberry; b) WULV (OK573479)-wheat; c) GULV (OP88631)-grapevine; and, d) PIULV (OL330774)-Poaceae liege.

The GenBank sequences at the identifiers given above are incorporated by reference. Unless specified otherwise, the reference sequence of CY1 is GenBank JX101610, which is SEQ ID NO:1 herein.

Some ULVs are described in detail in Structural Analysis and Whole Genome Mapping of a New Type of Plant Virus Subviral RNA: Umbravirus-Like Associated RNAs, Liu J. et al., Viruses 2021,13, 646. It was noted in this publication that some ULVs express a protein with movement protein-like motifs. Without intending to be limited by theory, the inventors currently believe that the proteins described in that paper are not movement proteins but may instead be coat proteins (it is known in the art that movement proteins of the 30K class and coat proteins can be hard to distinguish and are related to each other). Accordingly, they will typically be called coat proteins herein since further experimentation showed that they do not have conserved features and properties found in all 30K class movement proteins. However, the vectors described herein are not dependent on whether these are coat proteins, movement proteins or any other type of protein.

A description of some ULVs, an in particular Group 2/Class2 ULVs, is provided in −1 Programmed ribosomal frameshifting in Class 2 umbravirus-like RNAs uses multiple long-distance interactions to shift between active and inactive structures and destabilize the frameshift stimulating element, Mikkelsen A. et al., Nucleic Acids Res. 2023 Oct. 2; 51(19):10700-10718, which is incorporated by reference. In particular, this publication describes six tertiary interactions that are conserved in Class 2 ULVs and two tertiary interactions that are conserved in all Group 2 ULVs. Optionally, the presence of these structures and interactions when present in at least one conformation, optionally in combination with other indicia, may be used to identify ULVs. Some of the relevant structures and interactions can be summarized as: a) a Y-shaped folded region, in particular a Y-shaped region having a sequence and/or secondary structure similar to that of other ULVs, b) a hairpin-like region with a ribosome recoding area downstream of the Y-shaped region with an apical loop that interacts (by way of complementary bases) with a further downstream region, c) interaction (by way of complementary bases) of a hairpin like region upstream of the Y-shaped region with a hairpin like region downstream of the Y-shaped region.

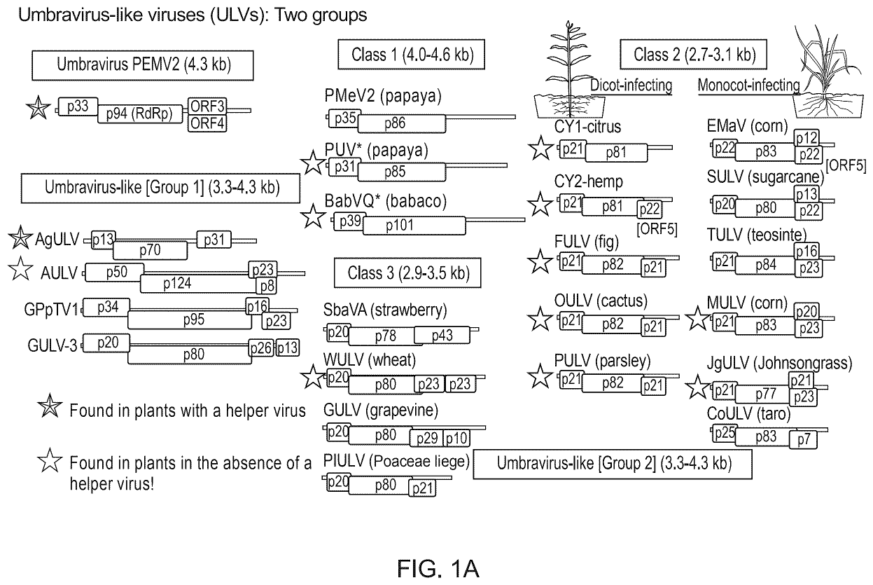

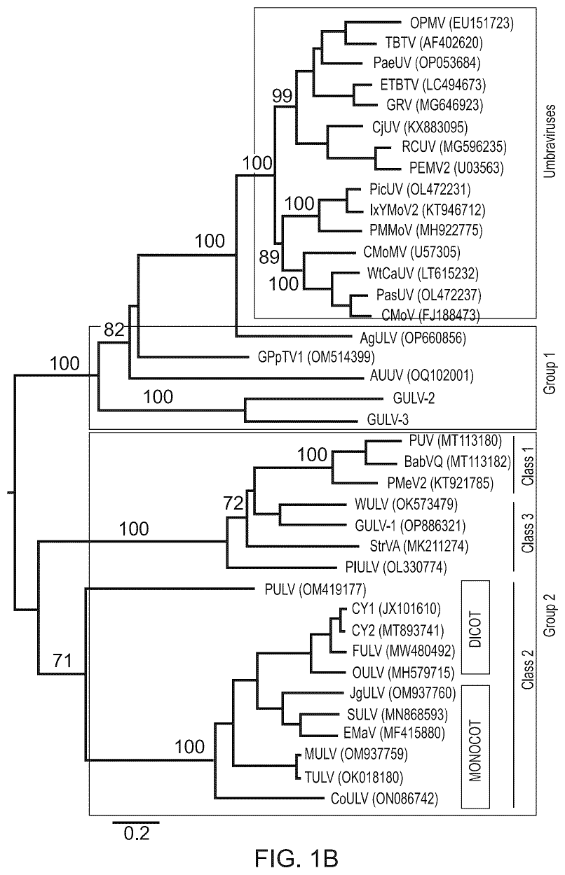

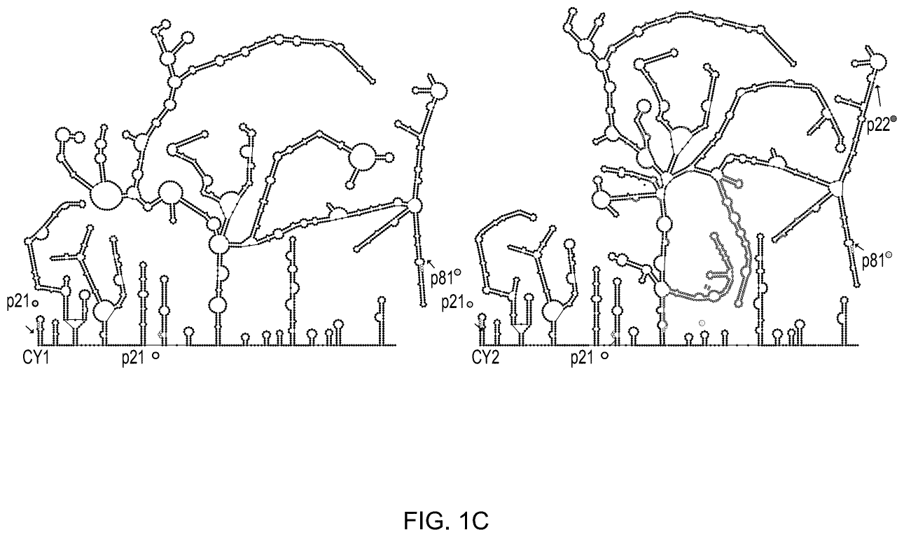

FIG. 1A shows the genome organization of some Group 2 ULVs. As indicated therein, some ULVs have only replication required ORFs (the first two ORFs). Class 2 ULVs have one or two additional ORFs without intergenic regions between the additional ORFs and the replication-required ORFs or between multiple non-replication required ORFs. Class 3 ULVs have one or two non-replication required ORFs without intergenic regions between the additional ORFs and the replication-required ORFs or between multiple non-replication required ORFs.

FIG. 1B shows a maximum-likelihood phylogenetic tree used to evaluate the relationships of ULVs to all known umbraviruses based on RdRp nucleotide sequences. Alignments were generated and trimmed in MEGA X [92]. General time reversible model with gamma distribution rates and invariable frequency (GTR+G+I) was determined to be the best-fit substitution model in MEGA X and was implemented to construct the tree. The following ULV sequences were used: PMeV2 (KT921785), PUV (MT113180), BabVQ (MT113182) [42, 93, 94], OULV (MH579715, MT909563, MT909562) [44], FULV (MW480492, MW480492)[60], CY1 (JX101610) [40], CY2 (MT893741) [77], WULV (OK573479) [95], GULV (OP886321) [96], PIULV (OL330774) [97], SbaVa (MK211274) [98], TULV (OK018180) [99], MULV (OM937759) [41], JgULV (OM937760) [41], SULV (MN868593) [100], EMaV (MF415880), AgULV (OP660856), GuiULV (OM514399), ArULV (OQ102001 [101]. To analyze the phylogenetic relationship of ORFS proteins with selected CPs and MPs, protein sequences were aligned through PROMALS3D [53] and trimmed in MEGA X. The LG model with gamma distribution rates and invariant frequency was determined to be the best-fit substitution model in MEGA X and was implemented to construct the tree.

FIG. 1C shows the secondary structure of CY1 and CY2. CY2 is the closest presently known relative of CY1, and might optionally be considered a strain of CY1. CY2 differs from CY1 in having a subgenomic promoter and two long segments not found in CY1. However, CY2 has over 85% or over 90% sequence identify with CY2 as measured by clustal W score (about 90.2% for CY2: MT893741 and CY1: JX101610). Other known dicot infecting Class 2 ULVs have over 70% sequence identity with CY1 except for PULV (found in parsley) which has over 54% sequence identity with CY1 (about 54.1%). Other known monocot infecting Class 2 ULVs also have over 54% sequence identity with CY1. In comparison, the umbravirus PEMV2, which is the closest known relative to CY1 that is not a ULV, has a sequence identity of only about 50% (50.3%) with CY1. Some Class 1 and Class 3 ULVs or Group 1 ULV may also have sequence identities with CY1 of less than 54%, or less than 50%, but are considered to be a ULV and not an umbravirus for example because they do not encode movement proteins similar to umbravirus movement proteins, because they do not have ORFs other than replication required ORFs, because they lack an intergenic region between replication required ORFS and any other ORFs, and/or because they have secondary structures or tertiary interactions similar to CY1 or other Class 2 ULVs. As many ULVs have not been extensively studied, their naming and classification may change over time. Some ULVs that are separately named herein may, in future, be considered to be strains of a single virus. In some examples, the backbone of a vector may have at least 54%, at least 70%, at least 85% or at least 90% identity with SEQ ID NO: 1.

ULVs infect and replicate in plants. Although ULVs do not have umbravirus-like movement proteins, and at least some have no movement proteins at all, they are able to move within the phloem of plants. In at least some ULV, this movement is believed to result from specific binding of a portion of the RNA genome with phloem protein 2 (PP2), a common protein made by many plants. The portion of the RNA genome responsible for PP2 binding is believed to be upstream of ORF2.

At least some ULV also do not encode a silencing suppressor. Without intending to be limited by theory, ULV may be able to survive without silencing suppressors by having a very high rate of replication. In at least some examples, ULVs may make about six times more polymerase for replication than other viruses. Given the high rate of replication, even through some of the progeny are cut by the DICER-like enzymes of the host plant, some of the progeny remain as full length sequences for further replication. When used as a vector, the high rate of replication combined with the action of the host plant DICER-like enzymes causes the production of non-coding exogenous inserts (for example siRNA) at high rate. In at least some examples, coding exogenous inserts may also be produced at a high rate.

ULVs are capable of phloem limited movement in plants despite, in at least some examples, not having movement proteins. CY1 clearly does not have a movement protein and yet moves through the phloem of many plants. In the case of Class 2 dicot infecting ULVs, as demonstrated by examples using CY2 and OULV, when the third ORF (ORF5) is disabled, the ULVs are still able to move thorough the phloem of the plant. Without intending to be limited by theory, movement through the plant may be enabled by binding of the ULV with phloem protein 2 (PP2), a common protein produced by many plants.

Defective RNA (D-RNA) of CY1 lacks nucleotides 672 to 2419. However, this D-RNA also binds to PP2. D-RNA of CY2, also contains portions of the 5′ and 3′ ends and is predicted to also bind to PP2.

The present disclosure also describes the use of ULVs other than CY1, for example other Class 2 dicot ULVs, as vectors. In some embodiments, CY2 is used as a vector. In other embodiments, a ULV infecting opuntia (OULV) is used as a vector. However, given the high degree of similarity between ULVs, and in particular between Class 2 dicot ULVs, other ULVs including other Class 2 dicot ULVs may be similarly converted into vectors and are expected to bind to PP2 and move within the phloem of plants.

PP2 binds to all RNA to some extent. References to binding to PP2 herein refer to specific binding, or to binding that is more efficient than the binding between PP2 and random RNA sequences.

CY1 does not produce virions whereas as CY2 does produce virus-like particles. However, CY1 produces agglomerates with PP2 in host plant sap. Since CY2 in the absence of ORF5 also moves systemically with similar timing, tropism, and symptoms as CY1, it likely also binds to PP2 in plants and has been shown to bind to PP2 in vitro using Northwestern assays. In addition to providing movement, binding to PP2 may improve the response of the host plant to diseases, such as citrus greening, wherein harm to the plant may be related to excess PP2 production and/or PP2 sieve plugging in response to the pathogen.

Without intending to be limited by theory, current hypotheses regarding ORF5, the movement of ULVs, and the evolution of some plant viruses include the following:
1. ULVs, with a few exceptions, encode their own RNA polymerase and capsid-like protein.
2. Group 2 ULVs, and particularly Class 2 ULVs, generate copious amounts of polymerase by −1PRF; silencing suppressors may not be needed.
3. Unlike umbraviruses, ULVs are with few exceptions found in the absence of a helper virus, possibly because they do not need one as some of them encode their own capsid protein.
4. ORF5 produces a capsid-like protein related to the P3 coat protein of luteoviruses, poleroviruses, enamoviruses, and sobemoviruses (the helper viruses for umbraviruses).
5. CY1 has lost the ability to encode the capsid-like protein and might no longer be transmitted to new plants since transmission vectors (e.g., insects like aphids) require virions.
6. With few exceptions, ULVs use host movement proteins for movement.
7. For CY1, CY2 and other Class 2 ULVs, the host movement protein is likely PP2, which is the same movement protein used by the hop stunt viroid.
8. Ancient plant viruses may have systemically infected plants without encoded movement proteins.
9. Movement proteins may be the progenitors for some coat proteins.

It is currently unknown if or how CY1 may have been transmitted from plant to plant in nature. Since CY1 has only been found 4 times in nature, all in same grove of trees at the same time, it is possible that CY1 is a fragment of another virus that has not been located. However, CY1 and other ULV are capable of replication and movement when introduced into a plant, for example by grafting, transfer through dodder or agroinfiltration (agroinfection).

CY1 was originally found in limequat trees and readily infects citrus trees and some other host plants. Some previous publications suggest that CVEV is a helper virus for CY1, but CY1 is no longer considered to have, or at least not to require, a helper virus.

CY1 contains two open reading frames, ORF1 and ORF2, both of which are required for replication. The ORF2 protein is generated as a fusion protein with the ORF1 protein after a ribosome −1 frameshift.

CY1 replication in plants naturally produces two major long non-coding fragments, F281 having nucleotides 1-281 and F671 having nucleotides 1-671. Without intending to be limited by theory, F671 may be RdRp-derived and F281 may be exonuclease-derived. Other fragments may also be produced. Defective RNA (D-RNA) are also produced having, for example, nucleotides 1-671 and 2420-2692. The D-RNA is replicated by the CY1 polymerase and moves in host plants. Surprisingly, the D-RNA can also replicate in the absence of CY1 and can move systemically at low levels independent of CY1. Roots may act as a repository of full-length RNA while D-RNA are a significant fraction in the whole plant.

The polynucleotide sequence (bases 1 to 2692) of the CY1 isolate at GenBank JX101610 is presented below (SEQ ID NO:1):

| | |
|---|---|
| ggguaaauau ggauccuuca ucuuugcccc gugccuguug gcaucaugcc | 50 |
| agacaggugu uucgagcauc aacuagcuuc ucaagagagg ugguucgcgc | 100 |
| ugcucguaga uggguuacca ugcccaccag ucgccaugca uaugacuuuu | 150 |
| caacgagucu aggcauugug auugcugagc cugcagcucg uuuacgacgc | 200 |
| cgucugcccu cuguacgaaa gugcgcagag aaguuaguag uccacaagca | 250 |
| agucgacacu uugguggacg aauggugcuc uggaauuccc aacccugaua | 300 |
| ucguagaagu ugguugggca cuccgucuga gggaccguuu cggucuuccu | 350 |
| cccgcuucug agccuacccg gcucaguggu gagagauggg ugcucaaaca | 400 |
| acucaauggg guagauccug agucauggaa ugcugaucuu gguaggucag | 450 |
| uucauaucca aggagacuac gccccaggga ggaaugccca uaucgcucag | 500 |
| gucgcggcga ccuugugguu aacuaggacc uugcaugaca aggccuuggc | 550 |
| ucgccaccag gguuuucgcg auuugcagug auuggggucg acgggcuaga | 600 |
| ggcaaaagca gugccucuag cuucuggacu ccgacugcuu ccgguuccgc | 650 |
| gacccggaca aagucgacga cugucucaga ccuuguuacu uccaacaccu | 700 |
| cgugcucaau ucgugaauca cgcgugcucg gcuaacaacc uuggacgugu | 750 |
| gaugaccaca cguguguugc aguacaaggg ccgagauccg auccuucccu | 800 |
| cuucugaagc ccuucaccga cuuaaccuuc ggauagcuga gcuauauagg | 850 |
| ucuagaccuu cuaccgucua uccauuaagu uaugaagggu uucucaauug | 900 |
| cuaugaaggc cgacagcgua cucguuacgc ccaagccguc gagcaguuga | 950 |
| ugcgguccac ucuugagccg aaagaugcgc gaguugaaac guucauuaag | 1000 |
| aacgagaaau uugacugggc guugaaaggg gaggaggcug auccucgagc | 1050 |
| aauccaacca aggaagccga aauauuuggc ugagguugga cggugguuca | 1100 |
| aaccuuugga gcgaaucauc uacaaggauc ucaguaaaag guuguauggu | 1150 |
| gagggugcug agccguguau cgccaaaggc cuaaaugcau uagaaucugg | 1200 |
| agcgacuuug aggcgcaaau gggagaaguu uucuucucca guuugcguuu | 1250 |
| cucucgacgc uuccagguuc gaccugcaug uaagcguugg caugcuaaag | 1300 |
| uucacacaca agcuauauga cuauuacugu aagucuccca cucuccagcg | 1350 |
| cuaucucaaa uggacacucc gcaaccaugg cgucgccucc ugcaaagaau | 1400 |
| ugucauauga guaugagguu guuggccgga gaaugagugg ugacauggac | 1450 |
| acugcauugg gcaacugcgu cauuaugucg auacuuacau gguuuaugcu | 1500 |
| uagugaacuu ggcauuaagc augaauuauu cgauaauggu gacgauuguu | 1550 |
| uguucauuug cgagucucac gacgucccca gccccgaggu aauuacaaac | 1600 |
| ugguuuucgg acuuuggguu ugugguuagg uuggaaggg ucacguccgu | 1650 |
| guuugagcgu auugaguuuu gccaaacuuc cccaguaugg acugagaggg | 1700 |
| guuggcugau guguaggaau auuaagucau ugaguaaaga ccuuacgaau | 1750 |
| guuaauucgu gcacgggcuc cacgauugaa uauacccacu gguugaaagc | 1800 |
| agugggaaag ugcgggucaa uacucaaugc ugguguaccu auauuucagu | 1850 |
| ccuuucacaa caugcuggaa aggcuuggca cuaacucucg uauugaucga | 1900 |
| gggguuuucu ucaaaucagg gcuaguuaau cucauucgug ggauggacag | 1950 |
| gcagccugac guugacauca cuacuuccgc ucggcuuucu uucgaagugg | 2000 |
| cauucgggau aacacccggg augcaauugg cuauugaacg guacuaugac | 2050 |
| ucugucaugg gcucgcugag uaaaauagaa acaacuaagu ggccaauuga | 2100 |
| acuaagaaag gaauacgaac acggaaguga gugguacgag gacuuaggcg | 2150 |
| uccuaggaug aauaggguca uugguuuacc gaugauaccu guucagaaua | 2200 |
| ggauugcucg agcuucguug guuaggguaa cucacauacc uucuuccaua | 2250 |
| acuggaaaag gucgugugag caaccuaacc aguuaaugua ggugucuuuc | 2300 |
| cguaucuagu cacgauggua agcaacccgu uuaucuguac ggcgcucacc | 2350 |
| cguggguagg aaggugaagg uuuugugucc uuuaggucuu ggacagucug | 2400 |
| cgggcuuggg aacgacgccc cgcuagcaac guacugcucu ccuaccggac | 2450 |
| ugguagcuua auugucaucu uggagcgaua gcacuguggg ccucacccuu | 2500 |
| cgcgcguugg acguguugcg ugccccccac agauuuguga aacucuaugg | 2550 |
| agcaguuccg cgagccagaa gggaggaugg ccgccuggcg uaauccagga | 2600 |
| gcucuggggg gcuuguacuc agaguagcau ucugcuuuag acuguuaacu | 2650 |
| uuaugaacca cgcgugucac guggggagag uuaacagcgc cc | 2692 |

The 3′ end of CY1 includes bases 2468 to 2692. The 3′ Cap Independent Translation Enhancer (3′ CITE) of CY1 includes bases 2468-2551. ORF1 which encodes p21 includes bases 9 to 578. ORF2 which encodes p81 includes bases 752 to 2158.

The amino acid sequence of protein p21 is presented below (SEQ ID NO:2):

MDPSSLPRACWHHARQVFRASTSFSREVVRAARRWVTMPTSRHAYDFST
SLGIVIAEPAARLRRRLPSVRKCAEKLVVHKQVDTLVDEWCSGIPNPDI
VEVGWALRLRDRFGLPPASEPTRLSGERWVLKQLNGVDPESWNADLGRS
VHIQGDYAPGRNAHIAQVAATLWLTRTLHDKALARHQGFRDLQ

The amino acid sequence of protein p81 is presented below (SEQ ID NO:3):

MTTRVLQYKGRDPILPSSEALHRLNLRIAELYRSRPSTVYPLSYEGFLNCYEGRQRTR
YAQAVEQLMRSTLEPKDARVETFIKNEKFDWALKGEEADPRAIQPRKPKYLAEVGR
WFKPLERIIYKDLSKRLYGEGAEPCIAKGLNALESGATLRRKWEKFSSPVCVSLDASR
FDLHVSVGMLKFTHKLYDYYCKSPTLQRYLKWTLRNHGVASCKELSYEYEVVGRR
MSGDMDTALGNCVIMSILTWFMLSELGIKHELFDNGDDCLFICESHDVPSPEVITNWF
SDFGFVVRLEGVTSVFERIEFCQTSPVWTERGWLMCRNIKSLSKDLTNVNSCTGSTIE
YTHWLKAVGKCGSILNAGVPIFQSFHNMLERLGTNSRIDRGVFFKSGLVNLIRGMDR
QPDVDITTSARLSFEVAFGITPGMQLAIERYYDSVMGSLSKIETTKWPIELRKEYEHGS
EWYEDLGVLG

The polynucleotide sequences of recoding frameshift sites of CY1 is presented below:

(SEQ ID NO: 4)
ucgcucaggucgcggcgaccuugugguuaacuaggaccuugcaugacaa
ggccuuggcucgccaccaggguuuucgcgauuugcagugauuggggucg
acgggcuagaggcaaaagcagugccucuagcuucuggacuccgauguug
guucgcgacccgga
(SEQ ID NO: 5)
caaagucgacgacugucucagaccu
(SEQ ID NO: 6)
aggucuuggacagucugcgggcuugggaacgacg

CY2 was originally found in hemp but also infects some other host plants. CY2 is related to CY1 but differs from CY1 in having an additional open reading frame (FIG. 19A), a subgenomic promoter, two long segments not found in CY1, and other changes from CY1 that keep the ORF open (FIG. 19B). The additional open reading frame is optionally called “ORF5” (FIG. 19A), the designations of ORF3 and ORF4 having been reserved for movement proteins of umbraviruses in some previous publications comparing CY1 to umbraviruses. As discussed herein, this protein encoded by the additional open reading frame is currently believed to be a coat protein, although it might alternatively or additionally have another activity.

CY2, when infecting as host plant, produces a D-RNA, and sgRNA and at least one major non-coding fragments. Roots may act as a repository of full length RNA while D-RNA are a significant fraction in the whole plant.

CY2 (and other ULV) contains two open reading frames, ORF1 and ORF2, similar to those of CY1, both of which are required for replication. The ORF2 protein is only generated as a fusion protein with the ORF1 protein after a ribosome −1 frameshift. Expression of the ORF5 protein in CY2 (and possible other ULVs) is most likely from the sgRNA.

The sequence of the CY2 used in experimental examples is a modification (believed to be a correction) of the sequence in GenBank MT893741 and is presented below (SEQ ID NO: 7):

GGGTAAATATGGAATCTTCATCTTTGCCCCGTGCCTGCTGGCAAAAGGCCAGGCA
GGTGTTGCGAGCACCAGCTACCTTC
TCGAGAGAGGTGGTCAAAGCTGCCCGCAAATGGGTCACCATGCCCAGTAACCAC
CGTGTCTACGACTACTCCACTAGTCT
GGGCATTGTGATTGCTGAGCCTGTGGCTCGTCTGCGACGCAGTCTACCCTCCGTA
CGAAAGTGCGCAGAGAAGATAGTAG
TCCACCAGGGAATCGACACCTTGGTGGACGATTGGTGTACAGGACTTCCTAACCC
TGATATAGTGGAAGTTGGTTGGGCA
CTCCGTCTGAGGGACCGTTTCGGTCTTCCTCCCGCCTCTGAGCCTACTAGGCTTAG
TGGTGAGAGATGGGTGCTCAAACA
ACTCAATGGGGTAGATCCCGAGTCATGGAATGCTGATCTGGGTAAACCAGTTCAC
GTCCAGGGAGACTACGCCCCAGGGA
GGAATGCCCACATCGCGCAGGTCGCGGCGACCTTGTGGCTAACTAAGACCTTGTC
TGACAAGGCCTTGGCCCGCCACCAG
GGTTTTCGCGATTTGCAGTGATTGGGGTCGACGGGCTAGAGGCTAAAGCAGTGCC
TCTAGCTGCAGGACTCCGACTGCTT
CCGGTTCCGCGGCCCGGACAAAGCCGACGGCTGTCTCAGACCTTGTTACTACCAA
CCCCTCGTGCACAATTTGTGAATCA
CGCGAACTCGGCTAATAACCTTGGGCGCGTGATGACCACACGAGTGATGCAGTA
CAAAGGCCGAGACCCGATCCAACCCT
CTCAAGAAGCCCTAACCAAACTTAACCTTCGGATAGCCGAGCTATATAAGTCGA
GACCATCCACCGTCTATCCGTTGAGT
TATGAAGGGTTTCTCAACTGCTACGAAGGCCGACAGCGTACTCGTTACGCTCAAG
CCGTCGAGCAGCTGTTGAGGTCCAC
TCTAGAACCTAAAGATGCGAGAGTTGAAACGTTCATTAAGAACGAGAAGTTTGA
TTGGGCGTTGAAAGGGGAGGAGGCTG
ATCCTCGTGCAATCCAACCAAGGAAGCCGAAATATCTGGCTGAGGTTGGACGGT
GGTTTAAACCTTTGGAGCGAATCATC
TACAAGGATCTTAGTAAGCGGTTGTATGGCGAGGGTGGTGAACCTTGTATTGCCA
AAGGCTTAAATGCACTAGAATCTGG
GGCGACTTTGAGGCGCAAATGGGAGAAGTTTTCTTCTCCTGTTTGCGTCTCTCTTG
ACGCTTCCAGGTTCGACCTGCATG
TAAGTGTTGGCATGCTTAAGTTTACACACAAGCTGTACGACTACTACTGCAAGTC
TCCCACCCTCCAACGTTATCTCAAA
TGGACACTCCGCAATCACGGCACTGCCTCCTGTAAAGAATTGTCATATGGTTATG
AGGTAGAAGGCCGAAGAATGAGTGG
TGACATGGACACCGCATTGGGCAACTGCGTCATCATGTCGATACTAACATGGTTT
ATGCTTAGTGAGCTTGGCATTAAGC
ATGAATTATTCGACAATGGTGACGACTGCTTGTTCATTTGCGAGTCAAAAGACGT
CCCCAGCCCCGAGGTGATCACGAAC
TGGTTTTCGGACTTTGGGTTTGTGGTCAGGTTGGAAGGCGTCACGTCCGTGTTTG
AGCGTATAGAGTTTTGCCAAACTTC
CCCAGTATGGACTGAGAGGGGTTGGCTGATGTGTAGGAACATTAAGTCGTTGAG
TAAGGACCTTACGAATGTCAATTCGT
GCACGGGCTCCACAATTGAATATACTCACTGGTTAAAAGCCGTGGGGAAGTGCG
GGTCAATACTCAATGCTGGTGTGCCT
ATATTTCAGTCCTTTCACAACATGCTGGAAAGGCTTGGTACTAACTCTCGTATAG
ATCGCGGGGTGTTCTTTAAATCTGG
GCTAGTCAATCTCATTCGTGGGATGGACAGACAACCTGACGTTAAGATCACCACT
TCCGCTCGTCTTTCTTTCGAAGTGG
CATTCGGGATTACACCTGGAATGCAATTGGCTATTGAACGGTACTATGACTCAGT
CATGGGCCCGCTGGGTAAAATAGAA
ACAACTAAATGGCCAATAGAACTAAGAAAGGAATACGAGTACGGAAGCGAGTG
GTACGAAGACTTAGGCGTCCTAGGATG
AGCAGGGACATCGGTTTACCGATGGCCCCCGTTCAGAATAGGATGGCTAGGGCT
TCGTTGGTTAGGGAAACCTATATACC
TTCTTCCACCACAACTGGTAAGGAGGCTGTGTGGGCAACCTGGCCAATTAATGTA
GGTGTCTTTCCGCAACTAGCCACGA
TAGTAAGCAACCCGTTATACTGGCGTGTGACCAGTGTGCAAGTGGCAATGGAGC
CAGCAACGTCAACCTCTACCCAACTC
TGTGGTGTAGGTCTCAGTACATCTGGGGCTTACAACTCAGGGGAAGGGTTCGGG
AACACGTTTAAGCTCTTGCGCTCTTG
TAACTATACGAGACGCTCACCCGTGGGAGGGAACGTGATGGTCAAGTGGCCCAT
CAGCATGCCCTACATTCTTAATGATG
ATGCGCACAAGACCACAGGTCTAACAGCTTGTGTGGTCATTGCTGTCACCAATCC
AGGTGCCATCACTGGCCAATCTTGG
GCGGAGATTCAATTGAATGTAGAGTACGTAGTGGGTACTTAGACAGTCTGCGGG
CTTGGGAACGACGCCCCGCTAGCAAC
GTACTGCTCTCCTACCGGACTGGTAGCCGAGCTGTTATCTTGGAGCGATAGCACT
GTGAGCCTCACCCTGCGCGCGATGG
ACGTGTTGCGTGACCCTCACAGATTTGTGAAACTCTATAGAGTAGTTCCGCGAGC
CAGAAGGGAGGATGGCGACCTGGCG
AAATCCAGGAGCTCTGGGGGGCTTGTACTCAGAGTAGCAATACTGCTTTAGACTG
TTAGTTTGATGAACCACGCGTGTCA
CGTGGGGAGAACTAACAGCGCCC

OULV was originally found in opuntia (a cactus) and a nearly identical version also infects hemp. OULV has over 70% sequence identity with CY1 and CY2.

The RNA vector described herein may comprise a backbone, typically derived from a wild type virus, and one or more heterologous segments inserted in the backbone. The backbone may include the genomic sequence of a virus, optionally with one or more modifications. The backbone may contain all or substantially all of the genomic RNA sequence of a wild type virus. The backbone may have 60% or more, 70% or more, 80% or more or 90% or more or 95% or more or 98% or more sequence identity with a wild type virus that the vector is derived from.

The backbone may be a wild type virus or a sequence derived from a wild type virus. Derivation implies the addition, removal or substitution of one or more nucleotides of the wild type virus. The wild type virus may be referenced by the sequence of an isolate of the virus or a consensus sequence. The derivation may include any modifications known in the art to produce suitable vectors.

The backbone typically maintains the ability to replicate in a host. In some ULV examples, the backbone retains the replication required ORFs, and optionally a PP2 binding site, of a reference virus in a functional form. However, the backbone may have modifications or deletions. In some embodiments, the deletions may include the removal of some or all of a hairpin-like region. In some embodiments, the modifications may include point mutations or short sequence modifications relating to the folding or thermodynamic stability of the vector. In some embodiments, the modifications may include attenuation or disabling of non-replication ORFs, which may be accomplished by methods known in the art.

Other modifications may include, for example, deletion or attenuation of portions of the sequence not required for replication and/or movement, deletion or attenuation of portions related to the safety of the vector such as production of symptoms in the host or transmission between hosts, or minor modifications for example to adjust the fitness of the virus or the stability of the secondary structure of the virus. In most cases, a sufficient portion of the wild type virus is retained such that the vector retains the native ability of the wild type virus to replicate in a host and, optionally, to also move within a host plant. The vector may also differ from the wild type virus in the way that isolates of a virus normally vary from other isolates or from a consensus sequence for a virus or subviral RNA. In some cases, the vector will have a sequence identify of at least 80%, at least 90% or at least 95% of the wild type virus or a dimer, trimer or other isomer thereof. The vector may also have multiple heterologous inserts, including inserts other than the inserts described herein.

Modifications to the backbone can include, for example, point mutations, small deletions or truncations, or removal or replacement of hairpin-like regions. Modifications to the backbone may include fitness or reverse fitness related modifications or deletion or truncation of hairpin like regions, either without replacement or with replacement with a heterologous hairpin like region, as described in International Publication Number WO 2023/215782, RNA Vectors with Hairpin-like Inserts, published on Nov. 9, 2023, describes.

In an optional aspect of reverse fitness, a hairpin-like or other structure of the wild type virus is removed in combination with adding one or more inserts. The insert or inserts may be added all in other locations, or an insert may be added in the former location of the structure that was removed. For example, a hairpin-like structure of the wild type virus may be removed and replaced with an heterologous hairpin-like structure. Optionally, the heterologous hairpin-like structure is similar in one or more aspects to the hairpin-like structure that was removed, for example in length, minimum free energy (alternatively called MFE or ΔG), average positional entropy, or secondary structure. Alternatively, the heterologous hairpin-like structure is unlike the hairpin-like structure that was removed, but is similar in one or more ways (e.g. length, minimum free energy (ΔG), average positional entropy, or secondary structure) to another hairpin-like structure in the wild type virus, or a relative of the wild type virus, or the heterologous hairpin-like structure is otherwise a hairpin-like structure as described herein.

The factors described herein may also be applied separately to a hairpin-like structure included as part of a Y-shaped, V-shape, or T-shaped insert or other insert with a junction.

Based on the observation of wild type hairpin-like structures and experiments involving heterologous hairpin-like structures, various additional parameters may be useful for insert stability: (1.1) the length limit of a fully base-paired region; (1.2) the length limit of the entire hairpin-like structures; (1.3) the length limit of consecutive G:C pairs; (1.4) the maximum number of non-paired bases in a symmetrical or asymmetrical internal loop; and, (1.5) the largest average positional entropy of a cluster (i.e. 10 per side) of base-paired nucleotides or an entire base-paired segment. For an RNA vector derived from a wild type virus, a heterologous segment may be used that does not exceed one or more of these parameters, or other parameters described herein, as determined by the wild type virus. In some examples, parameters derived from a wild type virus may be modified, for example by 50%, by 40%, by 30% or more.

In some embodiments, the heterologous segment further complies with one or more design guidelines or parameters. These guidelines or parameters for the heterologous segment may include: the average positional entropy (APE) is in the range of 0.01 to 0.75; the length of the heterologous segment is 300 nt or less; the maximum length of a base-paired region is 19 base pairs or 17 base pairs; the maximum APE of a base-paired region is 0.8; the maximum number of consecutive G:C pairs in a base-paired region is 4; the maximum number of bases in a non-base-paired region is 21 or 20; the ΔG of the exogenous segment is within a range of +20 to −20 kcal/mol, or −5 to +15 kcal/mol, or within 10 kcal/mol (+ or −) of the ΔG of a naturally occurring hairpin of similar length; the standard deviation of PE is less than 0.5, less than 20% or less than 15% of bases have a PE greater than 1; the largest PE of any base is not greater than 2.0; and the insert, not considering the apical loop, is 65-90% base-paired. Although heterologous segments are optionally hairpin-like structures, a hairpin may be designed according to one or more of the design guidelines or parameters described herein.

Accordingly, in some embodiments, the heterologous segment has a minimum free energy similar to the corresponding structure of the wild-type RNA virus. In some embodiments, the heterologous segment has one or more parameters within (or within 50% of the limits of) a range of the parameter in one or more hairpin-like structures of a wild type virus, the one or more parameters selected from the group consisting of: average positional entropy (APE); secondary structure; maximum length of a base-paired region; maximum number of consecutive G:C base pairs; maximum number of A:U base pairs, maximum number of bases on one side or collectively on both sides in a non-base paired region; maximum APE of a base-paired region; minimum free energy relative to length; and maximum length of hairpin-like segment.

Designing hairpin-like structures that have the appropriate attributes, such as average positional entropy, may start with design of the active or targeting sequence on one side of the hairpin. This is matched by a partially complementary sequence on the other side that will produce the correct values, e.g. average positional entropy values and or ΔG relative to length, for the hairpin-like structure. The partially complementary sequence may have, for example 65-90% or 70-85% complementarity with the targeting sequence. Optionally, the partially complementary sequence may be designed to result in a hairpin-like structure similar in shape to a natural hairpin-like structure being mimicked and/or incorporate other parameters described herein.

In some embodiments, the heterologous segment has an average positional entropy (APE) less than 0.75, 0.65, 0.5, 0.4, 0.39, 0.36 or 0.32. In some embodiments, the APE is greater than 0.01, 0.02 0.03, 0.04, 0.05, 0.06 or 0.07. In some embodiments, the APE is from 0.01 to 0.75, 0.01 to 0.65, 0.01 to 0.5, 0.01 to 0.4, 0.01 to 0.39, 0.01 to 0.36 or 0.01 to 0.32. In some embodiments, the APE is from 0.02 to 0.75, 0.02 to 0.65, 0.02 to 0.5, 0.02 to 0.4, 0.02 to 0.39, 0.02 to 0.36 or 0.02 to 0.32. In some embodiments, the APE is from 0.03 to 0.75, 0.03 to 0.65, 0.03 to 0.5, 0.03 to 0.4, 0.03 to 0.39, 0.03 to 0.36 or 0.03 to 0.32. In some embodiments, the APE is from 0.04 to 0.75, 0.04 to 0.65, 0.04 to 0.5, 0.04 to 0.4, 0.04 to 0.39, 0.04 to 0.36 or 0.04 to 0.32. In some embodiments, the APE is from 0.05 to 0.75, 0.05 to 0.65, 0.05 to 0.5, 0.05 to 0.4, 0.05 to 0.39, 0.05 to 0.36 or 0.05 to 0.32. In some embodiments, the APE is from 0.06 to 0.75, 0.06 to 0.65, 0.06 to 0.5, 0.06 to 0.4, 0.06 to 0.39, 0.06 to 0.36 or 0.06 to 0.32. In some embodiments, the APE is from 0.07 to 0.75, 0.07 to 0.65, 0.07 to 0.5, 0.07 to 0.4, 0.07 to 0.39, 0.07 to 0.36 or 0.07 to 0.32.

A hairpin-like insert may have one or more of: no more than 19, no more than 17, no more than 15, no more than 13, or no more than 10 consecutive fully paired bases; no more than 300 nt, no more than 200 nt, or no more than 198 nt bases; no more than 4 or no more than 3 continuous G:C pairs; no more than 20 or no more than 15 bases in a loop (i.e. counting bases on both sides of a loop); an APE of no more than 0.8 or no more than 0.6 in any base-paired region. Optionally, a hairpin-like structure as described immediately above for insertion into a ULV may be inserted into a vector derived from another wild type virus.

In some embodiments, the heterologous segment mimics the secondary structure of the wild type hairpin-like structure by having a similar arrangement of base-paired regions and non-base-paired regions.

Regarding the maximum number of consecutive fully paired bases, the presence of A-U base pairs may allow for larger values. In CY1, the largest base-paired region (13 base pairs) had an A-U rich region. Stacks without A-U rich regions in naturally occurring hairpin-like structures in CY1 had no more than 10 consecutive paired bases.

In some embodiments, the base-paired regions of the heterologous segment have 19 or less base pairs or 17 or less base pairs or 13 or less base pairs or 10 or less base pairs.

In some embodiments, the base-paired region of the heterologous segment has no more than 4, or no more than 3 consecutive G:C base pairs.

In some embodiments, the heterologous segment has no more than 21 bases or no more than 20 bases or no more than 15 bases collectively on both sides in a non-base paired region (i.e. counting bases on both sides of a loop).

In some embodiments, the heterologous segment mimics the secondary structure of the wild-type hairpin-like structure by having a similar ΔG relative to length.

In some embodiments, a) each base-paired region of the heterologous segment has an APE of less than 0.8 or less than 0.6 and/or b) the DG of a region including two non-base paired regions and one base-paired region between them is not positive.

In some embodiments, the heterologous segment has a minimum free energy within 10 kcal/mol, or within 5 kcal/mol, or within 20 kcal/mol for an insert of at least 150 nucleotides, of a naturally occurring hairpin-like structure of similar size, i.e. of a size within 10% of the size of the heterologous segment, or a minimum free energy in a range of −5 to +15 of −0.44 multiplied by the length of the insert (# of nt)−1.89, or a minimum free energy in a range of −20 to +20 of −0.44 multiplied by the length of the insert (# of nt)−1.89 for an insert of 150 nucleotides or more.

In other embodiments, the heterologous segment has one or more of a) a minimum free energy within 10 kcal/mol, or within 5 kcal/mol of a wild-type hairpin-like structure of similar length or a line of best fit between multiple wild-type hairpin-like structures, b) a length within 4 bases, or within 2 bases, or the same as a hairpin-like structure of the wild type virus or the sub-viral RNA, and c) an arrangement of base-paired (paired) and non base-paired (looped) regions that is substantially the same as, or the same as, the hairpin-like structure of the wild type virus or the sub-viral RNA.

In some embodiments, the heterologous segment has between 40 and 300, 40 and 200, 60 and 300 or 60 and 200 bases.

The limit on the APE of the entire heterologous segment to be inserted tends to result in inserts having at most a small number of bases with high positional entropy. Alternatively or additionally, one or more other guidelines may be used, including: a) the insert does not have any bases with positional entropy greater than 2.0 or greater than 1.5, optionally with an exception that a large insert (e.g. an insert with more than 100 nucleotides) may have a small number (e.g. 1 or 2 or 3) of nucleotides with PE greater than 1.5, b) the insert does not have more than 15%, or does not have more than 10%, of bases with positional entropy greater than 1.0, and c) the standard deviation of the positional entropy of the bases in an insert is 0.5 or less or 0.4 or less, for example in a range of 0.1 to 0.4.

In some embodiments, the heterologous segment has a standard deviation of PE less than 0.5 or less than 0.4. In some embodiments, a) less than 20% or less than 15% or less than 10% of the bases of the heterologous segment have a PE of greater than 1.0 or b) the largest PE of any base of the heterologous segment is not greater than 2.0 or not greater than 1.5, or both.

A mimicked heterologous segment may also follow one or more of the guidelines or parameters described above. Alternatively, a mimicked heterologous segment may follow one or more analogous guidelines or parameters derived from a study of hairpin-like structures in a wild type virus that the vector is derived from, or one or more relatives of that wild type virus.

An insert is typically designed by designing an active or targeting sequence, which provides most or all of a first side of the insert (typically not including the apical loop as part of either side). A second side of the insert, on the opposite side of the apical loop, is designed to be mostly, but not entirely, complementary to the first side. For example, the second side may be 65-90% or 70-85% complementary with the first side. The lack of complete complementarity provides alternating base-paired regions and non-base-paired regions. Each of the base paired regions may have 19 or less, 17 or less, 13 or less or 10 or less base pairs. Each of the base paired regions should also not have more than 4 consecutive G-C base pairs, and optionally not more than 4 consecutive A-U base pairs. Direction of the base pairings is not considered, for example a G-C pairing followed by a C-G pairing is considered to be two consecutive G-C base pairings. Although other parameters described herein may be considered, in many examples hairpin-like structures are stable when designed considering only 65-90% or 70-85% complementary between the first side and the second side of the insert; each of the base paired regions having 19 or less, 17 or less, 13 or less or 10 or less base pairs; and each of the base paired regions have no more than 4 consecutive G-C pairs or no more than 3 consecutive G-C base pairs, and optionally no more than 4 consecutive A-U base pairs. If a hairpin-like structure designed according to these principles is unstable, one or more other parameters or guidelines described herein may be considered to produce a more stable insert.

In some examples, an active or targeting sequence may extend into the apical loop or across the apical loop from one side of the insert to the other. However, this type of structure may not be possible for all targeting sequences, and might not be beneficial (despite the potentially increased length of targeting sequence) with all targeting sequences, i.e. siRNA targeting sequences.

The limit on the APE of a base-paired region helps to avoid clustering of high positional entropy bases, e.g. bases with positional entropy of more than 1.0. Although the presence of individual high positional entropy bases may not result in a high APE, inserts are less stable when the high positional entropy bases are numerous or clustered near each other. Other factors that may be derived, for example from an analysis of wild type hairpin-like structures and/or examples of stable exogenous hairpin-like structures, to indicate clustering include, for example, the percentage of nucleotides with a high positional entropy (e.g. a positional entropy greater than 1.0); the standard deviation of positional entropies, and the number of bases with very high positional entropy.

In some embodiments, the heterologous segment is inserted in the location of, and as a replacement for, a hairpin or hairpin-like structure that the heterologous element mimics in the wild type virus. In some embodiments, the heterologous segment is inserted in the location of, and as a replacement for, a hairpin or hairpin-like structure that the heterologous element does not mimic in the wild type virus. In some embodiments, the heterologous segment is inserted at a location that previously did not have any hairpin or hairpin-like structure in the wild type virus.

The present disclosure is also related to a method of making a viral vector, and to a plant or animal infected with a non-naturally occurring population of a virus, to a method of introducing a therapeutic agent into a plant or animal, to the use of a vector to treat a plant or animal, and to a method of treating a disease of a plant or animal.

In some embodiments, the heterologous segment includes an RNA bioactive molecule. The RNA bioactive molecules may be separated from progeny of the vector in the plant or remain part of progeny of the vector. In some embodiments, the vector may be used as a virus induced gene silencing (VIGS) vector or in a manner analogous to a VIGS vector, for example wherein small RNA segments such as siRNA generated from the heterologous segment of the vector interact with the host plant or a pathogen. In some embodiments, the small RNA segment, or a derivative thereof produced in the plant, interacts with a pathogenic virus to prevent replication of the virus rather than binding to plant cellular mRNA to silence expression of a host gene, or there is cross-kingdom transfer into a pathogen to target expression of a pathogen gene. The vector optionally replicates in the host plant. In some embodiments, progeny of the vector made in host plant cells enter the phloem where they may be transported long distances to other phloem-associated cells. In some embodiments, the progeny may be degraded by the host plant's DICER mechanism into small RNA segments including some or all of the heterologous RNA segment inserted into the vector. These heterologous small RNA segments may include an RNA bioactive molecule, for example siRNA which interfere with expression of a gene of the host plant or interfere with expression of a gene of a plant pathogen.

siRNAs can be used to target bacteria in plants, for example the Candidus Liberibacter asiaticus (CLas) bacteria. Plant pathogenic bacteria can be targeted using siRNAs that are produced in plants, taken up by the bacteria, and directly reprogram gene expression in the bacteria as described for example by Singla-Rastogi et al. (2019) Plant small RNA species direct gene silencing in pathogenic bacteria as well as disease protection, bioRxiv preprint post, Dec. 3, 2019, doi: https://www.biorxiv.org/content/10.1101/863902v1. In some implementations, CY1 or another ULV based vector is provided that contains siRNA hairpins that target a bacteria such as Candidus Liberibacter asiaticus and render the bacteria non-pathogenic. For example, an siRNA hairpin provided to a plant by a ULV based vector may be taken up the CLas or another bacteria in the plant and control gene expression in the bacteria, thereby killing the bacteria and/or inhibiting an increase of the bacterial population.

It is commonly believed that bacteria do not take up siRNA. However, as mentioned above, Singla-Rastogi et al. (2019) describes examples in which small interfering RNA targeted against some specific genes were taken up by Pseudomonas syringae and cause a 50% reduction in the population of Pseudomonas syringae. Small RNA are also taken up by other bacteria. In particular, bacteria are taken up by Xylella, Erwinia amylovora and Liberibacter crescens. These three bacteria are all gram negative bacteria that infect plants. However, since these three bacteria are otherwise unrelated to each other, they indicate that small RNA can be taken up by bacteria that infect plants generally, or at least by gram negative bacteria that infect plants. P. syringae is a plant pathogen that causes, for example, bacterial canker in almond trees. Erwinia amylovora is a plant pathogen that causes, for example, fire blight in apple trees, pear trees and some other trees in the Rosaceae family. Liberibacter crescens is a relative of Liberibacter asiaticus and, based on their experiments with Liberibacter crescens, the inventors believe that Liberibacter asiaticus will also take up small RNA.

An siRNA is typically designed to be a complement to a part of RNA or DNA associated with the target organism intended to be treated or controlled by the siRNA (“specific siRNA”). For example, Xylella, Pseudomonas syringae, Erwinia amylovora and Liberibacter crescens can all be controlled by small RNA that are complements of genes (including complements of messenger RNA) of the bacteria. In particular, these bacteria were controlled, for example by 1000 fold reductions in their population in infected plants, by specific siRNA that complement the adenylate kinase (ADK) or gyrase subunit A (GyrA) genes of the bacteria.

The present disclosure is also related to a vector comprising a hairpin-like heterologous segment targeted against a plant susceptibility gene (S-gene). The vector may be derived from a ULV, CTV or TRV. The S-gene may be, for example, MLO6 or other S-genes, such as DMR6, DND1 or ML07.

Optionally, the heterologous segment of an RNA vector may contain a non-coding RNA insert that is therapeutic either as part of the vector, when separated from the vector, or in progeny of the vector, but does not produce siRNA.

Optionally, the heterologous segment of an RNA vector may contain a coding insert, for example an insert that encodes a peptide such as an antimicrobial peptide.

In some embodiments, the action of the vector is primarily related to replication of the vector in the host plant cell rather than, for example, the stable transfection of the host plant genome. The vector is not a stable transfection vector although infection by the vector may be initiated by a transient transfection. The vector does not produce a recombinant host. The host does not express a heterologous insert of the vector by way of RNA or peptide expression of a modified host genome.

The heterologous segment of the vector can include an RNA bioactive molecule that is separated in the host plant, for example by way of a DICER mechanism, from the vector before having a therapeutic effect. While the viral vector may be limited to the phloem, a separated RNA bioactive molecule may move into and out of the phloem. Alternatively, the RNA bioactive molecule may have a therapeutic effect while part of progeny of the vector.

The heterologous inserts are designed to have an effect related to the health of the host plant or animal. For example, a heterologous insert may be targeted against the activity of a pathogen or the activity of a gene of a host plant involved in responding to a pathogen. The word heterologous does not necessarily require a sequence derived from another organism. However, in various examples a heterologous (or exogenous) RNA may include an active portion similar to (or similar to a complement of) an RNA in, or produced by, another organism. For example, the heterologous insert may target RNA produced by another organism. Similarly, a heterologous (or exogenous) segment may encode a peptide that is similar to a peptide, or a portion of a peptide, normally produced by another organism. In this context, the word “similar” may mean having a sequence identity of at least 70%, at least 80% or at least 90% with the reference sequence, being able to base-pair with the reference sequence, and/or having substantially the same activity as the reference sequence.

Also provided herein is the heterologous segment, such as the hairpin-like segment, disclosed herein. In some embodiments, the hairpin-like segment comprises, a first side, a second side, and an apical loop between the first side and the second side, wherein the first side has an a active portion and a sequence of the second side being less than 90% or less than 85% but more than 65% or more than 75% complementary with a sequence of the first side.

In some embodiments, the hairpin-like segment has one or more of:
a) 19 or less base pairs or 17 or less base pairs or 13 or less base pairs or 10 or less base pairs in base-paired regions of the hairpin-like segment,
b) no more than 4, or no more than 3, consecutive G:C base pairs in the hairpin-like segment,
c) no more than 4 consecutive A:U base pairs in the hairpin-like segment,
d) no more than 21, or nor more than 20, bases cumulatively on both sides of non-base paired regions of the hairpin-like segment, and
e) between 40 and 300, 40 and 200, 60 and 300 or 60 and 200 bases.

In some embodiments, the hairpin-like segment disclosed herein is inserted into an RNA segment derived from a virus described herein, for example a segment from a ULV, CTV or TRV.

Further provided herein is a ribonucleic acid (RNA) molecule derived from an RNA virus whose nucleotide sequence has been modified to comprise a heterologous RNA segment, wherein said heterologous RNA segment: (A) comprises a length of between about 40-300 nucleotides; and, (B) is capable of forming a hairpin-like structure, wherein: (1) the nucleotides of said hairpin-like structure exhibit an Average Positional Entropy (APE) similar to the APE of one or more naturally occurring hairpin-like structure of said virus or another RNA virus; and/or (2) said hairpin-like structure exhibits a minimum free energy (ΔG) relative to length that is within 10 kcal/mol, or within a range between −5 and +15 kcal/mol, or within 20 kcal/mol for an insert of 150 nucleotides or more, of the minimum free energy relative to length exhibited by said one or more naturally occurring hairpin-like structures of said virus.

In some embodiments, the heterologous segment has a minimum free energy within 10 kcal/mol, or within a range between −5 and +15 kcal/mol, or within 20 kcal/mol for an insert of 150 nucleotides or more, of a naturally occurring hairpin-like structure of said virus that is within 10% of the size of the heterologous segment.

In some embodiments, the heterologous segment has a minimum free energy within 10 kcal/mol, or within a range between −5 and +15 kcal/mol, or within 20 kcal/mol for an insert of 150 nucleotides or more, of the minimum free energy predicted by the length of the heterologous segment and a line of best fit though a plot of minimum free energy vs. length for a plurality of naturally occurring hairpin-like structures of said virus.

The vector may be derived from a virus. In some embodiments, the virus is a plus-sense RNA plant virus. In some embodiments, the virus is a ULV, CTV or TRV. In other embodiments, the virus is an RNA animal virus, such as poliovirus.

Additional characteristics and features of the present disclosure will be further understood through reference to the following additional examples and discussion, which are provided by way of further illustration and are not intended to be limiting of the present disclosure.

### Examples

### Brief Description of Materials and Methods

Exemplary plasmid constructs—pUC-CY1, containing CY1 cDNA cloned into pUC19 downstream of a T7 RNA polymerase promoter, and pCB301-CY1, the CY1 cDNA-containing binary vector for Agrobacterium tumefaciens infiltration, were previously described in Liu J, Carino E, Bera S, Gao F, May J P, Simon A E. 2021. Structural analysis and whole genome mapping of a new type of plant virus subviral RNA: umbravirus-like associated RNAs. Viruses: 13:646. N. benthamiana PDS gene (GenBank accession number LC543535.1) fragments PDS60 and PDS500 (positions 441-500 and 441-940, respectively), were amplified from cDNA synthesized from total RNA extracted from N. benthamiana leaves. Nicotiana tabacum magnesium-chelatase subunit I gene (GenBank accession number NM_001324838.1) fragments ChlI81 and ChlI215 (positions 527-607 and 527-747, respectively), were similarly amplified. Nano-Luciferase (n-Luc) sequence (GenBank 507 accession number OK048808.1) was amplified from a previously constructed plasmid. pUC CY1 and pCB301-CY1 were linearized using insertion site-generating primer pairs and subsequently assembled with the desired inserts using ligation-independent cloning (49). Natural hairpins H1, H2, H3, and H4 were amplified from CY1 cDNA. Ftsz160, Ftsz159, and Ftszrc160 sequences were synthesized by Synbio Inc. Inserts were assembled into linearized pCB301-CY1 using the NEBuilder HiFi DNA Assembly kit following manufacturer's instructions (NEB). All constructs were verified by Sanger sequencing.

In vitro translation—pUC-CY1-derived plasmids were linearized with HindIII and used as templates for in vitro transcription with T7 RNA polymerase, yielding RNA transcripts with the exact 5′ end and a single non-templated nucleotide at the 3′ end. The synthesized RNAs were purified by lithium chloride precipitation and normalized using a DeNovix DS-II FX spectrophotometer (50). RNA templates (0.5 μmol) were added to a 10 μl wheat germ extract (WGE) reaction mix (Promega) containing ³⁵S-methionine, 5 μl WGE, 0.8 μl 1 mM amino acid mixture (without methionine), and 100 mM potassium acetate. The translation mixture was incubated at 25° C. for 45 min. Following incubation, 10 μl of 2×SDS loading buffer (10% glycerol, 2% SDS, 90 mM Tris-HCl 524 pH 6.8, 0.1% bromophenol blue, 0.1% 2-mercaptoethanol) was added to the reaction mixture and the samples were subjected to 10% SDS-PAGE. The gel was dried, exposed to a phosphorimager screen, and scanned the next day using a Typhoon image analyzer (Amersham). Bands were quantified using GelQuantNet software.

Plant Growth—N. benthamiana plants were grown at 25° C. with a 12-hour light period and 70% humidity. Citrus trees were grown at 30° C. with a 16-hour light period and 70% humidity.

Vacuum Infiltration of Agrobacterium tumefaciens and RNA extractions—A. tumefaciens strain GV3101 was transformed with PCB301-CY1 constructs and cultured in LB medium containing appropriate antibiotics at 28° C. with shaking overnight. The culture was centrifuged at 3000 rpm for 15 min, and pelleted bacteria resuspended in infiltration buffer (10 mM MES, 10 mM MgCl₂, and 100 μM acetosyringone). The OD₆₀₀ was adjusted to 1.0 and mixed in a 1:1 ratio with A. tumefaciens containing pothos latent virus p14 gene silencing suppressor-expressing plasmid at OD₆₀₀ of 0.4 and incubated at room temperature for 2.5 hours prior to vacuum infiltration. N. benthamiana plants with 6 to 8 fully expanded leaves were submerged upside down in the cell suspension and a vacuum of −25 PSI applied for 30 sec and released. Vacuum infiltration for citrus trees was performed similarly, but with 2 min vacuum exposure, which was repeated until leaves were fully infiltrated. Total RNA from agroinfiltrated plants was extracted using TRIzol reagent (Invitrogen) according to the manufacturer's instructions.

RT-PCR screening for insert retention—Total RNA was treated with RQ-1 DNase (Promega) prior to reverse transcription. First-strand cDNA was synthesized using random hexamers and M-MuLV reverse transcriptase (NEB). Primers 5′-CTGTCATGGGCTCGCTGAGT-3′ and 5′-CCACAGTGCTATCGCTCCAA-3′, which are located external to all insert sites, were used to amplify positions 2052-2489. PCR products were subjected to electrophoresis through 1% agarose gels, and further analyzed on 2% gels if small deletions appeared to be present. Some of the PCR products were subjected to batch sequencing following gel-purification.

Minimum free energy (MFE or DeltaG), positional entropy (PE) and average positional entropy values—The RNAfold WebServer (http://rna.tbi.univie.ac.at/cgi-bin/RNAWebSuite/RNAfold.cgi; 2020-2024) was used to obtain structure and thermodynamic information for inserts. Bracket and dot notations, ΔG values, and EPS file were obtained for the MFE structures. For the EPS file, the option “MFE structure drawing encoding positional entropy” was selected (Gruber A R, Lorenz R, Bernhart S H, Neubock R, Hofacker I L. 2008. The Vienna RNA websuite. Nucleic Acids Res 36:W70-W74). Bracket and dot notations were used to draw RNA structures using the RNAcanvas web app (Johnson P Z, Simon A E. 2023. RNAcanvas: interactive drawing and exploration of nucleic acid structures. Nucleic Acids Res 51:W501-W508; https://rnacanvas.app; 2023-2024). PE values for individual nucleotides were extracted from the EPS files and applied to RNAcanvas-drawn structures using the constructed program PJNTrophy for clear to red residue color coding (see data availability). APE values for hairpins and regions of CY1 secondary structure were calculated based on PE values of the individual residues. In this study, the maximum index value for PE was set to 2.6, as this was the highest PE for any residue among all the structures.

Random hairpin generation and analysis—Using automated string manipulation in Python (version 3.12.3), 38,577 hairpins were randomly generated using various stem lengths (10, 15, 30, 45, 60, 75, or 90 bps), an apical loop of 5 randomly chosen nucleotides, and varying degrees of complementarity (100, 90, 80, 70, 60, 50, 40, 30, 20, 10 or 0%). For 100% complementary hairpins, each nucleotide on the 5′ end of the stem was paired with its Watson-Crick partner on the 3′ end of the stem. For less complementary hairpins, bases on the 3′ end of the stem were either perfectly complementary to the corresponding 5′ base or were randomly selected regardless of the 5′ end such that the overall complementarity could only be confirmed for a given percentage of the bases. For the 0% complementarity hairpins, the 5′ and 3′ ends were randomly generated independent of each other. Each hairpin was then folded in silico using the ViennaRNA package (version 2.6.4) and the thermodynamic properties were compared to the hairpins found in CY1.

Analysis of hairpins found in other +RNA viruses, DNA viruses, plant mRNA, and ribosomal RNA—Secondary structures for TBSV (Wu B, Grigull J, Ore M O, Morin S, White K A. 2013. Global organization of a positive 677 strand RNA virus genome. PLoS Pathog 9:e1003363), HCV (Mauger D M, Golden M, Yamane D, Williford S, Lemon S M, Martin D P, Weeks K M. 2015. 671 Functionally conserved architecture of hepatitis C virus RNA genomes. Proc Natl Acad Sci 672 USA 112:3692-3697), and human 16S (URS00000C35B7_9606) and 28S (URS0000ABD8B3_9606) ribosomal RNA were searched for hairpin-like regions and the corresponding sequences were folded in silico using the ViennaRNA package (version 2.6.4). Plant mRNA from both Arabidopsis thaliana and Nicotiana tobacum (NM_001035540.3, NM_001085318.5, NM_001125865.3, NM_001203084, NM_001337428.1, NM_001341019.1, NM_001342214.1, NM_001342703.1, NM_001343254.1, NM_001345462.1, NM_111130.3, NM_115393.3, NM_118738.6, NM_119712.6, NM_119996.2, NM_120281.3, NM_120667.5, NM_121392.3, NM_124089.5, NM_125860.5, NM_126153.4, V01481, X54430.1, X56263.1, X60058.1, X77950.1, XM_016577897.1, XM_016621179.1, XM_016652471.1, Z48977.1) and the coding regions from DNA viruses (Human papillomavirus 11 JN644142.1 E1a, E1b, E2, E5, 595 E6, E7a, E7b, L1a, L1b, L2; BK polyomavirus LC029411.1 VP1, VP2, small T-antigen, large T antigen; chickpea chlorotic dwarf virus HG934858.1: MP, CP, Rep, Rep-A; tomato yellow leaf curl virus HG969271.1 CP, REn, TrAP, V2, Rep, C4; wheat dwarf virus MN594280.1 MP, CP, Rep-A; chili leaf curl virus MT800761.1 U2, CP, AL2, AL3, Rep) were first folded as complete structures, which were then searched for simple hairpins and analyzed using the same method as the +RNA virus hairpins.

The program PJNTrophy and all data found in FIGS. 40A-40B is available at 10.6084/m9.figshare.27214308 Generating a CY1 Vector

To generate the first CY1 vector, selective 2′-hydroxyl acylation analyzed by primer extension (SHAPE) RNA structure probing and phylogenetic structure comparisons were used to solve the secondary structure of full-length CY1 (2.7 kb). Once the structure was known, the 3′UTR was deletion mapped to find sites that did not disrupt important functions. Using a model plant that is a host for most viruses (Nicotiana benthamiana) and infecting plants via Agrobacteria tumefaciens-mediated vacuum infiltration, the stability of CY1 with hairpin inserts in various compatible locations was determined using RT-PCR and direct sequencing. Stability may be measured by the period of time post infection until the majority of CY1 accumulating in the plant are missing all or part of the insert. The period of stability required may vary with the application.

In order to determine where inserts are tolerated downstream of the sgRNA promoter in CY1, an evaluation of where critical elements exist in the 3′ UTR of CY1 was conducted, so that such elements are avoided when inserting heterologous sequences. The 3′ CITE for CY1 was identified, as well as several additional 3′ proximal hairpins that are highly conserved in umbraviruses and known to be critical for replication and translation. Using deletions/point mutations, the sequence from the start of an sgRNA promoter-like region (positions 2068-2092 of SEQ ID NO: 1) and upstream of the 3′ CITE (positions 2468-2551 of SEQ ID NO: 1) was investigated for regions that do not materially impact either accumulation in protoplasts or systemic movement in N. benthamiana. A similar strategy was previously utilized by the present inventors to identify regions in the 3′ UTR of TCV that can accommodate hairpins targeted by RNase III-type enzymes (Aguado, L. C. et al. (2017). Rnase III nucleases from diverse kingdoms serve as antiviral effectors. Nature 547:114-117).

Inserts in CY1 may be located at one or more of positions 2083 (optionally preceded by a STOP codon) 2250, 2301, 2304, 2317, 2319, 2330, 2331, 2336, 2372, 2375 and 2426. All of these locations are in the 3′ UTR from the start of an sgRNA promoter (or promoter-like) region, typically resembling the carmovirus consensus sequence (CCS: GGGUAAAAUA) but optionally with one or two base substitutions, to the start of a 3′ Cap Independent Translation Enhancer (3′ CITE). These position numbers are in relation to SEQ ID NO: 1. Deletions or substitutions may also be tolerated in this region. The deletions or substitutions may include replacement of entire hairpin-like in or overlapping with this region with hairpin like regions designed according to “mimic” principles as described elsewhere in this application or in International Publication Number WO 2023/215782, RNA Vectors with Hairpin-like Inserts, published on Nov. 9, 2023. In CY1, for example, the hairpin-like regions at 1962-2134 or 2220-2280 may be replaced with heterologous segments.

Inserts, deletions or substitutions are expected to be stable in analogous or homologous positions in other ULVs with reference to analogous positions in CY1, for example by comparing the secondary structure of the ULV with the secondary structure of CY1. Since most ULV have expanded 3′ UTR relative to CY1, additional insert, deletion or substitution locations may be present in other ULVs. Techniques similar to those described herein may be used to locate insert, deletion or substitution positions in other ULV.

The identification of the region described above is not intended to preclude the possibility of a vector having inserts, deletions or substitutions in other parts of the ULV backbone.

### Introduction to Development of Hairpin-Like Inserts

Before factors involved in insert instability could be understood, more information was needed on the basic biology of CY1 and other ULVs. Like many viruses, ULVs use −1 ribosomal frameshifting for synthesis of their RdRp. In vitro translation using wheat germ extracts revealed an astonishing 30% frameshifting rate for CY1, compared with 2 to 5% for other members of the Tombusviridae. It was hypothesized that the high frameshifting rate for CY1 is required to synthesize a large number of progeny given the absence of a protecting RNA silencing suppressor. Moreover, all CY1 containing unstable inserts had reduced frameshifting levels, even though the frameshift site is located 1.7 kb upstream of the nearest insertion site. In addition, a correlation was found between mostly stable inserts and more modest reductions in frameshifting rates. When mapping sequences important for the high rate of frameshifting, critical sequences were found that spanned over 80% (2.2 kb) of the CY1 genome, with four long-distance tertiary interactions and two proximal tertiary interactions connecting local and distal sequences with the recoding site. These interactions contributed to multiple, highly conserved alternative RNA conformations in the recoding region. Initial efforts to increase stability of constructs with hairpin inserts centered on enhancing the stability of the active conformation of the recoding site, and while stability of inserts was significantly enhanced, low levels of deleted variants were still detectable by RT-PCR. To understand why inserts were affecting frameshifting, high-throughput SHAPE RNA structure probing of full-length WT CY1 and CY1 with inserts of varying stabilities were compared. Even small, fully base-paired hairpins caused substantial flexibility changes in residues throughout the entire genome, with unstable hairpins causing significantly more flexibility changes than ones that were mostly stable. Many flexibility changes were located in unpaired internal and terminal loops and in key RNA structures, including those at the recoding site, the cap-independent translation enhancer located in the 3′UTR and a highly conserved Y-shaped structure upstream of the recoding site that participates in one of the tertiary interactions. These results led to one of two paradigm-shifting hypotheses to explain why foreign hairpins in RNA virus vectors are not entirely stable: The conformation of the entire CY1 genome has evolved to be metastable and highly interactive through canonical and non-canonical tertiary interactions, and hairpin-like structures throughout the dynamic genome structure are perfectly tuned thermodynamically to maintain the genome-wide conformation required for critical virus functions. In other words, natural hairpin-like structures in the genome “backbone” are evolved to maintain a genome-wide metastable conformation. Insertion of a foreign (and thus non-optimized) hairpin stresses this integrative structure, leading to cascading destabilization that impacts critical tertiary interactions necessary to perform required functions such as frameshifting. The natural ability of error prone, non-processive RdRp to delete all or part of the interfering structure re-establishes viral genome homeostasis and thus enhances fitness.

To begin testing this hypothesis, exact duplicates of four natural CY1 hairpin-like structures from coding and non-coding regions were inserted into 3′UTR locations that had “kicked out” some foreign hairpins. These hairpin-like structures ranged in size from 33 to 198 nt (labeled 1 to 4 in FIG. 39). Stability of the vectors after 5 to 8 weeks in plants was measured by: (1) RT-PCR using primers outside of the insert; (2) batch sequencing the vector in newly emerged systemic leaves; and (3) direct sequencing of up to 10 cloned CYVaV1 vectors (FIGS. 11A-11E). In every case, vectors containing any of these four duplicated hairpin-like structures were completely stable and no insert loss was detected. Next hairpin-like structures were designed that would “mimic” structures 1 and 4 for insertion in the vector. This involved generating hairpin-like structures with different sequences while maintaining elements of the secondary structure or conformation (i.e., the positions of paired bases and loops, apical loop size) and approximate minimum free energy (delta G or ΔG) (as measured by RNA folding programs) of the hairpin being mimicked. Designed hairpin-like structures typically had an experimental targeting sequence (the one desired in the RISC complex) on the 5′ side of the hairpin-like structure, and 3′ side nucleotides that would fulfil the desired shape and ΔG parameters. In one exception a targeting sequence extended from a region of the 5′ side, through the apical loop, to a region of the 3′ side. Of 52 vectors containing different mimic hairpin-like structures that were tested for stability in N. benthamiana, 50 were completely stable at the assayed time point (5 to 8 weeks after infiltration). Indeed, one such insert was found to still be stable after over a year in citrus (FIGS. 11A-11E).

It was next endeavored to define properties associated with insert stability. The four natural inserts that were stable (which we used as models for mimicked inserts); four non-mimic inserts that were unstable; one non-mimic sequence of 198 nt that was unstable; 50 mimic hairpin-like structures that were stable; and two mimic structures that was unstable were examined. All stable hairpin-like structures were composed of nucleotides that together gave a low, but non-zero, average positional entropy (APE) value for the hairpin (FIGS. 12A-12B). The formula for calculating the positional entropy of an individual nucleotide (i) is S(i)=−Σpᵢⱼ log (pᵢⱼ). The positional entropy of each nucleotide in a naturally occurring or exogenous sequence can be determined, for example, using the perl script “replot.pl” from the RNAfold program in the Vienna RNA package (https://www.tbi.univie.ca.at/RNA/). The APE is calculated by adding the positional entropy of the individual nucleotides (e.g. as extracted from the EPS file produced by the RNAfold program) and dividing by the number of nucleotides. For naturally occurring hairpin-like structures in CY1, individual nucleotide entropy values vary from 0 to 2.6, where nucleotides that are always predicted to be either unpaired or paired with the same partner have a positional entropy value of 0 and nucleotides with more than one possible pairing partner, or that may be paired or un-paired, have higher entropy values. Positional entropy values were extracted for all nucleotides present in each CY1 segment of interest using the RNAFold web server. Individual nucleotides were color coded from white (entropy value=0) to darkest gray (entropy value=2.6) (FIG. 12A). Positional information was then sorted and the average positional entropy (APE) for each hairpin-like structure was determined (FIG. 12B) with the following results.

The four natural model hairpin-like structures that were all stable had APE values of: 0.19 (Hairpin-like structure 1); 0.10 (Hairpin-like structure 2); 0.12 (Hairpin-like structure 3); and 0.15 (Hairpin-like structure 4). All 50 stable mimic hairpin-like structures had APE values ranging from 0.07 to 0.32, which was substantially lower than nearly all of the unstable inserts. Most of the stable hairpin-like exogenous segments also had minimum free energy (DG) similar to (i.e. within 10 of) a naturally occurring hairpin-like structure in CY1 of similar (e.g. within 10%) length.

Four unstable hairpin-like inserts had APE values of 1.3, 1.16, 0.83, and 0.39. The unstable insert with APE=0.39 (referred to as M2250gfp30ext herein) was noted to have a 9 high positional entropy (PE greater than 1.0) and 4 very high positional entropy (PE greater than 1.5) nucleotides, with 8 (out of 66 nucleotides total) clustered near the apical loop, which may have contributed to the lack of stability. A deleted (stable) variant found accumulating in plants had a deletion of the apical loop, allowing the high positional entropy residues to form a new apical loop. Another unstable insert (198 nt) was not designed to assume any particular structure and had an APE value of 0.90. An unstable insert with low APE value (0.07; two asterisks in FIG. 12B) had three consecutive G:C pairs at the stem base and five consecutive G:C pairs in the apical region. Deleting 2 nt from the 3′ side of this hairpin (i.e., deletions did not affect the target sequence on the 5′ side) converted the hairpin from unstable to stable (APE increased slightly to 0.15).

These results strongly suggest that insert APE, optionally together with insert ΔG relative to the insert size, represent important parameters for designing stable inserts. Furthermore, results may be optionally improved by considering one or more additional stability parameters, for example the number of consecutive G:C pairs and number or clustering of high entropy nucleotides. Without intending to be limited by theory, these findings led to the second hypothesis to explain insert instability of VIGS vectors: High hairpin APE values, which imply that the hairpin is metastable and likely assuming multiple conformations with similar high ΔG values negatively impacts the processivity of the RdRp leading to polymerase pausing, which enhances the probability of a recombination event that excises some or all of the insert, thereby leading to unstable inserts. Without intending to be limited by theory, the overarching hypothesis then can be summarized as follows: Hairpin-like structures in viral genomes have evolved to have low APE values, possibly along with specific ΔG relative to the size of the hairpin-like structure, to maintain the tertiary structure of the metastable full-length genome and processivity of the replicating polymerase. Heterologous segments inserted into a viral vector must conform to these properties, which may or may not be virus-specific, to maintain viral vector fitness.

While vector stability for replication is also important in many applications, the ability to design stable inserts for virus vectors is important for allowing VIGS to be used in long-lived plants (e.g. trees and vines) for extended times against pathogens.

It is expected that the parameters for stable inserts described herein extend to inserts used in other RNA vectors derived from other viruses. Very little is known about the secondary structure of most viruses. For all but three viruses, the secondary structure is known for only portions of the virus. However, to the extent that secondary structures are known, naturally occurring viruses generally do not contain long fully based-paired regions. Considering the experiments and observations described herein, it seems likely that stable inserts for other virus-derived vectors will follow the principles described herein. In the event that one or more numerical ranges described herein do not apply to vectors derived from another virus, it is expected that the general technique of mimicking a natural-occurring hairpin-like structure will be useful. For example, the ΔG relative to length and/or APE and/or secondary structure of a wild-type hairpin-like structure may be used as a guide to designing a stable insert for a vector derived from the same wild-type virus. Given the similarities that have been observed between different Class 2 ULV, and the stability of a hairpin-like structure from one Class 2 ULV (OULV) when inserted into another Class 2 ULV (CY1), it is expected both the general technique of mimicking and the numerical parameters described herein to apply throughout Class 2 ULV. For example, it is expected that an insert described herein that is stable in CY1 will also be stable when inserted at least into any other ULV or any other Class 2 ULV. Further, given the observations and examples described herein regarding TRV and CTV, it is expected that an insert described herein that is stable in CY1 will also be stable when inserted at least into CTV and TRV.

The secondary structure of RNA hairpin-like structures includes one or more of base-paired stacks, internal loops (symmetric or asymmetric), apical loops, and junctions (FIG. 13). It is hypothesized that thermodynamic features of hairpin-like and other structures contribute to the stability of the entire CY1 genome, and structures of natural hairpin-like and other regions reflect the end point of long-term thermodynamic evolution. By understanding parameters that have influenced virus secondary structure evolution, foreign hairpin-like structures can be designed with similar features for insertion into virus vectors without loss of fitness or RdRp processivity that otherwise would lead to insert loss.

Solving the secondary structure of CY1 revealed: (1) the longest fully base-paired region was found in Structure 2 and has 13 continuous base pairs. Structure 2 also has the largest (7 base) apical loop and smallest DG (−13.1 kcal/mol) (FIGS. 12A-12B), (2) the length of the longest hairpin-like structure is 198 nt (Structure 1); (3) the length of the longest sequence without local Watson-Crick (canonical) or Watson-Crick (wobble) base-pairing is 21 nt (this sequence is not part of a hairpin-like structure); and (4) the highest number of consecutive G:C pairs in a stack in the CYVaV1 genome is three. Moreover, as described above, the APE of four natural hairpin-like sequences that were studied ranged from 0.10 to 0.19, and the APE of stable hairpin-like mimics ranged from 0.07 to 0.32, values that were lower than those of nearly all unstable inserts. As used herein, the term “base-paired” includes Watson-Crick (canonical) base pairs (i.e. C:G and A:U pairs) and Watson-Crick (wobble) base pairs (i.e. G:U base pairs) when G:U pairing is predicted by the minimum free energy structure.

One hairpin-like insert with a comparatively low APE (GFPmmck63, APE of 0.25) was unstable. However, this insert had five continuous G-C pairs. Another hairpin-like structure with a comparatively low APE (M2250gfp30ext, APE of 0.39) was also unstable. However, this insert had 9 bases (13.8%) with high entropy (PE greater than 1.0), 4 bases with PE over 1.5, and a standard deviation of entropy of 0.52. The instability of these examples may not indicate the maximum APE that may results in a stable hairpin-like insert. A hairpin-like insert with a similar or higher APE, but without the continuous G-C pairs, large percentage of high entropy bases and/or high standard deviation of PE, might be stable. Stable hairpin-like inserts may have a maximum APE of 0.75, 0.65. 0.5, 0.4, 0.39, 0.36 or 0.32. Minimum APE of a stable hairpin-like structure may be 0.01, 0.02, 0.03, 0.04, 0.05, 0.06 or 0.07. A range of APE may optionally be provided by any combination of these minimum and maximum APE values, or any APE values described in the examples.

An insert may exceed one or more of the parameters described above or derived from a wild type virus. For example, one hairpin-like mimic was stacked onto the majority of Structure 3 (in its natural location), resulting in a stable hairpin-like structure of 247 nt. The entirety of this hairpin-like structure was not heterologous, however it suggests that the maximum length of a natural hairpin-like structure might be exceeded. A stable insert with 4 consecutive G:C pairs was also introduced into CY1, whereas a maximum of 3 are found in natural CY1 hairpin-like structures. An insert with 5 consecutive G:C pairs was not stable. Further, heterologous inserts with APE greater than the APE of natural CYVaV hairpin-like structures were used. The examples above suggest that in some examples a natural parameter may be exceeded, for example by up to one half, or by up to one third.

U.S. Pat. No. 7,229,829, Tobacco rattle virus and related compositions and methods, which is incorporated herein by reference, describes RNA1 and RNA2 sequences of tobacco rattle virus (TRV). Other TRV vector sequences are also known. As is known in the art, TRV RNA1 and RNA2 sequences, or portions thereof, can be modified to include a heterologous RNA segment for delivery to a plant.to achieve, for example, host plant gene downregulation (i.e. as a VIGS vector). TRV vectors are used, for example, to test the effects of proposed genetic modifications of plants. However, in this context, the heterologous RNA segment is only required to remain in progeny of the vector for a short period of time. Folding the first 2400 nt of tobacco rattle virus (TRV) RNA2 revealed a low entropy hairpin-like region from positions 1727 to 1925. The APE value for this hairpin-like region is 0.32 and the ΔG is −61.5 kcal/mol. This can be compared to similarly sized Structure 1 of CY1 (APE=0.19; ΔG=−89.2 kcals/mol).

International Publication Number WO 2022/133186, Create Huanglongbing tolerance by silencing a citrus negative immune regulator, which is incorporated herein by reference, describes RNAi vectors (e.g. VIGS vectors) derived from citrus tristeza virus (CTV). A heterologous RNA segment is inserted in one of four regions of the CTV genome. In another example, a naturally occurring hairpin-like structure was located in CTV and inserted into CY1 and infected into a benthamina plant (see further below). The resulting vector was stable when assayed after three-weeks.

In combination with further examples related to poliovirus and coxsackievirus B3 (see further below), these observations suggest that inserts designed with reference to other wild type virus may be used in CY1 or other ULV, and that inserts designed with reference to CY1 or other ULV, or otherwise as described herein, may be useful in other viral molecules, for example other RNA molecules derived from plus-sense RNA viruses generally, other RNA plant virus generally, and other enteroviruses or other RNA animal viruses, as well as CTV, TRV, poliovirus and CXVB3 in particular. Even if the resulting vectors are not as stable as CY1-derived vectors, they may be more stable for replication than comparable vectors with conventional (i.e., fully base-paired) hairpins.

The inventors decided to test if inserting duplicates of natural hairpin-like structures into the insert sites would be stable. Despite the size of some of these structures, all four of the duplicate hairpin-like structures used were stable in the insert sites. Next, the inventors discovered that mimicking the secondary structures and approximate DG of the natural hairpin-like structures with heterologous sequences also resulted in vectors that did not lose the inserted sequences. The inventors then investigated the properties of the natural hairpin-like structures, finding that various parameters such as the positional entropy of individual nucleotides, average positional entropy of the overall hairpin, and DG were relevant to stability. Unstable inserts typically had either high positional entropy, higher ΔG, multiple consecutive G:C pairs, or high positional entropy nucleotides that were clustered together. Using these principles, exogenous inserts with targeting sequences were designed that could be inserted into vectors and that were stable (i.e. retained in progeny) even without any reverse fitness modifications described herein. However, one or more reverse fitness modifications may be optionally used in combination with otherwise stable inserts.

It is commonly believed that bacteria do not take up siRNA. However, Singla-Rastogi et al. (2019) describes examples in which small interfering RNA targeted against some specific genes were taken up by Pseudomonas syringae and cause a 50% reduction in the population of Pseudomonas syringae. The inventors have confirmed that the conventional belief is at least partially correct. For example, in experiments conducted by the inventors, E. coli did not take up siRNA. However, as described in the examples herein, small RNA are taken up by some bacteria. In particular, bacteria are taken up by Pseudomonas syringae, Erwinia amylovora and Liberibacter crescens. These three bacteria are all gram negative bacteria that infect plants. However, since these three bacteria are otherwise unrelated to each other, they indicate that small RNA can be taken up by bacteria that infect plants generally, or at least by gram negative bacteria that infect plants. P. syringae is a plant pathogen that causes, for example, bacterial canker in almond trees. Erwinia amylovora is a plant pathogen that causes, for example, fire blight in apple trees, pear trees and some other trees in the Rosaceae family. Liberibacter crescens is a relative of Liberibacter asiaticus and, based on their experiments with Liberibacter crescens, the inventors believe that Liberibacter asiaticus will also take up small RNA.

An siRNA is typically designed to be a complement to a part of RNA or DNA associated with the target organism intended to be treated or controlled by the siRNA (“specific siRNA”). For example, as shown in the examples herein, Pseudomonas syringae, Erwinia amylovora and Liberibacter crescens can all be controlled by small RNA that are complements of genes (including complements of messenger RNA) of the bacteria. In particular, these bacteria were controlled, for example by 1000 fold reductions in their population in infected plants, by specific siRNA that complement the adenylate kinase (ADK) or gyrase subunit A (GyrA) genes of the bacteria.

Sequences for growth inhibition of Erwinia amylovora in vitro and in vivo are presented below. Two Erwinia genes were targeted: MurA and GyrA.

Erwinia MurA (in vitro) (SEQ ID NO: 59):
gaactggtgaaaaccatgcgcgcctcgatttgggcattgggcccgctggtggcacgttttggccaggggcaagtatcactgcccggt
ggttgcgctatcggcgcacggccggttgatcttcatatcaccggccttgagcagctcggcgccgagatcaaactggaagaaggttac
gttaaagcctctgtcgcgggtcgcctgaaaggggcgcatatcgttatggataaggtcagcgtgggtgcaaccgtcactatcatgagtg
cggcgacgctggcaacgggcaccaccgttatcgagaatgctgcgcgtgagccggaaattgtcgacactgccaacttcctcaacacg
cttggggcgaaaatcaccggtgccggcagcgatcgtatcaccatcgaaggtgttgatcgccttggtggcggtgtttatcgcgtacttcct
gaccgcatcgaaaccggtactttcctggtgggggagcgatttccggcggtaaggttacctgccgtgcggcgcagcccgatacgctg
gatgctgtactggctaagctacgcgaagccggtgcggacatcgagatgggagaagactggataagcctggacatgcacggtaagcg
gcctaaagcggtcaatttacgcacagegccgcatcccggtttcccaaccgatatgcaggcgcagttcagtttgttgaacctggtggctg
aaggcacgggggtgattactgaaaccatcttcgaaaaccgctt

### Erwinia MurA (In Vivo):

Fragment 1 (SEQ ID NO: 59, residues 1-402):
gaactggtgaaaaccatgcgcgcctcgatttgggcattgggcccgctggtggcacgttttggccaggggcaagtatcactgcccggt
ggttgcgctatcggcgcacggccggttgatcttcatatcaccggccttgagcagctcggcgccgagatcaaactggaagaaggttac
gttaaagcctctgtcgcgggtcgcctgaaaggggcgcatatcgttatggataaggtcagcgtgggtgcaaccgtcactatcatgagtg
cggcgacgctggcaacgggcaccaccgttatcgagaatgctgcgcgtgagccggaaattgtcgacactgccaacttcctcaacacg
cttggggcgaaaatcaccggtgccggcagcgatcgtatcaccatcgaaggtgtt
Fragment 2 (SEQ ID NO: 59, residues 343-743):
aacacgcttggggcgaaaatcaccggtgccggcagcgatcgtatcaccatcgaaggtgttgatcgccttggtggcggtgtttatcgcg
tacttcctgaccgcatcgaaaccggtactttcctggtggcgggagcgatttccggcggtaaggttacctgccgtgcggcgcagcccga
tacgctggatgctgtactggctaagctacgcgaagccggtgcggacatcgagatgggagaagactggataagcctggacatgcacg
gtaagcggcctaaagcggtcaatttacgcacagcgccgcatcccggtttcccaaccgatatgcaggcgcagttcagtttgttgaacctg
gtggctgaaggcacgggggtgattactgaaaccatcttcgaaaaccgctt

### Erwinia GyrA (In Vitro) (SEQ ID NO:60):

atcgtcaacctgctgccgctggaagccaacgagcgtattactgcaattctgccggtgcgtgaatatgccgaaggctggaatatctttatg
gctaccgccagcggaacggtgaagaaaaccgcgctgactgacttcagccgaccgcgcagcgccggtattattgccgtcaatctgcgt
gatgacgatgaactgatcggcgtgtcgctgacgaacggtagtgatgaagcgatgctgttttctgccgccggtaaagtggtgcgattcgc
ggagagcgcggtgcgtacgatgggccgtaccgcctccggcgtacgcggcatcaagctggctgaaggcgaccgcgtggtgtcgctg
atcgtgccgcgtgatgatggcgctatcatgaccgtgacgcaaaacggctacggtaaacgaacggctaacgtcgaatatcccaccaag
tcgcgtgcgactcagggggttatctcgatcaaggtaaccgagcgtaacgggccggttatcggtgcggtgcaggtggtcgatggcgat
cagatcatgatgatcaccgatgccggcacgctggtacgtacccgcgtgtccgaggtcagcgtggtagggcgtaacacccagg

### Erwinia GyrA (In Vivo):

Fragment 1 (SEQ ID NO: 60, residues 2-416):
tcgtcaacctgctgccgctggaagccaacgagcgtattactgcaattctgccggtgcgtgaatatgccgaaggctggaatatctttatgg
ctaccgccagcggaacggtgaagaaaaccgcgctgactgacttcagccgaccgcgcagcgccggtattattgccgtcaatctgcgtg
atgacgatgaactgatcggcgtgtcgctgacgaacggtagtgatgaagcgatgctgttttctgccgccggtaaagtggtgcgattcgcg
gagagcgcggtgcgtacgatgggccgtaccgcctccggcgtacgcggcatcaagctggctgaaggcgaccgcgtggtgtcgctga
tcgtgccgcgtgatgatggcgctatcatgaccgtgacgcaaaacggctacggtaaacgaacggc
Fragment 2 (SEQ ID NO: 60, residues 207-607):
gacgaacggtagtgatgaagcgatgctgttttctgccgccggtaaagtggtgcgattcgcggagagcgcggtgcgtacgatgggccg
taccgcctccggcgtacgcggcatcaagctggctgaaggcgaccgcgtggtgtcgctgatcgtgccgcgtgatgatggcgctatcat
gaccgtgacgcaaaacggctacggtaaacgaacggctaacgtcgaatatcccaccaagtcgcgtgcgactcagggggttatctcgat
caaggtaaccgagcgtaacgggccggttatcggtgcggtgcaggtggtcgatggcgatcagatcatgatgatcaccgatgccggca
cgctggtacgtacccgcgtgtccgaggtcagcgtggtagggcgtaacacccagg

Gene sequences of P. syringae tabaci_Gyrase subunit A (Pst_GY), P. syringae tabaci_Adenylate Kinase (Pst-ADK), and GFPuv were utilized for specific siRNA targets are presented below (wherein sequences underlined in solid line are forward primers for dsRNA synthesis, and sequences underlined in dashed line are reverse primers for dsRNA synthesis):

P. syringae tabaci_Gyrase subunit A (Pst_GY) (SEQ ID NO: 61):
atgggcgaactggccaaagaaatcctcccggtcaatatcgaagacgagctgaagcagtcctacctcgactacgcgatgagcgtaatc
gtcggtcgagcactgcccgatgcgcgcgacggcttgaagcccgtgcaccggcgcgtgttgttcgcaatgagcgagctgggtaacga
ctggaacaagccgtacaagaaatccgcccgtgtggttggtgacgtgatcggtaagtatcacccgcacggcgatacagccgtgtacga
caccatcgttcgtatggctcagccattctcgctacgctacctgctggtagacggtcagggcaacttcggttcggtcgatggcgacaacg
ctgcggccatgcgatacaccgaagtgcgcatgaccaagctgggcacgagctgctggccgacctgcacaaggaaaccgtggactg
ggtgccgaactacgacggcaccgaaatgatccccgcggtcatgccgacccgtattcccaacctggtggtcaacggttccagcggtat
cgccgtgggcatggcgaccaacattccgccgcacaaccttggtgaagtcatcgacggttgcctggcactgatcgacaaccccgagtt
gacgatcgatgagctgatgcagtacatccccggcccggatttcccgacagcggcgatcatcaacggccgtgcgggtatcatcgaagc

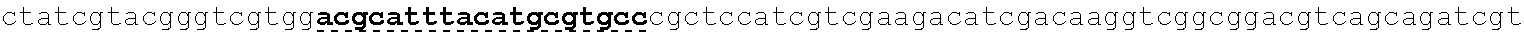

catcaccgaactgccgtaccagcttaacaaggcacgtctgatcgagaagatcgccgagctggtcaaggaaaagaaactcgaaggca
ttaccgagctgcgtgacgagtccgacaaggacggtatgcgcgtggtcatcgagctgcgtcgtggcgaagtgccagaggttgttctca
acaacctttatgcccagacccagctgcaaagcgttttcggtatcaacatcgttgccttgattgacggtcgtccacgcatcctgaacctcaa
ggacctgttggaagcctttgttcgtcaccgtcgcgaagtggttacccgccgtaccgtattcgagctgcgcaaggcgcgtgagcgtggc
catatccttgaaggtcaggctgttgcgttgtccaacatcgacccggtcatcgccctcatcaaggcctctccgacacctgcagaagccaa
ggaggcgttgatcaagacgccttgggaatcaagcATGCGCGTGATTCTGCTAGGAgcagtggtcgaaatggtcg
agcgtgcaggtgccgattcgtgccgccctgagaacctggacccgcaatacggtctgcgtgaaggcaagtatttcctgtcaccggaac
aggctcaggccattctggaactgcgtctgcatcgcctgaccggtctggaacacgagaagctgctgggcgagtaccaggaaatcctca
accagatcggcgagctgatccgcatcctcaacagcgcaacgcgcctgatggaagtgatccgcgaagagctggaagtgatccgctcc
gagtacggcgatgcccgtcgtaccgagattctcgatgcacgtctggacctgaccctgggtgacctgatcaccgaagaagagcgtgtg
gtcaccatctcccatggcggctatgccaagacccagccattggcggtctatcaggctcagcgtcgtggcggcaagggcaagtcggc
caccggcatcaaggatgaggattacattgctcacctgctggtcgccaacagccacacgacactgctgatgttctccagcaagggcaa
ggtgtactggctcaagacctatgagatcccggaagcgtcccgcgctgcccgtggtcgtccgttggtcaacctgttgccgctgagcgac
ggcgaatacatcaccaccatgctgccggttgacctcgaagccatgcgcaagcgtgccgacgaagaaggcgaagccctcgaaggcg
agctggacgacgcggaaaacagcagcgagaccgaagaagagcgcaaggcccgtatcaaggccgccgacaagaagaaggctcc
gttcatcttcatgtccaccgccaacggtaccgtcaagaagaccccgctggttgcattcagccgtcaacgcagttcgggcctcattgccct
tgagctggacgagggcgacatcctgatctccgctgccattaccgatggcgaacaggaaatcatgctgttctccgatggcggcaaagtg
acccgcttcaaggaatccgatgtgcgcgccatggggcgtaccgctcgcggcgtgcgtggcatgcgtctgccagaagggcaaaagct
catttcgatgctgatcccggaagaaggcagccagatcctcaccgcttccgagcgcggttacggcaagcgtacggccatttccgagttc
cccgagtacaagcgcggcggtcagggtgtcatcgccatggtcagcaacgagcgtaacggccgtctggttggcgcagttcaggtgctt
gatggcgaagaaatcatgctgatttccgatcagggcacgctggtgcgtacccggggggcgaagtgtccagtctgggccgtaacact
cagggtgtgaccctgatcaagctggccagcgacgagaaactggtcggtctggagcgtgttcaggagccgtcggaagtcgaaggcg
aagagcttgaaggcgaagaagttatcgacggcgtgattgtcgatgccgctgaagctgaagtgggcgacgccggtgaagacctgcaa
gcggacgctgcgccagacgaagacgaaccgcagaactga
P. syringae tabaci_Adenylate Kinase (Pst-ADK) (SEQ ID NO: 62):
atgcgcgtgattctgctaggagctcccggggccggtaaaggtactcaggcaaaattcatcactgaaaatttcggcatcccgcaggtttc
gacaggcgacatgctgcgcgctgcagtcaaggctgaaaccgagcttggcctgaaggccaagagcgtcatggactcgggtggtctg
gtttccgatgacctgatcattggtctgatcaaggatcgtctggcccagccggattgtgcgaacggcgttctgttcgacggcttcccgcgc
accattcctcaggccgaagccctgttgaaagcaggtctggaaatcgaccacgtgctggaaatcgccgttgatgacgaagaaatcgtca
agcgcatgtcgggccgccgggttcacgaaggctctggtcgcatctatcacaccattttcaacccgccgaaagtcgagggtatcgatga
tgtgactggtgaaccgctgttgcagcgcaaggacgacgtcgaagaaaccgtgcgtcatcgcctgtcggtctaccatgcccagaccaa
gccgctggtcgagttctacagcaagctggaagcaaagaacggcaagcccaagtgcagccatattccaggtgttggctcggttgaaga

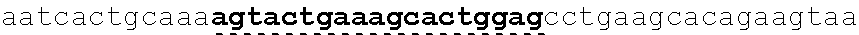

GFPuv (SEQ ID NO: 63):
atgagtaaaggagaagaacttttcactggagttgtcccaattcttgttgaattagatggtgatgttaatgggcacaaattttctgtcagtgga
gagggtgaaggtgatgcaacatacggaaaacttacccttaaatttatttgcactactggaaaactacctgttccatggccaacacttgtca
ctactttctcttatggtgttcaatgcttttcccgttatccggatcatatgaaacggcatgactttttcaagagtgccatgcccgaaggttatgta
caggaacgcactatatctttcaaagatgacgggaactacaagacgcgtgctgaagtcaagtttgaaggtgatacccttgttaatcgtatc
gagttaaaaggtattgattttaaagaagatggaaacattctcggacacaaactcgagtacaactataactcacacaatgtatacatcacg
gcagacaaacaaaagaatggaatcaaagctaacttcaaaattcgccacaacattgaagatggatcagttcaactagcagaccattatca

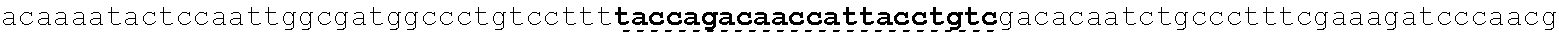

aaaagcgtgaccacatggtccttcttgagtttgtaactgctgctgggattacacatggcatggatgagctctacaaataa

Enhanced Stabilization: The ability to achieve a sufficiently stable insert in a viral vector has eluded prior researchers due in part to a lack of expertise in RNA structure. Some researchers attempted to use an empirical trial-by-error approach to choose insert locations, but without a sufficient understanding of the effect of the insert on the stability of the vector. In accordance with the present disclosure, the core principles behind what makes inserts stable were investigated and utilized to optimize the process of insert location and structure. First, an excellent correlation was discovered between vector instability and numerous structural alterations throughout the viral genome as assayed by SHAPE structure probing, which results in poor in vitro translation of the polymerase that requires a −1 ribosomal frameshift. The following hypothesis was then tested: what if RNA viruses can discern “self” from “non-self”? In other words, what if RNA viruses have evolved their genomes such that every portion of the genome is maximally structured to support the most fit virus? This implies that every hairpin emanating from the genomic RNA nucleic acid “backbone” is maximally stable for its size and shape and properly configured for a successful virus.

It was determined that the insertion of haphazard hairpins whose design is based solely on siRNA generation, and which do not resemble in length, structure and/or stability endogenous, evolved hairpins, stresses and destabilizes the entire structure of a viral genome. Since RNA viral genomes are dynamic, with active and inactive structures precisely balanced to control key viral functions like frameshifting, destabilizing the genome can have adverse repercussions that are relieved if the inserts are deleted through recombination. If the genome contains foreign structures, the deletion or removal of inserts or portions thereof (e.g., replicase-mediated events) will eventually occur, thereby leading to the non-functionality of the commercial RNA vector product. Such deletion and/or removal of inserts or portions thereof increases the possibility of generating a more fit virus (though not necessarily functioning to target the desired pathogens/host gene expression), as the virus with a destabilizing insert is more poorly fit.

Following from the proposed hypothesis, designing inserts that “mimic” the structure (i.e., length and/or the size and location of looped or non-base-paired regions) and/or stability (i.e., the ΔG) of endogenous hairpins (which optionally include hairpin-like structures and structures with multiple hairpins or hairpin structures) should increase stability of the insert within the vector, particularly when inserted into suitable locations. Mimicked hairpins are designed by using the sequence of interest (i.e., an siRNA) as some or all of one side, for example the 5′ side, of the hairpin and an artificial sequence on the other side, for example the 3′ side, that provides the corresponding nucleotides for the base-pairs and loops, resulting in a structure that mimics an endogenous (natural) hairpin at some location on the wild-type vector or a related wild-type vector. A mimic insert may be inserted in the same location, i.e., as a replacement for the wild-type hairpin that was mimicked. Alternatively, a mimic insert may be inserted into a different location, with the wild-type hairpin either retained or deleted, if not required for viability of the vector.

In accordance with the present disclosure, hairpin inserts were engineered to have a structure that mimics the structure of a natural endogenous hairpin structure of the vector of interest or a relative thereof (e.g., a CY1 vector having an insert that mimics a hairpin structure of CY1 or another ULV, for example a Class 2 ULV such as OULV). As noted above, providing and maintaining stability of sequences inserted into the CY1 vector, which are subsequently converted into siRNAs when the RNA virus vector is targeted by the RNA silencing defense system in infected plants, raises various challenges. While some insertions are randomly more stable than others (i.e., the inserts remain in the vector for a longer time), little was previously understood about parameters required for stabilization. Solving the secondary structure of the CY1 vector revealed an extremely compact structure with few extensive single-stranded regions, which would generally be considered the most suitable locations for making insertions. Various locations (noted above) were identified for accepting particular hairpin inserts (e.g., that targeted nuclear-encoded GFP), although other hairpin structures had different stabilities in all of these particular locations. Thus, producing a vector that allows for little or no discernable insert loss (e.g., assayed first by PCR and then by sequencing the cloned population after four to six weeks) proved difficult. This indicated that these hairpins decreased the fitness of the vector and that deletion of almost all (but not precisely all as surrounding vector sequences could also be deleted) or part of the insert increased fitness.

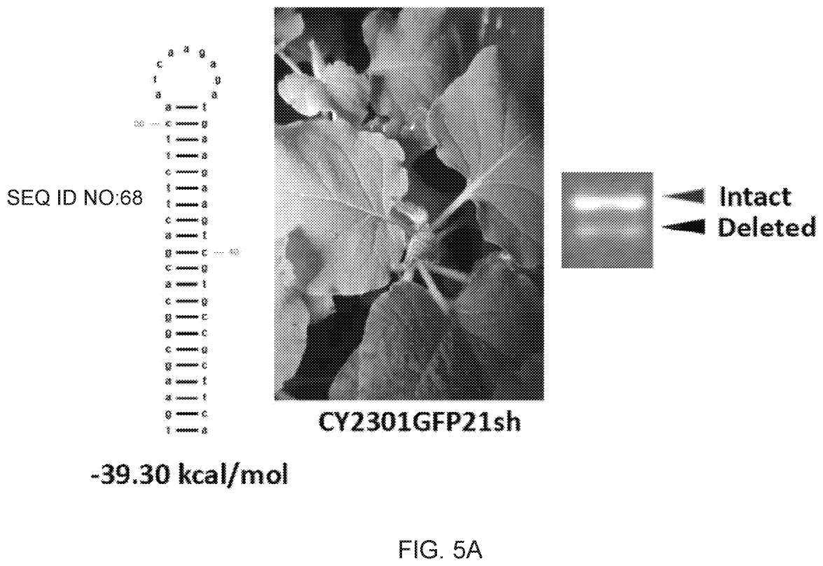

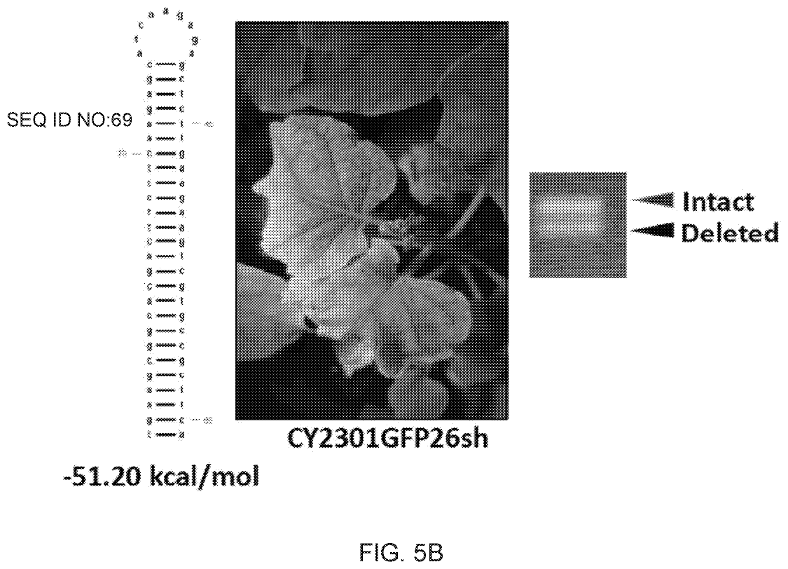

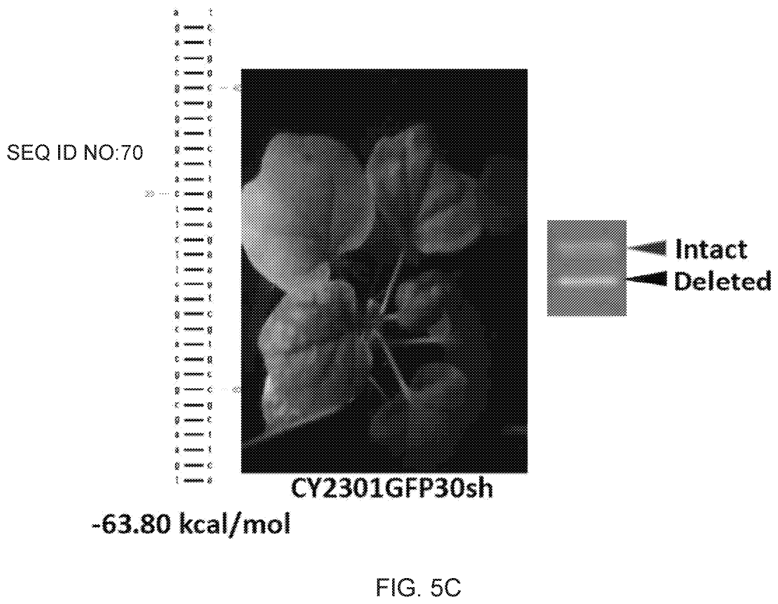

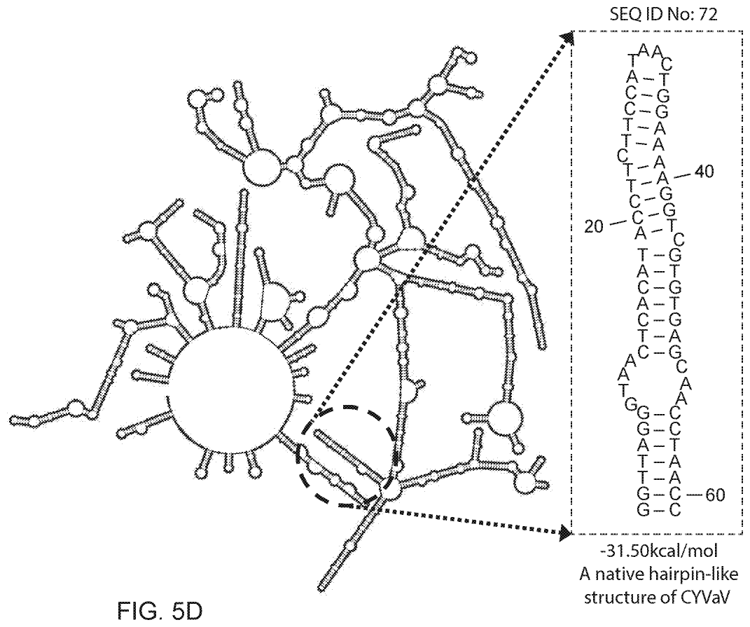

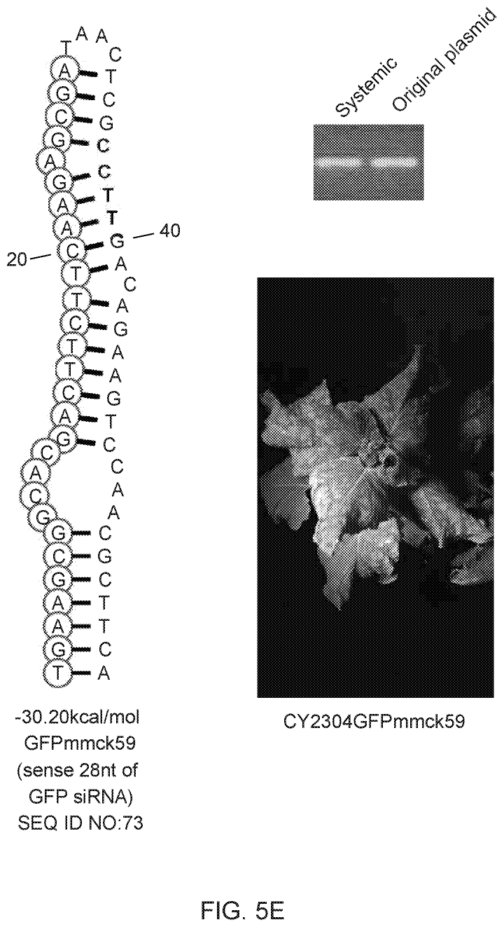

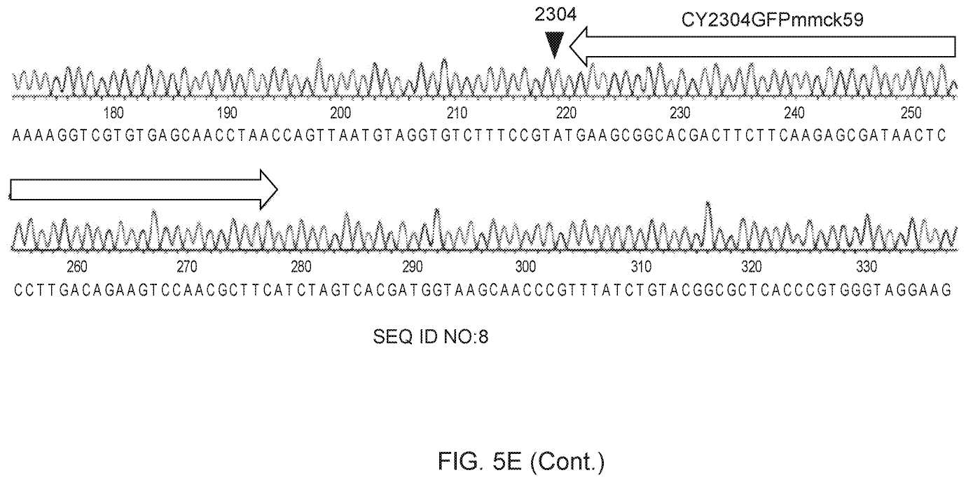

Intuitively, the more stable the structure of the hairpin insert (i.e., the higher or ‘more negative’ the ΔG), the less it should interfere with important viral structures (generally thought to be the reason for vector instability), providing that the polymerase is still able to melt the hairpin during replication. Surprisingly, it was discovered that the opposite is true (see FIGS. 5A-5C). In particular, it was discovered that structurally stable hairpins were rapidly lost, as were inserted sequences with minimal structural stability. Also tested were non-structured inserts that were placed atop RNA scaffolds (e.g., lock and dock structures). While the lock and dock stabilized unstructured inserts and resulted in some very stable inserts, some inserts generated a small but discernable population of CYVaV that had lost various amounts of the insert and scaffold, particularly vectors with two such inserts.

An RNA vector (e.g., CY1 vector) including such mimic hairpins exhibited substantially increased stability as compared to a vector that included a hairpin structure that did not mimic the natural hairpin structure. The structure and sequence of a natural hairpin was determined; a mimic hairpin having a substantially similar structure and/or sequence and/or ΔG to the natural hairpin was then inserted into the vector molecule. Studies demonstrated that vectors possessing such mimic hairpin(s) inserted at the same location as the corresponding natural hairpin(s) (i.e., replacing the natural hairpin(s) at such location) were stable. Further studies demonstrated that vectors possessing such mimic hairpin(s) inserted at one or more different locations from the corresponding natural hairpin(s), but possessing substantially the same structure(s) and/or sequence(s) and/or ΔG as compared to the natural hairpin(s), were stable. It was found that hairpin structures having relatively conserved sequences across iRNAs (e.g., CY1 and relatives thereof, other RNA molecules having least 50% or at least 70% identity with CY1) were particularly suitable as mimic hairpin structures. However, it was also found that a wild-type hairpin structure in a CY1 relative, for example another Class 2 ULV such as OULV, could be used to design a stable CY1 insert even if the wild-type hairpin structure is not present in CY1.

Referring to FIG. 5E, a mimicked hairpin (wherein the natural yet unnecessary hairpin at position 2219 was replaced with a mimic hairpin that resembles the natural hairpin) produced an extremely stable virus vector with no discernable loss of insert for the life of the plant. For example, random events that may lead to insert loss may generate a less fit virus, with such virus(es) lost from the population. The process of replacing a natural hairpin with an engineered mimic hairpin was repeatedly tested, with several mimics for each of three different natural hairpins, including one that is fully base-paired, and the mimic hairpins inserted into positions 2219 and 2304. In every case, the mimicked hairpin replacement at positions 2219 and 2304 resulted in an extremely stable virus vector. It was determined that the closer the ΔG of the mimicked hairpin is to the endogenous hairpin being mimicked, the more stable the insert. RNA structure probing clearly showed far fewer structural disruptions throughout the vector genome from the presence of a mimic hairpin as compared to random hairpins inserted at the same locations. This further demonstrated the importance of mimicry to achieve a vector that maintains a structure close to (though generally not identical to) the wild-type RNA genome structure.

Mimicked hairpins were inserted in one of three different locations, either singly or with a second mimic hairpin. All tested structures were extremely stable, including constructs including two inserts (see FIGS. 6A-6D). A wide variety of mimicked hairpins have been tested in a variety of locations, including constructs having mimic hairpins based on natural hairpin structures of CY1. In one implementation, relatively large hairpins of CY1 were used (e.g., having a length of about 200 nt or more) in order to stack or incorporate multiple siRNAs into a single insert.

Referring to FIGS. 6A-6D, the two inserts mimic Structure 4 of CY1. The ΔG of Structure 4 is −30.00 kcal/mol. The APE of Structure 4 is 0.15. The ΔG of insert CS7-V2.2 is −32.50 kcal/mol and the APE is 0.26. The ΔG of insert Mmck6.1 is −28.40 and the APE is 0.15. The sequence of Mmck6.1 is (SEQ ID NO:9):

ggaagugauggacgaaauuaaugauuccauaacuggaacauuacauuuc
gucaacacuucc

The first 24 nt of Mmck6.1, shown in bold above, are an siRNA sequence targeted at CTV. Insert CS7-V2.2 is a targeted against the callose synthase gene of the host plant. The sequence of CS7-V2.2 is (SEQ ID NO:10):

gguaucaaugggcagacgaagauuuggcauaacugccaaucuuccgucu
gcaaaugauacc

The first 27 nt of CS7-V2.2, shown in bold above, are an siRNA sequence targeted at the callose synthase gene of N. benthamiana.

The (relatively) unstable insert shown in FIG. 6D is CY2301GFP30sh, a conventional (i.e. fully base-paired) hairpin which is also shown in FIG. 5C and further described by SEQ ID NO:88. Insertion of CY2301GFP30sh was shown to produce SHAPE changes and to effect stability of the RNA structure throughout the CY1 genome (FIGS. 6A-6D). CY2301GFP30sh was inserted at location 2301 of CY1. After 3-4 weeks of infection, a PCR assay identified a band corresponding to CY1 without the CY2301GFP30sh insert. The batch sequencing of CY2301GFP30sh show in FIG. 6D shows heterogeneity, further indicating the presence of replicates without the insert. The APE, and positional entropy of individual bases, of CY2301GFP30sh is very low. However, whereas low positional entropy provides stability for inserts with hairpin-like structure, this factor does not predict stability of a conventional (i.e. fully base-paired) insert. The instability of conventional hairpins appears to be related to other considerations.

As demonstrated by the disclosed data, the mimic hairpin inserts are extremely stable within the vector (e.g., a CY1-based vector) and thus are particularly well suited for use as a vector, for example a VIGS vector, for targeting pathogens and endogenous gene expression to control diseases in long lived plants. As known in the art, VIGS is a post-transcriptional gene silencing (PTGS)-based technique that exploits the natural defense mechanisms employed by plants to protect against a viral pathogen. See, e.g., Pantaleo et al. (2007) Molecular Bases of Viral RNA Targeting by Viral Small Interfering RNA-Programmed RISC, J Virol 81(8):3797-3806; Ramegowda et al. (2014) Virus-induced gene silencing is a versatile tool for unraveling the functional relevance of multiple abiotic-stress-responsive genes in crop plants, Plant Genetics and Genomics, Vol. 5, Art. 323; Mei et al. (2016) A Foxtail mosaic virus Vector for Virus-Induced Silencing in Maize, Plant Physiol 171:760-772).

Conventional VIGS vectors have not been suitable for long-lived trees. Viruses have relatively limited host ranges, requiring the need to develop a different virus vector for each tree or vine (and sometimes requiring more than one virus vector for the same crop). As such, the cost for the development and approval of a virus that infects a single or limited crop type may be prohibitive. In addition, any virus utilized as a vector should be relatively mild or asymptomatic, thus eliminating numerous viruses as suitable vectors. In addition, sequences inserted into conventional viral vectors are generally unstable, with the insert typically remaining intact only for several days or weeks. However, stability is needed for many years for some hosts such as tree and vine crops.

CY1 and CY1-like molecules are particularly well suited for use as a VIGS vector, or vector having an siRNA or other insert, particularly for use in treating tree and vine pathogens requiring long-term stability. CY1 exhibits an exceptionally wide host range due to its lack of endogenous movement proteins and use of host movement proteins and little or no detrimental symptoms to the host, may be engineered to include extremely stable inserted sequences, and rarely (if ever) unintentionally spread from plant to plant. In addition, CY1 does not encode any RNA silencing suppressor.

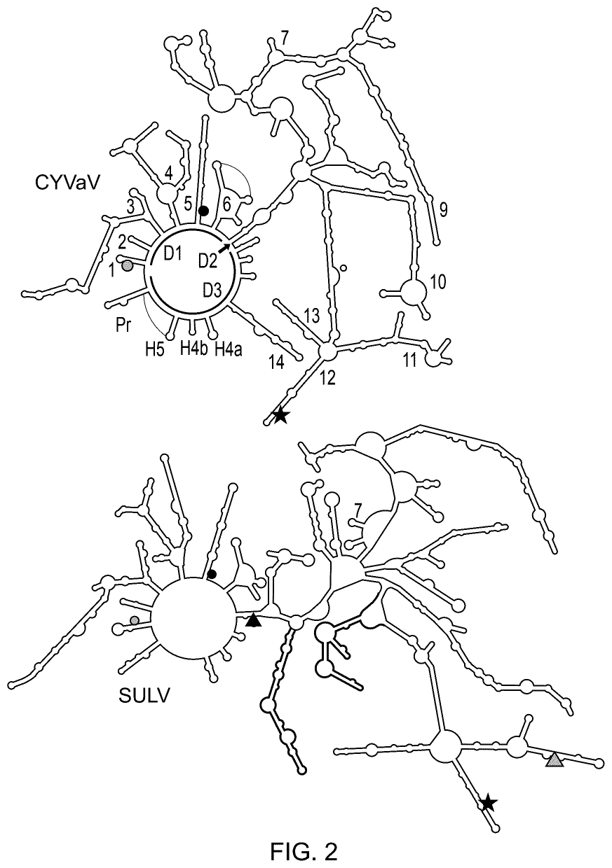

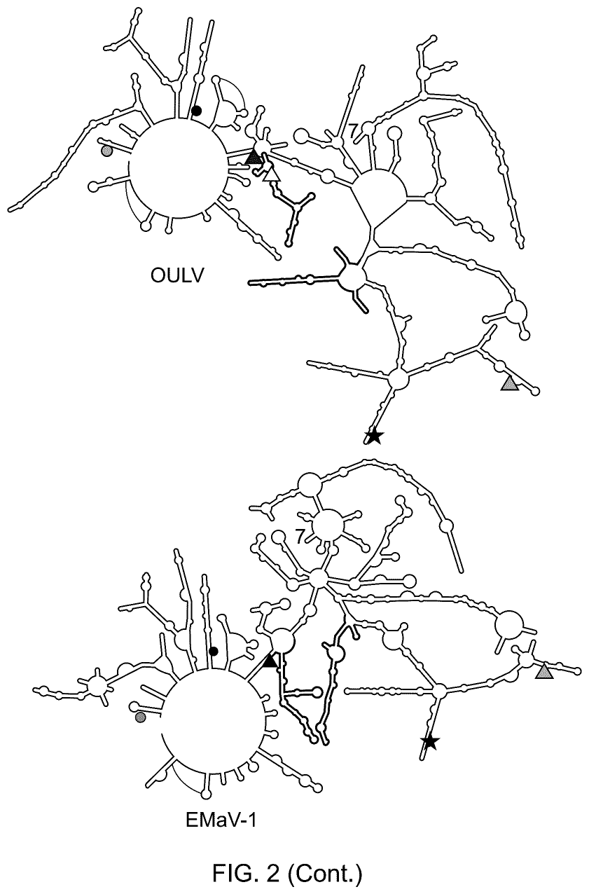

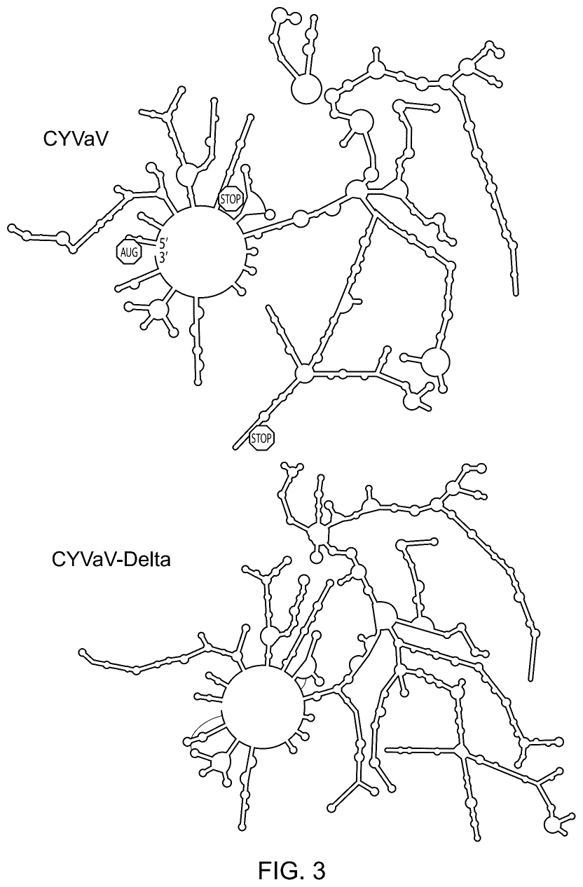

The full length structure of CY1 was determined by SHAPE structure probing and phylogenetic comparisons with the CY1 relatives in Opuntia, Fig and Corn (FIGS. 1C and 2).

### A. Determination of Positional Entropy and Average Positional Entropy

Determining the positional entropy of the bases in an insert starts with determining the secondary structure, i.e. the arrangement of paired and unpaired bases, of the insert. Although RNA folding software may be inaccurate for large sequences, the output of RNA folding software is typically accurate for small sequences, for example inserts of less than 300 nt or less than 200 nt. Additional methods such as SHAPE reactivity may be used to determine the secondary structure of large or difficult inserts, or to confirm the accuracy of or modify a software-produced secondary structure drawing.

Using the RNAfold software as an example, the sequence of an insert is entered into the program. The available results include a minimum free energy (MFE) prediction, an optimal secondary structure of the insert sequence in the MFE state in dot-bracket notation, and a graphical drawing of the dot-bracket notation of the MFE structure. Selecting “EPS” in the download options for the MFE structure drawing encoding positional entropy produces a file with a list of the positional entropy of each base in the insert. The positional entropy of the individual bases are added together and divided by the number of bases to produce the average positional entropy (APE).

Unless stated otherwise, the values of DG, PE and APE described herein are produced using RNAfold version 2.4.18 available from, or accessed through, the University of Vienna. The program is further described in Mathews D H, Disney M D, Childs J L, Schroeder S J, Zuker M, Turner D H. (2004) Incorporating chemical modification constraints into a dynamic programming algorithm for prediction of RNA secondary structure. Proc Natl Acad Sci USA 101(19):7287-92; Gruber A R, Lorenz R, Bernhart S H, Neubock R, Hofacker I L. The Vienna RNA Websuite. Nucleic Acids Research, Volume 36, Issue suppl_2, 1 Jul. 2008, Pages W70-W74, DOI: 10.1093/nar/gkn188; and, Lorenz R, Bernhardt S. H., Honer zu Siederdissen C, Tafer H, Flamm C, Stadler P F and Hofacker I L, “ViennaRNA package 2.0”, Algorithms for Molecular Biology, 6:1 pages 26, 2011.

### B. Stability Assay

Stability of the insert is determined after allowing the insert to replicate in plants for a period of time. The vector is then harvested from the plant and assayed through RT-PCR. A stable insert will show only one band indicative of the intact vector. The appearance of a second band indicates vector replicates that have deleted the insert, and the insert is not entirely stable. In an entirely stable vector, batch sequencing will show no heterogeneity.

### C. Natural Hairpin-Like Structures in CYVaV1

FIG. 39 shows the secondary structure of full-length CY1. Each “dot” represents a nucleotide. Numbers denote natural CY1 hairpin-like structures that served as templates for the mimic hairpins. When duplicated and inserted into different locations in the CY1 3′UTR, these hairpin-like structures were stably maintained at all time points assayed until senescence of the host plant.

Structures 1 to 4 in FIG. 39 are identified, based on the position numbers of their nucleotides, as CY1132-1329; CY1493-1525; CY2136-2218 and CY2220-2280 respectively. CY1132-1329 has 6 (3%) of nucleotides with positional entropy of over 1.0 and a standard deviation of positional entropies of 0.30. The average APE is 0.19 and the highest positional entropy (PE) is 1.49. The DG is −87.9 kcal/mol. CY1493-1525 has 1 nucleotide (3%) with positional entropy of over 1.0 and a standard deviation of positional entropies of 0.25. The average APE is 0.10 and the highest PE is 1.00. The DG is −13.1 kcal/mol. CY2136-2218 has 1 (1.2%) of nucleotides with positional entropy of over 1.0 and a standard deviation of positional entropies of 0.25. The average APE is 0.11 and the highest PE is 1.12. The DG is −41.9 kcal/mol. CY2220-2280 has no nucleotides (0%) with positional entropy of over 1.0 and a standard deviation of positional entropies of 0.23. The average APE is 0.15 and the highest PE is 0.80. The DG is −29.9 kcal/mol.

The sequence of CY1132-1329 (structure 1, 198 nucleotides) is (SEQ ID NO:64):

cagtaaaaggttgtatggtgagggtgctgagccgtgtatcgccaaaggc
ctaaatgcattagaatctggagcgactttga

The sequence of CY1493-1525 (structure 2, 33 nucleotides) is (SEQ ID NO:65):

tttatgcttagtgaacttggcattaagcatgaa

The sequence of CY2136-2218 (structure 3, 83 nucleotides) is (SEQ ID NO:66):

acgaggacttaggcgtcctaggatgaatagggtcattggtttaccgatg
atacctgttcagaataggattgctcgagctt

The sequence of CY2220-2280 (structure 4, 61 nucleotides) is (SEQ ID NO:67):

ggttagggtaactcacataccttcttccataactggaaaaggtcgtgtg
agcaacctaacc

In various examples: CY1132-1329 was inserted at 2219/2281 of CYVaV; CY1493-1525 was inserted at 2319/2320 and 2304/2305 of CYVaV (in two separate examples); CY2136-2218 was inserted at 2304/2305 of CYVaV; and, CY2220-2280 was inserted at 2304/2305 of CYVaV. In each case, the resulting vector was stable in planta at the end of a 10-week trial.

A line of best fit through these four hairpin-like structures has the formula DG in kcal/mol=−0.44×length of the structure in nt−1.89. When comparing the DG of a synthetic construct to the DG of a natural hairpin-like structure of similar length, this formula (or a different formula derived from another wild type vector) may be used when a specific natural structure of similar length does not exist. In some examples, stable hairpin-like structures of a selected length are stable when their DG is in a range of +/−10 kcal/mol, or −5 to +15 kcal/mol, or +/−20 kcal/mol for inserts of 150 nucleotides or more, of the DG determined by the formula for the selected length. A line of best fit through 58 stable synthetic inserts and the four hairpin-like like structures of CY1 has the formula DG in kcal/mol=−0.43×length of the structure in nt+1.17. Optionally, hairpin-like structures can be made in a range of −10 to +10 kcal/mol, or −5 to +15 kcal/mol, or −20 to +20—for inserts of 150 nucleotides or more, of the DG determined by this formula for the selected length.

### D. Example of a Stable Insert Targeted Against CVEV

Two inserts were prepared including siRNA sequences targeted against regions of the CVEV genome. Insert mmck15 (FIG. 14B) has an siRNA sequence of 29 nt complementary to bases 4308-4338 of CVEV. Insert mmck8 (FIG. 14C) has an siRNA sequence of 28 nt complementary to bases 766-793 of CVEV. Sequences are given below. SiRNA portions of the sequences are at the beginning of the sequences and shown in bold type and inside a box in the figure.

The sequence of the wt structure is (SEQ ID NO:35):

ggttagggtaactcacataccttcttccataactggaaaaggtcgtgtg
acaacctaacc

The sequence of mmck15 is (SEQ ID NO:36):

gtcgcaatcaaagacgaagaaatcgtccataactggacatttccttcg
tctcaattgcgac

The sequence of mmck8 is (SEQ ID NO:37):

ggttgcttggaacccatacgaatgttgcttaacagcaaattcgctatg
ggtgatagcaacc

Wild type structure 4 (FIG. 14A) has a ΔG of −30.00 kcal/mol and an APE of 0.15. Mmck15 has a ΔG of −29.80 kcal/mol, an APE of 0.18, a standard deviation of PE of 0.28, 1.6% of nucleotides with a PE over 1, and a highest PE of any nucleotide of 1.0. Mmck8 has a ΔG of −30.30 kcal/mol, an APE of 0.16, a standard deviation of PE of 0.24, 1.6% of nucleotides with a PE over 1, and a highest PE of any nucleotide of 1.0. Both mimics were stable after 4 months (the entire duration of the experiment) in N. benthamiana. Secondary structure diagrams of wild type structure 4 (FIG. 14A), mmck 15 (FIG. 14B) and mmck (FIG. 14C) are shown in FIGS. 14A-14C.

N. benthamiana plants were agroinfiltated with CY1_{CVEV}. After 21 days, the plants were agroinfiltrated with CVEV. After 10 days, the CVEV levels in the CY1_{CVEV} treated plants were 17.6 and 29.5 relative to an untreated control (100).

### E. Further Examples of Inserts Mimicking Structure 4

Referring to FIGS. 14A-14E, the wild type Structure 4 is shown as FIG. 14A. FIG. 14D is an insert called mmckpsvD with a 29 nt siRNA targeting bases 174-202 of peanut stunt virus. Its ΔG is −30.40 kcal/mol and it has an APE of 0.17, a standard deviation of PE of 0.28, 3.3% of nucleotides with a PE over 1, and a highest PE of any nucleotide of 1.0. FIG. 14E is an insert called mmckpsvE with a 29 nt siRNA targeting bases 231-259 of peanut stunt virus. Its ΔG is −30.50 kcal/mol and has an APE of 0.12, a standard deviation of PE of 0.25, 1.6% of nucleotides with a PE over 1, and a highest PE of any nucleotide of 1.0. Both mimics were stable after 4 months in N. benthamiana (the entire duration of the experiment). SiRNA portions of the sequences are boxed in the figure.

The sequence of mmckpsvD (SEQ ID NO:38) is shown below. The first 29 nucleotides are the targeting sequence.

gggtgaagtcatgaaagaagctgccttcttaacagaagcagctctctt
tcacaattcaccc

The sequence of mmckpsvE (SEQ ID NO:39) is shown below. The first 29 nucleotides are the targeting sequence.

ccgtaggtaagacaccattgacaagttcttaacagaactgtcacatgg
tgtgaccctacgg

Referring to FIGS. 15A-15C, the wild type Structure 4 is again shown as FIG. 15A. FIG. 15B is an insert called PRSVmmck1 with an siRNA targeting papaya ring spot virus. Its ΔG is −24.20 kcal/mol and has an APE of 0.13, a standard deviation of PE of 0.27, 1.6% of nucleotides with a PE over 1, and a highest PE of any nucleotide of 1.0. FIG. 15C is an insert called PRSVmmck2 with an siRNA targeting papaya ring spot virus. Its ΔG is −29.00 kcal/mol and it has APE of 0.14, a standard deviation of PE of 0.31, 3.3% of nucleotides with a PE over 1, and a highest PE of any nucleotide of 1.6. Both mimics were stable after 4 months in N. benthamiana (the entire duration of the experiment). SiRNA portions of the sequences are boxed in the figure.

The sequence of PRSVmmck1 (SEQ ID NO:40) is shown below. The first 29 nucleotides are the targeting sequence.

atggtctgaatgataatgaaatgcaagtgtaaccacttcatttccatt
atccaaagaccat

The sequence of PRSVmmck2 (SEQ ID NO:41) is shown below. The first 29 nucleotides are the targeting sequence.

gcagaagcatatattgcaaagagaaatgctaacgcattctcttctgca
atacaacttctgc

### F. Examples of Inserts Mimicking Structure 1

FIGS. 16-20B each show the wild type Structure 1 found at 1132-1329 of CY1 in Panel A. Its ΔG is −87.9 kcal/mol and its APE is 0.19.

FIG. 16B shows PDS-mmck-1 and FIG. 16C shows PDS-mmck-2. These inserts target phytoene desaturase (PDS). PDS is a widely used reporter gene to demonstrate silencing an endogenous gene in plants. Silencing the PDS gene produces a photobleached phenotype. ΔG of PDS-mmck-1 is −81.80 kcal/mol and has an APE of 0.16, a standard deviation of PE of 0.27, 3.0% of nucleotides with a PE over 1, and a highest PE of any nucleotide of 1.6. ΔG of PDS-mmck-2 is −82.2 kcal/mol and it has an APE of 0.21, a standard deviation of PE of 0.32, 4.0% of nucleotides with a PE over 1, and a highest PE of any nucleotide of 1.5. SiRNA portions of the sequences are boxed in the figure.

The sequence of the wt structure is (SEQ ID NO:42):

cagtaaaaggttgtatggtgagggtgcttgagccgtgtatcgccaaag
gcctaaatgcattagaatctggagcgactttgaggcgcaaatgggaga
agttttcttctccagtttgcgtttctctcgacgcttccaggttcgacc
tgcatgtaagcgttggcatgctaaagttcacacacaagctatatgact
attactg

The sequence of the PDS-198mmck-1 insert (SEQ ID NO:43) is shown below (target sequence shown in bold):

cacgaaacagaagtacttggcttcaatggaaggtgctgtcttatcagg
aaagctttgtgcacaagctattgtacaggattacgagttacttcttgg
cttttgccaagatgtaactcgtaaacaaattacaaatagcttgtcaaa
caaaactgtcggataaacagtaaacttccttgcctacaagtaagtcta
ttcgtg

The sequence of the PDS-198mmck-2 insert (SEQ ID NO:44) is shown below (target sequence shown in bold):

gttgctcagtgtgtacgctgacatgtctgttacatgtaaggaatatta
caaccccaatcagtctatcttggaattggtatttgcacccgcagaaga
gttttctcttctacgggtgcaaataagtgtttcacaacatagaaaata
ttggagtcgtctattcttacccaataacaacaacaaagcgtacacaca
agcaac

FIG. 17B shows LcrGyrA-mmck5 and FIG. 17C shows LcrGyr-mmck3. These inserts target the GyrA gene of Liberibacter crescens. ΔG of LcrGyrA-mmck5 is −82.5 kcal/mol and it has an APE of 0.32, a standard deviation of PE of 0.40, 6.1% of nucleotides with a PE over 1, and a highest PE of any nucleotide of 1.9. ΔG of LcrGyrA-mmck3 is −69.4 kcal/mol (by RNAfold) or −58.20 kcal/mol (by mfold*) and it has an APE of 0.32, a standard deviation of PE of 0.47, 13.6% of nucleotides with a PE over 1, and a highest PE of any nucleotide of 1.9. LcrGyrA-mmck3 is unusual in that its MFE when calculated by two different programs (mfold and RNAfold) varies, whereas for most hairpin-like structures the MFE calculated by different programs is similar. Values described herein are from RNAfold unless stated otherwise. LcrGyrA-mmck3 also had a higher standard deviation of PE and more high PE bases than most other hairpin-like structures tested. Further, LcrGyrA-mmck3 has an MFE that is 19.6 kcal/mol greater than the MFE predicted by the formula (−0.44 multiplied by the length of the insert (# of nt)−1.89) and 18.5 kcal/mol greater than the wildtype CY1 hairpin-like region of the same length (198 nucleotides). LcrGyrA-mmck3 suggests that a larger positive variance in MFE may be acceptable for larger inserts, for example insets of 150 nucleotides or more. It is not known whether a difference in stability between, for example, LcrGyrA-mmck3 and LcrGyrA-mmck5 may have emerged after a longer experiment. SiRNA portions of the sequences are boxed in the figure.

The sequence of the LcrGyrA-mmck5 insert (SEQ ID NO:45) is shown below (target sequence shown in bold):

acctggtactgttcggcgaaataaattatctgattttctccatgtgaa
ccgtaatcgtaagattccaatgaaattcgaggaaaatgatgagattgt
tttttaacaatcgcatcattttccccattcgctcatgtgcaatgacca
ccatcgttgtaacatgacaactcacagataataccatcgccgaacacc
ccaggt

The sequence of the LcrGyrA-mmck3 insert (SEQ ID NO:46) is shown below (target sequence shown in bold):

atgatgagattgtttcagtagaaacttgtactgaggaccatgatgttt
tgctaacaacagaatttggtcagtgtattcgattcccagtttctaatg
tttttacattagcaactgtgaatcccacacacctgacccatttaaacg
ttgtcaaaaaccattaggtcaaagagtacagtggaaactgaaacaacc
catcat

FIG. 18B shows Clas-GyrAmmck-5′ and FIG. 18C shows Clas-GyrAmmck-3′. These inserts target the GyrA gene of Liberibacter asiaticus. ΔG of Clas-GyrAmmck-5′ is −75.3 kcal/mol and it has an APE of 0.23, a standard deviation of PE of 0.36, 5.1% of nucleotides with a PE over 1, and a highest PE of any nucleotide of 1.2. ΔG of Clas-GyrAmmck-3′ is −87.3 kcal/mol and it has an APE of 0.21, a standard deviation of PE of 0.35, 3.0% of nucleotides with a PE over 1, and a highest PE of any nucleotide of 1.6. SiRNA portions of the sequences are boxed in the figure.

The sequence of the Clas-GyrAmmck-5′ insert (SEQ ID NO:47) is shown below (target sequence shown in bold):

gcatggcaatgtacggcgtaataaactttctgattttattcaaatcaa
tcgtagtggtaagattgcgatgaaattagattcaagagatgagattct
tttttaagaatcccatctcttgaaacattccctcatgcgcaataacca
ccacccatattatttgataacccacagaaggttcaaatgctgtacacc
ccatgc

The sequence of the Clas-GyrAmmck-3′ insert (SEQ ID NO:48) is shown below (target sequence shown in bold):

gagatgagattctttccgttgaaacctgtacacaagaaaatgatatat
tgttgactactaaacttggacaatgtgtccgctttccgatttctgcta
tttttatagcagcaatcggaaagctaacacacttgtcccaagtggccg
tagttccaaactatcatttcgccctgtacggtaaacacggaaagaaaa
catctc

FIG. 19B shows CS7. This insert targets the callose synthase 7 gene of Nicotiana benthamiana. ΔG of CS7 is −86.4 kcal/mol and it has an APE of 0.24, a standard deviation of PE of 0.36, 5.6% of nucleotides with a PE over 1, and a highest PE of any nucleotide of 1.5. The targeted sequence of the CS7 gene is similar to a sequence of the orthologue from citrus (CscalS7), which is responsible for producing callose in sieve elements in response to pathogens. SiRNA portions of the sequences are boxed in the figure.

The sequence of the CS7 insert (SEQ ID NO:49) is shown below:

agaattatggagcggtcaagctcacacatcgtcacactttttatgtgg
tgggctcagccaaaactgtatgtaggaagaggcttgcatgagggcatg
tttttacatgccatcatgcaagccagacgactatcatacagttgaccg
ctgatccaactcataaaagtcaaaatgattgtacaattgattgctctc
aattct

FIG. 20B shows insert Erwinia GyrA. This insert targets the GyrA gene of Erwinia. ΔG of Erwinia GyrA is −90.8 kcal/mol and it has an APE of 0.13, a standard deviation of PE of 0.29, 4.0% of nucleotides with a PE over 1, and a highest PE of any nucleotide of 1.5. SiRNA portions of the sequences are boxed in the figure.

The sequence of the Erwinia GyrA insert (SEQ ID NO:50) is shown below (target sequence shown in bold):

ccgaccgcgcagcgccggtattattgccgtcaatctgcgtgatgacga
tgaactgatcggcgtgtcgctgacgaacggtagtgatgaagcgatgct
gttttcagcatcacttcatcactaaaaagacgtacagtgacacataag
atcacttaatatcatccgtatacatgatgcaaaccaattggtgttgta
ggttgg

All of the inserts mimicking structure 1 described in this section were inserted between 2219 and 2281 of CYVaV after removal of 2220-2280 of CYVaV. The inserts were stable in N. benthamiana after 16 weeks (the entire duration of the experiment) except that Erwinia GyrA was stable for 8 weeks (which was the entire duration of the experiment).

### G. Example of a Stable Insert Targeted Against CTV

CTV is composed of two capsid proteins and with a genome of more than 19 kb. 76 CTV isolates have been characterized, which all contain regions of conserved nucleotides. Two sequence portions (18 and 6) of a CTV isolate are identified in Table 3 below, showing fully conserved polynucleotides (underlined below) as well as less-conserved nucleotides (in bold) with other nucleotides present in some isolates (listed as identified and bolded nucleotides in each sequence from left to right). For example, in the sequence portion for CTV18 shown in Table 3, the 3 non-conserved nucleotides include, from left to right: guanine (G) which position instead includes adenine (A) in 10 CTV isolates; cytosine (C) which position instead includes uracil (U) in about half of the CTV isolates; and G which position instead includes A in 6 CTV isolates. In the sequence portion for CTV6, the 6 non-conserved nucleotides include, from left to right: G which position instead includes A in 1 CTV isolate; G which position instead includes A in 3 CTV isolates; U which position instead includes C in 3 CTV isolates; A which position instead includes G in 9 CTV isolates; U which position instead includes C in 1 CTV isolate; and A which position instead includes G in 1 CTV isolate.

TABLE 1
Sequence Portions of CTV Isolates

| | | Sequence (conserved | Non-conserved |
|---|---|---|---|
| | CTV | nucleotides in known | nucleotides (bolded in |
| # | Position | CTV isolates underlined): | sequence): |
| 18 | 15173 | 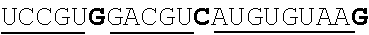 | G: A in 10 isolates |
| | | (SEQ ID NO: 89) | C: U in ~half isolates |
| | | | G: A in 6 isolates |
| 6 | 17856 | 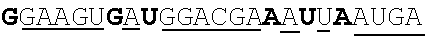 | G: A in 1 isolate |
| | | (SEQ ID NO: 90) | sequence): |
| | | | G: A in 3 isolates |
| | | | U: C in 3 isolates |
| | | | A: G in 9 isolates |
| | | | U: C in 1 isolate |
| | | | A: G in 1 isolate |

FIG. 11A shows the sequence and secondary structure of a hairpin-like structure called mmck6.2. This insert includes a sequence targeting CTV, which is the 27 bases extending on the right hand side of the insert from the bottom of the insert to just below the apical loop as the insert is shown in FIG. 11A. The MFE (alternatively called ΔG) of the insert is −29.64 kcal/mol as determined by the RNAfold software. The APE is 0.23.

The sequence of mmck6.2 is shown below (SEQ ID NO:91). The targeting sequence (intended to produce siRNA) is underlined.

gguuucaguauauuucgucaucaccuuccuaacggaagugauggacgaa
auuaaugaaacc

To investigate the stability of mmck6.2 in CY1, mmck6.2 was inserted into the CY1 genome by removing the original viral sequence between positions 2220 to 2280 and inserted mmck6.2 between positions 2219 and 2281. The resulting recombinant CY1 was then inoculated into Mexican lime citrus plants by vacuum-infiltration. Three months post-inoculation, RT-PCR analysis was performed of the systemic leaves of the inoculated plants to identify recombinant CY1 positive plants. The viral fitness and the stability of the insert in the positive plants was monitored over a long period of time (30 months at the time of writing).

After one year post-inoculation, RT-PCR analysis and Sanger sequencing were performed of the systemic leaves of the positive plants. The results (FIG. 11B) showed that all 4 positive plants have a single RT-PCR product. The Sanger sequencing of the RT-PCR product confirmed that the insert is intact. This suggests that mmck 6.2 is stable in the CYVaV1 genome and can be maintained over an extended period of time in citrus plants. The polynucleotide sequences shown in FIG. 11B are SEQ ID NO:92 (Analysis of #7) and SEQ ID NO:93 (Analysis of #20). The sequence of mmck6.2 (SEQ ID NO:91) is underlined:

SEQ ID NO: 69:
ttcgttgguuucaguauauuucgucaucaccuuccuaacggaagugaug
gacgaaauuaaugaaaccagttaatg
SEQ ID NO: 70:
agcttcgttgguuucaguauauuucgucaucaccuuccuaacggaagug
auggacgaaauuaaugaaaccagttaatgta

FIG. 11C shows: Top: Northern blot of RNA extracted from two infected citrus plants after ~one year of infection. Middle_(−RNA): Reverse-transcriptase (RT)-PCR was used to amplify a segment of the infecting VIGS vector. For this plant, 10 leaves were assayed. Only a single product of the correct size for the vector with the insert was found. Bottom: Total RNA from young leaves of the infected plant were subjected to batch sequencing of the insert region. Data is from 15-month citrus showing no indication of single “double peaks” that would denote base changes or multiple double peaks that would indicate a portion of the population contain deletions. In addition, at least 10 clones were generated for individual sequencing and no changes from the initial VIGS vector must be found. SEQ ID NO: 35 shown in FIG. 11D is missing the first G of the insert only because of the boundaries of the screenshot. The first peak in the insert band (not included in the sequence) is this missing G. FIG. 11E: A photograph of a Mexican lime plant infected with recombinant CY1 including mmck6.2 showing a lack of symptoms of infection in the plant.

Sequencing was repeated at 21 months and at 30 months post infection. At 21 months post infection, three of four mother trees still containing progeny of the CY1 vector with the Mmck6.2 insert. In one of the three mother trees, the insert had a single point mutation. At 30 months post-inoculation, samples were collected from different locations from four mother trees. Loss of insert was observed in samples taken near the bottom of two mother trees but inserts were recovered with varying degrees of mutation from samples taken near the top and bottom of the trees. Two other mother trees retained the insert all locations, with the inserts recovered from one tree showing a few base mutations and two positive samples from the other tree exhibiting minimal base mutation.

Real time quantification by stem-loop RT-PCR (using purified LMW RNA) was conducted to confirm the presence of the intended siRNA product in two N. benthamiana plants infect by CY1 vectors carrying a Mmck6.1 insert and a Mmck6.2 insert and in four citrus plants infected with a CY1 vector carrying the Mmck6.2 insert. The citrus plants had been infected for about 15 months at the time of the assay. Sequence fragments detected by this assay must end with the correct last six bases at the 3′ end, which is TAATGA. Given that RNA viruses mainly generate 21-24 nucleotide siRNA's, it is expected that sequences detected by this assay are the intended siRNA. Other effective siRNA may also be present but are not detected by the assay. For the results provided in FIG. 29, leaves were collected randomly from various parts of the plants and combined for siRNA analysis. A mock vector and wild type CY1 were used as negative controls. As shown in FIG. 29, the intended siRNA were detected in both citrus and N. bentamiana, with the amount detected being higher in N. bentamiana. For the results provided in FIG. 30, young and mature citrus leaves were collected and analyzed separately. There was no consistent correlation between leaf stage and siRNA levels. Wild type CY1 and PsyE (a CY1 vector carrying an unrelated siRNA insert) were used as negative controls.

### H. APE of Exemplary Inserts

FIG. 12A show a flow chart for how the data in FIG. 12B was generated. The predicted structure is determined by entering the sequence for the insert into RNAfold. The RNAfold software produces a list of positional entropy values of the individual bases. The positional entropy values are given a grey scale and sorted from highest PE (darkest grey) to lowest PE (light grey)

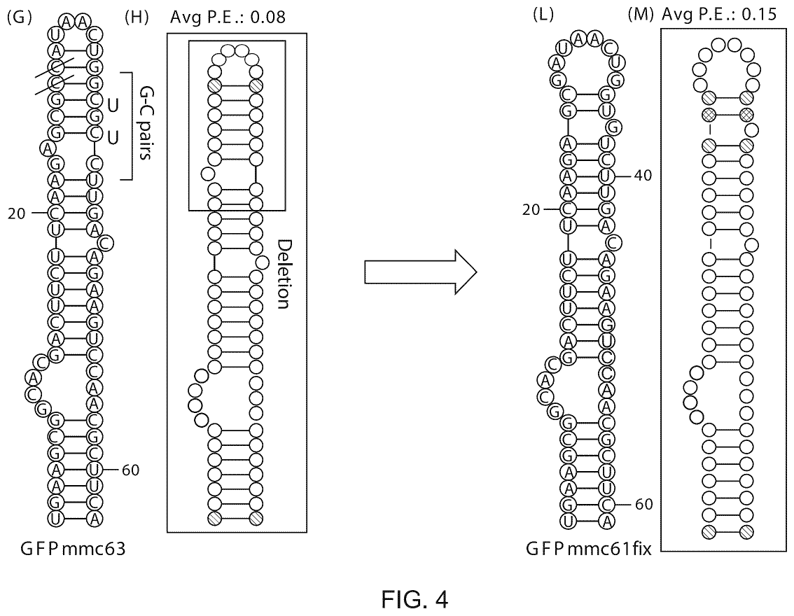

APE values for various hairpin-like structures (and some additional non-hairpin-like structures) inserted into CY1 are shown in FIG. 12B. Sorted positional entropy is shown for each insert (dark grey to lightest grey). The first four hairpin-like structures (boxed) were duplicates of naturally occurring hairpin-like structures 1 through 4 of CY1 (see FIG. 39). Other hairpin-like structures and non-hairpin-like inserts are arranged in order of descending APE (from 1.30 to 0.07). Mimic hairpin-like structures begin with a number (1 or 4) that identifies the hairpin-like structure of CY1 being mimicked. Thick lines beneath each lane denote the length of each insert (number of nucleotides shown at right). Single and double asterisks denote inserts that were not stable in the VIGS vector. The double asterisk denotes an unstable hairpin-like structure that had three consecutive G:C pairs at the base and five consecutive G:C pairs in the apical region contributing to the very low APE of 0.07. Eliminating two nucleotides on the 5′ side and changing 2 nucleotides from C to U on the 3′ side in the apical region generated a stable hairpin-like structure (FIG. 4) without 5 consecutive apical G:C pairs and the APE value changed to 0.15 from 0.08. The thick black arrow denotes unstable hairpin-like structure (M2250gfp30ext) with a relatively low APE value of 0.39 that contained a clustering of higher entropy nucleotides.

### I. Example of a Stable Hairpin-Like Structure (GFPmmck59)

FIG. 23 shows the sequence and secondary structure of GFPmmck59. This insert has 59 nucleotides including a region targeted against green fluorescent protein (GFP) expression. Some plants or recombinant pathogens used in experimental trials have been modified to express GFP. Suppressing GFP expression can indicate replication, movement or effectiveness of a vector with a GFP suppressing insert.

This insert is stable. The minimum free energy (MFE) is −30.20 kcal/mol. GFPmmck59 has 0 (0%) bases with positional entropy (PE) greater than 1.0. The APE is 0.07 and the highest PE of any base is 0.995. The standard deviation of PE is 0.21.

The sequence of GFPmmck59 is shown below (SEQ ID NO:53). The first 27 bases of the sequence are the targeting sequence.

tgaagcggcacgacttcttcaagagcgataactcgccttgacagaagtc
caacgcttca

J. Example of an Unstable Hairpin-Like Structure with Low APE (GFPmmck63)

FIG. 24 shows the sequence and secondary structure of GFPmmck63. This insert has 63 nucleotides including a region targeted against green fluorescent protein (GFP) expression. Despite having an APE of 0.08, no bases with PE greater than 1, and a standard deviation of PE of 0.22, this insert is relatively unstable. The MFE is −36.60 kcal/mol.

One region of this insert has 5 consecutive G-C pairs. Deletion of two nucleotides on the 3′ side and changing two nucleotides from C to U on the 3′ side in the apical region eliminated the 5 consecutive G-C pairs (FIG. 4). The resulting insert, GFPmmck61fix, had fewer G-C pairs and was stable. GFPmmck61fix has an APE of 0.15, a maximum PE of 1.01, 61 nucleotides, one base (1.6%) with PE greater than 1, a standard deviation of PE of 0.28 and an MFE of −26.4 kcal/mol. This example suggests that a large number (e.g. more than 4) consecutive G-C pairs leads to instability, even in a hairpin-like insert meeting other guidelines.

The sequence of GFPmmck63 is shown below (SEQ ID NO:54). The first 30 bases of the sequence are the targeting sequence.

Tgaagcggcacgacttcttcaagagcgccataactggcgccttgacaga
agtccaacgcttca

### K. Second Example of an Unstable Hairpin-Like Structure (M2250GFP30ext)

FIG. 22 shows the sequence and secondary structure of M2250GFP30ext. This insert is relatively unstable. The MFE is −27.3 kcal/mol. M2250GFP30ext has 9 (13.8%) bases with positional entropy (PE) greater than 1.0. The APE is 0.39 and the highest PE of any base is 1.97. The standard deviation of PE is 0.52.

The sequence of M2250GFP30ext is shown below (SEQ ID NO:52):

ggttagggtaactcactgaagcggcacgacttcttcaagagcgccattc
agtgagcaacctaacc

L. Third Example of an Unstable Hairpin-Like Structure with Low APE

FIG. 27 shows the sequence and secondary structure of BBLv2-1. BBLv2-1 has 198 nucleotides. 3 nucleotides (1.5%) have PE greater than 1. The APE is 0.18 and the standard deviation of PE is 0.3 but this insert is unstable.

The sequence of BBLv2-1 is shown below (SEQ ID NO:57):

cuggaaaauucgauuacucaaagugccuaaaaucguaacaacuuguaug
aucuauaggccuggauccaaacaauacguggcucaaauacuugagaaau
uuuuuucucauguauuugagccuaaaccuugauuuggauccaucuccua
ucgaacaccaaguguuaaacuuuuuagcagagggaguaaucgaaauucc
ag

The sequence of BBLv2-1 after deletion is shown below (SEQ ID NO:58):

cuggaaaauucgauuacucaaagugucgaaauuccag

An analysis of BBLv2-1 indicated that modification of the vector in an infected plant involved removal of a region having a bulge (no bases on one side, 1 base on the other side), 5 A-T base pairs (A-U base pairs are included as A-T base pairs), an asymmetric loop with 7 bases (counting both sides) and 3 more A-T base pairs. The DG of the asymmetric loop was positive (2.7 kcal/mol) and the DG of the region from the bulge to the asymmetric loop (including both the bulge and the asymmetric loop) had a positive DG of 0.4 kcal/mol. Without intending to be limited by theory, instability of this inert may have been caused by the locally positive DG of this region.

This example suggests a number of potential design parameters such as that the number of consecutive A-U base pairs should be less than 5 or that the DG of a region including two loops and the stack between them should not be positive.

### M. Example of a Hairpin-like Structure of CTV.

The stability of hairpin-like structures that mimic hairpin-like structures in the CY1 and OULV genomes has been previously demonstrated by their ability to remain stable, for example when inserted to replace the sequence between 2220 to 2280 (i.e. inserted at 2219/2281) or inserted at 2304/2305, 2319/2320 or 2330/2331 of CY1. To investigate whether this rule also applies to hairpins that originate from an unrelated virus, every 2 kb of the CTV genome was subjected to secondary RNA structure prediction using mfold software obtained from the “UNAFold Web Server” (http://www.unafold.org/mfold/applications/ma-folding-form-v2.php) in search of hairpins that resemble a naturally-occurring hairpin of CYVaV1 in terms of size and shape. As a result, a 128 nt hairpin-like structure, CTV-insert-natural-V2.2, was identified which forms a stack-loop structure with internal asymmetrical loops. The overall structure of this hairpin resembles a smaller version of a hairpin-like structure 1 of CY1. CTV-insert-natural-V2.2 has a ΔG of −43.6 kcal/mol (mfold) or −46.96 (RNAfold) and APE of 0.359.

The sequence of CTV-insert-natural-V2.2 is (SEQ ID NO:55):

ggggguuuauguuuggcaaagaaaguguuggaacuguuagucaagcggg
ugguugaaucguuuucucguuugaagcggaaaaccgcucguuuaacguc
cuucgcuaauuuguugcuugcgaggcucuc

FIG. 25 shows the sequence and secondary structure of CTV-insert-natural-V2.2 as predicted by “mfold” and drawn by “RNA2Drawer.”

To test the stability of CTV-insert-natural-V2.2 in CY1, this hairpin-like structure was inserted into the CY1 genome by replacing the original viral sequence between 2220 to 2280 of CY1 with CTV-insert-natural-V2.2. The resulting recombinant CY1 was then inoculated into N. benthamiana plants by syringe infiltration and viral fitness as well as the stability of the insert was monitored over a period of time. Three weeks post-inoculation, RT-PCR analysis and Sanger sequencing were performed of the systemic leaves of all symptomatic plants. The results (FIG. 26) show that all 9 symptomatic plants tested have a single RT-PCR product that migrates slower than CYVaV1 wt control, suggesting the presence of recombinant CY1 with CTV-insert-natural-V2.2. The Sanger sequencing of the RT-PCR product confirmed that the insert is intact in all 9 symptomatic plants.

### N. Example of a Hairpin-like Structure Encoding a Peptide.

FIG. 28 shows modifications at the end of a hairpin-like region in the CY1 p81 (RdRp) ORF to remove the region from 2159-2197 (including the p81 stop codon at 2160-2162) and insert a T2A self-cleavage peptide (including a T2A cleavage site) followed by a peptide to be expressed in the host plant, a new stop codon, and additional nucleotides to provide a stable hairpin-like insert.

The sequence of the wild type CY1 2136-2218 region shown in the Figures, which is a hairpin-like structure including the C-terminal of the RdRp (p81) ORF, is presented below (SEQ ID NO: 94):

ACGAGGACUUAGGCGUCCUAGGAUGAAUAGGGUCAUUGGUUUACCGAUG
AUACCUGUUCAGAAUAGGAUUGCUCGAGCUU
CGU

The underlined UGA in SEQ ID NO: 72 is the native CY1 stop codon.

The sequence of the modified CY1 shown as P81T2A in the FIG. 28, starting at position 2136 of CY1, is presented below (SEQ ID NO: 95):

ACGAGGACUUAGGCGUCCUAGGAggaagcggagagggcagaggaagucu
gcuaacaugcggugacgucgaggagaauccugaccuugacgauccgaug
uucuaaccgaagucaccgaaggccagcggacccccucugaccuaccuga
uuccugAUAGGAUUGCUCGAGCUUCGU

The underlined UGA in SEQ ID NO: 95 is the P81T2A stop codon.

The sequence of the T2A self-cleavage peptide shown in the Figures, is presented below (SEQ ID NO: 74):

G S G E G R G S L L T C G D V E E N P G/P

The “/” in SEQ ID NO: 74 indicates the T2A cleavage site.

The sequence of the modified CY1 shown as P81T2A_PEP6His-tag in the figures is presented below (SEQ ID NO: 75):

ACGAGGACUUAGGCGUCCUAGGAggaagcggagagggcagaggaagucu
gcuaacaugcggugacgucgaggagaauccuggaccuuuuaggcuaaag
uuucaucaccaccaccaucaccacugaccggugauaguguuggugauga
aacuuuaggauaaacgauccgauguucuaaccgaagucaccgaaggcca
gcggacccccucugaccuaccugauuccugAUAGGAUUGCUCGA
GCUUCGU

The underlined uuuagacuaaaguuucau in SEQ ID NO: 75 encodes the PEP6 peptide. The underlined uga in SEQ ID NO: 75 is a stop codon. The intervening caccaccaccaucaccac in SEQ ID NO: 75 encodes the (6×) His-tag.

The sequence of the T2A self-cleavage peptide and the cleaved protein shown in the Figures, is presented below (SEQ ID NO: 76):

G S G E G R G S L L T C G D V E E N P G P F R L K
F H H H H H H H

The “/” in SEQ ID NO: 76 indicates the T2A cleavage site. The underlined F R L K F H in SEQ ID NO: 76 is the PEP6 peptide. The downstream H H H H H H in SEQ ID NO: 76 is the (6×) His-tag. The complete cleaved peptide is a P-PEP6His-tag.

In the sequences above, uppercase letters indicate parts of the native CYVaV sequence. Lowercase letters indicate parts of the heterologous (inserted) sequence.

N. benthamiana plants were infected with a CY1 vector with the P81T2A_PEP6His-tag insert by way of agroinfection. The vector was stable (replicated in the host plant without losing the heterologous insert) for the duration of a 6 week trial. The T2A functionality was confirmed in-vivo using CY1 with P81-His and CY1 with P81T2APEP6His-tag. In Western blots, His-tagged p81 was detected but p81T2APEP6His-tag was not detected. This indicates that the T2A-fusion protein functioned properly in the plants. The P-PEP6histag peptide was selected to facilitate experimental assays but may be replaced with another antimicrobial peptide.

The P81T2A_PEP6His-tag insert was designed according to “mimic” principles. The first 201 nucleotides of the inserted sequence form a hairpin-like structure, with the final 2 nucleotides (UG) corresponding in position to the bases at 2157 and 2158 of wild type CY1, which were left unpaired with the deletion of the 2159-2197 region. The stop codon of the insert is placed in the apical loop. The structure formed by the first 201 nucleotides has an MFE of −98.6 kcal/mol as calculated by the RNAfold web server. The average positional entropy (APE) is 0.115.

The entire hairpin-like structure that replaces the structure formerly at positions 2136-2218 of CY1 has a length of 247 nt, an APE of 0.13, a maximum P.E. of 1.1, one base (0.4%) with a PE >1, a standard deviation of PE of 0.21, 17 consecutive base pairs in one region, and an MFE of −123.3 kcal/mol, which deviates from the formula presented herein by −12.7 kcal/mol. Another long insert, CY2_2163T2APEP6His, had 284 nucleotides, an APE of 0.22, a maximum P.E. of 1.8, 21 bases (7.4%) with a PE >1, a standard deviation of PE of 0.39, and an MFE of −144.5 kcal/mol, which deviates from the formula presented herein by −17.7 kcal/mol. Both of these inserts are stable. In contrast, an unstable T2A peptide insert had 120 nucleotides and an MFE which deviated from the formula by −31.5 kcal/mol. Collectively these examples suggest that long inserts, e.g. with 150 nucleotides or more, may have an MFE that deviates from the formula by about 20 kcal/mol, particularly by being more negative.

### O. Targeting the Plant Susceptibility Gene ML06 for Disease Resistance.

Plant pathogens pose a significant threat to crop yield and productivity by exploiting the susceptibility (S) genes inherent in plants to facilitate their infection. Well-studied S genes include Botrytis-induced kinase1 (BIK1), Sugars Will Eventually be Exported Transporters (SWEETs), Lateral Organ Boundaries 1 (LOB1), Mildew Locus 0 (MLOs) such as MLO6 and MLO7, Downy mildew Resistance (DMRs) such as DMR6, Defense No Death (DNDs) genes such as DND1, Translation Initiation Factors (eIF4E), DspA/E-interacting proteins from Malus (DIPM1-4), and Enhanced Disease Resistance 1 (EDR1). Perturbation of these S genes through mutagenesis, genetic engineering, or exploitation of natural variants confers resistance to a wide range of plant diseases. However, complete knockout of these genes can result in unexpected stunt phenotypes, leading to significant yield and quality loss. To address this challenge, a VIGS vector was used to partially silence host susceptibility genes. In the examples described herein, a CY1 based vector is engineered to target an MLO or DMR gene.

In the examples described herein a heterologous RNA segment is added to the CY1 backbone. In particular, a wildtype hairpin-like region from 2220-2296 of the CY1 backbone as presented herein is removed and replaced with the heterologous RNA segment. In other examples, a different insertion site may be used, with or without deletion of a wildtype hairpin-like region from the backbone. In other examples, a different viral backbone may be used as appropriate for a host plant. For example a CTV vector may be used to treat citrus trees or a TRV vector may be used to treat various plants that TRV is known to infect. In other examples, other susceptibility genes may be targeted with a ULV, Class 2 ULV or CY1 vector.

One of the most pervasive plant diseases, impacting a wide range of plant species (e.g. over 650 species of powdery mildew fungi that affect about 10,000 plant species). Grapevine Powdery Mildew is a predominant fungal disease affecting grapevines globally and can lead to yield losses of up to 75-100%, particularly during high disease-pressure years. Numerous grapevine varieties lack genetic resistance, necessitating extensive fungicide programs in viticulture, with an annual average of 19.5 kg/ha of active ingredients. The collective expense of fungicide application ranges between 9% and 20% of the total cultural cost. This costly and necessary practice heightens the risk of resistance development in E. necator populations.

A universal weapon to defeat Powdery Mildew disease is to silence Mildew Resistance Locus 0 genes (MLO). MLO serves a vital role as a negative regulator, influencing defense activation by impeding the transport of defense compounds to nascent papillae. This interference assists germinated Powdery Mildew pathogen spores in preventing the formation of a Haustorium for nutrient uptake.

Knocking down or knocking out one or more specific MLO genes represents a strategy for protecting plants against Powdery Mildew infections.

Stable inserts were designed to target the MLO6 gene in N. Benthamiana. The N. benthamiana MLO6 gene was selected to validate this approach. This approach involved harnessing the CY1-induced host MLO gene to mitigate Powdery Mildew Disease in the model plants.

Three CY1 constructs containing the inserts target at MLO6 expression were engineered and used to infect host plants, which were then evaluated for their disease resistance again the tobacco powdery mildew isolate Golovinomyces cichoracearum (Gc) SICAU1. Visual inspection of leaves for symptoms (white fungal mass) after 8 weeks indicated that plants infected with the CY1 constructs containing inserts targeted against NbMlo6 had enhanced resistance to powdery mildew relative to plants infected with a control (wildtype CY1).

The three CY1-based RNAi vectors were designed by using a modified −97 nucleotide RNA sequence from the N. benthamiana MLO6 gene, which were engineered based on the hairpin mimic structure described herein (FIG. 32). The CY1 wt DG was −77 kcal/mol. The DG of CY1.NmMloJH was −71.10 kcal/mol. The DG of CY1.NmMlo1 was −72.90 kcal/mol. The DG of CY1.NmMlo2 was −70.50 kcal/mol.

NbMloJH has an average APE of 0.25; maximum PE of 1.6; length of 198 nucleotides; 13 bases (6.6%) with PE greater than one, a standard deviation of PE of 0.39 and an MFE of −80.7 kcal/mol. The sequence of NbMloJH is SEQ ID NO: 77:

ACCTGGGGATGACCTCTTTTGGTTTAATCGTCCTCGTCTCATTCTTTAT
CTCATTAATTTTGTCCTCTTTCAGAATGCTTTTCAATTGGCCTTCTTTT
TTTAAAGAAGCCCAATTGAAAACTTAAACTGTAAAGAGGACTCTTATTA
AGGAAATTAGAATAGACCACCGACGA TAA
GAGTAAAGAGGTCATGCCAGGT

NbMlo1 has an average APE of 0.23; maximum PE of 1.6; length of 198 nucleotides; 13 bases (6.6%) with PE greater than one, a standard deviation of PE of 0.38 and an MFE of −82.8 kcal/mol. The sequence of NbMlo1 is SEQ ID NO: 78:

ATGACCTCTTTTGGTTTAATCGTCCTCGTCTCCTTCTTTATCTCATTAA
TTTTGTCCTCTTTCAGAATGCTTTTCAATTGGCCTTCTTTGCTTGGACT
TTTGTCCAAGAAAAGAAGGCCACCACACAAATGCATTCTGACACAAGGA
CCAACTTCTGAGAAAAGCTCAGAGACAGGCAACTTAAACCAAAACGGTC
AT

NbMlo2 has an average APE of 0.17; maximum PE of 0.97; length of 198 nucleotides; 0 bases (0%) with PE greater than one, a standard deviation of PE of 0.26 and an MFE of −79.8 kcal/mol. The sequence of NbMlo2 is SEQ ID NO: 79:

GTTGATTACTTAACCCTAAGGCATGGATTTATCATCGCGCATTTGGCACC
TCAGAGCCACGTAAAGTTTGATTTCCAAAAGTATATCAACAGGTCACTTT
TGTGACCTATTGATATACTTCAGACTAATACAAACTTTAAACCGGCTCAG
ACGTACAAATCGCGCCAAATAAACCAACAATTAGGGTTAAGCATCAAC

The DMR6 gene is also an effective susceptibility gene. Targeting DMR6 and sequences of DMR6 in various plants are described, for example, in US 2021/0071195, Soybean Plants Resistant to Phytophthora Sojae, which is incorporated herein by reference.

In experiments, three inserts were designed targeting the N. benthamiana DMR6 gene (NbDMR6). The three inserts, DMR6.1, DMR6.2 and DMR6.3, are shown in FIG. 31. Other inserts target at DMR6 genes in citrus, grape and cannabis are also shown in the FIG. 31.

Eighteen N. bentamian plants for each CY1 construct were agroinfiltrated with CY1 containing inserts to produce NbDMR6 silencing siRNA, employing hairpin-like structures as described herein. Three weeks after agroinfiltration, the expression levels of the NbDMR6 gene were quantified using real-time PCR (qPCR). The data analysis revealed that NbDMR6 gene expression was reduced by about 55% in plants infected by CY1-DMR6 compared to the control plants.

Three systemic leaves on each plant, previously infected with CY1 carrying NbDMR6 silencing inserts, were subsequently challenged with 0.0001 (OD600) of P. syringae pv tabaci through infiltration in a 10 mM MgCL₂ solution (three plants for each CY1 siRNA construct). As a control, plants infected with wild-type CY1 were also subjected to bacterial infiltration. Four days after the challenge, the disease caused by P. syringae was assessed by visual inspection. Leaves in plants treated by CY1-DMR6 shows minimal symptoms whereas leaves in plants treated with a wildtype CY1 control showed severe necrosis (large areas of white discoloration). The bacterial titer in the infiltrated areas was determined by plating. The results demonstrated that the bacterial levels in plants treated with the CY1-DMR vector were reduced by nearly 10.9 fold relative to the control plants.

The sequence of DMR6.1 (SEQ ID NO: 80) is:

GGCCTCAATTTACCAGTAGAAGAGAAACTAAAATTATATTCAGATGATC
CTTCAAAGACCATGAGATTATCAACAAGTTTTAATGTTAGGAAGGAGACA
TTTTTGTCTCCGTCCTAACATTATGTTAATGTATGATAATCTGGCGGGTC
TGTGTAGTTCATCGAATCCCCATTTTGTTAAAATTCTACTGGTACTTGAG
GCC

The sequence of DMR6.2 (SEQ ID NO: 81) is:

CTCTAGAGAAGTATGCTCCTGAATGGCCTTCTAATCCATCATCTTTCAG
GGAAATCGTGAGCAGATATTGCAGGGAAATTCGTCAACTCGGATTTAGT
TTTCTAAATCGGAGTTGACGAACCAAAACTGCCAATATCTGAAAAACGA
TATCACTTAAGATATGGTAAGAGAAGCCAAAATGGAGCATATTTACTAG
AG

The sequence of DMR6.3 (SEQ ID NO: 82) is:

GCCATAGCAGAAAGCCTGGGGTTAGATAAAGAGTGTATAAAAGATGTAT
TGGGTGAACAAGGACAACATATGGCTATCAATTATTATCCTCCTTGTCT
TTTGACAAGGTGGATAATAATTCTCCCTCCATTATGTTGTCAAAAGTTC
AGCCGATGATCATTATAATTATCTTTTCTCCAACCAGGCTTTCTATATG
GC

The sequence of DMR6.CITRUS (SEQ ID NO: 83) is:

GACCCAAATGCCCTTACCATTTTATTGCAAGATTTAGAAGTGGCAGGTC
TTCAAGTTCTCAAAGACGACAAATGGGTTGCTGTTAATCCCCTTCCTAT
TTTTAGGAAGCGGATTAACAGCTTTGAAATTATGTTGTCTTCCTTGAAT
TCGATGAATGTCATTCTTTCTTCTTGAAATCTCCTGGTAAGGGTAATGG
GTC

The sequence of DMR6.GRAPE (SEQ ID NO: 84) is:

CCTCAACATGGTTGAAGGGAGTAAAAGATTTTTCGAGCTCCCTTTCGAG
GAGAGATCCAAGTACATGTCGACTGATATGTATGCCCCAGTTAGGTATT
TTTATATCTATCTGGGGTATACCCACAAAGTACGATATGTAAAACGGAT
CACTACTAAAAGGAGCTAATTAAAATATTTGGGACCCTTCAACCACTTG
AGG

The sequence of DMR6.CANNABIS (SEQ ID NO: 85) is:

CTTCCACATGAGGAGAGAGCTAAGTACATGTCCTCTGATATGAAAGCTA
AAGTGCGATATGGAACAAGCTTTAACCAAACTAAAGACAAAGTCTTTTT
TTTAAAAGACATTGTCTTTAGTAACCCATAAAGCTTGTTCTTAAATCGC
TCTATAATTTCAATCACCTCGACATTACAATCCTCTCTCCTCAATGGAA
G

### P. Insert Stability for Animal Vectors—Poliovirus and Coxsackievirus B3 (CVB3) Live Attenuated Vaccines.

Live attenuated vaccines induce the most complete and durable immune response, including local and systemic responses, as they closely recapitulate the life cycle of the pathogenic viruses. In addition, they are relatively easy to propagate and administer. Currently, live attenuated vaccines against poliovirus (oral poliovirus vaccine, OPV) are applied on a global scale, and they are envisioned as the cornerstone of the Global Polio Eradication Initiative.

Unfortunately, the currently available anti-poliovirus vaccines, including the newly developed OPV strains (nOPV), have suboptimal properties which in combination with the administrative difficulties of organizing effective vaccination campaigns has led to repeated missed deadlines for poliovirus eradication. The original OPV developed by Albert Sabin is a combination of three attenuated poliovirus strains. In addition to inducing gut mucosal immunity required for the interruption of viral transmission, it is inexpensive, can be administered by minimally trained personnel, and the replicating vaccine may spread beyond the primary vaccine recipient increasing the vaccination coverage, which makes this vaccine the vaccine of choice in limited resource settings. Yet, in about 1 per million of the primary vaccine recipients it induces vaccine-associated paralytic poliomyelitis (VAPP), and OPV-derived viruses can establish circulation in under-immunized populations, generating so-called circulating vaccine-derived polioviruses (cVDPV) which may also cause the disease. In fact, currently most of the registered cases of paralytic poliomyelitis are attributed to cVDPV rather than to the remaining circulation of the wild type viruses.

Thus, the development of improved live polio vaccine strains is considered a high priority. The current approaches include a combination of the stabilization of a secondary structure element in the non-translated genome region, rearrangement of an important cis-acting RNA signal, and the introduction of mutations improving the fidelity of the viral RNA polymerase. These features decrease the possibility of reversion to the pathogenic phenotype and the recombination with other enteroviruses. Recently, a newly developed vaccine strain of poliovirus type 2 with several features for increased stability was introduced into immunization practice (nOPV2). Yet, in spite of the promising clinical trial results, the recent data register both the emergence of nOPV2-derived cVDPV and the associated cases of paralytic disease. Thus, the strict requirements for stable and safe live poliovirus vaccine strains have not been met.

Data herein demonstrate that unprecedented stability of inserts in the 5′ non-translated region of enteroviruses, including poliovirus and CVB3. Poliovirus and Coxsackie B 3 virus (also an important human pathogen) were generated with inserts harboring target sequences for miRNAs specifically expressed in neuronal and cardio tissues. Introducing miRNA target sequences in the viral genome allows cell-type-specific inhibition of replication. This is a promising direction for the design of safe live attenuated vaccine strains provided that methods, for example as described herein, for stabilizing the miRNA targeted inserts are applied.

The stabilized inserts were 99-100% stable over at least 10 passages of these viruses in cell culture, i.e. over multiple replication cycles. Control inserts designed without considering the insert stability parameters were lost within the first passages, in accordance with previous reports.

The approach of stabilizing inserts in the genomes of RNA viruses was tested on two enteroviruses, poliovirus and Coxsackievirus B3 (CVB3) (family Picornaviridae, genus Enterovirus, species C and B, respectively). Enteroviruses are positive-strand RNA viruses with the genomes of ~7500 nt. The enterovirus RNA codes for one polyprotein and contains an extensive 5′ non-translated region (5′UTR). There is a poorly conserved stretch of −100 nucleotides between the end of IRES and the beginning of the viral ORF (FIG. 33), which is believed to be scanned by the ribosomal 40S subunit during translation initiation.

Hairpin-like inserts containing target sequences were designed for the neuronal and cardio tissue-specific miRNAs (hsa-miR-9 and hsa-miR-150) for insertion into this region in poliovirus and CVB3 genomes, respectively. The insertion of miRNA target sequences is a promising approach for vaccine strain attenuation but is difficult to implement in positive RNA viruses because they readily removed foreign inserts in the genome in previous studies. For both viruses, two insertions were designed—one according to the methods described herein which includes adjustment of the hairpin stability and in these examples surrounding sequence modification, and the control constructs with straightforward hairpin insertion in the viral genome (FIG. 33).

The corresponding RNAs were synthesized using a T7 transcription system and transfected into HeLa cells. All viruses were viable and showed plaque phenotypes similar to that of the wildtype viruses, except for the CVB3 control construct which produced noticeably smaller plaques.

The viruses were passaged 10 times in HeLa cells (each passage comprised multiple cycles of replication). The total viral RNAs from passages 0 (the recovered virus after RNA transfection), 5, and 10 were extracted, and the region encompassing the inserted sequences was amplified by RT-PCR and sequenced.

### Poliovirus

The secondary structure of the insert (has-miR-9-5p MIMAT0000441), the backbone polio virus and the modified polio virus (after replacement of wildtype bases 620-679 with the insert has-miR-9-5p MIMAT0000441) is shown in FIG. 38. Insert has-miR-9-5p MIMAT0000441 has an APE of 0.08 (0.06 without the AU pair end), a length of 52 bases, and an MFE of −24.80 kcal/mol. For the control poliovirus construct, sequence heterogeneity was observed already in passage 0, showing extreme instability of the hairpin insert (FIG. 34). In passages 5 and 10 of the control construct only wt sequence was recovered (not shown).

On the contrary, for the designed insert in the poliovirus genome, only one nucleotide change was observed after 10 passages (FIG. 35).

The region of the vector including the stabilized heterologous insert (in bold) shown in the figures is presented below as SEQ ID NO: 86:

ACAATCACAGATTGTTATCATAAAGCGAATTCTTTGGTTATCTAGCTGT
ATGAatcacaTCATCCAGCCCGATAACCAAAGATTGAGTGTGTTTACTC
TAAGTACAATTTCAACAGTTATTTCAATCAGACAATTGTATCATAATGG
GTGCTCAGGTTTCATCACAGAAAGTGGGCGCACATGAAAACTCAAATAG
AGCGTATGGTGGTTCTACCATTAATTAC

In some figures, the sequencing test data differs from SEQ ID NO 87 by way of mutations or deletion apparent from the figures.

### CVB3

The secondary structure of the insert (hsa-miR-150-5p MIMAT0000451), the backbone polio virus and the modified polio virus (after replacement of wildtype bases 648-676 with the insert hsa-miR-150-5p MIMAT0000451) is shown in FIG. 37. Insert hsa-miR-150-5p MIMAT0000451 has an APE of 0.05, a length of 49 bases, and an MFE of −19.20 kcal/mol. The control insert in CVB3 genome was more stable than that in poliovirus RNA, but at passage 5 we observed a significant deletion, and at passage 10 all the insert and part of the surrounding sequence was absent (FIG. 36 Part A). Remarkably, the designed sequence was absolutely stable in the CVB3 genome during 10 passages (FIG. 65 part B).

The region of the vector including the heterologous stabilized insert (in bold) shown in the is presented below as SEQ ID NO: 87:

ATTGGCCATCCGGTGACCTCTCCCAACCCTTGTACCAGTGtatatCACT
AGCACAAGCATTGGGAGCGTTTATACCACTTAGCTTGAAAGAGGTTAAA
ACATTACAATTCATTGTTAAGTTGAATACAGCAAAATGGGAGCTCAAGT
ATCAA

In some figures, the sequencing test data differs from SEQ ID NO 87 by way of mutations or deletion apparent from the figures.

Thus, the results demonstrate that the method of insert stabilization can be broadly applied to positive-strand RNA viruses. The method may be used, for example, for designing stable vaccine strains with insert-derived attenuation properties.

### Relationships Between MFE and Length in Other Wild-Type Nucleotides

Randomly generating 38,577 substructures of varying lengths, nucleic acid compositions, and complementarity suggested that the relationship between ΔG and substructure length is affected by the number of base-pairs within a substructure (FIG. 40A). CY1 substructures have a relationship between ΔG and substructure length with a slope (−0.42) that resembles the slopes for randomly generated substructures with only 50-60% complementarity. Complementarity as determined for the randomly generated substructures differs from complementarity as appears from CY1 substructure drawings herein primarily due to differences in the treatment of G:U pairing. However, the trends in the data suggest a relationship between ΔG and length for CY1 that is a consequence of the balance between paired and unpaired regions. Graphing the ΔG/length ratio by the APE of the randomly generated hairpins shows a logarithmic relationship between the two variables such that APE rapidly increases as ΔG/length increases (FIG. 40B). Natural hairpins H1-H4 of CY1 possess nearly the highest MFE/length ratio possible without entering the region where APE rapidly increases. The substructures of randomly generated substructures quickly break down when their complementarity is less than 50%, as indicated by the increasing number of apical loops predicted in these structures (FIG. 40C). Based on the MFE/length relationship, CY1 and other +RNA virus substructures may have nearly as little complementarity (and nearly as high MFE) as possible while still reliably forming hairpin-like structures. This suggests CY1 and other +RNA virus evolved to have hairpin-like structures with particular thermodynamic qualities rather than having thermodynamic properties that are the result of random folding.

This relationship between substructure ΔG/length found for CY1 may also apply for other +RNA viruses but not for structures of large RNAs in general. An analysis of simple substructures found in the full-length structures determined for tomato bushy stunt virus (TBSV) (Wu B, Grigull J, Ore M O, Morin S, White K A. 2013. Global organization of a positive strand RNA virus genome. PLoS Pathog 9:e1003363) and Hepatitis C virus (HCV) (Mauger D M, Golden M, Yamane D, Williford S, Lemon S M, Martin D P, Weeks K M. 2015. Functionally conserved architecture of hepatitis C virus RNA genomes. Proc Natl Acad Sci USA 112:3692-3697) produced very similar relationships between ΔG and hairpin length as found for CY1 (FIG. 40D). In contrast, hairpin-like structures found in 16S and 28S ribosomal RNA produced a steeper slope, suggesting a higher percentage of high complementary hairpin-like structures and more stable structures. In contrast, hairpin-like structures predicted by in silico folding of plant mRNAs, as well as transcripts from plant and animal DNA viruses, produced less steep slopes similar to that of unstructured randomly generated hairpins (<30% complementarity) (FIG. 40E). Together, these observations suggest that +RNA viruses have evolved RNA structures that fit between the extremely structured ribosomal RNA and the much less structured mRNA from both plants and DNA viruses.

FIG. 40F shows linear regressions of MFE to length for hairpin-like structures found in the predicted structures of CY1 and other +RNA viruses. The linear regression for CY1 differs slightly from FIG. 40D because all hairpin-like structures in CY1 with more than 13 bases were used in FIG. 40F whereas only the four exemplary hairpin-like structures were used in Panel D. Similar linear regressions for other +RNA plant and animal viruses (TCV, YFV, SARS-CoV, EVA71, PVX, GULV, PEMV2, DENV2, WNV, and VEEV) were also determined. The linear regressions for these viruses are within the linear regressions shown in FIG. 40F but for SARS-CoV2, PVX, and DENV2 which had slightly lower slopes in the range of −0.27 to −0.30. Considering that poliovirus is near the upper boundary of the plant and animal viruses shown in FIG. 40F and listed in this paragraph, it can be predicted that inserts designed according to one or more principles described herein will be stable in plant and animal +RNA viruses generally. For example, inserts for a +RNA virus, including the 16 viruses described herein, may have a minimum free energy in kcal/mol in a range of about −12 to +15, or −20 to +20 if the insert has more than 150 nucleotides, from a linear regression derived from hairpin-like structures in a wild type CY1 virus. Alternatively, and considering that individual hairpin-like structures vary from the linear regressions, it may be predicted that inserts designed with MFEs in kcal/mol in a range −0.27x to −0.49x, where x is the number of bases in the insert, or optionally in a range of (−0.27x plus 10) to (−0.49x minus 10) kcal/mol, optionally in combination with one or more principles described herein, will be stable in plant and animal +RNA viruses generally, including the 16 viruses described herein.

One possibility for why inserts are rapidly deleted if they contain unacceptable thermodynamic properties (high APE, low ΔG/length) is that these properties create RdRp recombination hotspots within the CY1 genome. Recombination hotspots have been studied in many RNA viruses and are often found adjacent to stem-loop structures or within unstructured regions with low base-pairing probability. Regions of low ΔG/low plasticity may cause polymerase pausing, promoting the premature release of the RNA template. If the RdRp maintains its association with the truncated nascent strand, it can resume transcription at a second location on the same or a different template. If the restart site is close to the initial release site, a local deletion occurs, aligning with our observations for GFP52, GFP59, GFP66, and Cal7-66 insertions. There is no definitive explanation for why RdRp may dissociate from the template at sequences with high PE, but increased structural plasticity in the region may lead to formation of an ensemble of secondary structures, including ones that disrupt RdRp processivity.

Since DCL endonucleases cleave dsRNA, it is also possible that alternation between relatively short consecutively base-paired regions and unpaired regions reduces recognition by antiviral defense mechanisms, which target fully double-stranded RNA. Conversely, large regions of single-stranded RNA may be inherently less stable and provide a better target for the RISC complex. Under this model, the trends we have observed in the thermodynamics of CY1 hairpins may not directly contribute to stability, but instead may describe properties of hairpins that are resistant to host antiviral mechanisms. Even for +RNA viruses with linear regressions having lower slopes than CY1, it may be advantageous to use inserts designed according to CY1 properties to benefit from this immune system avoiding property. Inserts designed according to CY1 properties also have practical advantages of readily forming the predicted substructure without interfering with the secondary structure of the host viruses around the insert site.

All therapeutic siRNA approaches for controlling pathogens suffer from rapid pathogen evolution that reduces siRNA complementarity. A major advantage of VIGS technology over traditional genetic modification (GMO) approaches is its flexibility (45), with updated versions ready for deployment within weeks. The evolution of resistant pathogen strains can also be delayed by including multiple siRNA targeting sequences in the VIGS vector and/or designing inserts that target conserved regions of metabolically critical components, which the pathogen cannot easily modify. However, the flexibility of VIGS requires the design of insertions that are likely to be stable. Therefore, establishing a strategy for designing stable hairpin inserts for viral vectors marks a significant breakthrough in biotechnology. Our new approach for improving the CY1 VIGS vectors is likely applicable to other VIGS vectors. This would enable detailed gene expression studies and offer innovative solutions for controlling pests and pathogens. Expanding the use of VIGS for tree and vine protection will hopefully encourage researchers to adapt many of the 50 known VIGS vectors to longer-lived plant hosts that were previously considered unfeasible. This advancement should have a wide-reaching impact, driving economic growth and increasing global agricultural output.

The applicability of the stability factors described above to other plus sense RNA viruses was confirmed using TRV and PVX vectors and hairpin-like structures designed to be either stable or unstable according to the findings with CY1. These results demonstrate that plus sense RNA viruses have evolved to contain substructures with specific thermodynamic properties, and hairpin-like structures containing these properties are stable when inserted into non-functional regions of the virus genome. Among other applications, stable inserts may increase the utility of VIGS for long-lived trees and vines.

FIG. 41 shows the genomic arrangement of a Tobacco Rattle Virus (TRV) vector, insert locations, and a method of infecting plants with the vector. FIG. 42 shows the genomic arrangement of a Potato Virus X (PVX) vector, insert locations, and a method of infecting plants with the vector.

FIG. 43 shows the conformation, APE and MFE of four hairpin-like structures, H1-H4, from CY1 and two synthetic hairpin-like structures Ftsz102 (alternatively called Ftsz 160) and Ftsz69 (alternatively called Ftsz 159). As indicated therein, Ftsz69 has an MFE/length near a line of best fit through H1-H4 and is stable whereas Ftsz102 is distant from that line and unstable. H1-H4 are the same as structures 1-4 shown in FIG. 39 and with SEQ ID Nos: 64-67 given further above. Ftsz102/Ftsz160 and Ftsz69/Ftsz159 are described, and their sequences provided, further below.

Referring to the upper panels of FIG. 44, hairpins inserted into the PVX vector are usually found to be unstable after 2-3 weeks of infection. However, hairpin-like structures were inserted into two locations in PVX vectors. The hairpin-like structures were H1 (natural stable CY1 hairpin-like structure), Ftsz69 (hairpin-like structure designed with parameters to be stable), and Ftsz102 hairpin-like structure designed to be unstable). After 6 weeks in plants, H1 and Ftsz69 were stable, whereas Ftsz102 shows multiple bands indicating deletions (asterisk). These are similar to the results that we got with CY1 as a vector. This shows that the same thermodynamic parameters work for both CY1 and PVX, which are completely unrelated viruses.

Referring to the lower panels of FIG. 44, TRV is the most stable VIGS vector known, though still subject to deletions. Hairpin-like structures were inserted into the single cloning site in the TRV vector. The hairpin-like structures were natural stable CY1 hairpins H1, H2, H3, H4, Ftsz69 (hairpin-like structure designed with parameters to be stable), and Ftsz102 (hairpin-like structure designed to be unstable). After 12 weeks in plants, H1-H4 and Ftsz69 were stable, whereas Ftsz102 shows multiple bands indicating deletions (asterisk). These are similar to the results that we got with CY1 as a vector. This shows that the same thermodynamic parameters work for both CY1 and TRV, which are completely unrelated viruses.

FIG. 45 shows the conformation, APE and MFE of three more synthetic hairpin-like structures Ftsz160, Ftsz159 and Ftszrc160. As indicated therein, Ftsz150 and Ftszrc160 have an MFE/length near the line of best fit through H1-H4 and were stable at 10 weeks post infection whereas Ftsz 160 is distant from that line and unstable at 3 weeks post infection. The sequences of these inserts are below:

Ftsz160
(SEQ ID NO: 96)
CTAAGCAGATTATTCAACTAGGATCTGGAATAACCGAAGGTTTGGGAGC
AGGATCTCATCCAGAAGTTGGTCGTGCTGCTACCAGCGCGACCAACGTC
TCGATGCGATCATGCTCACAAACCTTCAATTATTCCAGATCCTAGCCGA
ATAATATGCTTAG
Ftsz159
(SEQ ID NO: 97)
CTAAGCAGATTATTCAACTAGGATCTGGAATAACCGAAGGTTTGGGAGC
AGGATCTCATCCAGAAGTTGGTCGTGCTGCTACAAGCGTGATCAACGTC
TACGATGCGATCATGCTACGAGTCTTCATTATAACAGATCTTAGCGAAT
AATAATGCTTAG
Ftszrc160
(SEQ ID NO: 98)
CTAGGCATTATTATTTGACTAAGATCTGTTATAGTTGAAGGCAGGCCGT
ATGATTTCATTTACGGGTTGATTGTGCTTGTAGCAGCACGACCAACTTC
TGGATGAGATCCTGCTCCCAAACCTTCGGTTATTCCAGATCCTAGTTGA
ATAATCTGCTTAG

FIG. 46 shows results of testing H1-H4, Ftszrc160 and Ftsz160 as inserts in a tobacco rattle virus (TRV) vector. The TRV vector is one of the most commonly used vectors in labs. It has the nematode transmission ORF replaced with a multicloning site (MCS). We placed the natural CY1 H1, H2, H3, and H4 hairpins (all stable when duplicated into new sites CY1) and the artificial Ftszrc160 (stable in CY1) and Ftsz160 (unstable in CY1) into the multicloning site. All hairpin-like structures that were stable in CY1 were stable in TRV at 12 weeks post infection. The unstable hairpin-like structure was unstable at 12 weeks post infiltration (bands indicating deletions marked with asterisks).

FIG. 47 shows results of testing H1, Ftszrc160 and Ftsz160 in a potato virus X (PVX) vector. The PVX vector is another one of the most commonly used vectors in labs. It has a new subgenomic promoter upstream of a multicloning site (MCS) inserted downstream of the “triple block” location. We placed the natural CY1 H1 hairpin (stable when duplicated into new sites CY1) and the artificial Ftszrc160 (stable in CY1) and Ftsz160 (unstable in CY1) into the multicloning site. All hairpin-like structures that were stable in CY1 were stable in PVX at 6 weeks post infection. The unstable hairpin-like structure was unstable at 6 weeks post infiltration (marked by asterisk). This demonstrates that thermodynamic parameters that allow hairpins to be stable in CY1 also work for these two unrelated plant virus vectors as well, and thus these parameters are not CY1 specific.

FIG. 48 shows results of an experiment that examined small incremental differences in hairpin-like structure thermodynamic stability and their effect on maintenance in virus CY1. The DG of the hairpin-like structure Ftsz159 was modified by changing nucleotides to close the internal unpaired loops. Results showed that increasing the DG from −70 kcals/mole to −82.5 maintained stability in CY1. However, increasing the DG to −87 generated loss of the hairpin-like structures. Increasing the DG further caused a greater percentage of deletions to occur. Since a possible caveat is that we created a longer base-paired region in the hairpin-like structures, which could have been better templates for double-stranded RNA-binding proteins that could have affected stability somehow, the next figure shows what happens when unstable “C3” is altered to maintain the double-strand but weaken the thermodynamic stability.

Referring to FIG. 49, here we show what happens when unstable “C3” is altered to maintain the double-strand while weakening the thermodynamic stability. The sequences in the upper portion of hairpin-like structures C3, C3A and C3B are shown. Weakening the thermodynamic stability while maintaining the base-paired region caused the hairpin-like structures C3-A and C3-B to now be maintained at 8 weeks post-infiltration. Note that there are some faint lower bands for C3A at 3-WPI but these could not be cloned and were absent at 8 weeks. Faint bands were also in the control plasmid lane. Sequences for the base-paired regions of C3, C3-A and C3-B are below:

C3:
(SEQ ID NO: 99)
CUAAGCAGAUUAUUCAACUAGGAUCUGGAAUAACCGAAGGUUUGGGAGC
AGGAUCUCAUC
C3-A:
(SEQ ID NO: 100)
CUAAGCAGAUUAUUCAACUAGGAUCUGGAAUAACCGAAGGUUUGGGAGC
AGGAUCUCAUC
C3-B:
(SEQ ID NO: 101)
CUAAGCAGAUUAUUCAACUAGGAUCUGGAAUAACCGAAGGUUUGGGAGC
AGGAUCUCAUC
C3:
(SEQ ID NO: 102)
CAGAAGUUGGUCGUGCUGCUACAAGCGUGAUCAACUUCUGGAUGAGAU
CAUGCUACGAGU
C3-A;
(SEQ ID NO: 103)
UAGGAUUUGGUCGUGCUGCUACAAGCGUGAUCAGGUUCUGGGUGAGAU
CAUGCUACGAGU
C3-B:
(SEQ ID NO: 104)
UAGGAGUUGGUCGUGCUGCUACAAGCGUGAUCAGCUUCUGGGUGAGAU
CAUGCUACGAGU
C3:
(SEQ ID NO: 104)
CUUCAUUAUAACAGAUCUUAGCGAAUAAUAAUGCUUAG
C3-A:
(SEQ ID NO: 105)
CUUCAUUAUAACAGAUCUUAGCGAAUAAUAAUGCUUAG
C3-B:
(SEQ ID NO: 106)
CUUCAUUAUAACAGAUCUUAGCGAAUAAUAAUGCUUAG

Referring to FIG. 50, previously we examined the effects on stability of increasing the thermodynamic stability of the inserted hairpin-like structure (making the DG more negative). Here we examine the effects of making the hairpin-like structures gradually less thermodynamically stable (while maintaining a low value for the average positional entropy). Hairpin-like structures were inserted into two locations in PVX: the normally used central location and at the beginning of the 3′ UTR. Two hairpin-like structures previously examined (unstable Ftsz160 [labeled as “1” ] and stable Ftszrc160 [labeled as “2”) were used as controls. In both locations, all hairpin-like structures that were less thermodynamically stable (hairpin-like structures 3-5) were not 100% maintained. Interestingly, the hairpin-like structures were maintained better in the 3′UTR location (though still with deletions apparent at 14 days post infection). This shows that there are both upper and lower bounds for DG/length that hairpin-like structures require to not be lost.

Referring to FIG. 51, the thermodynamic properties (DG, APE) of hairpin-like structures is the primary feature for keeping hairpin-like structures intact in virus vectors. However, when a hairpin-like structure does not have the proper thermodynamic properties (and will thus be unstable), we have noticed that deletions occur frequency at repeated sequences of 3-5 bases. The major deletion that we get with unstable hairpin C3 is at a repeat “CUGGA”. We therefore wanted to know if we alter one of the two sequences, will there be fewer deletions associated with the unstable hairpin. We changed one of the CUGGA to CUGGGA to create C3-A29G (alternatively called C3-G29A). While the hairpin-like structure still appeared to be unstable there were significantly fewer deletions accumulating. This shows that another secondary (i.e. beneficial but not necessarily essential) parameter (DG/length and APE being the primary parameters) is the lack of 4-5 base repeated sequences.

Referring to FIG. 52, we stacked three hairpin-like structures that individually are all stable in CY1. These were natural CY1 hairpin-like structures (H4+H1) and artificially designed hairpin-like structure (Ftsz159), and inserted the giant hairpin-like structure at position 2219A (deletion of the natural hairpin-like structure at this location in CY1 and insertion of this very long hairpin-like structure). This 418 nt hairpin-like structure was stable at 3-wpi and 8-wpi. This is the largest hairpin-like structure that we have stabilized to 8 weeks post infection at the time of writing. Panel (A) shows the secondary structure of H4+H1+Ftsz159 with PE profile. Panel (B) shows RT-PCR results from infected plants by H4+H1+Ftsz159_{2219Δ} at 3-wpi and 8-wpi. The PCR targets 2052-2489 region, 28 cycles. Panel (C) shows sequence alignment between parental sequence and batch sequencing from systemic leaf. Panel (D) shows chromatogram of the batch sequencing result. The sequence of H4+H1+Ftsz159 is below:

(SEQ ID NO: 107)
GGTTAGGGTAACTCACATACCTTCTTCCATACAGTAAAAGGTTGTATGG
TGAGGGTGCTGAGCCGTGTATCGCCAAAGGCCTAAATGCATTAGAATCT
GGAGCGACTTTGAGGCGCAAATGGGAGAAGTTCTAAGCAGATTATTCAA
CTAGGATCTGGAATAACCGAAGGTTTGGGAGCAGGATCTCATCCAGAAG
TTGGTCGTGCTGCTACAAGCGTGATCAACGTCTACGATGCGATCATGCT
ACGAGTCTTCATTATAACAGATCTTAGCGAATAATAATGCTTAGTTCTT
CTCCAGTTTGCGTTTCTCTCGACGCTTCCAGGTTCGACCTGCATGTAAG
CGTTGGCATGCTAAAGTTCACACACAAGCTATATGACTATTACTGACTG
GAAAAGGTCGTGTGAGCAACCTAACC

Referring to FIG. 53, we have observed deletions of parts of hairpin-like structures with high APE, either in an absolute sense or relative to the APE of the whole hairpin-like structure. These hairpin-like structures may have violated one of the secondary parameters described herein, or a further secondary parameter may be that no region of the hairpin-like structure should have a high APE.

FIGS. 54A-54B show the stability of the stacked form of a fully base paired structure (but for the apical loop) that defined as stable previously. (FIG. 54A) the native CYVaV structure within the 1493-1525 region (left) alongside its extended, stacked form (right). (FIG. 54B) RT-PCR result for CY2250stk1493-1525 and CY2304stk1493-1525 derived from infected plants. (C) Schematic illustration to show the insertion sites (2319//2281 and 2304) for stk1493-1525. (D) and (E) Alignment of the sequencing result of the PCR products f or CY2250stk1493-1525 (also called Double H2 2219) and CY2304stk1493-1525 (also called Double H2 2304) in panel (B) respectively. The stacked structure was stable.

FIG. 55 shows another fully base-paired (but for the apical loop) hairpin PDS51 that was found to be stable for 3 weeks when inserted at position 2301 of CY1 while the other hairpins shown were not. The natural hairpin H2 of CY1 is also stable as an insert. The examples of FIGS. 55 and 56 indicate that the thermodynamic properties described herein may be applicable to fully base paired structures. However, stable fully base-paired inserts are difficult to design as longer inserts (e.g. longer than PDS51 or the stacked hairpin described above) with an effective engineered targeting sequence. The sequence of PDS51 is given below:

(SEQ ID NO: 108)
AAGGATTCGTACTCCAAGTGCTTCAAGAGAGCACTTGGAGTACGAATCC
TT

The inventors have also observed that the CY2 subgenomic RNA promoter region (CY2 positions 1976-2135) can be inserted in position 2219 of CY1. This example suggests that branched inserts may be stable if they mimic naturally occurring branched structures. However, synthetic branched inserts with stability similar to that of un-branched inserts have not yet been designed.

International application number PCT/US2023/066542, RNA Vectors with Hairpin-Like Inserts, filed on May 3, 2024 and published as WO 2023/215782 on Nov. 9, 2023, is incorporated herein by reference. International application number PCT/US2024/027884, RNA Vectors with Hairpin-Like Inserts, filed on May 3, 2024 and published on Nov. 7, 2024 as WO 2024/229446, is incorporated herein by reference. In some embodiments, the insert is not an insert described in either of the two international applications described in this paragraph. The paper, Hairpin inserts in viral genomes are stable when they conform to the thermodynamic properties of viral RNA substructures, Jang et. al., Journal of Virology, April 2025, Volume 99, Issue 4, pages 1-24 and the software, data and supplemental material described therein are incorporated herein by reference.

### Summary of Insert Design Principles

Application. The following design principles may be used to design inserts for vectors derived from plus sense single stranded RNA viruses. By being “derived from” a virus, the vector comprises a backbone that is the same as or similar to the genomic RNA of a wild type virus, optionally with modifications that do not affect essential functions of the virus required for its use as a vector. Vector backbones derived from some wild type viruses have already been developed and described in the art, for example vectors derived from TRV, PVX, CY1 and CTV, and others are likely to be developed in future. In some examples, the backbone of the vector (i.e. the viral genomic RNA that the insert is inserted into) retains at least 90% or at least 95% sequence identity with the wild-type genomic RNA. The insert is added to the backbone at a location that does not materially interfere with essential functions of the virus required for use as a vector. Such locations are known for many viruses, and additional sites can be determined or sites can be determined for new viruses using similar principles. Without intending to be limited by theory, one aspect of the insert design principles described herein may relate to the ability of the RNA-dependent RNA polymerase (RdRp) to replicate the vector. Accordingly, the principles may be most applicable to RNA viruses that encode or are replicated by an RdRp. For wild type viruses with multi-part genomic RNAs, for example TRV where the RdRp is only encoded by one of the two genomic RNAs (TRV-1), the design principles may be useful in designing inserts for the TRV-1 RNA and the TRV-2 RNA. In some embodiments, the design principles are applied to a sequence for insertion in a vector derived from a Group IV positive-sense single-stranded (+ssRNA) virus according to the Baltimore classification. In some embodiments, the design principles are applied to a sequence for insertion in an RNA sequence that comprises an RdRp derived of a vector derived from a Group IV positive-sense single-stranded (+ssRNA) virus according to the Baltimore classification.

Relationship Between Some Parameters. Without intending to be limited by theory, complementarity, MFE/length and APE may be correlated, either mathematically or in nature.

For example, organizing data from hairpin-like structures with 50% GC content randomly generated in silico into a plot of complementarity against MFE/length and drawing a line of best fit suggests that a formula (Y−0.25)/−1.05, wherein Y is the slope of MFE to length, produces a corresponding percentage complementarity with a variance of about +/−5. Applied to CY1, an MFE/length of −0.42 corresponds with 64% complementarity, or a range of about 60-70%. Since some plus-sense single stranded RNA viruses have a lower MFE/length, a design range may extend downwards, optionally to 50% complementarity. Further, experimental data and analysis of hairpin-like structures in wild-type CY1 suggests that inserts with higher complementarity, for example up to about 80% or about 90%, can be stable. Thus a design process may start with a target complementarity in the range of 50-90% or 60-80%. However, starting the design process with a target complementarity in a range of about 60-70%, for example 64%, has produced many examples of preliminary designs that require only small modifications to achieve a target MFE/length and APE. Optionally, a target percentage complementarity may be produced by entering the MFE/length (Y) of the virus that the vector is derived from (e.g. from Table 1) in the formula in this paragraph, or by determining an average or range of percentage complementarity in a representative number of hairpin-like regions in the virus that the vector is derived from.

In the discussion above, percentage complementarity is defined as the percentage of bases on the targeting-side sequence that are base-paired in the companion strand. In contrast, the definition of complementarity used in FIG. 40 was the percent of bases in the insert that are base-paired, which includes bulges on the companion side and the apical loop into the calculation and partially accounts for the lower value in FIG. 40. The revised definition (which is not affected by the apical loop) is more readily applied to a design process, although either definition may be used with appropriate adjustments to the percentage complementarity values.

While design using percentage complementarity alone can produce stable inserts, designs that are within a targeted range of MFE/length or APE, or preferably both, are more likely to be acceptable regardless of whether they have a specific percentage complementarity (though they will typically be within a range of 60-90%). However, percentage of complementarity is easier to use than MFE or APE to generate an initial design that is likely to have a satisfactory MFE/length and/or APE or that will require minimal adjustment to have a satisfactory MFE/length and/or APE. For example, selecting 64% complementarity as a design target implies that, starting with a targeting-side sequence to be used on one side of the insert, roughly two thirds of the bases selected for the other side of the insert, when the complete sequence of the insert is folded around its apical loop, should result in base pairs. Some base pairing possibilities cannot be predicted with complete certainty before analyzing the complete sequence by way of software or laboratory techniques). However, for a preliminary design intended to come close to a target percentage complementarity, the canonical Watson-Crick base pairs (A pairs with U; G pairs with C) can be assumed to result in base pairs, the non-canonical base pair (G pairs with U) can be assumed to result in base pairs, and all other combinations (A:G, C:U, A:C) can be assumed not to result in base pairs. While it is not necessary for the non-targeting-side sequence to have the same number of bases as the targeting-side sequence, in an initial design it can be easier to use the same number of bases on both sides and thereby produce a pattern of paired stems and symmetrical loops. Preferably, the length of stems is randomized or at least varied.

Whether a proposed insert design folds to produce a hairpin-like structure or not, and produces the selected percentage complementarity value, can be checked by entering the proposed sequence into Vienna RNA or similar software and viewing the predicted secondary structure. The base pairings are modified if a folded structure does not form. In a trial and error process which typically does not require many iterations, the optional non-Watson Crick base can be used across from an A or C on the targeting side, GC pairs may become GU pairs, bulges may be made non-symmetrical, or the length, number or arrangement of loops and stems may be varied.

In addition to providing an insert that is likely to be more stable than a fully base-paired insert, designing for less than 100% complementarity can provide freedom to choose sequences on either side of the insert according to other goals, for example to prevent distortion of the backbone vector around the insertion site, to avoid the creation of large or full length complements of a targeting sequence elsewhere in the virus vector or in a host, and/or to allow for the creation of inserts from targeting sequences that would be highly unstable if used in fully base paired inserts. In some embodiments, a stable insert is one wherein material deletions are not detected, for example in electrophoresis of RT-PCR products using primers outside of the insert, in a majority (e.g. 60% or more or 80% or more) of laboratory plants (e.g. N. benthamiana) 3 weeks post infection. This standard of stability was selected for screening vectors for potential use to treat durable infections or environmental conditions in plants, for example VIGS vectors or plant pathogen-targeting vectors, and is higher than the standard required, or conventionally used, in some other applications. Accordingly, in some embodiments, a stable insert is one that is more stable than a fully base-paired insert with the same targeting-side sequence, or more stable than an insert with the same targeting-side sequence known in the art.

Alternatively, target percentage complementarity can be used to proactively guide the design of the non-targeting-sequence side, for example by predicting whether the next base should be a paired base or not. In one example, having designed the targeting-side sequence and starting from a paired base adjacent the apical loop or on the opposite end of the insert, whether the next base will be selected to pair with the targeting-side sequence or not is determined by generating a random number between 0 and 1. If the random number is less than a selected percentage complementarity, the next base selected is paired. If the random number is higher than the selected percentage complementarity, the next base selected is unpaired. Various other generative design algorithms may be developed. If the resulting design does not produce a folded hairpin-like structure, then the process may be repeated.

An apical loop may be added to the proposed design before or after designing the non-targeting-side sequence.

FIG. 40 suggests that any correlation between APE and MFE/length is non-linear. A logarithmic transformation suggests that log(APE) has a generally linear relationship to MFE/length, with some deviation from a direct linear relationship. Accordingly, an acceptable MFE/length tends to produce an acceptable APE. However, as the relationship includes some variation, it may be preferable to check a proposed insert design for both MFE/length and APE. Since the relationship is logarithmic, it may be preferable to check and adjust MFE/length first before checking and adjusting APE. An adjustment to APE is not likely to disturb an acceptable MFE, whereas an adjustment to MFE might disturb an acceptable APE.

In combination, while using any one or two of percentage complementarity, MFE/length and APE, can result in acceptable insert designs in many cases, a design process optionally uses percentage complementary to create an initial design, which is then checked from acceptable MFE/length, and then checked for acceptable APE.

PE Clustering. Previous work by some of the present inventors has described various design rules intended to prevent clusters of high, or in some cases low, PE bases. The inventors have observed that such clusters rarely occur if the APE is kept within a narrower range. Accordingly, while an insert design may permit a broader range of APE if the anti-clustering rules are applied, an alternative approach is to require a narrower range of APE. An APE range of 0.07 to 0.32, or about 0.1 to 0.3, has been found to produce stable inserts and avoid PE clustering issues in most embodiments.

Selection of Target Parameters. Referring to FIG. 40, individual viruses have a characteristic MFE/length that can be determined by analyzing the folded regions of their genomic RNA. However, the MFE/length of plus sense single stranded RNA of various viruses tends to fall within a range. FIG. 40 shows a line of best fit for several viruses. In creating a line of best fit, Y-intercepts may be either ignored as they tend to be small or a line of best fit may be forced to pass through the origin. The range of MFE/length of naturally occurring viruses is wider than the range indicated in FIG. 40 Panel F by the deviation of individual folded regions from the line of best fit.

One method of selecting a target MFE for a vector is to analyze a sufficient number of folded regions of the corresponding wild type genomic RNA as described herein and determine a line of best fit of MFE/length for those folded regions and a degree of deviation therefrom. In the case of CY1, analysis of four folded regions produced a relationship of MFE/length very close to the MFE/length determined considering all folded regions, suggesting that the minimum number of folded regions required for the analysis may be as low as four. A higher minimum number, for example 5 or more or 10 or more, might be preferable. An analysis of the individual folded regions can then be used to determine a range around the line of best fit, for example by considering a range of 1-2 standard deviations from the line of best fit. Optionally, if prior information is more readily available, the MFE/length data from a virus with a similar MFE/length, determined for example by FIG. 40 Panel F or could be used.

A representative MFE/length for various plus-sense single stranded RNA viruses, as determined by a line of best fit through wild type hairpin-like regions of the genomic RNA of the virus, is given below in Table 1. Considering the variation in MFE of wild type hairpin-like regions, the MFE/length values in Table 1 can be used with a range of plus or minus 0.1 or 0.05 from the value given in Table 1, optionally while maintaining a maximum of −0.28 or −0.30 and/or a minimum of −0.66, −0.55 or −0.49.

TABLE 1

| Virus Name | Host | MFE/length |
|---|---|---|
| Dengue Virus 2 (DENV2) | Animal | −0.28 |
| Potato Virus X (PVX) | Plant | −0.28 |
| Foxtail Mosaic Virus | Plant | −0.29 |
| SARS-CoV-2 | Animal | −0.30 |
| Tobacco Rattle Virus (TRV) | Plant | −0.30 |
| Enterovirus A71 (EVA71) | Animal | −0.31 |
| Venezuelan Equine Encephalitis Virus (VEEV) | Animal | −0.31 |
| Zika Virus | Animal | −0.31 |
| Citrus Tristeza Virus (CTV) | Plant | −0.32 |
| Poliovirus | Animal | −0.32 |
| West Nile Virus (WNV) | Animal | −0.34 |
| Coxsackie Virus B (CVB3) | Animal | −0.35 |
| Pea Enation Mosaic Virus 2 (PEMV2) | Plant | −0.38 |
| Turnip Crinkle Virus (TCV) | Plant | −0.38 |
| Powassan Virus | Animal | −0.39 |
| Yellow Fever Virus (YFV) | Animal | −0.39 |
| Grapevine Umbra-like Virus 1 (GULV1) | Plant | −0.40 |
| Tomato Bushy Stunt Virus (TBSV) | Plant | −0.40 |
| Citrus Yellow Vein Associated Virus 1 (CY1) | Plant | −0.43 |
| Hepatitis Virus C (HCV) | Animal | −0.49 |

Another method of selecting a target MFE/length for any plus sense single stranded RNA vector is to use the values determined for CY1. Referring to FIG. 40, Panel F, the MFE/length of CY1 is within the range of MFE/length of plus sense single stranded RNA viruses in general. CY1 is also has a very short genomic RNA, which can make viruses less tolerant of inserts compared to viruses with larger genomic RNA. Accordingly, without intending to be limited by theory, inserts that are stable in CY1 tend to be stable in other plus sense single stranded viral vectors.

Another method of selecting a target MFE for a vector is to use an approximate value. As indicated above, MFEs in kcal/mol in a range −0.27x to −0.49x, where x is the number of bases in the insert, or optionally in a range of (−0.27x plus 10) to (−0.49x minus 10), are predicted to produce acceptable inserts. However, while inserts with an MFE/length of −0.27 occur in predictions of folding in wild-type+ssRNA genomic RNA, synthetic designs more reliably form hairpins with an MFE/length greater than −0.3. For practical purposes, i.e. to reduce iterations in an experimental trial and error process, MFE/length may be kept above 0.3 for design purposes. Further, an analysis of many experimental examples suggests that inserts in a broader and/or more negative range extending to −0.55, or even −0.66 at least for insertion in some viruses with more negative MFE/length, are usually acceptable particularly when also checked for having an APE in a range of about 0.1 to 0.3 (optionally 0.07 to 0.32).

As discussed above, a target MFE/length value or range can be converted or correlated to a target percentage of complementarity. A target MFE/length may also be correlated to a target APE but this may produce a wide range of APE whereas the narrower range indicated above may be more robust as it is less sensitive to high PE base clustering.

Adjustments to a Proposed Design. Starting from a proposed insert design, optionally a proposed design that has a selected percentage complementarity, the MFE (as predicted for example using ViennaRNA or similar software) can be adjusted if it is not of a selected value. If the MFE is too high, to reduce MFE then GU or AU pairs can be turned into GC pairs, or unpaired bases within a loop may be paired. If the MFE is too low, to increase MFE then GC pairs may be converted to GU or AU, or paired bases in a stem may be unpaired. While other pairings may be manipulated, GC pairs most directly affect MFE. After every change, the sequence is re-entered into ViennaRNA (or similar software) to confirm that a hairpin-like structure will form and determine the adjusted MFE.

Starting from a proposed design, optionally a proposed design that has a selected MFE or MFE/length, an APE that is too high may be reduced by changing a high PE base, for example a base with a PE greater than 1.0, optionally the highest PE base, to another base. Often changing one high PE base will decrease the PE of a cluster of neighboring bases and is sufficient to reduce APE. However, the process may be repeated with another high PE base, optionally the highest PE base in the revised design, if necessary. Increasing APE is rarely required but could be accomplished by changing a low APE base, optionally the lowest APE base. After every change, the sequence is re-entered into ViennaRNA (or similar software) to confirm that a hairpin-like structure will form and determine the adjusted MFE.

Design Sequence. While a more iterative process may be used, a fairly straightforward process that typically produces acceptable inserts will be described. The order of steps in the description below may be varied, however when used in the order given the process often requires minimal iteration.

In a first step, a targeting side sequence is selected or designed based on the intended purpose of the insert. The targeting side sequence includes or more targeting sequences (e.g. sequences complementary to part of the RNA of a pathogen, host or messenger or other RNA) optionally with some additional bases added to the first side. The design of targeting-side sequences may be according to principles known in the art or that will be developed in the art.

In a second step, an apical loop is added to the targeting-side sequence. The apical loop may have, for example, 3-8 bases, typically 4. Larger apical loops may be used, for example up to 20 bases, but rarely provide advantages. Optionally, the bases in the apical loop are not counted in any calculation involving length herein, which is typically of minimal mathematical effect and not required when the apical loop is short but may be beneficial when the apical loop is long and/or the insert is short. Optionally, the design of the apical loop may be delayed until after the third step below, but an apical loop is required to test that a preliminary design folds into a hairpin-like structure. The design of an apical loop may be according to principles known in the art or that will be developed in the art.

In a third step, a second side of the insert is designed by selecting bases that produce a selected percentage complementarity. This process may be done manually. Optionally, the secondary structure of one or more hairpin-like regions in the virus that the vector is derived from, and/or of one or more previously designed inserts known to be stable, preferably of similar length to the proposed design, may be used as a reference for the location and/or distribution of base-paired regions and loops or bulges in the proposed design. Alternatively an automated process, for example using a random number generator as described above, may be used.

In a fourth step, the proposed insert (i.e. targeting-side sequence, apical loop and opposite side sequence) is inserted in software, for example ViennaRNA described above, to check that the sequence folds into a hairpin-like region. The software will also produce an MFE/length and APE value, and the MFE/length value can be used in the next step described below.

In a fifth step, the MFE/length of the insert is compared to a selected MFE/length vale and adjusted if necessary to produce the selected MFE/length value. The adjustment may be made as described further above. The adjusted insert is inserted in software, for example ViennaRNA described above, to check that the adjusted sequence still folds into a hairpin-like region and to produce an adjusted MFE/length value. The adjustment and checking procedure is repeated until the selected MFE/length is achieved. In the course of this step, the software will also produce an APE value that can be used in the step below.

In a sixth step, the APE of the design is compared to a selected MFE/length value and adjusted if necessary to produce the selected APE value. The adjustment may be made as described further above. The adjusted insert is inserted in software, for example ViennaRNA described above, to check that the adjusted sequence still folds into a hairpin-like region, still produces an acceptable MFE/length value and produces the selected APE value.

In some embodiments the resulting insert has an MFE/length in the range of from a minimum of −0.27 or −0.3 to a maximum of −0.66, −0.55 or −0.49; and an APE in the range of 0.1 to 0.3, or 0.07 to 0.32. The percentage complementarity of the final design may be different than the selected percentage complementarity used at the start of the process.

The parameters MFE/length or MFE are interchangeable when the length of an insert is known or selected and either parameter may be used in the design process described herein. The values used in the design process described herein may be individual values (though typically with some margin for the digital nature of the parameters or general practicality, for example within 5% of the selected individual value) or values that are a selected range of values, for example a range of the value describer herein or a sub-range within such a range.

Length and Configuration of Insert. Unbranched folded regions rarely occur in wild type plus sense single stranded RNA with more than 200 bases. Using design principles as described herein, a fully synthetic insert of 286 bases was stable for at least 6 weeks in CY1. A stable insert of 486 bases consisting of wild type CY1 folded region stacked together, which also had MFE/length and APE as described herein, was also stable. Useful inserts are generally at least 40 or 45 bases long in order to include an effective targeting sequence. Accordingly, the design principles used herein may be most applicable to inserts of 45 to 200 bases, but may have application within a larger range of 40-300 bases or 40-500 bases.

Most of the inserts described herein are linear, i.e. unbranched, inserts. However, some Y-shaped inserts, in some embodiments with length of close to 300 bases, have been designed with the MFE/length and APE parameters described herein and were stable.

One example of a branched insert is m2, shown in FIG. 56. This insert is derived from H1 with a bifurcated region replacing the normally linear apical end of H1. M2 has an APE of 0.22 and a MFE/length of −0.36. In one experiment, m2 was inserted before the 3′UTR in a potato virus X (PVX) infectious clone tagged with GFP between TGB and CP. This construct was tested in Nicotiana benthamiana and was stable, i.e. no deletions were detected, 3 weeks post inoculation.

The sequence of m2 is:

(SEQ ID NO: 109)
caguaaaagguuguauggugagggugcugagccguguaucgccaaaggc
cuaaaugcauuagaaucuggagcgacuuugagccgcaaaagcgccucug
aagcaacquauuguuauguucucucgagcuccagguucgaccugaugug
cguuggcaugcuaaaguucacacacaagcuauaugacuauuacu

Another example of a branched insert is H4+(H1/Ftsz159), shown in FIG. 57. This insert is derived from H4 with H1 and Ftsz159 added to the apical loop of H4 to form a Y-shaped branched structure. H4+(H1/Ftsz159) has an APE of 0.15 and a MFE/length of −0.46 kca./mol/b. In one experiment, H4+(H1/Ftsz159) was inserted into the CY1 vector replacing the wild-type structure between positions 2219 and 2280. This construct was inserted in Nicotiana benthamiana, using agroinfiltration after inserting the modified CY1 genome and a 35s promoter in the pCB301 pXT1 binary vector (plasmid) from NovoPro Bioscience Inc. The insert was, i.e. no deletions were detected in 4 of 5 plants, 3 weeks post inoculation.

The sequence of H4+(H1/Ftsz159) is:

(SEQ ID NO: 110)
GGTTAGGGTAACTCACATACCTTCTTCCATACAGTAAAAGGTTGTATGG
TGAGGGTGCTGAGCCGTGTATCGCCAAAGGCTAAATGCATTAGAATCTG
GAGCGACTTTGAGGCGCAAATGGGAGAAGTTTTCTTCTCCAGTTTGCGT
TTCTCTCGACGCTTCCAGGTTCGACCTGCATGTAAGCGTTGGCATGCTA
AAGTTCACACACAAGCTATATGACTATTACTGACTAAGCAGATTATTCA
ACTAGGATCTGGAATAACCGAAGGTTTGGGAGCAGGATCTCATCCAGAA
GTTGGTCGTGCTGCTACAAGCGTGATCAACGTCTACGATGCGATCATGC
TACGAGTCTTCATTATAACAGATCTTAGCGAATAATAATGCTTAGCTGG
AAAAGGTCGTGTGAGCAACCTAACC

All identified publications and references mentioned herein are hereby incorporated by reference to the same extent as if each such publication was specifically and individually indicated to be incorporated by reference in its entirety. While the disclosure has been described in connection with exemplary embodiments, it will be understood that it is capable of further modifications and this application covers any variations, uses, or adaptations following, in general, the principles of the disclosure and including such departures as come within known or customary practice within the art to which the disclosure pertains and as may be applied to the features hereinbefore set forth.

What is claimed is:

1. A synthetic RNA segment having a minimum free energy in kcal/mol in a range of (−0.27x plus 10) to (−0.49x minus 10), where x is the number of bases in the synthetic RNA segment.

2. The synthetic RNA segment of claim 1 having a minimum free energy in kcal/mol in a range of −0.27x to −0.49x, where x is the number of bases in the synthetic RNA segment.

3. The synthetic RNA segment of claim 1 which folds into an unbranched structure having an alternation of base paired regions and unpaired regions, wherein the base-paired regions have no more than 13 base-pairs and the unpaired regions have no more than 21 non-paired bases.

4. The synthetic RNA segment of claim 1 having an APE of less than 0.75, 0.65, 0.5, 0.4, 0.39, 0.36 or 0.32 and greater than 0.01, 0.02 0.03, 0.04, 0.05, 0.06 or 0.07.

5. A vector comprising a plus sense single stranded RNA viral backbone and the synthetic RNA segment of claim 1.

6. The vector of claim 5 wherein the viral backbone is derived from a virus selected from the group consisting of: TRV, CTV, TBSV, CY1, HCV, TCV, YFV, SARS-CoV, EVA71, PVX, GULV, PEMV2, DENV2, WNV, and VEEV.

7. The vector of claim 5 excluding any synthetic RNA segment or vector disclosed in either International Application No. PCT/US2024/027884 or International Application No. PCT/US2023/066542.

8. A method of designing a hairpin-like insert for a plus sense single stranded RNA vector comprising selecting a targeting-side sequence to be used on one side of the hairpin-like insert and selecting a series of only partially complementary bases for the other side of the hairpin-like insert such that the bases of the insert folds into a hairpin-like structure.

9. The method of claim 8 wherein the bases of the insert fold into a linear or Y-shaped hairpin-like insert of 40-300 RNA bases for a plus sense single stranded RNA and comprising the steps of,
selecting a sequence of bases forming a targeting side sequence to be used on a first side of the insert;
selecting a sequence of bases for a second side of the insert having a selected percentage complementarity with the bases of the targeting side sequence;
selecting the bases of an apical loop between the first side of the insert and the second side of the insert;
checking that the insert folds and has an MFE/length of a selected MFE/length value;
altering bases of the second side of the insert to achieve the selected MFE/length value;
checking that the insert has an APE of a selected value; and,
altering bases of the second side of the insert if required to achieve the selected APE value.

10. The method of claim 9 wherein the selected percentage complementarity is: a) a range of 60% to 80% or a value or sub-range within such range or b) in the range of (Y−0.25)/−1.05+/−5 wherein Y is the slope of a line of best fit through a plot of MFE versus length for at least four naturally occurring hairpin-like regions in a wild type genomic RNA of a virus that the vector is derived from, or a value or sub-range within such range.

11. The method of claim 9 wherein the selected MFE/length value is a) a range of −0.3 to −0.66, or −0.3 to −0.55, or (−0.27x plus 10) to (−0.49x minus 10), optionally calculated not counting the bases in the apical loop in the inset length, or a value or sub-range within such range or b) a range defined by a line of best fit through a plot of MFE versus length for at least four naturally occurring hairpin-like regions in a wild type genomic RNA of a virus that the vector is derived from with an offset of 1-2 standard deviations or +/−15 kcal/mol from such line of best fit, or a value or sub-range within such range.

12. The method of claim 9 wherein the selected APE value is a range of about 0.1 to 0.3, optionally 0.05 to 0.34 or 0.07 to 0.32, or a value or sub-range within such range.

13. A linear or Y-shaped hairpin-like insert of 40-300 RNA bases comprising,
a sequence of bases forming a targeting-side sequence;
a sequence of bases forming a second side sequence;
an apical loop between the targeting-side sequence and the second side sequence;
an MFE/length that is a) below a maximum of −0.27 or −0.3, b) above a minimum of −0.66, −0.55 or −0.49 or b) of a value defined by a line of best fit through a plot of MFE versus length for at least four naturally occurring hairpin-like regions in a wild type genomic RNA of a virus that the vector is derived from, with an offset of 1-2 standard deviations, +/−5%, or +/−15 kcal/mol from such line of best fit; and,
an APE in a range of about 0.1 to 0.3, or 0.07 to 0.32.

14. The insert of claim 13 for insertion in a vector derived from a Group IV positive-sense single-stranded (+ssRNA) virus according to the Baltimore classification.

15. The insert of claim 14 for insertion in an RNA sequence that comprises an RdRp.

16. The insert of claim 13 wherein a) material deletions are not detected in electrophoresis of RT-PCR products in 60% or more of plants 3 weeks post infection, b) the insert is more stable in an application than a fully base-paired insert with the same targeting-side sequence, or c) the insert is more stable in an application than an insert with the same targeting-side sequence known in the art.


SEQUENCE LISTING (120 sequences)

SEQ ID NO: 1 - RNA, 2692 nt, unidentified
  source 1..2692: mol_type = viral cRNA; note = Citrus yellow vein-associated virus [CYVaV]
gggtaaatatggatccttcatctttgccccgtgcctgttggcatcatgccagacaggtgtttcgagcatcaactagcttctcaagagaggtggttcgcgctgctcgtagatgggttaccatgcccaccagtcgccatgcatatgacttttcaacgagtctaggcattgtgattgctgagcctgcagctcgtttacgacgccgtctgccctctgtacgaaagtgcgcagagaagttagtagtccacaagcaagtcgacactttggtggacgaatggtgctctggaattcccaaccctgatatcgtagaagttggttgggcactccgtctgagggaccgtttcggtcttcctcccgcttctgagcctacccggctcagtggtgagagatgggtgctcaaacaactcaatggggtagatcctgagtcatggaatgctgatcttggtaggtcagttcatatccaaggagactacgccccagggaggaatgcccatatcgctcaggtcgcggcgaccttgtggttaactaggaccttgcatgacaaggccttggctcgccaccagggttttcgcgatttgcagtgattggggtcgacgggctagaggcaaaagcagtgcctctagcttctggactccgactgcttccggttccgcgacccggacaaagtcgacgactgtctcagaccttgttacttccaacacctcgtgctcaattcgtgaatcacgcgtgctcggctaacaaccttggacgtgtgatgaccacacgtgtgttgcagtacaagggccgagatccgatccttccctcttctgaagcccttcaccgacttaaccttcggatagctgagctatataggtctagaccttctaccgtctatccattaagttatgaagggtttctcaattgctatgaaggccgacagcgtactcgttacgcccaagccgtcgagcagttgatgcggtccactcttgagccgaaagatgcgcgagttgaaacgttcattaagaacgagaaatttgactgggcgttgaaaggggaggaggctgatcctcgagcaatccaaccaaggaagccgaaatatttggctgaggttggacggtggttcaaacctttggagcgaatcatctacaaggatctcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgtaagtctcccactctccagcgctatctcaaatggacactccgcaaccatggcgtcgcctcctgcaaagaattgtcatatgagtatgaggttgttggccggagaatgagtggtgacatggacactgcattgggcaactgcgtcattatgtcgatacttacatggtttatgcttagtgaacttggcattaagcatgaattattcgataatggtgacgattgtttgttcatttgcgagtctcacgacgtccccagccccgaggtaattacaaactggttttcggactttgggtttgtggttaggttggaaggcgtcacgtccgtgtttgagcgtattgagttttgccaaacttccccagtatggactgagaggggttggctgatgtgtaggaatattaagtcattgagtaaagaccttacgaatgttaattcgtgcacgggctccacgattgaatatacccactggttgaaagcagtgggaaagtgcgggtcaatactcaatgctggtgtacctatatttcagtcctttcacaacatgctggaaaggcttggcactaactctcgtattgatcgaggggttttcttcaaatcagggctagttaatctcattcgtgggatggacaggcagcctgacgttgacatcactacttccgctcggctttctttcgaagtggcattcgggataacacccgggatgcaattggctattgaacggtactatgactctgtcatgggctcgctgagtaaaatagaaacaactaagtggccaattgaactaagaaaggaatacgaacacggaagtgagtggtacgaggacttaggcgtcctaggatgaatagggtcattggtttaccgatgatacctgttcagaataggattgctcgagcttcgttggttagggtaactcacataccttcttccataactggaaaaggtcgtgtgagcaacctaaccagttaatgtaggtgtctttccgtatctagtcacgatggtaagcaacccgtttatctgtacggcgctcacccgtgggtaggaaggtgaaggttttgtgtcctttaggtcttggacagtctgcgggcttgggaacgacgccccgctagcaacgtactgctctcctaccggactggtagcttaattgtcatcttggagcgatagcactgtgggcctcacccttcgcgcgttggacgtgttgcgtgccccccacagatttgtgaaactctatggagcagttccgcgagccagaagggaggatggccgcctggcgtaatccaggagctctggggggcttgtactcagagtagcattctgctttagactgttaactttatgaaccacgcgtgtcacgtggggagagttaacagcgccc

SEQ ID NO: 2 - protein, 190 aa, synthetic construct
MDPSSLPRACWHHARQVFRASTSFSREVVRAARRWVTMPTSRHAYDFSTSLGIVIAEPAARLRRRLPSVRKCAEKLVVHKQVDTLVDEWCSGIPNPDIVEVGWALRLRDRFGLPPASEPTRLSGERWVLKQLNGVDPESWNADLGRSVHIQGDYAPGRNAHIAQVAATLWLTRTLHDKALARHQGFRDLQ

SEQ ID NO: 3 - protein, 469 aa, synthetic construct
MTTRVLQYKGRDPILPSSEALHRLNLRIAELYRSRPSTVYPLSYEGFLNCYEGRQRTRYAQAVEQLMRSTLEPKDARVETFIKNEKFDWALKGEEADPRAIQPRKPKYLAEVGRWFKPLERIIYKDLSKRLYGEGAEPCIAKGLNALESGATLRRKWEKFSSPVCVSLDASRFDLHVSVGMLKFTHKLYDYYCKSPTLQRYLKWTLRNHGVASCKELSYEYEVVGRRMSGDMDTALGNCVIMSILTWFMLSELGIKHELFDNGDDCLFICESHDVPSPEVITNWFSDFGFVVRLEGVTSVFERIEFCQTSPVWTERGWLMCRNIKSLSKDLTNVNSCTGSTIEYTHWLKAVGKCGSILNAGVPIFQSFHNMLERLGTNSRIDRGVFFKSGLVNLIRGMDRQPDVDITTSARLSFEVAFGITPGMQLAIERYYDSVMGSLSKIETTKWPIELRKEYEHGSEWYEDLGVLG

SEQ ID NO: 4 - RNA, 166 nt, unidentified
  source 1..166: mol_type = viral cRNA; note = CY1
tcgctcaggtcgcggcgaccttgtggttaactaggaccttgcatgacaaggccttggctcgccaccagggttttcgcgatttgcagtgattggggtcgacgggctagaggcaaaagcagtgcctctagcttctggactccgactgcttccggttccgcgacccgga

SEQ ID NO: 5 - RNA, 25 nt, unidentified
  source 1..25: mol_type = viral cRNA; note = CY1
caaagtcgacgactgtctcagacct

SEQ ID NO: 6 - RNA, 34 nt, unidentified
  source 1..34: mol_type = viral cRNA; note = CY1
aggtcttggacagtctgcgggcttgggaacgacg

SEQ ID NO: 7 - RNA, 2983 nt, unidentified
  source 1..2983: mol_type = viral cRNA; note = Citrus yellow vein-associated virus
gggtaaatatggaatcttcatctttgccccgtgcctgctggcaaaaggccaggcaggtgttgcgagcaccagctaccttctcgagagaggtggtcaaagctgcccgcaaatgggtcaccatgcccagtaaccaccgtgtctacgactactccactagtctgggcattgtgattgctgagcctgtggctcgtctgcgacgcagtctaccctccgtacgaaagtgcgcagagaagatagtagtccaccagggaatcgacaccttggtggacgattggtgtacaggacttcctaaccctgatatagtggaagttggttgggcactccgtctgagggaccgtttcggtcttcctcccgcctctgagcctactaggcttagtggtgagagatgggtgctcaaacaactcaatggggtagatcccgagtcatggaatgctgatctgggtaaaccagttcacgtccagggagactacgccccagggaggaatgcccacatcgcgcaggtcgcggcgaccttgtggctaactaagaccttgtctgacaaggccttggcccgccaccagggttttcgcgatttgcagtgattggggtcgacgggctagaggctaaagcagtgcctctagctgcaggactccgactgcttccggttccgcggcccggacaaagccgacggctgtctcagaccttgttactaccaacccctcgtgcacaatttgtgaatcacgcgaactcggctaataaccttgggcgcgtgatgaccacacgagtgatgcagtacaaaggccgagacccgatccaaccctctcaagaagccctaaccaaacttaaccttcggatagccgagctatataagtcgagaccatccaccgtctatccgttgagttatgaagggtttctcaactgctacgaaggccgacagcgtactcgttacgctcaagccgtcgagcagctgttgaggtccactctagaacctaaagatgcgagagttgaaacgttcattaagaacgagaagtttgattgggcgttgaaaggggaggaggctgatcctcgtgcaatccaaccaaggaagccgaaatatctggctgaggttggacggtggtttaaacctttggagcgaatcatctacaaggatcttagtaagcggttgtatggcgagggtggtgaaccttgtattgccaaaggcttaaatgcactagaatctggggcgactttgaggcgcaaatgggagaagttttcttctcctgtttgcgtctctcttgacgcttccaggttcgacctgcatgtaagtgttggcatgcttaagtttacacacaagctgtacgactactactgcaagtctcccaccctccaacgttatctcaaatggacactccgcaatcacggcactgcctcctgtaaagaattgtcatatggttatgaggtagaaggccgaagaatgagtggtgacatggacaccgcattgggcaactgcgtcatcatgtcgatactaacatggtttatgcttagtgagcttggcattaagcatgaattattcgacaatggtgacgactgcttgttcatttgcgagtcaaaagacgtccccagccccgaggtgatcacgaactggttttcggactttgggtttgtggtcaggttggaaggcgtcacgtccgtgtttgagcgtatagagttttgccaaacttccccagtatggactgagaggggttggctgatgtgtaggaacattaagtcgttgagtaaggaccttacgaatgtcaattcgtgcacgggctccacaattgaatatactcactggttaaaagccgtggggaagtgcgggtcaatactcaatgctggtgtgcctatatttcagtcctttcacaacatgctggaaaggcttggtactaactctcgtatagatcgcggggtgttctttaaatctgggctagtcaatctcattcgtgggatggacagacaacctgacgttaagatcaccacttccgctcgtctttctttcgaagtggcattcgggattacacctggaatgcaattggctattgaacggtactatgactcagtcatgggcccgctgggtaaaatagaaacaactaaatggccaatagaactaagaaaggaatacgagtacggaagcgagtggtacgaagacttaggcgtcctaggatgagcagggacatcggtttaccgatggcccccgttcagaataggatggctagggcttcgttggttagggaaacctatataccttcttccaccacaactggtaaggaggctgtgtgggcaacctggccaattaatgtaggtgtctttccgcaactagccacgatagtaagcaacccgttatactggcgtgtgaccagtgtgcaagtggcaatggagccagcaacgtcaacctctacccaactctgtggtgtaggtctcagtacatctggggcttacaactcaggggaagggttcgggaacacgtttaagctcttgcgctcttgtaactatacgagacgctcacccgtgggagggaacgtgatggtcaagtggcccatcagcatgccctacattcttaatgatgatgcgcacaagaccacaggtctaacagcttgtgtggtcattgctgtcaccaatccaggtgccatcactggccaatcttgggcggagattcaattgaatgtagagtacgtagtgggtacttagacagtctgcgggcttgggaacgacgccccgctagcaacgtactgctctcctaccggactggtagccgagctgttatcttggagcgatagcactgtgagcctcaccctgcgcgcgatggacgtgttgcgtgaccctcacagatttgtgaaactctatagagtagttccgcgagccagaagggaggatggcgacctggcgaaatccaggagctctggggggcttgtactcagagtagcaatactgctttagactgttagtttgatgaaccacgcgtgtcacgtggggagaactaacagcgccc

SEQ ID NO: 8 - RNA, 167 nt, synthetic construct
  source 1..167: mol_type = other RNA; note = GFPmmck59 Inserted at CYVaV Position 2304
aaaaggtcgtgtgagcaacctaaccagttaatgtaggtgtctttccgtatgaagcggcacgacttcttcaagagcgataactcgccttgacagaagtccaacgcttcatctagtcaggatggtaagcaacccgtttatctgtacggcgctcacccgtgggtaggaag

SEQ ID NO: 9 - RNA, 61 nt, synthetic construct
  source 1..61: mol_type = other RNA; note = Mmck6.1
ggaagtgatggacgaaattaatgattccataactggaacattacatttcgtcaacacttcc

SEQ ID NO: 10 - RNA, 61 nt, synthetic construct
  source 1..61: mol_type = other RNA; note = CS7-V2.2
ggtatcaatgggcagacgaagatttggcataactgccaatcttccgtctgcaaatgatacc

SEQ ID NO: 11 - RNA, 155 nt, synthetic construct
  source 1..155: mol_type = other RNA; note = F3-C3#3
actagaggaagtgttgacgaaatgtaatgttccagttatggaatcattaatttcgtccatcacttcctacggaaagacacctacattaactggtatcatttgcagacggaagattggcagttatgccaaatcttcgtctgcccattgataccaac

SEQ ID NO: 12 - RNA, 83 nt, synthetic construct
  source 1..83: mol_type = other RNA; note = Polynucleotide Insert into CYVaV
ttggttagggtaactcacataccttcttccatatgaagcggcacgactatctagtcannntggtaagcannncgtttatctgt

SEQ ID NO: 13 - RNA, 240 nt, synthetic construct
  source 1..240: mol_type = other RNA; note = Polynucleotide Sequence of CBC460, CBC461 and CBC462
taaccagttaatgtaggtgtctttccgtatctagtcacgatggtaagcaacccgtgcgtgttacgtcagtgacaatcgccatggaaccggcaatgtctacctctacccaacactgtggggtaggactcgctactagcggggcctactcttcaggtgagggttttggtaacttgttcaagtacaagcgcgcttgtaactttatctgtacggcgctcacccgtgggtaggaaggtgaaggtt

SEQ ID NO: 14 - RNA, 144 nt, synthetic construct
  source 1..144: mol_type = other RNA; note = Polynucleotide Sequence of OULV-insert
tgcgtgttacgtcagtgacaatcgccatggaaccggcaatgtctacctctacccaacactgtggggtaggactcgctactagcggggcctactcttcaggtgagggttttggtaacttgttcaagtacaagcgcgcttgtaact

SEQ ID NO: 15 - RNA, 240 nt, synthetic construct
  source 1..240: mol_type = other RNA; note = Polynucleotide Sequence of CY2220 (1132-1329)2280-1 and CY2220 (1132-1329)2280-2
attgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgcagttaatgtaggtgtctttccgt

SEQ ID NO: 16 - RNA, 198 nt, synthetic construct
  source 1..198: mol_type = other RNA; note = Wt:29/44
cagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactg

SEQ ID NO: 17 - RNA, 300 nt, synthetic construct
  source 1..300: mol_type = other RNA; note = Polynucleotide Sequence CBC135
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 18 - RNA, 300 nt, synthetic construct
  source 1..300: mol_type = other RNA; note = Polynucleotide Sequence CBC139
ggtcattggtttaccgatgatacctgttcagaataggattgctcgagcttcgttgcagtaaaaggttgtatggtgatggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 19 - RNA, 300 nt, synthetic construct; identical to SEQ ID NO: 18
  source 1..300: mol_type = other RNA; note = Polynucleotide Sequence CBC140

SEQ ID NO: 20 - RNA, 300 nt, synthetic construct
  source 1..300: mol_type = other RNA; note = Polynucleotide Sequence CDE967
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgttcctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 21 - RNA, 300 nt, synthetic construct
  source 1..300: mol_type = other RNA; note = Polynucleotide Sequence CDE974
ggtcattggtttaccgatgatacctgttcagaataggattgctcgagcttcgttgcagtaaaaggttgtatggtgagggtgctgagccgtgtatcgccaaaggcctaaatgcattacaatctggagcgactttgaggcgcaaatgggagaagttttcttctccagtttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 22 - RNA, 300 nt, synthetic construct
  source 1..300: mol_type = other RNA; note = Polynucleotide Sequence CDE977
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagcttgcgtttccctcgacgcttccaggttcgacctgcatgtaagcgttggcatgctaaagctcacacacaagctatatgactattactgcagttaatgtaggtgtctttccgtatctagtcacgatggtaagcaac

SEQ ID NO: 23 - RNA, 300 nt, synthetic construct
  source 1..300: mol_type = other RNA; note = Polynucleotide Sequence CDE978
ggtcattggtttaccgatgatacctgttcagaataggattgctcgagcttcgttgcagtaaaaggttgtatggtgagggtgctgagccgcgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 24 - RNA, 300 nt, synthetic construct
  source 1..300: mol_type = other RNA; note = Polynucleotide Sequence CDE980
ggtcattggtttaccgatgatacctgttcagaataggattgctcgagcttcgttgcagtaaaaggttgtatggtgagggtgctgagccgtgtatcgccaaaggtctaaatgcattagaatctggagcgactttgaggcgcaaatgggagaagttttcttctccagcttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 25 - RNA, 300 nt, synthetic construct
  source 1..300: mol_type = other RNA; note = Polynucleotide Sequence CDF015
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcatacacaagctatatgactattactgcagttaatgtaggtgtctttccgtatctagtcacgatggtaagcaac

SEQ ID NO: 26 - RNA, 300 nt, synthetic construct
  source 1..300: mol_type = other RNA; note = Polynucleotide Sequence CDF016
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgtttctctcaacgcttccaggttcgacctgcatgtaagcgttggcatgctaaagttcacacacaagctatatgactattactgcagttaatgtaggtgtctttccgtatctagtcacgatggtaagcaac

SEQ ID NO: 27 - RNA, 300 nt, synthetic construct
  source 1..300: mol_type = other RNA; note = Polynucleotide Sequence CDF017
ggtcattggtttaccgatgatacctgttcagaataggattgctcgagcttcgttgcagtaaaaggttgtatggtgagggtgctgagccgtgtatcgccaaaggcctaaatgtattagaatctggagcgactttgaggcgcaaatgggagaagttttcttctccagtttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 28 - RNA, 300 nt, synthetic construct
  source 1..300: mol_type = other RNA; note = Polynucleotide Sequence CDF020
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcacgtaagcgttggcatgctaaagttcacacacaagctatatgactattactgcagttaatgtaggtgtctttccgtatctagtcacgatggtaagcaac

SEQ ID NO: 29 - RNA, 300 nt, synthetic construct; identical to SEQ ID NO: 20
  source 1..300: mol_type = other RNA; note = Polynucleotide Sequence CDF037

SEQ ID NO: 30 - RNA, 300 nt, synthetic construct
  source 1..300: mol_type = other RNA; note = Polynucleotide Sequence CDF041
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatacgactattactgcagttaatgtaggtgtctttccgtatctagtcacgatggtaagcaac

SEQ ID NO: 31 - RNA, 300 nt, synthetic construct
  source 1..300: mol_type = other RNA; note = Polynucleotide Sequence CDF047
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagttcgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 32 - RNA, 300 nt, synthetic construct
  source 1..300: mol_type = other RNA; note = Polynucleotide Sequence CDF049
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagtttccttctccagtttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 33 - RNA, 61 nt, synthetic construct
  source 1..61: mol_type = other RNA; note = Mmck6.2
ggtttcagtatatttcgtcatcaccttcctaacggaagtgatggacgaaattaatgaaacc

SEQ ID NO: 34 - RNA, 58 nt, synthetic construct
  source 1..58: mol_type = other RNA; note = Insertion of Mmck6.2 into CYVaV1
gtttcagtatttcgtcatcaccttcctaacggaagtgatggacgaaattaatgaaacc

SEQ ID NO: 35 - RNA, 60 nt, synthetic construct
  source 1..60: mol_type = other RNA; note = WT Insert Structure
ggttagggtaactcacataccttcttccataactggaaaaggtcgtgtgacaacctaacc

SEQ ID NO: 36 - RNA, 61 nt, synthetic construct
  source 1..61: mol_type = other RNA; note = Mmck15 Insert
gtcgcaatcaaagacgaagaaatcgtccataactggacatttccttcgtctcaattgcgac

SEQ ID NO: 37 - RNA, 61 nt, synthetic construct
  source 1..61: mol_type = other RNA
ggttgcttggaacccatacgaatgttgcttaacagcaaattcgctatgggtgatagcaacc

SEQ ID NO: 38 - RNA, 61 nt, synthetic construct
  source 1..61: mol_type = other RNA; note = MmckpsvD Insert
gggtgaagtcatgaaagaagctgccttcttaacagaagcagctctctttcacaattcaccc

SEQ ID NO: 39 - RNA, 61 nt, synthetic construct
  source 1..61: mol_type = other RNA; note = MmckpsvE Insert
ccgtaggtaagacaccattgacaagttcttaacagaactgtcacatggtgtgaccctacgg

SEQ ID NO: 40 - RNA, 61 nt, synthetic construct
  source 1..61: mol_type = other RNA; note = PRSVmmck1 Insert
atggtctgaatgataatgaaatgcaagtgtaaccacttcatttccattatccaaagaccat

SEQ ID NO: 41 - RNA, 61 nt, synthetic construct
  source 1..61: mol_type = other RNA; note = PRSVmmck2 Insert
gcagaagcatatattgcaaagagaaatgctaacgcattctcttctgcaatacaacttctgc

SEQ ID NO: 42 - RNA, 199 nt, unidentified
  source 1..199: mol_type = viral cRNA; note = Citrus yellow vein-associated virus [CYVaV]; note = Wild Type Structure 1 (1132-1329) of CYVaV
cagtaaaaggttgtatggtgagggtgcttgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactg

SEQ ID NO: 43 - RNA, 198 nt, synthetic construct
  source 1..198: mol_type = other RNA; note = PDS-198mmck-1 Insert
cacgaaacagaagtacttggcttcaatggaaggtgctgtcttatcaggaaagctttgtgcacaagctattgtacaggattacgagttacttcttggcttttgccaagatgtaactcgtaaacaaattacaaatagcttgtcaaacaaaactgtcggataaacagtaaacttccttgcctacaagtaagtctattcgtg

SEQ ID NO: 44 - RNA, 198 nt, synthetic construct
  source 1..198: mol_type = other RNA; note = PDS-198mmck-2 Insert
gttgctcagtgtgtacgctgacatgtctgttacatgtaaggaatattacaaccccaatcagtctatgttggaattggtatttgcacccgcagaagagttttctcttctacgggtgcaaataagtgtttcacaacatagaaaatattggagtcgtctattcttacccaataacaacaacaaagcgtacacacaagcaac

SEQ ID NO: 45 - RNA, 198 nt, synthetic construct
  source 1..198: mol_type = other RNA; note = LcrGyrA-Mmck5 Insert
acctggtactgttcggcgaaataaattatctgattttgtgcatgtgaaccgtaatggtaagattgcaatgaaattggaggaaaatgatgagattgttttttaacaatcgcatcattttccccattcgctcatgtgcaatgaccaccatcgttgtaacatgacaactcacagataataccatcgccgaacaccccaggt

SEQ ID NO: 46 - RNA, 198 nt, synthetic construct
  source 1..198: mol_type = other RNA; note = LcrGyrA-Mmck3 Insert
atgatgagattgtttcagtagaaacttgtactgaggaccatgatgttttgctaacaacagaatttggtcagtgtattcgattcccagtttctaatgtttttacattagcaactgtgaatcccacacacctgacccatttaaacgttgtcaaaaaccattaggtcaaagagtacagtggaaactgaaacaacccatcat

SEQ ID NO: 47 - RNA, 198 nt, synthetic construct
  source 1..198: mol_type = other RNA; note = Clas-GyrAmmck-5 prime Insert
gcatggcaatgtacggcgtaataaactttctgattttattcaaatcaatcgtagtggtaagattgcgatgaaattagattcaagagatgagattcttttttaagaatcccatctcttgaaacattccctcatgcgcaataaccaccacccatattatttgataacccacagaaggttcaaatgctgtacaccccatgc

SEQ ID NO: 48 - RNA, 198 nt, synthetic construct
  source 1..198: mol_type = other RNA; note = Clas-GyrAmmck-3 prime insert
gagatgagattctttccgttgaaacctgtacacaagaaaatgatatattgttgactactaaacttggacaatgtgtccgctttccgatttctgctatttttatagcagcaatcggaaagctaacacacttgtcccaagtggccgtagttccaaactatcatttcgccctgtacggtaaacacggaaagaaaacatctc

SEQ ID NO: 49 - RNA, 198 nt, synthetic construct
  source 1..198: mol_type = other RNA; note = CS7 Insert
agaattatggagcggtcaagctcacacatcgtcacactttttatgtggtgggctcagccaaaactgtatgtaggaagaggcttgcatgagggcatgtttttacatgccatcatgcaagccagacgactatcatacagttgaccgctgatccaactcataaaagtcaaaatgattgtacaattgattgctctcaattct

SEQ ID NO: 50 - RNA, 198 nt, synthetic construct
  source 1..198: mol_type = other RNA; note = Erwinia GyrA Insert
ccgaccgcgcagcgccggtattattgccgtcaatctgcgtgatgacgatgaactgatcggcgtgtcgctgacgaacggtagtgatgaagcgatgctgttttcagcatcacttcatcactaaaaagacgtacagtgacacataagatcacttaatatcatccgtatacatgatgcaaaccaattggtgttgtaggttgg

SEQ ID NO: 51 - RNA, 66 nt, synthetic construct
  source 1..66: mol_type = other RNA; note = Hairpin Targeting GFP (CYVaV-GFPhp2301)
tgaagcggcacgacttcttcaagagcgccagaattctggcgctcttgaagaagtcgtgccgcttca

SEQ ID NO: 52 - RNA, 65 nt, synthetic construct
  source 1..65: mol_type = other RNA; note = M2250GFP30ext
ggttagggtaactcactgaagcggcacgacttcttcaagagcgccattcagtgagcaacctaacc

SEQ ID NO: 53 - RNA, 59 nt, synthetic construct
  source 1..59: mol_type = other RNA; note = GFPmmck59
tgaagcggcacgacttcttcaagagcgataactcgccttgacagaagtccaacgcttca

SEQ ID NO: 54 - RNA, 63 nt, synthetic construct
  source 1..63: mol_type = other RNA; note = GFPmmck63
tgaagcggcacgacttcttcaagagcgccataatcggcgccttgacagaagtccaacgcttca

SEQ ID NO: 55 - RNA, 128 nt, Citrus tristeza virus
  source 1..128: mol_type = viral cRNA; note = CTV-insert-natural-V2.2
gggggtttatgtttggcaaagaaagtgttggaactgttagtcaagcgggtggttgaatcgttttctcgtttgaagcggaaaaccgctcgtttaacgtccttcgctaatttgttgcttgcgaggctctc

SEQ ID NO: 56 - RNA, 138 nt, synthetic construct
  source 1..138: mol_type = other RNA; note = CYVaV1 with Insert-Natural-V2.2
tcgttgggggtttatgtttgcaaagaaagtgttggaactgttagtcaagcgggtggttgaatcgtttctcgtttgaagcggaaaaccgctcgtttaacgtccttcgctaatttgttgcttgcgaggctctcagttaat

SEQ ID NO: 57 - RNA, 198 nt, synthetic construct
  source 1..198: mol_type = other RNA; note = BBLv2-1
ctggaaaattcgattactcaaagtgcctaaaatcgtaacaacttgtatgatctataggcctggatccaaacaatacgtggctcaaatacttgagaaatttttttctcatgtatttgagcctaaaccttgatttggatccatctcctatcgaacaccaagtgttaaactttttagcagagggagtaatcgaaattccag

SEQ ID NO: 58 - RNA, 37 nt, synthetic construct
  source 1..37: mol_type = other RNA; note = BBLv2-1
ctggaaaattcgattactcaaagtgtcgaaattccag

SEQ ID NO: 59 - RNA, 743 nt, Erwinia amylovora
  source 1..743: mol_type = transcribed RNA; note = Polynucleotide Encoding Erwinia amylovora MurA
gaactggtgaaaaccatgcgcgcctcgatttgggcattgggcccgctggtggcacgttttggccaggggcaagtatcactgcccggtggttgcgctatcggcgcacggccggttgatcttcatatcaccggccttgagcagctcggcgccgagatcaaactggaagaaggttacgttaaagcctctgtcgcgggtcgcctgaaaggggcgcatatcgttatggataaggtcagcgtgggtgcaaccgtcactatcatgagtgcggcgacgctggcaacgggcaccaccgttatcgagaatgctgcgcgtgagccggaaattgtcgacactgccaacttcctcaacacgcttggggcgaaaatcaccggtgccggcagcgatcgtatcaccatcgaaggtgttgatcgccttggtggcggtgtttatcgcgtacttcctgaccgcatcgaaaccggtactttcctggtggcgggagcgatttccggcggtaaggttacctgccgtgcggcgcagcccgatacgctggatgctgtactggctaagctacgcgaagccggtgcggacatcgagatgggagaagactggataagcctggacatgcacggtaagcggcctaaagcggtcaatttacgcacagcgccgcatcccggtttcccaaccgatatgcaggcgcagttcagtttgttgaacctggtggctgaaggcacgggggtgattactgaaaccatcttcgaaaaccgctt

SEQ ID NO: 60 - RNA, 607 nt, Erwinia amylovora
  source 1..607: mol_type = transcribed RNA; note = Polynucleotide Encoding Erwinia amylovora GyrA
atcgtcaacctgctgccgctggaagccaacgagcgtattactgcaattctgccggtgcgtgaatatgccgaaggctggaatatctttatggctaccgccagcggaacggtgaagaaaaccgcgctgactgacttcagccgaccgcgcagcgccggtattattgccgtcaatctgcgtgatgacgatgaactgatcggcgtgtcgctgacgaacggtagtgatgaagcgatgctgttttctgccgccggtaaagtggtgcgattcgcggagagcgcggtgcgtacgatgggccgtaccgcctccggcgtacgcggcatcaagctggctgaaggcgaccgcgtggtgtcgctgatcgtgccgcgtgatgatggcgctatcatgaccgtgacgcaaaacggctacggtaaacgaacggctaacgtcgaatatcccaccaagtcgcgtgcgactcagggggttatctcgatcaaggtaaccgagcgtaacgggccggttatcggtgcggtgcaggtggtcgatggcgatcagatcatgatgatcaccgatgccggcacgctggtacgtacccgcgtgtccgaggtcagcgtggtagggcgtaacacccagg

SEQ ID NO: 61 - RNA, 2808 nt, Pseudomonas syringae
  source 1..2808: mol_type = transcribed RNA; note = P. syringae tabaci_Gyrase subunit A (Pst_GY)
atgggcgaactggccaaagaaatcctcccggtcaatatcgaagacgagctgaagcagtcctacctcgactacgcgatgagcgtaatcgtcggtcgagcactgcccgatgcgcgcgacggcttgaagcccgtgcaccggcgcgtgttgttcgcaatgagcgagctgggtaacgactggaacaagccgtacaagaaatccgcccgtgtggttggtgacgtgatcggtaagtatcacccgcacggcgatacagccgtgtacgacaccatcgttcgtatggctcagccattctcgctacgctacctgctggtagacggtcagggcaacttcggttcggtcgatggcgacaacgctgcggccatgcgatacaccgaagtgcgcatgaccaagctggcgcacgagctgctggccgacctgcacaaggaaaccgtggactgggtgccgaactacgacggcaccgaaatgatccccgcggtcatgccgacccgtattcccaacctgctggtcaacggttccagcggtatcgccgtgggcatggcgaccaacattccgccgcacaaccttggtgaagtcatcgacggttgcctggcactgatcgacaaccccgagttgacgatcgatgagctgatgcagtacatccccggcccggatttcccgacagcggcgatcatcaacggccgtgcgggtatcatcgaagcctatcgtacgggtcgtggacgcatttacatgcgtgcccgctccatcgtcgaagacatcgacaaggtcggcggacgtcagcagatcgtcatcaccgaactgccgtaccagcttaacaaggcacgtctgatcgagaagatcgccgagctggtcaaggaaaagaaactcgaaggcattaccgagctgcgtgacgagtccgacaaggacggtatgcgcgtggtcatcgagctgcgtcgtggcgaagtgccagaggttgttctcaacaacctttatgcccagacccagctgcaaagcgttttcggtatcaacatcgttgccttgattgacggtcgtccacgcatcctgaacctcaaggacctgttggaagcctttgttcgtcaccgtcgcgaagtggttacccgccgtaccgtattcgagctgcgcaaggcgcgtgagcgtggccatatccttgaaggtcaggctgttgcgttgtccaacatcgacccggtcatcgccctcatcaaggcctctccgacacctgcagaagccaaggaggcgttgatcaagacgccttgggaatcaagcatgcgcgtgattctgctaggagcagtggtcgaaatggtcgagcgtgcaggtgccgattcgtgccgccctgagaacctggacccgcaatacggtctgcgtgaaggcaagtatttcctgtcaccggaacaggctcaggccattctggaactgcgtctgcatcgcctgaccggtctggaacacgagaagctgctgggcgagtaccaggaaatcctcaaccagatcggcgagctgatccgcatcctcaacagcgcaacgcgcctgatggaagtgatccgcgaagagctggaagtgatccgctccgagtacggcgatgcccgtcgtaccgagattctcgatgcacgtctggacctgaccctgggtgacctgatcaccgaagaagagcgtgtggtcaccatctcccatggcggctatgccaagacccagccattggcggtctatcaggctcagcgtcgtggcggcaagggcaagtcggccaccggcatcaaggatgaggattacattgctcacctgctggtcgccaacagccacacgacactgctgatgttctccagcaagggcaaggtgtactggctcaagacctatgagatcccggaagcgtcccgcgctgcccgtggtcgtccgttggtcaacctgttgccgctgagcgacggcgaatacatcaccaccatgctgccggttgacctcgaagccatgcgcaagcgtgccgacgaagaaggcgaagccctcgaaggcgagctggacgacgcggaaaacagcagcgagaccgaagaagagcgcaaggcccgtatcaaggccgccgacaagaagaaggctccgttcatcttcatgtccaccgccaacggtaccgtcaagaagaccccgctggttgcattcagccgtcaacgcagttcgggcctcattgcccttgagctggacgagggcgacatcctgatctccgctgccattaccgatggcgaacaggaaatcatgctgttctccgatggcggcaaagtgacccgcttcaaggaatccgatgtgcgcgccatggggcgtaccgctcgcggcgtgcgtggcatgcgtctgccagaagggcaaaagctcatttcgatgctgatcccggaagaaggcagccagatcctcaccgcttccgagcgcggttacggcaagcgtacggccatttccgagttccccgagtacaagcgcggcggtcagggtgtcatcgccatggtcagcaacgagcgtaacggccgtctggttggcgcagttcaggtgcttgatggcgaagaaatcatgctgatttccgatcagggcacgctggtgcgtacccgggtgggcgaagtgtccagtctgggccgtaacactcagggtgtgaccctgatcaagctggccagcgacgagaaactggtcggtctggagcgtgttcaggagccgtcggaagtcgaaggcgaagagcttgaaggcgaagaagttatcgacggcgtgattgtcgatgccgctgaagctgaagtgggcgacgccggtgaagacctgcaagcggacgctgcgccagacgaagacgaaccgcagaactga

SEQ ID NO: 62 - RNA, 663 nt, Pseudomonas syringae
  source 1..663: mol_type = transcribed RNA; note = P. syringae tabaci_Adenylate Kinase (Pst-ADK)
atgcgcgtgattctgctaggagctcccggggccggtaaaggtactcaggcaaaattcatcactgaaaatttcggcatcccgcaggtttcgacaggcgacatgctgcgcgctgcagtcaaggctgaaaccgagcttggcctgaaggccaagagcgtcatggactcgggtggtctggtttccgatgacctgatcattggtctgatcaaggatcgtctggcccagccggattgtgcgaacggcgttctgttcgacggcttcccgcgcaccattcctcaggccgaagccctgttgaaagcaggtctggaaatcgaccacgtgctggaaatcgccgttgatgacgaagaaatcgtcaagcgcatgtcgggccgccgggttcacgaaggctctggtcgcatctatcacaccattttcaacccgccgaaagtcgagggtatcgatgatgtgactggtgaaccgctgttgcagcgcaaggacgacgtcgaagaaaccgtgcgtcatcgcctgtcggtctaccatgcccagaccaagccgctggtcgagttctacagcaagctggaagcaaagaacggcaagcccaagtgcagccatattccaggtgttggctcggttgaagaaatcactgcaaaagtactgaaagcactggagcctgaagcacagaagtaa

SEQ ID NO: 63 - RNA, 717 nt, unidentified
  source 1..717: mol_type = transcribed RNA; note = GFPuv
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatcagttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataa

SEQ ID NO: 64 - RNA, 80 nt, unidentified
  source 1..80: mol_type = viral cRNA; note = Citrus yellow vein-associated virus [CYVaV]; note = CY1132-1329 (structure 1)
cagtaaaaggttgtatggtgagggtgctgagccgtgtatcgccaaaggcctaaatgcattagaatctggagcgactttga

SEQ ID NO: 65 - RNA, 33 nt, synthetic construct
  source 1..33: mol_type = other RNA; note = CY1493-1525 (structure 2)
tttatgcttagtgaacttggcattaagcatgaa

SEQ ID NO: 66 - RNA, 80 nt, synthetic construct
  source 1..80: mol_type = other RNA; note = CY2136-2218 (strucuture 3)
acgaggacttaggcgtcctaggatgaatagggtcattggtttaccgatgatacctgttcagaataggattgctcgagctt

SEQ ID NO: 67 - RNA, 61 nt, synthetic construct
  source 1..61: mol_type = other RNA; note = CY2220-2280 (structure 4)
ggttagggtaactcacataccttcttccataactggaaaaggtcgtgtgagcaacctaacc

SEQ ID NO: 68 - RNA, 51 nt, synthetic construct
  source 1..51: mol_type = other RNA
tgaagcggcacgacttcttcaatcaagagatgaagaagtcgtgccgcttca

SEQ ID NO: 69 - RNA, 61 nt, synthetic construct
  source 1..61: mol_type = other RNA
tgaagcggcacgacttcttcaagagcatcaagagagctcttgaagaagtcgtgccgcttca

SEQ ID NO: 70 - RNA, 32 nt, synthetic construct
  source 1..32: mol_type = other RNA
tgaagcggcacgacttcttcaagagcgccaga

SEQ ID NO: 71 - RNA, 32 nt, synthetic construct
  source 1..32: mol_type = other RNA
tctggcgctcttgaagaagtcgtgccgcttca

SEQ ID NO: 72 - RNA, 61 nt, synthetic construct; identical to SEQ ID NO: 67
  source 1..61: mol_type = other RNA

SEQ ID NO: 73 - RNA, 59 nt, synthetic construct; identical to SEQ ID NO: 53
  source 1..59: mol_type = other RNA

SEQ ID NO: 74 - protein, 21 aa, synthetic construct
GSGEGRGSLLTCGDVEENPGP

SEQ ID NO: 75 - RNA, 247 nt, synthetic construct
  source 1..247: mol_type = other RNA
acgaggacttaggcgtcctaggaggaagcggagagggcagaggaagtctgctaacatgcggtgacgtcgaggagaatcctggaccttttaggctaaagtttcatcaccaccaccatcaccactgaccggtgatagtgttggtgatgaaactttaggataaacgatccgatgttctaaccgaagtcaccgaaggccagcggaccccctctgacctacctgattcctgataggattgctcgagcttcgt

SEQ ID NO: 76 - protein, 33 aa, synthetic construct
GSGEGRGSLLTCGDVEENPGPFRLKFHHHHHHH

SEQ ID NO: 77 - RNA, 198 nt, synthetic construct
  source 1..198: mol_type = other RNA
acctggggatgacctcttttggtttaatcgtcctcgtctcattctttatctcattaattttgtcctctttcagaatgcttttcaattggccttctttttttaaagaagcccaattgaaaacttaaactgtaaagaggactcttattaaggaaattagaatagaccaccgacgataagagtaaagaggtcatgccaggt

SEQ ID NO: 78 - RNA, 198 nt, synthetic construct
  source 1..198: mol_type = other RNA
atgacctcttttggtttaatcgtcctcgtctccttctttatctcattaattttgtcctctttcagaatgcttttcaattggccttctttgcttggacttttgtccaagaaaagaaggccaccacacaaatgcattctgacacaaggaccaacttctgagaaaagctcagagacaggcaacttaaaccaaaacggtcat

SEQ ID NO: 79 - RNA, 198 nt, synthetic construct
  source 1..198: mol_type = other RNA
gttgattacttaaccctaaggcatggatttatcatcgcgcatttggcacctcagagccacgtaaagtttgatttccaaaagtatatcaacaggtcacttttgtgacctattgatatacttcagactaatacaaactttaaaccggctcagacgtacaaatcgcgccaaataaaccaacaattagggttaagcatcaac

SEQ ID NO: 80 - RNA, 202 nt, synthetic construct
  source 1..202: mol_type = other RNA
ggcctcaatttaccagtagaagagaaactaaaattatattcagatgatccttcaaagaccatgagattatcaacaagttttaatgttaggaaggagacatttttgtctccgtcctaacattatgttaatgtatgataatctggcgggtctgtgtagttcatcgaatccccattttgttaaaattctactggtacttgaggcc

SEQ ID NO: 81 - RNA, 198 nt, synthetic construct
  source 1..198: mol_type = other RNA
ctctagagaagtatgctcctgaatggccttctaatccatcatctttcagggaaatcgtgagcagatattgcagggaaattcgtcaactcggatttagttttctaaatcggagttgacgaaccaaaactgccaatatctgaaaaacgatatcacttaagatatggtaagagaagccaaaatggagcatatttactagag

SEQ ID NO: 82 - RNA, 198 nt, synthetic construct
  source 1..198: mol_type = other RNA
gccatagcagaaagcctggggttagataaagagtgtataaaagatgtattgggtgaacaaggacaacatatggctatcaattattatcctccttgtcttttgacaaggtggataataattctccctccattatgttgtcaaaagttcagccgatgatcattataattatcttttctccaaccaggctttctatatggc

SEQ ID NO: 83 - RNA, 199 nt, synthetic construct
  source 1..199: mol_type = other RNA
gacccaaatgcccttaccattttattgcaagatttagaagtggcaggtcttcaagttctcaaagacgacaaatgggttgctgttaatccccttcctatttttaggaagcggattaacagctttgaaattatgttgtcttccttgaattcgatgaatgtcattctttcttcttgaaatctcctggtaagggtaatgggtc

SEQ ID NO: 84 - RNA, 199 nt, synthetic construct
  source 1..199: mol_type = other RNA
cctcaacatggttgaagggagtaaaagatttttcgagctccctttcgaggagagatccaagtacatgtcgactgatatgtatgccccagttaggtatttttatatctatctggggtatacccacaaagtacgatatgtaaaacggatcactactaaaaggagctaattaaaatatttgggacccttcaaccacttgagg

SEQ ID NO: 85 - RNA, 197 nt, synthetic construct
  source 1..197: mol_type = other RNA
cttccacatgaggagagagctaagtacatgtcctctgatatgaaagctaaagtgcgatatggaacaagctttaaccaaactaaagacaaagtcttttttttaaaagacattgtctttagtaacccataaagcttgttcttaaatcgctctataatttcaatcacctcgacattacaatcctctctcctcaatggaag

SEQ ID NO: 86 - RNA, 224 nt, synthetic construct
  source 1..224: mol_type = other RNA
acaatcacagattgttatcataaagcgaattctttggttatctagctgtatgaatcacatcatccagcccgataaccaaagattgagtgtgtttactctaagtacaatttcaacagttatttcaatcagacaattgtatcataatgggtgctcaggtttcatcacagaaagtgggcgcacatgaaaactcaaatagagcgtatggtggttctaccattaattac

SEQ ID NO: 87 - RNA, 152 nt, synthetic construct
  source 1..152: mol_type = other RNA
attggccatccggtgacctctcccaacccttgtaccagtgtatatcactagcacaagcattgggagcgtttataccacttagcttgaaagaggttaaaacattacaattcattgttaagttgaatacagcaaaatgggagctcaagtatcaa

SEQ ID NO: 88 - RNA, 66 nt, synthetic construct; identical to SEQ ID NO: 51
  source 1..66: mol_type = other RNA; note = Hairpin Targeting GFP (CYVaV-GFPhp2301)

SEQ ID NO: 89 - RNA, 21 nt, synthetic construct
  source 1..21: mol_type = other RNA
tccgtggacgtcatgtgtaag

SEQ ID NO: 90 - RNA, 24 nt, synthetic construct
  source 1..24: mol_type = other RNA
ggaagtgatggacgaaattaatga

SEQ ID NO: 91 - RNA, 61 nt, synthetic construct; identical to SEQ ID NO: 33
  source 1..61: mol_type = other RNA; note = Mmck6.2

SEQ ID NO: 92 - RNA, 75 nt, synthetic construct
  source 1..75: mol_type = other RNA; note = Mmck6.2
ttcgttggtttcagtatatttcgtcatcaccttcctaacggaagtgatggacgaaattaatgaaaccagttaatg

SEQ ID NO: 93 - RNA, 80 nt, synthetic construct
  source 1..80: mol_type = other RNA; note = Mmck6.2
agcttcgttggtttcagtatatttcgtcatcaccttcctaacggaagtgatggacgaaattaatgaaaccagttaatgta

SEQ ID NO: 94 - RNA, 83 nt, synthetic construct
  source 1..83: mol_type = other RNA
acgaggacttaggcgtcctaggatgaatagggtcattggtttaccgatgatacctgttcagaataggattgctcgagcttcgt

SEQ ID NO: 95 - RNA, 174 nt, synthetic construct
  source 1..174: mol_type = other RNA
acgaggacttaggcgtcctaggaggaagcggagagggcagaggaagtctgctaacatgcggtgacgtcgaggagaatcctgaccttgacgatccgatgttctaaccgaagtcaccgaaggccagcggaccccctctgacctacctgattcctgataggattgctcgagcttcgt

SEQ ID NO: 96 - RNA, 160 nt, synthetic construct
  source 1..160: mol_type = other RNA
ctaagcagattattcaactaggatctggaataaccgaaggtttgggagcaggatctcatccagaagttggtcgtgctgctaccagcgcgaccaacgtctcgatgcgatcatgctcacaaaccttcaattattccagatcctagccgaataatatgcttag

SEQ ID NO: 97 - RNA, 159 nt, synthetic construct
  source 1..159: mol_type = other RNA
ctaagcagattattcaactaggatctggaataaccgaaggtttgggagcaggatctcatccagaagttggtcgtgctgctacaagcgtgatcaacgtctacgatgcgatcatgctacgagtcttcattataacagatcttagcgaataataatgcttag

SEQ ID NO: 98 - RNA, 160 nt, synthetic construct
  source 1..160: mol_type = other RNA
ctaggcattattatttgactaagatctgttatagttgaaggcaggccgtatgatttcatttacgggttgattgtgcttgtagcagcacgaccaacttctggatgagatcctgctcccaaaccttcggttattccagatcctagttgaataatctgcttag

SEQ ID NO: 99 - RNA, 159 nt, synthetic construct
  source 1..159: mol_type = other RNA
ctaagcagattattcaactaggatctggaataaccgaaggtttgggagcaggatctcatccagaagttggtcgtgctgctacaagcgtgatcaacttctacgatgcgatcatgctacgagtcttcattataacagatcttagcgaataataatgcttag

SEQ ID NO: 100 - RNA, 158 nt, synthetic construct
  source 1..158: mol_type = other RNA
ctaagcagattattcaactaggatctggaataaccgaaggtttgggagcaggatctcatccagaagttggtcgtgctgctacaagcgtgatcaacttctggatgcgatcatgctacgagtcttcattataacagatcttagcgaataataatgcttag

SEQ ID NO: 101 - RNA, 158 nt, synthetic construct
  source 1..158: mol_type = other RNA
ctaagcagattattcaactaggatctggaataaccgaaggtttgggagcaggatctcatccagaagttggtcgtgctgctacaagcgtgatcaacttctggatgagatcatgctacgagtcttcattataacagatcttagcgaataataatgcttag

SEQ ID NO: 102 - RNA, 158 nt, synthetic construct
  source 1..158: mol_type = other RNA
ctaagcagattattcaactaggatctggaataaccgaaggtttgggagcaggatctcatccagaagttggtcgtgctgctacaagcgtgatcaacttctggatgagatcctgctacgagtcttcattataacagatcttagcgaataataatgcttag

SEQ ID NO: 103 - RNA, 159 nt, synthetic construct
  source 1..159: mol_type = other RNA
ctaagcagattattcaactaggatctggaataaccgaaggtttgggagcaggatctcatccagaagttggtcgtgctgctacaagcgtgatcaacttctggatgagatcctgctcccgagtcttcattataacagatcttagcgaataataatgcttag

SEQ ID NO: 104 - RNA, 158 nt, synthetic construct
  source 1..158: mol_type = other RNA
ctaagcagattattcaactaggatctggaataaccgaaggtttgggagcaggatctcatctaggatttggtcgtgctgctacaagcgtgatcaggttctgggtgagatcatgctacgagtcttcattataacagatcttagcgaataataatgcttag

SEQ ID NO: 105 - RNA, 158 nt, synthetic construct
  source 1..158: mol_type = other RNA
ctaagcagattattcaactaggatctggaataaccgaaggtttgggagcaggatctcatctaggagttggtcgtgctgctacaagcgtgatcagcttctgggtgagatcatgctacgagtcttcattataacagatcttagcgaataataatgcttag

SEQ ID NO: 106 - RNA, 158 nt, synthetic construct
  source 1..158: mol_type = other RNA
ctaagcagattattcaactaggatctgggataaccgaaggtttgggagcaggatctcatccagaagttggtcgtgctgctacaagcgtgatcaacttctggatgagatcatgctacgagtcttcattataacagatcttagcgaataataatgcttag

SEQ ID NO: 107 - RNA, 418 nt, synthetic construct
  source 1..418: mol_type = other RNA
ggttagggtaactcacataccttcttccatacagtaaaaggttgtatggtgagggtgctgagccgtgtatcgccaaaggcctaaatgcattagaatctggagcgactttgaggcgcaaatgggagaagttctaagcagattattcaactaggatctggaataaccgaaggtttgggagcaggatctcatccagaagttggtcgtgctgctacaagcgtgatcaacgtctacgatgcgatcatgctacgagtcttcattataacagatcttagcgaataataatgcttagttcttctccagtttgcgtttctctcgacgcttccaggttcgacctgcatgtaagcgttggcatgctaaagttcacacacaagctatatgactattactgactggaaaaggtcgtgtgagcaacctaacc

SEQ ID NO: 108 - RNA, 86 nt, synthetic construct
  source 1..86: mol_type = other RNA
ggcctcgacacgggaaggtagctgcatggaaagatgatgatggagattggtacgagactgggttgcacatattccgtgccgaggcc

SEQ ID NO: 109 - RNA, 40 nt, synthetic construct
  source 1..40: mol_type = other RNA
ggcctcgacacgggaaggtagcatattccgtgccgaggcc

SEQ ID NO: 110 - RNA, 65 nt, synthetic construct; identical to SEQ ID NO: 52
  source 1..65: mol_type = other RNA

SEQ ID NO: 111 - RNA, 57 nt, synthetic construct
  source 1..57: mol_type = other RNA
ggttagggtaactcactgaagcggcacgacttgcgccattcagtgagcaacctaacc

SEQ ID NO: 112 - RNA, 60 nt, synthetic construct
  source 1..60: mol_type = other RNA
tttatgcttagtgtttatgcttagtgaacattggcattaagcatgaacattaagcatgaa

SEQ ID NO: 113 - RNA, 51 nt, synthetic construct
  source 1..51: mol_type = other RNA
aaggattcgtactccaagtgcttcaagagagcacttggagtacgaatcctt

SEQ ID NO: 114 - RNA, 52 nt, synthetic construct
  source 1..52: mol_type = other RNA
tgaagcggcacgacttcttcaatcaagagattgaagaagtcgtgccgcttca

SEQ ID NO: 115 - RNA, 59 nt, synthetic construct
  source 1..59: mol_type = other RNA
tgaagcggcacgacttcttcaagagttcaagagactcttgaagaagtcgtgccgcttca

SEQ ID NO: 116 - RNA, 76 nt, synthetic construct
  source 1..76: mol_type = other RNA
cttggtcgaagcaaagcaaagtgcgccctcttctcgaattcgagaagagggcgcactttgctttgcttcgaccaag

SEQ ID NO: 117 - RNA, 197 nt, synthetic construct
  source 1..197: mol_type = other RNA
cagtaaaaggttgtatggtgagggtgctgagccgtgtatcgccaaaggcctaaatgcattagaatctggagcgactttgagccgcaaaagcgcctctgaacgcaacttattgttatgttctctcgacgcttccaggttcgacctgcatgtaagcgttggcatgctaaagttcacacacaagctatatgactattact

SEQ ID NO: 118 - RNA, 417 nt, synthetic construct
  source 1..417: mol_type = other RNA
ggttagggtaactcacataccttcttccatacagtaaaaggttgtatggtgagggtgctgagccgtgtatcgccaaaggctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgactaagcagattattcaactaggatctggaataaccgaaggtttgggagcaggatctcatccagaagttggtcgtgctgctacaagcgtgatcaacgtctacgatgcgatcatgctacgagtcttcattataacagatcttagcgaataataatgcttagctggaaaaggtcgtgtgagcaacctaacc

SEQ ID NO: 119 - RNA, 20 nt, synthetic construct
  source 1..20: mol_type = other RNA
ctgtcatgggctcgctgagt

SEQ ID NO: 120 - RNA, 20 nt, synthetic construct
  source 1..20: mol_type = other RNA
ccacagtgctatcgctccaa